SEQUENCE LISTING (10 sequences)

SEQ ID NO: 1 - DNA, 26 bp, Artificial Sequence
  Primer Sequence
gggccttgtgcaacccgggcgacttc

SEQ ID NO: 2 - DNA, 26 bp, Artificial Sequence
  Primer Sequence
ctaattttgggctagcatccaggcac

SEQ ID NO: 3 - DNA, 26 bp, Artificial Sequence
  Primer Sequence
ctgctccaatctagaagcgtaaggta

SEQ ID NO: 4 - DNA, 25 bp, Artificial Sequence
  Primer Sequence
ggtaatggtagcgcccggggctcag

SEQ ID NO: 5 - DNA, 26 bp, Artificial Sequence
  Primer Sequence
ctgctccaaagaaggagctcaaggta

SEQ ID NO: 6 - DNA, 25 bp, Artificial Sequence
  Primer Sequence
ggtaatggtagctagcggcgctcag

SEQ ID NO: 7 - DNA, 27 bp, Artificial Sequence
  Primer Sequence
caaaaatctcccaaagctttcgatatg

SEQ ID NO: 8 - DNA, 27 bp, Artificial Sequence
  Primer Sequence
cggcgtatgctgcagagaaacaattgg

SEQ ID NO: 9 - DNA, 20 bp, Artificial Sequence
  Primer Sequence
gcaacctggcttatcgaaat

SEQ ID NO: 10 - DNA, 20 bp, Artificial Sequence
  Primer Sequence
cgtaatacgactcactatag

(19) United States
(12) Patent Application Publication
(10) Pub. No.: US 2005/0260652 A1
(43) Pub. Date: Nov. 24, 2005

(54) Compositions and methods that modulate RNA interference

(75) Inventors: Gary Ruvkun, Newton, MA (US); Ravi Kamath, Brookline, MA (US); Harrison Gabel, Cambridge, MA (US); John Kim, Somerville, MA (US)

Correspondence Address: CLARK & ELBING LLP, 101 FEDERAL STREET, BOSTON, MA, 02110, US

(73) Assignee: The General Hospital Corporation, Boston, MA (US)

(21) Appl. No.: 11/106,238

(22) Filed: Apr. 14, 2005

Related U.S. Application Data
(60) Provisional application No. 60/657,895, filed on Mar. 2, 2005.
(60) Provisional application No. 60/576,141, filed on Jun. 2, 2004.
(60) Provisional application No. 60/562,499, filed on Apr. 15, 2004.

(51) Int. Cl. C12Q 1/68; A01K 67/033

(52) U.S. Cl. 435/6; 800/8

(57) ABSTRACT

The invention features compositions useful in modulating RNA interference in a wide variety of cell types for the treatment of virtually any disease or disorder related to the overexpression of a gene or genes, as well as methods of identifying and using such compositions.

### CROSS REFERENCE TO RELATED APPLICATIONS

This application claims the benefit of U.S. Provisional Application Nos. 60/562,499, 60/576,141, and 60/657,895, filed Apr. 15, 2004, Jun. 2, 2004, and Mar. 2, 2005, respectively, hereby incorporated by reference.

### BACKGROUND OF THE INVENTION

In general, the invention features methods and small molecules useful in modulating RNAi in a wide variety of cell types.

Exposure of many organisms to double stranded (ds) RNA causes the degradation of mRNA molecules containing sequences homologous to the trigger dsRNA. This process has been termed dsRNA-mediated interference (RNAi) in Caenorhabditis elegans, post-transcriptional gene silencing (PTGS) in plants, and quelling in fungi. RNAi is a natural defense mechanism that is thought to have evolved to protect organisms, including mammals, from viral diseases. Many viral genomes are composed of RNA. When such viruses infect a cell, they make double-stranded copies of their genetic material. Cells of many species combat such infections by targeting these dsRNAs for destruction.

dsRNAs are cleaved to small 20-25 bp interfering (si)RNAs by the RNase III enzyme dicer. These siRNAs hybridize to their cognate mRNAs, as part of a large polypeptide complex, and induce mRNA cleavage and degradation. In particular, RNAi has been used as a tool to investigate gene function in a wide range of species. With an increasing list of genes successfully knocked-down by RNAi in mammalian cells and improvements in the delivery of siRNAs to cells, including in vivo delivery to mice, RNAi is now emerging as a therapeutic tool useful for the treatment of virtually any disease or disorder linked to the overexpression of a gene or genes. RNAi is emerging as a potent therapy for the treatment of hyperproliferative disorders (e.g., neoplasms), infectious diseases, parasites, and some dominant genetic diseases.

While much has been learned concerning the mechanism underlying RNAi, many questions remain. The identification of genes and gene products required for RNAi present attractive therapeutic targets to enhance its efficacy.

### SUMMARY OF THE INVENTION

In general, the invention features methods and small molecules useful for modulating RNAi in a wide variety of cell types.

In one aspect, the invention features a method for identifying a gene encoding a polypeptide that is required for or enhances RNA interference (RNAi). The method includes providing a nematode including a dsRNA, contacting the nematode with an inhibitory nucleobase oligomer (e.g., a dsRNA, siRNA, or dsRNA mimetic) that targets a candidate gene, and detecting a decrease or increase (e.g., by monitoring the expression of a reporter gene) in RNAi in the nematode relative to a control nematode not contacted with the inhibitory nucleobase oligomer, where the decrease or increase indicates that the candidate gene encodes a polypeptide that is required for RNAi or enhances RNAi. For example, the nematode includes a mutation that enhances RNAi (e.g., a mutation in eri-1 or rrf-3).

In another aspect the invention features a method for identifying a candidate compound that modulates RNAi. The method includes providing a cell (e.g., a nematode cell, a cell in a nematode, a mammalian cell, or a plant cell) expressing an REG nucleic acid molecule, contacting the cell with a candidate compound (e.g., a member of a chemical library), and comparing the expression of the REG nucleic acid molecule in the cell contacted with the candidate compound with the expression of the REG nucleic acid molecule in a control cell, where an alteration in expression identifies the candidate compound as modulating RNAi. In one embodiment, the screening method identifies a compound that increases or decreases transcription of the nucleic acid molecule. In another embodiment, the screening method identifies a compound that increases or decreases translation of an mRNA transcribed from the nucleic acid molecule.

In another aspect, the invention features a method for identifying a candidate compound that modulates RNAi. The method includes providing a cell (e.g., a nematode cell, a cell in a nematode, a mammalian cell, or a plant cell) expressing an REG polypeptide (e.g., an endogenous polypeptide); contacting the cell with a candidate compound; and comparing the biological activity (e.g., monitored with an enzymatic or immunological assay) of the REG polypeptide in the cell contacted with the candidate compound to a control cell, where an increase or decrease in the biological activity of the REG polypeptide identifies the candidate compound as modulating RNAi.

In another aspect, the invention features yet another method for identifying a candidate compound that modulates RNAi. The method includes providing an REG polypeptide, contacting the polypeptide with a candidate compound (e.g., a member of a chemical library), and detecting binding of the REG polypeptide to the candidate compound. A compound that binds to the REG polypeptide is a candidate compound that modulates RNAi. The candidate compound may increase or decrease the biological activity of an REG polypeptide.

In another aspect, the invention features a method for inhibiting RNAi in an organism. The method includes contacting the organism (e.g., a plant, an mammal, or a pathogen selected from the group consisting of a bacteria, a virus, a fungus, an insect, and a nematode) with a nucleobase oligomer (e.g., an siRNA or an shRNA) including a duplex of at least eight but no more than thirty consecutive nucleobases of an REG nucleic acid in an amount sufficient to inhibit RNAi.

In another aspect the invention features a method for enhancing RNAi in an organism (e.g., a plant, a mammal, or a pathogen selected from the group consisting of a bacteria, a virus, a fungus, an insect, and a nematode). The method includes contacting the organism with an REG polypeptide in an amount sufficient to enable or enhance RNAi.

In another aspect, the invention features a pharmaceutical composition including at least one REG nucleobase oligomer and an excipient. In one embodiment, the pharmaceutical composition further includes at least one REG polypeptide or fragment thereof. In another embodiment, the pharmaceutical composition contains at least 1, 2, 3, 4, 5, 10, 15, 20, or 24 REG polypeptides or fragment thereof.

In another aspect, the invention features a method of enhancing RNAi in a subject. The method includes co-administering (e.g., simultaneous co-administration) to the subject an RNAi therapeutic and an REG cocktail including at least one REG polypeptide.

In another aspect, the invention features a method of enhancing RNAi in a subject. The method involves co-administering to the subject an RNAi therapeutic that targets a gene of interest and an REG inhibitory nucleic acid, wherein the REG inhibitory nucleic acid down regulates an RNAi therapeutic. If desired, the RNAi therapeutic may be administered systemically, and the REG inhibitory nucleic acid may be administered to a specific cell type, tissue, or organ.

In preferred embodiments of any of the above aspects, the REG polypeptide is selected from the following group: B0414.7b, B0414.7a, C04F12.1, C06A5.1, C06E1.10, C07E3.2, C12D8.1b.3, C12D8.1b.1, C12D8.1a, C12D8.1b.2, C15F1.4, C16A3.4, C26D10.1, C27H6.2, C29E4.2, C31H1.8, C55B7.5, D2089.1b.1, D2089.1b.2, D2089.1a, D2089.1b.3, D2096.8, E02H1.1, F02E9.4.1, F02E9.4.2, F09G2.4, F11A10.3, F22D6.6, F26A3.2, F26E4.4, F32E10.4, F37B12.4, F43G9.1, F43G9.10, F43G9.12, F43G9.5, F46A9.5.1, F46A9.5.2, F48E8.5.2, F48E8.5.3, F48E8.5.1, F49D11.1, F52B5.6, F52G2.2, F54H12.1a, F54H12.1c, F54H12.1b, F56A3.4, F56A8.6, F56D2.6a, F56D2.6b, F59A2.1b.2, F59A2.1b.1, F59A2.1a, F59A3.3, K07A.11, K07F5.13c, K07F5.13b, K07F5.13a, K08D10.4, K12B6.1, K12H4.5.3, K12H4.5.1, K12H4.5.4, K12H4.5.5, K12H4.5.6, K12H4.5.2, M03C11.3, R03D7.4, R06A4.4a, R06A4.4b, R06C1.1, R06F6.1, T01C3.8, T01G9.6a.2, T01G9.6b, T01G9.6a.1, T08G11.4, T09A5.10, T12D8.1.1, T19B10.4b, T19B10.4a, T19B4.5, T22B3.1, T22D1.10, T23B12.1, T23D8.3, T25G3.3, W04A4.5, W04C9.1, W05H7.4c, W05H7.4d, W05H7.4a, W05H7.4b, W06E11.1, W07E6.4, W10C8.2, Y110A7A.19, Y38A10A.6, Y38F2AL.3b, Y38F2AL.3a, Y48G1A.5, Y56A3A.17b, Y56A3A.17a, Y61A9LA.10, Y71F9B.4, Y71G10AL.1a, Y71G10AL.1b, ZC449.3b, ZC449.3a.2, ZC449.3a.1, ZK112.2, ZK1127.3, ZK1127.4, ZK1127.5, ZK1127.6.1, ZK1127.6.2, ZK1127.7, ZK1127.9e.2, ZK1127.9d, ZK1127.9c, ZK1127.9e.3, ZK1127.9b, ZK1127.9a, ZK1127.9e.1, ENSP00000257131, ENSP00000207451, ENSP00000277804, ENSP00000300291, ENSP00000243563, ENSP00000296702, ENSP00000251819, ENSP00000331699, ENSP00000265155, ENSP00000289371, TR:Q7Z4R6, ENSP00000248054, ENSP00000271095, ENSP00000227588, ENSP00000297332, ENSP00000262445, ENSP00000330758, ENSP00000294383, ENSP00000252172, ENSP00000234697, ENSP00000347325, ENSP00000353218, ENSP00000262189, ENSP00000299853, ENSP00000352140, ENSP00000354285, ENSP00000353284, ENSP00000336725, ENSP00000234553, ENSP00000353575, ENSP00000335644, ENSP00000340347, ENSP00000354863, ENSP00000326540, ENSP00000326654, ENSP00000261412, ENSP00000336712, ENSP00000297332, ENSP00000265125, ENSP00000199320, ENSP00000345895, ENSP00000347396, ENSP00000215793, ENSP00000216254, ENSP00000263028, ENSP00000353817, ENSP00000312530, ENSP00000355399, ENSP00000324804, ENSP00000354405, ENSP00000318177, ENSP00000351562, ENSP00000263214, ENSP00000278100, ENSP00000299130, ENSP00000336741, ENSP00000295561, ENSP00000348370, ENSP00000339659, ENSP00000229695, ENSP00000231487, ENSP00000331708, ENSP00000326806, ENSP00000239262, ENSP00000262982, ENSP00000343253, ENSP00000246071, ENSP00000252622, ENSP00000269577, ENSP00000350217, ENSP00000342944, ENSP00000254630, ENSP00000354379, ENSP00000304233, ENSP00000260129, ENSP00000264883, ENSP00000344052, ENSP00000265148, ENSP00000340737, ENSP00000267812, ENSP00000354525, ENSP00000271551, ENSP00000346913, ENSP00000278560, ENSP00000280560, ENSP00000351234, ENSP00000280559, ENSP00000313128, ENSP00000344339, ENSP00000311135, ENSP00000283195, ENSP00000334538, ENSP00000344032, ENSP00000284041, ENSP00000285415, ENSP00000328992, ENSP00000290178, ENSP00000340575, ENSP00000294520, ENSP00000316490, ENSP00000297487, ENSP00000298600, ENSP00000298875, ENSP00000299518, ENSP00000300291, ENSP00000304370, ENSP00000307525, ENSP00000353622, ENSP00000338617, ENSP00000310042, ENSP00000318297, ENSP00000345919, ENSP00000221413, ENSP00000334373, ENSP00000261182, ENSP00000313778, ENSP00000341800, ENSP00000347969 ENSP00000233156, ENSP00000342306, ENSP00000251739, ENSP00000307666, ENSP00000325582, ENSP00000350341, ENSP00000249297, ENSP00000257745, ENSP00000312379, ENSP00000327505, ENSP00000333986, ENSP00000335398, ENSP00000335599, ENSP00000337136, ENSP00000340699, ENSP00000297044, ENSP00000234697, ENSP00000230671, ENSP00000279247, ENSP00000323036, ENSP00000337471, ENSP00000263464, ENSP00000336833, ENSP00000034275, ENSP00000315894, ENSP00000341483, ENSP00000042931, ENSP00000168666, ENSP00000355031, ENSP00000266058, ENSP00000315005, ENSP00000263636, ENSP00000229452, ENSP00000265872, ENSP00000354989, ENSP00000263551, ENSP00000343108, ENSP00000262914, ENSP00000336725, ENSP00000353284, ENSP00000184772, ENSP00000352062, ENSP00000263519, ENSP00000343886, ENSP00000328157, ENSP0000015926, ENSP00000349958, ENSP00000261396, ENSP00000176763, ENSP00000261875, ENSP00000188312, ENSP00000264296, ENSP00000316347, ENSP00000301624, ENSP00000336783, ENSP00000316066, ENSP00000263646, ENSP00000314733, ENSP00000004980, ENSP00000344652, ENSP00000265713, ENSP00000348904, ENSP00000312769, ENSP00000287735, ENSP00000318259, ENSP00000334016, ENSP00000312809, ENSP00000264750, ENSP00000302830, ENSP00000264206, ENSP00000309262, ENSP00000304118, ENSP00000216044, ENSP00000334787, ENSP00000216181, ENSP00000338576, ENSP00000351200, ENSP00000216237, ENSP00000352891, ENSP00000216479, ENSP00000343745, ENSP00000217166, ENSP00000338974, ENSP00000262173, ENSP00000218364, ENSP00000299167, ENSP00000313504, ENSP00000283027, ENSP00000219789, ENSP00000220509, ENSP00000325074, ENSP00000221455, ENSP00000221482, ENSP00000346170, ENSP00000263257, ENSP00000262807, ENSP00000270066, ENSP00000222379, ENSP00000222539, ENSP00000249270, ENSP00000262177, ENSP00000223084, ENSP00000304593, ENSP00000350985, ENSP00000224050, ENSP00000225504, ENSP00000225729, ENSP00000240304, ENSP00000311535, ENSP00000263083, ENSP00000263084, ENSP00000263776, ENSP00000324948, ENSP00000336946, ENSP00000339876, ENSP00000344078, ENSP00000350470, ENSP00000228495, ENSP00000229204, ENSP00000266679, ENSP00000229239, ENSP00000229330, ENSP00000310928, ENSP00000337063, ENSP00000353916, ENSP00000230083, ENSP00000319152, ENSP00000311603, ENSP00000261812, ENSP00000265138, ENSP00000231368, ENSP00000308738, ENSP00000338141, ENSP00000264678, ENSP00000232603, ENSP00000232607, ENSP00000341587, ENSP00000234160, ENSP00000234420, ENSP00000251293, ENSP00000251544, ENSP00000353564, ENSP00000264515, ENSP00000251160, ENSP00000251159, ENSP00000339630, ENSP00000326111, ENSP00000237853, ENSP00000342723, ENSP00000346335, ENSP00000350579, ENSP00000311144, ENSP00000351100, ENSP00000337736, ENSP00000314075, ENSP00000240327, ENSP00000265346, ENSP00000311778, ENSP00000336687, ENSP00000351265, ENSP00000263222, ENSP00000263771, ENSP00000350422, ENSP00000352971, ENSP00000220592, ENSP00000348229, ENSP00000338281, ENSP00000352634, ENSP00000354445, ENSP00000350723, ENSP00000344234, ENSP00000355408, ENSP00000312086, ENSP00000244227, ENSP00000246071, ENSP00000246489, ENSP00000333912, ENSP00000334523, ENSP00000334618, ENSP00000341154, ENSP00000263697, ENSP00000261249, ENSP00000247843, ENSP00000336747, ENSP00000265302, ENSP00000263791, ENSP00000250454, ENSP00000323968, ENSP00000250635, ENSP00000340896, ENSP00000313818, ENSP00000351644, ENSP00000253363, ENSP00000344581, ENSP00000344700, ENSP00000351749, ENSP00000354437, ENSP00000253686, ENSP00000254090, ENSP00000254976, ENSP00000307341, ENSP00000255202, ENSP00000307496, ENSP00000326199, ENSP00000355371, ENSP00000255608, ENSP00000256339, ENSP00000339276, ENSP00000256897, ENSP00000257131, ENSP00000257191, ENSP00000300265, ENSP00000316051, ENSP00000350967, ENSP00000257261, ENSP00000278840, ENSP00000258418, ENSP00000310623, ENSP00000344584, ENSP00000258780, ENSP00000258975, ENSP00000260008, ENSP00000260746, ENSP00000264584, ENSP00000321997, ENSP00000338366, ENSP00000346444, ENSP00000352190, ENSP00000265148, ENSP00000266939, ENSP00000317987, ENSP00000280557, ENSP00000267229, ENSP00000327080, ENSP00000268043, ENSP00000268482, ENSP00000327179, ENSP00000339164, ENSP00000346989, ENSP00000269188, ENSP00000314602, ENSP00000337476, ENSP00000349955, ENSP00000341101, ENSP00000271588, ENSP00000271590, ENSP00000272402, ENSP00000273037, ENSP00000273668, ENSP00000274118, ENSP00000274712, ENSP00000259750, ENSP00000307357, ENSP00000275057, ENSP00000276395, ENSP00000348933, ENSP00000276461, ENSP00000335220, ENSP00000276546, ENSP00000313410, ENSP00000313983, ENSP00000346111, ENSP00000276651, ENSP00000278062, ENSP00000278198, ENSP00000278200, ENSP00000320187, ENSP00000336927, ENSP00000280665, ENSP00000280699, ENSP00000280700, ENSP00000307980, ENSP00000281092, ENSP00000281182, ENSP00000282018, ENSP00000334167, ENSP00000347087, ENSP00000350168, ENSP00000283882, ENSP00000284670, ENSP00000285106, ENSP00000287394, ENSP00000205214, ENSP00000289382, ENSP00000320768, ENSP00000290663, ENSP00000354512, ENSP00000354630, ENSP00000316054, ENSP00000334648, ENSP00000294189, ENSP00000294352, ENSP00000295066, ENSP00000345837, ENSP00000352368, ENSP00000335541, ENSP00000295315, ENSP00000346464, ENSP00000296389, ENSP00000296417, ENSP00000296456, ENSP00000296468, ENSP00000296581, ENSP00000296642, ENSP00000297330, ENSP00000346339, ENSP00000297540, ENSP00000298451, ENSP00000298452, ENSP00000304994, ENSP00000318506, ENSP00000346604, ENSP00000352712, ENSP00000342214, ENSP00000316023, ENSP00000298556, ENSP00000355367, ENSP00000298851, ENSP00000346956, ENSP00000354689, ENSP00000309577, ENSP00000312169, ENSP00000351514, ENSP00000353871, ENSP00000300291, ENSP00000300901, ENSP00000341880, ENSP00000352421, ENSP00000300917, ENSP00000307940, ENSP00000339435, ENSP00000301920, ENSP00000307545, ENSP00000320234, ENSP00000304903, ENSP00000338617, ENSP00000353622, ENSP00000304283, ENSP00000303117, ENSP00000302728, ENSP00000340734, ENSP00000305060, ENSP00000302886, ENSP00000302160, ENSP00000313350, ENSP00000306807, ENSP00000318085, ENSP00000308534, ENSP00000343005, ENSP00000000442, ENSP00000311648, ENSP00000342673, ENSP00000310596, ENSP00000320447, ENSP00000325616, ENSP00000332444, ENSP00000342323, ENSP00000348689, ENSP00000321029, ENSP00000314214, ENSP00000354018, ENSP00000313890, ENSP00000327376, ENSP00000327957, ENSP00000327957, ENSP00000333666, ENSP00000328998, ENSP00000340702, ENSP00000330442, ENSP00000333256, ENSP00000332995, ENSP00000328139, ENSP00000331310, ENSP00000340350, ENSP00000340823, ENSP00000351886, ENSP00000343344, ENSP00000344504, ENSP00000353350, ENSP00000354337, ENSP00000350911, ENSP00000351446, ENSP00000349156, ENSP00000353090, ENSP00000289032, ENSP00000347909, ENSP00000350071, ENSP00000351729, ENSP00000352331, ENSP00000296412, ENSP00000351492, ENSP00000348283, ENSP00000352069, ENSP00000347244, ENSP00000354561, ENSP00000353586, ENSP00000353578, and ENSP00000290009, or an ortholog thereof.

In preferred embodiments of any of the above aspects, the REG nucleic acid molecule is selected from the following group: C04F12.1, K12B6.1, Y38A10A.6, F43G9.5, K08D10.4, ZK1127.6, ZK1127.9, W05H7.4, T19B10.4, T23B12.1, M03C11.3, Y71G10AL.1, ZK1127.3, F02E9.4, R06C1.1, ZK112.2, T19B4.5, T22B3.1, T01C3.8, B0414.7, ZC449.3, Y56A3A.17, F37B12.4, F52G2.2, C31H1.8, C55B7.5, E02H1.1, F09G2.4, F26A3.2, F43G9.1, F43G9.12, F46A9.5, F49D11.1, R06F6.1, T25G3.3, W06E11.1, W07E6.4, Y71F9B.4, ZK1127.5, ZK1127.7, C06E1.10, C16A3.4, D2089.1, F11A10.3, F56A8.6, F56D2.6, T08G11.4, T23D8.3, K07A.1.11, T12D8.1, T22D1.10, C27H6.2, C07E3.2, C26D10.1, D2096.8, F59A2.1, F32E10.4, K07F5.13a, R06A4.4a, W04C9.1, Y38F2AL.3, Y48G1A.5, ZK1127.4, C15F1.4, F52B5.6, F59A3.3, Y61A9LA.10, F48E8.5, T01G9.6a, C06A5.1, C29E4.2, K12H4.5, W04A4.5, Y110A7A.19, F26E4.4, F43G9.10, F54H12.1, F56A3.4, T09A5.10, W10C8.2, ENSG0000085511, ENSG00000092847, ENSG00000164860, ENSG00000150990, ENSG00000188976, ENSG00000107164, ENSG00000070785, ENSG00000173545, ENSG00000180198, ENSG00000175792, ENSG00000025770, ENSG00000025770, ENSG00000105176, ENSG00000153914, ENSG00000187109, ENSG00000086189, ENSG00000169375, ENSG00000165934, ENSG00000185619, ENSG00000014503, ENSG00000186432, ENSG00000162402, ENSG00000166411, ENSG00000140259, ENSG00000159086, ENSG00000167005, ENSG00000113558, ENSG00000105568, ENSG00000168438, ENSG00000198242, ENSG00000100412, ENSG00000138778, ENSG00000101811, ENSG00000019606, ENSG00000153201, ENSG00000143314, ENSG00000162521, ENSG00000138750, ENSG00000125870, ENSG00000134698, ENSG00000011007, ENSG00000083312, ENSG00000116478, ENSG00000163950, ENSG00000111968, ENSG00000137574, ENSG00000006788, ENSG00000055609, ENSG00000183207, ENSG00000138785, ENSG00000135521, ENSG00000169251, ENSG00000149262, ENSG00000150967, ENSG00000058600, ENSG00000099995, ENSG00000081059, ENSG00000132300, ENSG00000067048, ENSG00000155097, ENSG00000124207, ENSG00000093000, ENSG00000165733, ENSG00000130332, ENSG00000065559, ENSG00000109654, ENSG00000107949, ENSG00000120158, ENSG00000113649, ENSG00000131747, ENSG00000113649, ENSG00000131747, ENSG00000113649, ENSG00000142751, ENSG00000169375, ENSG00000090686, ENSG00000011654, ENSG00000012241, ENSG00000012241, ENSG00000126698, ENSG00000164167, ENSG00000031823, ENSG00000117222, ENSG00000075089, ENSG00000166833, ENSG00000003502, ENSG00000108372, ENSG00000137460, ENSG00000196363, ENSG00000079387, ENSG00000114487, ENSG00000124222, ENSG00000139746, ENSG00000039987, ENSG00000100697, ENSG00000121067, ENSG00000067842, ENSG00000125870, ENSG00000125870, ENSG00000005483, ENSG00000164032, ENSG00000178997, ENSG00000105821, ENSG00000111727, ENSG00000122515, ENSG00000127337, ENSG00000170860, ENSG00000060339, ENSG00000154832, ENSG00000151065, ENSG00000131368, ENSG00000123908, ENSG00000152670, ENSG00000188342, ENSG00000064393, ENSG00000189060, ENSG00000112759, ENSG00000118985, ENSG00000126214, ENSG00000139726, ENSG00000131051, ENSG00000164902, ENSG00000145088, ENSG00000147419, ENSG00000129691, ENSG00000172915, ENSG00000158435, ENSG00000196792, ENSG00000182874, ENSG00000001395, ENSG00000124380, ENSG00000167005, ENSG00000153774, ENSG00000104762, ENSG00000144021, ENSG00000122692, ENSG00000049246, ENSG00000113141, ENSG00000146216, ENSG00000116138, ENSG00000119720, ENSG00000117543, ENSG00000154473, ENSG00000138709, ENSG00000127948, ENSG00000074696, ENSG00000105993, ENSG00000129518, ENSG00000159200, ENSG00000149925, ENSG00000069248, ENSG00000148834, ENSG00000121067, ENSG00000114491, ENSG00000148950, ENSG00000185787, ENSG00000179837, ENSG00000169898, ENSG00000111605, ENSG00000067842, ENSG00000112333, ENSG00000156802, ENSG00000147475, ENSG00000005483, ENSG00000067533, ENSG00000067048, ENSG00000083168, ENSG00000110455, ENSG00000104859, ENSG00000147548, ENSG00000100591, ENSG00000106355, ENSG00000112033, ENSG00000111640, ENSG00000122565, ENSG00000169217, ENSG00000167658, ENSG00000170364, ENSG00000084072, ENSG00000134759, ENSG00000177463, ENSG00000158950, ENSG00000115806, ENSG00000173153, ENSG00000198258, ENSG00000196597, ENSG00000182606, ENSG00000196474, ENSG00000163125, ENSG00000163159, ENSG00000103274, ENSG00000078902, ENSG00000177613, ENSG00000090316, ENSG00000116903, ENSG00000165704, ENSG00000169919, ENSG00000141076, ENSG00000172273, ENSG00000130299, ENSG00000140451, ENSG00000138778, ENSG00000116062, ENSG00000144559, ENSG00000151498, ENSG00000123908, ENSG00000110906, ENSG00000134480, ENSG00000171956, ENSG00000104142, ENSG00000171865, ENSG00000085978, ENSG00000164008, ENSG00000167720, ENSG00000197214, ENSG00000113369, ENSG00000135932, ENSG00000114209, ENSG00000132849, ENSG00000011007, ENSG00000175324, ENSG00000162377, ENSG00000146267, ENSG00000128829, ENSG00000167447, ENSG00000011194, ENSG00000123444, ENSG00000138175, ENSG00000105771, ENSG00000165659, ENSG00000100345, ENSG00000116663, ENSG00000108963, ENSG00000133958, ENSG00000175137, ENSG00000120314, ENSG00000105648, ENSG00000164062, ENSG00000121057, ENSG00000183955, ENSG00000014257, ENSG00000146007, ENSG00000008256, ENSG00000179036, ENSG00000108591, ENSG00000141425, ENSG00000167433, ENSG00000127946, ENSG00000055291, ENSG00000105821, ENSG00000003436, ENSG00000140829, ENSG00000095459, ENSG00000004766, ENSG00000157426, ENSG00000148948, ENSG00000139505, ENSG00000168288, ENSG00000136279, ENSG00000108848, ENSG00000014216, ENSG00000136463, ENSG00000165678, ENSG00000068400, ENSG00000151422, ENSG00000196188, ENSG00000143341, ENSG00000017201, ENSG00000196839, ENSG00000185141, ENSG00000134824, ENSG00000165630, ENSG00000147647, ENSG00000133243, ENSG00000162244, ENSG00000198399, ENSG00000196470, ENSG00000077147, ENSG00000076356, ENSG00000151065, ENSG00000102893, ENSG00000182162, ENSG00000100226, ENSG00000197894, ENSG00000016400, ENSG00000117620, ENSG00000134698, ENSG00000187697, ENSG00000162961, ENSG00000152207, ENSG00000131781, ENSG00000164073, ENSG00000126814, ENSG00000145476, ENSG00000054219, ENSG00000197894, ENSG00000111640, ENSG00000110693, ENSG00000011083, ENSG00000198373, ENSG00000104885, ENSG00000182551, ENSG00000113441, ENSG00000072786, ENSG00000169026, ENSG00000165915, ENSG00000078687, ENSG00000078687, ENSG00000182077, ENSG00000169750, ENSG00000104967, ENSG00000198477, ENSG00000066135, ENSG00000159479, ENSG00000164219, ENSG00000151092, ENSG00000136003, ENSG00000170515, ENSG00000188566, ENSG00000182655, ENSG00000189060, ENSG00000164749, ENSG00000132639, ENSG00000174607, ENSG00000052758, ENSG00000023445, and ENSG00000161202 or an ortholog thereof.

In another aspect, the invention features an isolated REG polypeptide including an amino acid sequence with at least 35% identity to the amino acid sequence encoded by a nucleic acid molecule selected from the following group: M03C11.3, F52G2.2, T23B12.1, F22D6.6, T19B10.4, Y71G10AL.1, F43G9.12, C16A3.4, T01C3.8, T19B4.5, ZK1127.3, Y110A7A.19, C06A5.1, F26E4.4, T23D8.3, W04A4.5, C29E4.2, K12H4.5, ENSG00000138785, ENSG00000109606, ENSG00000150990, ENSG00000086189, ENSG00000159086, ENSG00000173545, ENSG00000132300, ENSG00000164860, ENSG00000135521, ENSG00000149262, ENSG00000025770 ENSG00000142751, ENSG00000137460, ENSG00000122515, ENSG00000164902, ENSG00000147419, ENSG00000158435, ENSG00000182874, ENSG00000146216, ENSG00000116138, ENSG00000119720, ENSG00000117543, ENSG00000138709, ENSG00000129518, ENSG00000148950, ENSG00000179837, ENSG00000169898, ENSG00000156802, ENSG00000147475, ENSG00000067533, ENSG00000158950, ENSG00000196597, ENSG00000196474, ENSG00000163125, ENSG00000164008, ENSG00000197214, ENSG00000162377, ENSG00000146267, ENSG00000128829, ENSG00000167447, ENSG00000101194, ENSG00000123444, ENSG00000116663, ENSG00000133958, ENSG00000175137, ENSG00000120314, ENSG00000141425, ENSG00000167433, ENSG00000095459, ENSG00000004766, ENSG00000168288, ENSG00000185141, ENSG00000133243, ENSG00000162961, ENSG00000164073, ENSG00000169026, ENSG00000078687, and ENSG00000182077, or an ortholog thereof, wherein said polypeptide is required for or enables RNAi.

In another aspect, the invention features an isolated REG polypeptide with an amino acid sequence having at least 35% identity to the amino acid sequence selected from the following group: C06A5.1, C06E1.10, C16A3.4, C29E4.2, E02H1.1, F22D6.6, F26E4.4, F43G9.12, F52B5.6, F56D2.6a, F56D2.6b, K12H4.5.3, K12H4.5.1, K12H4.5.4, K12H4.5.5, K12H4.5.6, K12H4.5.2, M03C11.3, T01C3.8, T19B10.4b, T19B10.4a, T19B4.5, T23B12.1, T23D8.3, W04A4.5, Y110A7A.19, Y71G10AL.1a, Y71G10AL.1b, ZK1127.3, ENSP00000299821, ENSP00000355052, ENSP00000199320, ENSP00000336741, ENSP00000295561, ENSP00000254630, ENSP00000354379, ENSP00000304233, ENSP00000340737, ENSP00000346913, ENSP00000278560, ENSP00000311135, ENSP00000328992, ENSP00000290178, ENSP00000340575, ENSP00000297487, ENSP00000310042, ENSP00000250454, ENSP00000323968, ENSP00000307545, ENSP00000320234, ENSP00000255608, ENSP00000263791, ENSP00000276395, ENSP00000348933, ENSP00000349955, ENSP00000337476, ENSP00000269188, ENSP00000314602, ENSP00000276461, ENSP00000335220, ENSP00000295066, ENSP00000345837, ENSP00000278200, ENSP00000350579, ENSP00000346335, ENSP00000342723, ENSP00000311778, ENSP00000265346, ENSP00000339276, ENSP00000256339, ENSP00000251544, ENSP00000303117, ENSP00000350422, ENSP00000352971, ENSP00000263771, ENSP00000327376, ENSP00000251293, ENSP00000313890, ENSP00000287394, ENSP00000251739, ENSP00000307666, ENSP00000325582, ENSP00000350341, ENSP00000297540, ENSP00000289382, ENSP00000300901, ENSP00000341880, ENSP00000296468, ENSP00000264584, ENSP00000321997, ENSP00000352190, ENSP00000346444, ENSP00000338366, ENSP00000328139, ENSP00000333256, ENSP00000260008, ENSP00000311144, ENSP00000351100, ENSP00000341101, ENSP00000289032, ENSP00000259750, ENSP00000307357, ENSP00000301920, ENSP00000304118, ENSP00000352421, ENSP00000300917, ENSP00000217166, ENSP00000338974, ENSP00000296389, ENSP00000301624, ENSP00000336783, ENSP00000316066, ENSP00000275057, ENSP00000294352, ENSP00000251160, ENSP00000251159, ENSP00000339630, ENSP00000352368, ENSP00000335541, ENSP00000184772, and ENSP00000342673, or an ortholog thereof.

In another aspect the invention features an isolated nucleic acid molecule including a nucleotide sequence having at least 50% identity to the nucleotide sequence selected from the following group: M03C11.3, F52G2.2, T23B12.1, F22D6.6, T19B10.4, Y71G10AL.1, F43G9.12, C16A3.4, T01C3.8, T19B4.5, ZK1127.3, Y110A7A.19, C06A5.1, F26E4.4, T23D8.3, W04A4.5, C29E4.2, K12H4.5, ENSG00000138785, ENSG00000019606, ENSG00000150990, ENSG00000086189, ENSG00000159086, ENSG00000173545, ENSG00000132300, ENSG00000164860, ENSG00000135521, ENSG00000149262, ENSG00000025770 ENSG00000142751, ENSG00000137460, ENSG00000122515, ENSG00000164902, ENSG00000147419, ENSG00000158435, ENSG00000182874, ENSG00000146216, ENSG00000116138, ENSG00000119720, ENSG00000117543, ENSG00000138709, ENSG00000129518, ENSG00000148950, ENSG00000179837, ENSG00000169898, ENSG00000156802, ENSG00000147475, ENSG00000067533, ENSG00000158950, ENSG00000196597, ENSG00000196474, ENSG00000163125, ENSG00000164008, ENSG00000197214, ENSG00000162377, ENSG00000146267, ENSG00000128829, ENSG00000167447, ENSG00000011194, ENSG00000123444, ENSG00000116663, ENSG00000133958, ENSG00000175137, ENSG00000120314, ENSG00000141425, ENSG00000167433, ENSG00000095459, ENSG00000004766, ENSG00000168288, ENSG00000185141, ENSG00000133243, ENSG00000162961, ENSG00000164073, ENSG00000169026, ENSG00000078687, and ENSG00000182077, or an ortholog thereof, where expression of a polypeptide encoded by the nucleic acid molecule in an organism enhances RNAi in said organism. In a related aspect, the invention features a vector that includes one of these nucleic acid molecules (e.g., a vector including a promoter operably linked to the nucleic acid molecule and capable of driving the expression of the nucleic acid molecule in a specific cell type, tissue, or organ). In another related aspect, the invention also features a host cell that includes the nucleic acid molecule or vector.

In a further aspect, the invention features an antibody that specifically binds to an REG polypeptide or fragment thereof, where the polypeptide is encoded by a nucleic acid molecule selected from the following group: M03C11.3, F52G2.2, T23B12.1, F22D6.6, T19B10.4, Y71G10AL.1, F43G9.12, C16A3.4, T01C3.8, T19B4.5, ZK1127.3, Y110A7A.19, C06A5.1, F26E4.4, T23D8.3, W04A4.5, C29E4.2, K12H4.5, ENSG00000138785, ENSG00000019606, ENSG00000150990, ENSG00000086189, ENSG00000159086, ENSG00000173545, ENSG00000132300, ENSG00000164860, ENSG00000135521, ENSG00000149262, ENSG00000025770 ENSG00000142751, ENSG00000137460, ENSG00000122515, ENSG00000164902, ENSG00000147419, ENSG00000158435, ENSG00000182874, ENSG00000146216, ENSG00000116138, ENSG00000119720, ENSG00000117543, ENSG00000138709, ENSG00000129518, ENSG00000148950, ENSG00000179837, ENSG00000169898, ENSG00000156802, ENSG00000147475, ENSG00000067533, ENSG00000158950, ENSG00000196597, ENSG00000196474, ENSG00000163125, ENSG00000164008, ENSG00000197214, ENSG00000162377, ENSG00000146267, ENSG00000128829, ENSG00000167447, ENSG00000011194, ENSG00000123444, ENSG00000116663, ENSG00000133958, ENSG00000175137, ENSG00000120314, ENSG00000141425, ENSG00000167433, ENSG00000095459, ENSG00000004766, ENSG00000168288, ENSG00000185141, ENSG00000133243, ENSG00000162961, ENSG00000164073, ENSG00000169026, ENSG00000078687, and ENSG00000182077, or an ortholog thereof; or where the polypeptide is selected from the following group: C06A5.1, C06E1.10, C16A3.4, C29E4.2, E02H1.1, F22D6.6, F26E4.4, F43G9.12, F52B5.6, F56D2.6a, F56D2.6b, K12H4.5.3, K12H4.5.1, K12H4.5.4, K12H4.5.5, K12H4.5.6, K12H4.5.2, M03C11.3, T01C3.8, T19B10.4b, T19B10.4a, T19B4.5, T23B12.1, T23D8.3, W04A4.5, Y110A7A.19, Y71G10AL.1a, Y71G10AL.1b, ZK1127.3, ENSP00000299821, ENSP00000355052, ENSP00000199320, ENSP00000336741, ENSP00000295561, ENSP00000254630, ENSP00000354379, ENSP00000304233, ENSP00000340737, ENSP00000346913, ENSP00000278560, ENSP00000311135, ENSP00000328992, ENSP00000290178, ENSP00000340575, ENSP00000297487, ENSP00000310042, ENSP00000250454, ENSP00000323968, ENSP00000307545, ENSP00000320234, ENSP00000255608, ENSP00000263791, ENSP00000276395, ENSP00000348933, ENSP00000349955, ENSP00000337476, ENSP00000269188, ENSP00000314602, ENSP00000276461, ENSP00000335220, ENSP00000295066, ENSP00000345837, ENSP00000278200, ENSP00000350579, ENSP00000346335, ENSP00000342723, ENSP00000311778, ENSP00000265346, ENSP00000339276, ENSP00000256339, ENSP00000251544, ENSP00000303117, ENSP00000350422, ENSP00000352971, ENSP00000263771, ENSP00000327376, ENSP00000251293, ENSP00000313890, ENSP00000287394, ENSP00000251739, ENSP00000307666, ENSP00000325582, ENSP00000350341, ENSP00000297540, ENSP00000289382, ENSP00000300901, ENSP00000341880, ENSP00000296468, ENSP00000264584, ENSP00000321997, ENSP00000352190, ENSP00000346444, ENSP00000338366, ENSP00000328139, ENSP00000333256, ENSP00000260008, ENSP00000311144, ENSP00000351100, ENSP00000341101, ENSP00000289032, ENSP00000259750, ENSP00000307357, ENSP00000301920, ENSP00000304118, ENSP00000352421, ENSP00000300917, ENSP00000217166, ENSP00000338974, ENSP00000296389, ENSP00000301624, ENSP00000336783, ENSP00000316066, ENSP00000275057, ENSP00000294352, ENSP00000251160, ENSP00000251159, ENSP00000339630, ENSP00000352368, ENSP00000335541, ENSP00000184772, and ENSP00000342673, or an ortholog thereof.

In another aspect the invention features an organism (e.g., a Drosophila, S. pombe, a nematode, a mammal, or a plant) including a mutation in an REG nucleic acid sequence, where the mutation decreases RNAi in the organism.

In yet another aspect, the invention features an isolated nucleobase oligomer including a duplex of at least eight but no more than thirty consecutive nucleobases of an REG nucleic acid, where the duplex, when contacted with an REG expressing cell, reduces REG transcription or translation. In one preferred embodiment, the duplex includes a first domain containing between 12, 15, 16, 17, 18, 19, 20, 21, 22, 23, 24, and 25 nucleobases and a second domain that hybridizes to the first domain under physiological conditions, where the first and second domains are connected by a single stranded loop. In one example, the duplex includes a first domain with between 21 and 29 nucleobases and a second domain that hybridizes to the first domain under physiological conditions, where the first and second domains are connected by a single-stranded loop. The single-stranded loop is, for example, between 6 and 12 nucleobases (e.g., 8 nucleobases). In one embodiment, the nucleobase oligomer reduces the level of expressed REG polypeptide.

In another aspect, the invention features an expression vector encoding a nucleobase oligomer including a duplex including at least eight but no more than thirty consecutive nucleobases of an REG nucleic acid, which, when contacted with an REG-expressing cell, reduces REG transcription or translation.

In another aspect, the invention features an expression vector encoding a nucleobase oligomer including a first region of at least eight but no more than thirty consecutive nucleobases corresponding to an REG nucleic acid molecule, and a second region of at least eight but no more than thirty consecutive nucleobases complementary to the first region, and the oligomer, when contacted with an REG-expressing cell, reduces REG transcription or translation.

Preferred embodiments of the previous two aspects include an expression vector with a nucleic acid sequence encoding the nucleobase oligomer operably linked to a promoter (e.g., PolIII promoter, polymerase III H1 promoter) capable of directing expression in a specific cell type, tissue, or organ. In a further embodiment, a cell (e.g., a transformed human cell stably expressing the expression vector, a cell in vivo, or a human cell) includes the expression vector.

In another aspect, the invention features a transgenic organism (e.g., a mammal nematode, or plant) expressing a nucleic acid sequence encoding an REG nucleobase oligomer that inhibits expression of an endogenous REG nucleic acid sequence.

In another aspect, the invention features a transgenic organism (e.g., a mammal nematode, or plant) expressing a nucleic acid sequence encoding an REG nucleic acid, where its expression enables RNAi or enhances the efficacy of RNAi in the organism.

In another aspect, the invention features a double-stranded RNA corresponding to at least a portion (e.g., at least 12, 15, 16, 17, 18, 19, 20, 21, 22, 23, 24, or 25 nucleotides) of an REG nucleic acid molecule of an organism. The double-stranded RNA is capable of decreasing the level of REG polypeptide encoded by an REG nucleic acid molecule.

In another aspect, the invention features an antisense nucleic acid molecule complementary to at least to at least 6, 12, 15, 16, 17, 18, 19, 20, 21, 22, 23, 24, or 25 nucleotides of an REG nucleic acid molecule and capable of decreasing expression of an REG polypeptide from an REG nucleic acid molecule.

In preferred embodiments of any of the above aspects, the REG polypeptide is selected from the following group: C06A5.1, C06E1.10, C16A3.4, C29E4.2, E02H1.1, F22D6.6, F26E4.4, F43G9.12, F52B5.6, F56D2.6a, F56D2.6b, K12H4.5.3, K12H4.5.1, K12H4.5.4, K12H4.5.5, K12H4.5.6, K12H4.5.2, M03C11.3, T01C3.8, T19B10.4b, T19B10.4a, T19B4.5, T23B12.1, T23D8.3, W04A4.5, Y110A7A.19, Y71G10AL.1a, Y71G10AL.1b, ZK1127.3, ENSP00000299821, ENSP00000355052, ENSP00000199320, ENSP00000336741, ENSP00000295561, ENSP00000254630, ENSP00000354379, ENSP00000304233, ENSP00000340737, ENSP00000346913, ENSP00000278560, ENSP00000311135, ENSP00000328992, ENSP00000290178, ENSP00000340575, ENSP00000297487, ENSP00000310042, ENSP00000250454, ENSP00000323968, ENSP00000307545, ENSP00000320234, ENSP00000255608, ENSP00000263791, ENSP00000276395, ENSP00000348933, ENSP00000349955, ENSP00000337476, ENSP00000269188, ENSP00000314602, ENSP00000276461, ENSP00000335220, ENSP00000295066, ENSP00000345837, ENSP00000278200, ENSP00000350579, ENSP00000346335, ENSP00000342723, ENSP00000311778, ENSP00000265346, ENSP00000339276, ENSP00000256339, ENSP00000251544, ENSP00000303117, ENSP00000350422, ENSP00000352971, ENSP00000263771, ENSP00000327376, ENSP00000251293, ENSP00000313890, ENSP00000287394, ENSP00000251739, ENSP00000307666, ENSP00000325582, ENSP00000350341, ENSP00000297540, ENSP00000289382, ENSP00000300901, ENSP00000341880, ENSP00000296468, ENSP00000264584, ENSP00000321997, ENSP00000352190, ENSP00000346444, ENSP00000338366, ENSP00000328139, ENSP00000333256, ENSP00000260008, ENSP00000311144, ENSP00000351100, ENSP00000341101, ENSP00000289032, ENSP00000259750, ENSP00000307357, ENSP00000301920, ENSP00000304118, ENSP00000352421, ENSP00000300917, ENSP00000217166, ENSP00000338974, ENSP00000296389, ENSP00000301624, ENSP00000336783, ENSP00000316066, ENSP00000275057, ENSP00000294352, ENSP00000251160, ENSP00000251159, ENSP00000339630, ENSP00000352368, ENSP00000335541, ENSP00000184772, and ENSP00000342673, or an ortholog thereof.

In preferred embodiments of any of the above aspects, the REG nucleic acid molecule is selected from the following group: M03C11.3, F52G2.2, T23B12.1, F22D6.6, T19B10.4, Y71G10AL.1, F43G9.12, C16A3.4, T01C3.8, T19B4.5, ZK1127.3, Y110A7A.19, C06A5.1, F26E4.4, T23D8.3, W04A4.5, C29E4.2, K12H4.5, ENSG00000138785, ENSG00000109606, ENSG00000150990, ENSG00000086189, ENSG00000159086, ENSG00000173545, ENSG00000132300, ENSG00000164860, ENSG00000135521, ENSG00000149262, ENSG00000025770 ENSG00000142751, ENSG00000137460, ENSG00000122515, ENSG00000164902, ENSG00000147419, ENSG00000158435, ENSG00000182874, ENSG00000146216, ENSG00000116138, ENSG00000119720, ENSG00000117543, ENSG00000138709, ENSG00000129518, ENSG00000148950, ENSG00000179837, ENSG00000169898, ENSG00000156802, ENSG00000147475, ENSG00000067533, ENSG00000158950, ENSG00000196597, ENSG00000196474, ENSG00000163125, ENSG00000164008, ENSG00000197214, ENSG00000162377, ENSG00000146267, ENSG00000128829, ENSG00000167447, ENSG00000001194, ENSG00000123444, ENSG00000116663, ENSG00000133958, ENSG00000175137, ENSG00000120314, ENSG00000141425, ENSG00000167433, ENSG00000095459, ENSG00000004766, ENSG00000168288, ENSG00000185141, ENSG00000133243, ENSG00000162961, ENSG00000164073, ENSG00000169026, ENSG00000078687, and ENSG00000182077, or an ortholog thereof.

By “RNAi essential gene (REG) polypeptide” is meant a polypeptide that is required in an organism for RNA interference and having a BlastP E-value of at least 1.0 e⁻⁵, 1.0 e⁻¹⁰, 1.0 e⁻²⁵, 1.0 e⁻⁵⁰, 1.0 e⁻¹⁰⁰ or substantial identity to an REG polypeptide, or ortholog thereof, encoded by a nucleic acid selected from the following group: C04F12.1, K12B6.1, Y38A10A.6, F43G9.5, K08D10.4, ZK1127.6, ZK1127.9, W05H7.4, T19B10.4, T23B12.1, M03C11.3, Y71G10AL.1, ZK1127.3, F02E9.4, R06C1.1, ZK112.2, T19B4.5, T22B3.1, T01C3.8, B0414.7, ZC449.3, Y56A3A.17, F37B12.4, F52G2.2, C31H1.8, C55B7.5, E02H1.1, F09G2.4, F26A3.2, F43G9.1, F43G9.12, F46A9.5, F49D11.1, R06F6.1, T25G3.3, W06E11.1, W07E6.4, Y71F9B.4, ZK1127.5, ZK1127.7, C06E1.10, C16A3.4, D2089.1, F11A10.3, F56A8.6, F56D2.6, T08G11.4, T23D8.3, K07A1.11, T12D8.1, T22D1.10, C27H6.2, C07E3.2, C26D10.1, D2096.8, F59A2.1, F32E10.4, K07F5.13a, R06A4.4a, W04C9.1, Y38F2AL.3, Y48G1A.5, ZK1127.4, C15F1.4, F52B5.6, F59A3.3, Y61A9LA.10, F48E8.5, T01G9.6a, C06A5.1, C29E4.2, K12H4.5, W04A4.5, Y110A7A.19, F26E4.4, F43G9.10, F54H12.1, F56A3.4, T09A5.10, W10C8.2, ENSG00000085511, ENSG00000092847, ENSG00000164860, ENSG00000150990, ENSG00000188976, ENSG00000017164, ENSG00000070785, ENSG00000173545, ENSG00000180198, ENSG00000175792, ENSG00000025770, ENSG00000025770, ENSG00000015176, ENSG00000153914, ENSG00000187109, ENSG00000086189, ENSG00000169375, ENSG00000165934, ENSG00000185619, ENSG00000114503, ENSG00000186432, ENSG00000162402, ENSG00000166411, ENSG00000140259, ENSG00000159086, ENSG00000167005, ENSG00000113558, ENSG00000105568, ENSG00000168438, ENSG00000198242, ENSG00000001412, ENSG00000138778, ENSG00000011811, ENSG00000019606, ENSG00000153201, ENSG00000143314, ENSG00000162521, ENSG00000138750, ENSG00000125870, ENSG00000134698, ENSG00000011007, ENSG00000083312, ENSG00000116478, ENSG00000163950, ENSG00000111968, ENSG00000137574, ENSG00000006788, ENSG00000055609, ENSG00000183207, ENSG00000138785, ENSG00000135521, ENSG00000169251, ENSG00000149262, ENSG00000150967, ENSG00000058600, ENSG00000099995, ENSG00000081059, ENSG00000132300, ENSG00000067048, ENSG00000155097, ENSG00000124207, ENSG00000093000, ENSG00000165733, ENSG00000130332, ENSG00000065559, ENSG00000109654, ENSG00000107949, ENSG00000120158, ENSG00000113649, ENSG00000131747, ENSG00000113649, ENSG00000131747, ENSG00000113649, ENSG00000142751, ENSG00000169375, ENSG00000090686, ENSG00000101654, ENSG00000102241, ENSG00000102241, ENSG00000126698, ENSG00000164167, ENSG00000031823, ENSG00000117222, ENSG00000075089, ENSG00000166833, ENSG00000103502, ENSG00000108372, ENSG00000137460, ENSG00000196363, ENSG00000079387, ENSG000000114487, ENSG00000124222, ENSG00000139746, ENSG00000039987, ENSG00000100697, ENSG00000121067, ENSG00000067842, ENSG00000125870, ENSG00000125870, ENSG00000005483, ENSG00000164032, ENSG00000178997, ENSG00000105821, ENSG00000111727, ENSG00000122515, ENSG00000127337, ENSG00000170860, ENSG00000060339, ENSG00000154832, ENSG00000151065, ENSG00000131368, ENSG00000123908, ENSG00000152670, ENSG00000188342, ENSG00000064393, ENSG00000189060, ENSG00000112759, ENSG00000118985, ENSG00000126214, ENSG00000139726, ENSG00000131051, ENSG00000164902, ENSG00000145088, ENSG00000147419, ENSG00000129691, ENSG00000172915, ENSG00000158435, ENSG00000196792, ENSG00000182874, ENSG00000100395, ENSG00000124380, ENSG00000167005, ENSG00000153774, ENSG00000104762, ENSG00000144021, ENSG00000122692, ENSG00000049246, ENSG00000113141, ENSG00000146216, ENSG00000116138, ENSG000000119720, ENSG00000117543, ENSG00000154473, ENSG00000138709, ENSG000000127948, ENSG00000074696, ENSG00000105993, ENSG00000129518, ENSG00000159200, ENSG00000149925, ENSG00000069248, ENSG00000148834, ENSG00000121067, ENSG00000114491, ENSG00000148950, ENSG00000185787, ENSG00000179837, ENSG00000169898, ENSG00000111605, ENSG00000067842, ENSG00000112333, ENSG00000156802, ENSG00000147475, ENSG00000005483, ENSG00000067533, ENSG00000067048, ENSG00000083168, ENSG00000110455, ENSG00000104859, ENSG00000147548, ENSG00000100591, ENSG00000106355, ENSG00000112033, ENSG00000111640, ENSG00000122565, ENSG00000169217, ENSG00000167658, ENSG00000170364, ENSG00000084072, ENSG00000134759, ENSG00000177463, ENSG00000158950, ENSG00000115806, ENSG00000173153, ENSG00000198258, ENSG00000196597, ENSG00000182606, ENSG00000196474, ENSG00000163125, ENSG00000163159, ENSG00000103274, ENSG00000078902, ENSG00000177613, ENSG00000090316, ENSG00000116903, ENSG00000165704, ENSG000000169919, ENSG00000141076, ENSG00000172273, ENSG00000130299, ENSG00000140451, ENSG00000138778, ENSG00000116062, ENSG00000144559, ENSG00000151498, ENSG00000123908, ENSG00000110906, ENSG00000134480, ENSG00000171956, ENSG00000014142, ENSG00000171865, ENSG00000085978, ENSG00000164008, ENSG00000167720, ENSG00000197214, ENSG00000113369, ENSG00000135932, ENSG00000114209, ENSG00000132849, ENSG00000011007, ENSG00000175324, ENSG00000162377, ENSG00000146267, ENSG00000128829, ENSG00000167447, ENSG00000011194, ENSG00000123444, ENSG00000138175, ENSG00000015771, ENSG00000165659, ENSG00000001345, ENSG00000116663, ENSG00000108963, ENSG00000133958, ENSG00000175137, ENSG00000120314, ENSG00000105648, ENSG00000164062, ENSG00000121057, ENSG00000183955, ENSG00000014257, ENSG00000146007, ENSG00000008256, ENSG00000179036, ENSG00000018591, ENSG00000141425, ENSG00000167433, ENSG00000127946, ENSG00000055291, ENSG00000105821, ENSG00000003436, ENSG00000140829, ENSG00000095459, ENSG00000004766, ENSG00000157426, ENSG00000148948, ENSG00000139505, ENSG00000168288, ENSG00000136279, ENSG00000108848, ENSG00000014216, ENSG00000136463, ENSG00000165678, ENSG00000068400, ENSG00000151422, ENSG00000196188, ENSG00000143341, ENSG00000017201, ENSG00000196839, ENSG00000185141, ENSG00000134824, ENSG00000165630, ENSG00000147647, ENSG00000133243, ENSG00000162244, ENSG00000198399, ENSG00000196470, ENSG00000077147, ENSG00000076356, ENSG00000151065, ENSG00000102893, ENSG00000182162, ENSG00000001226, ENSG00000197894, ENSG00000106400, ENSG00000117620, ENSG00000134698, ENSG00000187697, ENSG00000162961, ENSG00000152207, ENSG00000131781, ENSG00000164073, ENSG00000126814, ENSG00000145476, ENSG00000054219, ENSG00000197894, ENSG00000111640, ENSG00000110693, ENSG00000011083, ENSG00000198373, ENSG00000104885, ENSG00000182551, ENSG00000113441, ENSG00000072786, ENSG00000169026, ENSG00000165915, ENSG00000078687, ENSG00000078687, ENSG00000182077, ENSG00000169750, ENSG00000104967, ENSG00000198477, ENSG00000066135, ENSG00000159479, ENSG00000164219, ENSG00000151092, ENSG00000136003, ENSG00000170515, ENSG00000188566, ENSG00000182655, ENSG00000189060, ENSG00000164749, ENSG00000132639, ENSG00000174607, ENSG00000052758, ENSG00000023445, and ENSG00000161202, or to a polypeptide selected from the following group: B0414.7b, B0414.7a, C04F12.1, C06A5.1, C06E1.10, C07E3.2, C12D8.1b.3, C12D8.1b.1, C12D8.1a, C12D8.1b.2, C15F1.4, C16A3.4, C26D10.1, C27H6.2, C29E4.2, C31H1.8, C55B7.5, D2089.1b.1, D2089.1b.2, D2089.1a, D2089.1b.3, D2096.8, E02H1.1, F02E9.4.1, F02E9.4.2, F09G2.4, F11A10.3, F22D6.6, F26A3.2, F26E4.4, F32E10.4, F37B12.4, F43G9.1, F43G9.10, F43G9.12, F43G9.5, F46A9.5.1, F46A9.5.2, F48E8.5.2, F48E8.5.3, F48E8.5.1, F49D11.1, F52B5.6, F52G2.2, F54H12.1a, F54H12.1c, F54H12.1b, F56A3.4, F56A8.6, F56D2.6a, F56D2.6b, F59A2.1b.2, F59A2.1b.1, F59A2.1a, F59A3.3, K07A1.11, K07F5.13c, K07F5.13b, K07F5.13a, K08D10.4, K12B6.1, K12H4.5.3, K12H4.5.1, K12H4.5.4, K12H4.5.5, K12H4.5.6, K12H4.5.2, M03C11.3, R03D7.4, R06A4.4a, R06A4.4b, R06C1.1, R06F6.1, T01C3.8, T01G9.6a.2, T01G9.6b, T01G9.6a.1, T08G11.4, T09A5.10, T12D8.1.1, T19B10.4b, T19B10.4a, T19B4.5, T22B3.1, T22D1.10, T23B12.1, T23D8.3, T25G3.3, W04A4.5, W04C9.1, W05H7.4c, W05H7.4d, W05H7.4a, W05H7.4b, W06E11.1, W07E6.4, W10C8.2, Y110A7A.19, Y38A10A.6, Y38F2AL.3b, Y38F2AL.3a, Y48G1A.5, Y56A3A.17b, Y56A3A.17a, Y61A9LA.10, Y71F9B.4, Y71G10AL.1a, Y71G10AL.1b, ZC449.3b, ZC449.3a.2, ZC449.3a.1, ZK112.2, ZK1127.3, ZK1127.4, ZK1127.5, ZK1127.6.1, ZK1127.6.2, ZK1127.7, ZK1127.9e.2, ZK1127.9d, ZK1127.9c, ZK1127.9e.3, ZK1127.9b, ZK1127.9a, ZK1127.9e.1, ENSP00000257131, ENSP00000207451, ENSP00000277804, ENSP00000300291, ENSP00000243563, ENSP00000296702, ENSP00000251819, ENSP00000331699, ENSP00000265155, ENSP00000289371, TR:Q7Z4R6, ENSP00000248054, ENSP00000271095, ENSP00000227588, ENSP00000297332, ENSP00000262445, ENSP00000330758, ENSP00000294383, ENSP00000252172, ENSP00000234697, ENSP00000347325, ENSP00000353218, ENSP00000262189, ENSP00000299853, ENSP00000352140, ENSP00000354285, ENSP00000353284, ENSP00000336725, ENSP00000234553, ENSP00000353575, ENSP00000335644, ENSP00000340347, ENSP00000354863, ENSP00000326540, ENSP00000326654, ENSP00000261412, ENSP00000336712, ENSP00000297332, ENSP00000265125, ENSP00000199320, ENSP00000345895, ENSP00000347396, ENSP00000215793, ENSP00000216254, ENSP00000263028, ENSP00000353817, ENSP00000312530, ENSP00000355399, ENSP00000324804, ENSP00000354405, ENSP00000318177, ENSP00000351562, ENSP00000263214, ENSP00000278100, ENSP00000299130, ENSP00000336741, ENSP00000295561, ENSP00000348370, ENSP00000339659, ENSP00000229695, ENSP00000231487, ENSP00000331708, ENSP00000326806, ENSP00000239262, ENSP00000262982, ENSP00000343253, ENSP00000246071, ENSP00000252622, ENSP00000269577, ENSP00000350217, ENSP00000342944, ENSP00000254630, ENSP00000354379, ENSP00000304233, ENSP00000260129, ENSP00000264883, ENSP00000344052, ENSP00000265148, ENSP00000340737, ENSP00000267812, ENSP00000354525, ENSP00000271551, ENSP00000346913, ENSP00000278560, ENSP00000280560, ENSP00000351234, ENSP00000280559, ENSP00000313128, ENSP00000344339, ENSP00000311135, ENSP00000283195, ENSP00000334538, ENSP00000344032, ENSP00000284041, ENSP00000285415, ENSP00000328992, ENSP00000290178, ENSP00000340575, ENSP00000294520, ENSP00000316490, ENSP00000297487, ENSP00000298600, ENSP00000298875, ENSP00000299518, ENSP00000300291, ENSP00000304370, ENSP00000307525, ENSP00000353622, ENSP00000338617, ENSP00000310042, ENSP00000318297, ENSP00000345919, ENSP00000221413, ENSP00000334373, ENSP00000261182, ENSP00000313778, ENSP00000341800, ENSP00000347969 ENSP00000233156, ENSP00000342306, ENSP00000251739, ENSP00000307666, ENSP00000325582, ENSP00000350341, ENSP00000249297, ENSP00000257745, ENSP00000312379, ENSP00000327505, ENSP00000333986, ENSP00000335398, ENSP00000335599, ENSP00000337136, ENSP00000340699, ENSP00000297044, ENSP00000234697, ENSP00000230671, ENSP00000279247, ENSP00000323036, ENSP00000337471, ENSP00000263464, ENSP00000336833, ENSP00000034275, ENSP00000315894, ENSP00000341483, ENSP00000042931, ENSP00000168666, ENSP00000355031, ENSP00000266058, ENSP00000315005, ENSP00000263636, ENSP00000229452, ENSP00000265872, ENSP00000354989, ENSP00000263551, ENSP00000343108, ENSP00000262914, ENSP00000336725, ENSP00000353284, ENSP00000184772, ENSP00000352062, ENSP00000263519, ENSP00000343886, ENSP00000328157, ENSP00000015926, ENSP00000349958, ENSP00000261396, ENSP00000176763, ENSP00000261875, ENSP00000188312, ENSP00000264296, ENSP00000316347, ENSP00000301624, ENSP00000336783, ENSP00000316066, ENSP00000263646, ENSP00000314733, ENSP00000004980, ENSP00000344652, ENSP00000265713, ENSP00000348904, ENSP00000312769, ENSP00000287735, ENSP00000318259, ENSP00000334016, ENSP00000312809, ENSP00000264750, ENSP00000302830, ENSP00000264206, ENSP00000309262, ENSP00000304118, ENSP00000216044, ENSP00000334787, ENSP00000216181, ENSP00000338576, ENSP00000351200, ENSP00000216237, ENSP00000352891, ENSP00000216479, ENSP00000343745, ENSP00000217166, ENSP00000338974, ENSP00000262173, ENSP00000218364, ENSP00000299167, ENSP00000313504, ENSP00000283027, ENSP00000219789, ENSP00000220509, ENSP00000325074, ENSP00000221455, ENSP00000221482, ENSP00000346170, ENSP00000263257, ENSP00000262807, ENSP00000270066, ENSP00000222379, ENSP00000222539, ENSP00000249270, ENSP00000262177, ENSP00000223084, ENSP00000304593, ENSP00000350985, ENSP00000224050, ENSP00000225504, ENSP00000225729, ENSP00000240304, ENSP00000311535, ENSP00000263083, ENSP00000263084, ENSP00000263776, ENSP00000324948, ENSP00000336946, ENSP00000339876, ENSP00000344078, ENSP00000350470, ENSP00000228495, ENSP00000229204, ENSP00000266679, ENSP00000229239, ENSP00000229330, ENSP00000310928, ENSP00000337063, ENSP00000353916, ENSP00000230083, ENSP00000319152, ENSP00000311603, ENSP00000261812, ENSP00000265138, ENSP00000231368, ENSP00000308738, ENSP00000338141, ENSP00000264678, ENSP00000232603, ENSP00000232607, ENSP00000341587, ENSP00000234160, ENSP00000234420, ENSP00000251293, ENSP00000251544, ENSP00000353564, ENSP00000264515, ENSP00000251160, ENSP00000251159, ENSP00000339630, ENSP00000326111, ENSP00000237853, ENSP00000342723, ENSP00000346335, ENSP00000350579, ENSP00000311144, ENSP00000351100, ENSP00000337736, ENSP00000314075, ENSP00000240327, ENSP00000265346, ENSP00000311778, ENSP00000336687, ENSP00000351265, ENSP00000263222, ENSP00000263771, ENSP00000350422, ENSP00000352971, ENSP00000220592, ENSP00000348229, ENSP00000338281, ENSP00000352634, ENSP00000354445, ENSP00000350723, ENSP00000344234, ENSP00000355408, ENSP00000312086, ENSP00000244227, ENSP00000246071, ENSP00000246489, ENSP00000333912, ENSP00000334523, ENSP00000334618, ENSP00000341154, ENSP00000263697, ENSP00000261249, ENSP00000247843, ENSP00000336747, ENSP00000265302, ENSP00000263791, ENSP00000250454, ENSP00000323968, ENSP00000250635, ENSP00000340896, ENSP00000313818, ENSP00000351644, ENSP00000253363, ENSP00000344581, ENSP00000344700, ENSP00000351749, ENSP00000354437, ENSP00000253686, ENSP00000254090, ENSP00000254976, ENSP00000307341, ENSP00000255202, ENSP00000307496, ENSP00000326199, ENSP00000355371, ENSP00000255608, ENSP00000256339, ENSP00000339276, ENSP00000256897, ENSP00000257131, ENSP00000257191, ENSP00000300265, ENSP00000316051, ENSP00000350967, ENSP00000257261, ENSP00000278840, ENSP00000258418, ENSP00000310623, ENSP00000344584, ENSP00000258780, ENSP00000258975, ENSP00000260008, ENSP00000260746, ENSP00000264584, ENSP00000321997, ENSP00000338366, ENSP00000346444, ENSP00000352190, ENSP00000265148, ENSP00000266939, ENSP00000317987, ENSP00000280557, ENSP00000267229, ENSP00000327080, ENSP00000268043, ENSP00000268482, ENSP00000327179, ENSP00000339164, ENSP00000346989, ENSP00000269188, ENSP00000314602, ENSP00000337476, ENSP00000349955, ENSP00000341101, ENSP00000271588, ENSP00000271590, ENSP00000272402, ENSP00000273037, ENSP00000273668, ENSP00000274118, ENSP00000274712, ENSP00000259750, ENSP00000307357, ENSP00000275057, ENSP00000276395, ENSP00000348933, ENSP00000276461, ENSP00000335220, ENSP00000276546, ENSP00000313410, ENSP00000313983, ENSP00000346111, ENSP00000276651, ENSP00000278062, ENSP00000278198, ENSP00000278200, ENSP00000320187, ENSP00000336927, ENSP00000280665, ENSP00000280699, ENSP00000280700, ENSP00000307980, ENSP00000281092, ENSP00000281182, ENSP00000282018, ENSP00000334167, ENSP00000347087, ENSP00000350168, ENSP00000283882, ENSP00000284670, ENSP00000285106, ENSP00000287394, ENSP00000205214, ENSP00000289382, ENSP00000320768, ENSP00000290663, ENSP00000354512, ENSP00000354630, ENSP00000316054, ENSP00000334648, ENSP00000294189, ENSP00000294352, ENSP00000295066, ENSP00000345837, ENSP00000352368, ENSP00000335541, ENSP00000295315, ENSP00000346464, ENSP00000296389, ENSP00000296417, ENSP00000296456, ENSP00000296468, ENSP00000296581, ENSP00000296642, ENSP00000297330, ENSP00000346339, ENSP00000297540, ENSP00000298451, ENSP00000298452, ENSP00000304994, ENSP00000318506, ENSP00000346604, ENSP00000352712, ENSP00000342214, ENSP00000316023, ENSP00000298556, ENSP00000355367, ENSP00000298851, ENSP00000346956, ENSP00000354689, ENSP00000309577, ENSP00000312169, ENSP00000351514, ENSP00000353871, ENSP00000300291, ENSP00000300901, ENSP00000341880, ENSP00000352421, ENSP00000300917, ENSP00000307940, ENSP00000339435, ENSP00000301920, ENSP00000307545, ENSP00000320234, ENSP00000304903, ENSP00000338617, ENSP00000353622, ENSP00000304283, ENSP00000303117, ENSP00000302728, ENSP00000340734, ENSP00000305060, ENSP00000302886, ENSP00000302160, ENSP00000313350, ENSP00000306807, ENSP00000318085, ENSP00000308534, ENSP00000343005, ENSP00000000442, ENSP00000311648, ENSP00000342673, ENSP00000310596, ENSP00000320447, ENSP00000325616, ENSP00000332444, ENSP00000342323, ENSP00000348689, ENSP00000321029, ENSP00000314214, ENSP00000354018, ENSP00000313890, ENSP00000327376, ENSP00000327957, ENSP00000327957, ENSP00000333666, ENSP00000328998, ENSP00000340702, ENSP00000330442, ENSP00000333256, ENSP00000332995, ENSP00000328139, ENSP00000331310, ENSP00000340350, ENSP00000340823, ENSP00000351886, ENSP00000343344, ENSP00000344504, ENSP00000353350, ENSP00000354337, ENSP00000350911, ENSP00000351446, ENSP00000349156, ENSP00000353090, ENSP00000289032, ENSP00000347909, ENSP00000350071, ENSP00000351729, ENSP00000352331, ENSP00000296412, ENSP00000351492, ENSP00000348283, ENSP00000352069, ENSP00000347244, ENSP00000354561, ENSP00000353586, ENSP00000353578, and ENSP00000290009, or an ortholog thereof. Accession numbers containing “ENSP,” “ENSG,” or preceded by the term “Ensemb” are publicly available in the Ensembl database which is maintained in a collaboration between the European Molecular Biology Laboratory (EMBL) and the SangerInstitute and funded by the Wellcome Trust.

Orthologs of REG nucleic acid and polypeptides also include those selected from the group consisting of GADFLY:CG7439-PB, SwissProt Database No. (SW) SW:SRMB_ECOLI, Saccharomyces Genome Database (SGD):YPL119C, GADFLY:CG9748-PA, SGD:YDR432W, GADFLY:CG6043-PA, SGD:YNL016W, GADFLY:CG112750-PA, SW:ACIN_MOUSE, GADFLY:CG7107-PA, SGD:YML074C, SGD:YNL136W, TrEMBL (TR) is a computer-annotated supplement of Swiss-Prot that contains all the translations of European Molecular Biology Laboratory nucleotide sequence entries not yet integrated in Swiss-Prot TrEMBL TR:Q9ZQW9, SGD:YOL004W, S. cerevisiae RPD3 SGD:YNL330C, GADFLY:CG10719-PA, TR:Q9DUM3, GADFLY:CG2158-PA. Accession numbers preceded by the term “Gadfly” are publicly available at the Berkeley Drosophila Genome Project GadFly: Genome Annotation Database of Drosophila (http://www.fruitfly.org/annot/).

By “REG nucleic acid” is meant a nucleic acid encoding an REG polypeptide or ortholog thereof.

By “Piwi domain protein” is meant a protein encoded by C04F 12.1, or an ortholog thereof, that functions in RNAi. Such orthologs include Drosophila AGO2 (Argonaute2) (GADFLY:CG7439-PB; BLASTP E-value of 6.1e-26 over 46.9% of the total protein length), and human eukaryotic translation initiation factor 2C (ENSEMBL:ENSP00000257131; eIF2C, BLASTP E-value of 1.1e-24 over 58.8% of the total protein length). By “Piwi domain protein” is also meant a protein encoded by K12B6.1, or an ortholog thereof. Such orthologs include Drosophila AGO2 (Argonaute2) (GADFLY:CG7439-PB; BLASTP E-value of 2.9e-27 over 50.4% of the total protein length) and human eukaryotic translation initiation factor 2C (ENSEMBL:ENSP00000207451; eIF2C, BLASTP E-value of 6.7e-27 over 62.7% of the total protein length).

By “DEAH/D-box helicase” is meant a protein encoded by Y38A10A.6, or an ortholog thereof, that functions in RNAi. Such orthologs include E. coli (SW:SRMB_ECOLI; BLASTP E-value of 2.5e-17 over 55.6% of the total protein length), S. cerevisiae (SGD:YPL119C; BLASTP E-value of 3e-18 over 82.1% of the total protein length), Drosophila (GADFLY:CG9748-PA; BLASTP E-value of 4.3e-17 over 69.2% of the total protein length), and human RHII/Gu (ENSEMBL:ENSP00000277804; BLASTP E-value of 7.4e-17 over 52.3% of the total protein length).

By “Pre-mRNA cleavage factor Iₘ subunit” is meant a protein encoded by F43G9.5, or an ortholog thereof, that functions in RNAi. Such orthologs include human pre-mRNA cleavage factor Iₘ (ENSEMBL:ENSP00000300291; BLASTP E-value of 6.9e-78 over 86.8% of the total protein length). Other orthologs include A. thaliana TR:Q94AFOTR; Q9MOK5TR; 065606TR; Q9SZQ4; B. malayi TR:Q81712; B. rerio TR:Q7T3C6; H. sapiens ENSEMBL:ENSP00000300291; M. musculus TR:Q9CQF3; D. melanogaster GADFLY:CG3689-PB; M. musculus TR:Q9CZQ0; A. thaliana TR:Q8GXS3; O. sativa TR:Q7XPV9.

By “RNA-binding protein” is meant an RNP-2 protein encoded by K08D10.4, or an ortholog thereof, that functions in RNAi. Such orthologs include human U1A small nuclear riboprotein A (ENSEMBL:ENSP00000243563; BLASTP E-value of 4.1e-54 over 98.5% of the total protein length). Other orthologs include O. sativa TR:Q94GW0; A. thaliana TR:022922TR:Q8LB63; X. laevis SW:RUIA_XENLA.

By “Transcriptional elongation factor” is meant a protein encoded by ZK1127.6 or ZK1127.9, or an ortholog thereof, that functions in RNAi. ZK1127.6 or ZK1127.9 share extensive homology (BLASTP E-value of 1.5e-201 over 89.9% of the total protein length of ZK1127.6). Their closest human ortholog is transcription elongation regulator 1 (CA150) (ENSEMBL:ENSP00000296702; BLASTP E-value of 1.5e-135 over 82.3% of the total protein length).

By “CCCH Zn-finger protein” is meant a protein encoded by W05H7.4, or an ortholog thereof, that functions in RNAi. Such orthologs include O. sativa U2 snRNP auxiliary factor small subunit (TR:Q9ZQW9; BLASTP E-value of 4.5e-06 over 22.2% of the total protein length), S. cerevisiae NPL3, an RNA-binding protein that carries poly(A)+ mRNA from the nucleus into the cytoplasm (SGD:YDR432W; BLASTP E-value of 1.3e-04 over 31.8% of the total protein length), Drosophila CG6043, which has also been associated with intracellular transport and regulation of transcription from the Pol II promoter (GADFLY:CG6043-PA; BLASTP E-value of 1.9e-06 over 35.4% of the total protein length), and human megakaryocyte stimulating factor (ENSEMBL:ENSP00000251819; BLASTP E-value of 4.7e-08 over 39.4% of the total protein length).

By “PQN-70” is meant an RNA-binding protein, U1A, that is encoded by T19B10.4, or an ortholog thereof, that functions in RNAi. Such orthologs include S. cerevisiae PUB1 (SGD:YNL016W; BLASTP E-value 4.3e-08 over 49.8% of the total protein length), which is thought to play a role in the NMD pathway in yeast, and human hypothetical protein FLJ32119 (ENSEMBL:ENSP00000331699; BLASTP E-value 1.e-05 over 63.0% of the total protein length).

By “PHD-finger protein” is meant a protein encoded by T23B12.1, or an ortholog thereof, that functions in RNAi. Such orthologs include human hypothetical protein (ENSEMBL:ENSP00000265155; BLASTP E-value of 1.3e-06 over 41.9% of the total protein length).

By “Acinus protein” is meant a protein encoded by M03C11.3, or an ortholog thereof, that functions in RNAi. Such orthologs include Drosophila CG12570 (GADFLY:CG12750-PA; BLASTP E-value of 1.9e-08 over 57.8% of the total protein length), mouse Acinus (SW:ACIN_MOUSE; apoptotic chromatin condensation inducer in the nucleus) (BLASTP E-value of 1.5e-05 over 59.6% of the total protein length), and human translation initiation factor IF-2 (ENSEMBL:ENSP00000289371; BLASTP E-value of 0.0073 over 18.8% of the total protein length).

By “Nucleosomal protein” is meant a protein encoded by Y71G10AL.1, or an ortholog thereof, that functions in RNAi. Such orthologs include Drosophila CG7107 (GADFLY:CG7107-PA; BLASTP E-value of 1.6e-05 over 32.1% of the total protein length), S. cerevisiae FPR3 (SGD:YML074C; BLASTP E-value of 2.2e-05 over 20.0% of the total protein length), and human hypothetical protein (TR:Q7Z4R6; BLASTP E-value of 6.6e-06 over 15.2% of the total protein length).

By “HAT subunit” is meant ZK1127.3, or an ortholog thereof, that functions in RNAi. Such orthologs include S. cerevisiae EAF7 (Esa1-associated factor) (SGD:YNL136W; BLASTP E-value of 7.7e-05 over 40.3% of the total protein length), a putative subunit of the NuA4 histone acetyltransferase complex that specifically acetylates nucleosomal histone H4.

By “PQN-28/SIN3 HDAC complex subunit” is meant a protein encoded by F02E9.4, or an ortholog thereof, that functions in RNAi. Such orthologs include S. cerevisiae SIN3 (SGD:YOL004W; BLASTP E-value of 5.6e-47 over 45.3% of the total protein length), related proteins in Drosophila, Xenopus, and mice, and human Sin3b (ENSEMBL:ENSP00000248054; BLASTP E-value of 5.8e-55 over 44.7% of the total protein length).

By “HDAC” is meant a protein encoded by R06C1.1, or an ortholog thereof, that functions in RNAi. Such orthologs include S. cerevisiae RPD3 (reduced potassium dependency) (BLASTP E-value of 3.2e-136 over 90.1% of the total protein length), similar proteins in Drosophila, Xenopus, and mice (GADFLY:CG8815-PA, TR:Q9W6S7, SW:SN3B_MOUSE), and human histone deacetylase 1 (ENSEMBL:ENSP00000271095; BLASTP E-value of 1.2e-160 over 96.8% of the total protein length). R06C1.1 was given the locus name hda-3 due to its predicted function as a histone deacetylase. Other orthologs of R06C1.1 include Z. mays TR:Q94F82; P. polycephalum TR:Q8T7M1; A. thaliana SW:HDAC_ARATH; TR:Q8HOW2; S. cerevisiae SGD:YNL330C; G. gallus SW:HDA3_CHICK; S. cerevisiae SGD:YNL330C G. gallus; S. pombe SW:CLR6_SCHPO; H. sapiens ENSEMBL:ENSP00000302967; TR:Q9H368; SW:Q9BY41; ENSEMBL:ENSP00000316586; D. melanogaster GADFLY:CG2128-PA; GADFLY:CG6170-PA C. briggsae BP:CBP18408BP:CBP09368; S. cerevisiae SGD:YGL194C; SGD:YPR068C; SGD:YNL021W; and C. elegans WP:CE01472.

By “NCL-1” is meant a protein encoded by ZK112.2, or an ortholog thereof, that functions in RNAi. Such orthologs include Drosophila gene brat (brain tumor) (GADFLY:CG10719-PA; BLASTP E-value of 7.91e-163 over 89.4% of the total protein length), and human splice isoform alpha a 075382 tripartite motif protein 3 (ENSEMBL:ENSP00000227588; BLASTP E-value of 9.6e-48 over 59.5% of the total protein length).

By “KSHV LANA” is meant a protein encoded by T19B4.5, or an ortholog thereof, that functions in RNAi. Such orthologs include Kaposi's sarcoma-associated herpesvirus (KSHV) latent nuclear antigen (LANA) (TR:Q9DUM3; BLASTP E-value of 4.5e-10 over 94.8% of the total protein length).

By “DPY-20” is meant a protein encoded by T22B3.1, or an ortholog thereof, that functions in RNAi.

By “MTK-1 MAPKKK” is meant a protein encoded by B0414.7, or an ortholog thereof, that functions in RNAi. Such orthologs include human MTK1/MEKK4 (ENSEMBL:ENSP00000297332; BLASTP E-value of 1.6e-64 over 73.2% of the total protein length).

By “MAPKK” is meant a protein encoded by ZC449.3, or an ortholog thereof, that functions in RNAi. Such orthologs include human MAPKK 4 (MKK4) (ENSEMBL:ENSP00000262445; BLASTP E-value of 7.5e-56 over 81.2% of the total protein length).

By “Ribosome inactivating protein domain” is meant a protein encoded by T01C3.8, or an ortholog thereof.

By “NPP-16” is meant a protein encoded by Y56A3A.17, which has RAN-binding protein activity or acts as a nuclear pore protein, or an ortholog thereof. Such orthologs include Drosophila CG2158 (GADFLY:CG2158-PA; BLASTP E-value of 8.3e-14 over 63.7% of the total protein length) and human nucleoporin (ENSEMBL:ENSP00000330758; NUP50 or NPAP60L) (BLASTP E-value of 7.3e-12 over 48.0% of the total protein length).

By “Ubiquitin carboxyl-terminal hydrolase” is meant a protein encoded by F37B12.4 or an ortholog thereof, that functions in RNAi. Such orthologs include human ubiquitin carboxyl-terminal hydrolase 24 (ENSEMBL:ENSP00000294383; BLASTP E-value of 1.8e-54 over 32.9% of the total protein length).

By “F52G2.2” is meant a protein encoded by F52G2.2, or an ortholog thereof, that functions in RNAi.

By “REG cocktail” is meant a pharmaceutical composition comprising at least one and typically multiple REG polypeptides in an excipient.

By “anti-sense” is meant a nucleic acid sequence, regardless of length, that is complementary to the coding strand or mRNA of a nucleic acid sequence. Desirably the anti-sense nucleic acid is capable of decreasing the expression or biological activity of a nucleic acid or amino acid sequence. In a desirable embodiment, the decrease in expression or biological activity is at least 10%, relative to a control, more desirably 25%, and most desirably 50% or more. The anti-sense nucleic acid may contain a modified backbone, for example, phosphorothioate, phosphorodithioate, or other modified backbones known in the art, or may contain non-natural internucleoside linkages.

“Cell” as used herein may be a single-cellular organism, cell from a multi-cellular organism, or it may be a cell contained in a multi-cellular organism.

By “derived from” is meant isolated from or having the sequence of a naturally occurring sequence (e.g., a cDNA, genomic DNA, synthetic, or combination thereof).

By “differentially expressed” is meant having a difference in the expression level of a nucleic acid or polypeptide. This difference may be either an increase or a decrease in expression, when compared to control conditions.

By “double stranded RNA” is meant a complementary pair of sense and antisense RNAs regardless of length. In one embodiment, these dsRNAs are introduced to an individual cell, tissue, organ, or to a whole animals. For example, they may be introduced systemically via the bloodstream. Desirably, the double stranded RNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more.

By “duplex” is meant a domain containing paired sense and antisense nucleobase oligomeric strands. For example, a duplex comprising 29 nucleobases contains 29 nucleobases on each of the paired sense and antisense strands.

By “hybridize” is meant pair to form a double-stranded complex containing complementary paired nucleobase sequences, or portions thereof, under various conditions of stringency. (See, e.g., Wahl. and Berger, Methods Enzymol 152:399 (1987); Kimmel, Methods Enzymol 152:507 (1987)) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180 (1977)); Grunstein and Hogness (Proc Natl Acad Sci USA 72:3961 (1975)); Ausubel et al. (Current Protocols in Molecular Biology, Wiley Interscience, New York (2001)); Berger and Kimmel (Guide to Molecular Cloning Techniques, Academic Press, New York, (1987)); and Sambrook et al. (Molecular Cloning: A Laboratory Manual, Cold Spring Harbor Laboratory Press, New York). Preferably, hybridization occurs under physiological conditions. Typically, complementary nucleobases hybridize via hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “immunological assay” is meant an assay that relies on an immunological reaction, for example, antibody binding to an antigen. Examples of immunological assays include ELISAs, Western blots, immunoprecipitations, and other assays known to the skilled artisan.

By an “inhibitory nucleobase oligomer” is meant a dsRNA, siRNA, shRNA, or mimetic thereof that inhibits the expression of a target gene. An inhibitory nucleobase oligomer typically reduces the amount of a target mRNA, or protein encoded by such mRNA, by at least 5%, more desirable by at least 10%, 25%, 50%, or even by 75%, 85%, or 90% relative to an untreated control. Methods for measuring both mRNA and protein levels are well-known in the art; exemplary methods are described herein.

Preferably a nucleobase oligomer of the invention includes from about 8 to 30 nucleobases. A nucleobase oligomer of the invention may also contain, for example, an additional 20, 40, 60, 85, 120, or more consecutive nucleobases that are complementary to a polynucleotide of interest. The nucleobase oligomer (or a portion thereof) may contain a modified backbone. Phosphorothioate, phosphorodithioate, and other modified backbones are known in the art. The nucleobase oligomer may also contain one or more non-natural linkages.

By “isolated polynucleotide” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “mutagenized” is meant comprising a mutation. Mutations may be naturally occurring or induced by contacting a cell or organism with any agent that induces a break or alteration in a nucleic acid, preferably a genomic nucleic acid. Such agents are known to the skilled artisan and include radiation (e.g., U.V., gamma, and X-rays) and chemical agents (e.g., ethylmethanesulfonate (EMS), aflatoxin B1, nitrosoguanidine).

“Microarray” means a collection of nucleic acid molecules or polypeptides from one or more organisms arranged on a solid support (for example, a chip, plate, or bead). These nucleic acid molecules or polypeptides may be arranged in a grid where the location of each nucleic acid molecule or polypeptide remains fixed to aid in identification of the individual nucleic acid molecules or polypeptides. A microarray may include, for example, nucleic acid molecules representing all, or a subset, of the open reading frames of an organism, or of the polypeptides that those open reading frames encode. A microarray may also be enriched for a particular type of gene.

By “nucleic acid” is meant an oligomer or polymer of ribonucleic acid or deoxyribonucleic acid, for example, a dsRNA, siRNA, shRNA, or mimetic thereof. This term includes oligomers consisting of naturally occurring bases, sugars, and intersugar (backbone) linkages as well as oligomers having non-naturally occurring portions which function similarly. Such modified or substituted oligonucleotides are often preferred over native forms because of properties such as, for example, enhanced cellular uptake and increased stability in the presence of nucleases.

Specific examples of some preferred modified nucleic acids or nucleobases envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton and Weller, U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497 (1991)). Other preferred oligonucleotides may contain alkyl and halogen-substituted sugar moieties comprising one of the following at the 2′ position: OH, SH, SCH₃, F, OCN, O(CH₂)ₙNH₂ or O(CH₂)ₙCH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. Oligonucleotides may also have sugar mimetics such as cyclobutyls in place of the pentofuranosyl group.

Other preferred embodiments may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines. Each of the above is referred to as a “modification” herein.

By a nucleobase oligomer that “reduces the expression” of a target gene is meant one that decreases the amount of a target mRNA, or polypeptide encoded by such mRNA, by at least about 5%, more desirable by at least about 10%, 25%, or even 50%, relative to an untreated control. Methods for measuring both mRNA and polypeptide levels are well-known in the art; exemplary methods are described herein.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “ortholog” is meant a polypeptide or nucleic acid molecule of an organism that is highly related to a reference polypeptide, or nucleic acid sequence, from another organism. An ortholog is functionally related to the reference polypeptide or nucleic acid sequence. In other words, the ortholog and its reference molecule would be expected to fulfill similar, if not equivalent, functional roles in their respective organisms. It is not required that an ortholog, when aligned with a reference sequence, have a particular degree of amino acid sequence identity to the reference sequence. A polypeptide ortholog might share significant amino acid sequence identity over the entire length of the polypeptide, for example, or, in another embodiment, might share significant amino acid sequence identity (e.g., at least 20%, 25%, 30%, 40%, more preferably, at least 50%, 60%, 75%, or most preferably, at least 85%, 90%, or 95%) over only a single functionally important domain of the polypeptide. Such functionally important domains may be defined by genetic mutations or by structure function assays. Orthologs may be identified using methods provided herein. The functional role of an ortholog may be assayed using methods well known to the skilled artisan, and described herein. For example, function might be assayed in vivo or in vitro using a biochemical, immunological, or enzymatic assays; transformation rescue, or in a bioassay for the effect of gene inactivation on nematode phenotype as described herein. In another embodiment, bioassays may be carried out in tissue culture; function may also be assayed by gene inactivation (e.g., by RNAi, siRNA, or gene knockout), or gene over-expression, as well as by other methods.

By “pathogen” is meant a bacteria, virus, fungus, nematode, insect, tick, arachnid or other creature which is capable of infecting or infesting host, and in particular, a plant or vertebrate animal.

By “polypeptide” is meant any chain of amino acids, or analogs thereof, regardless of length or post-translational modification (for example, glycosylation or phosphorylation).

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “promoter” is meant a polynucleotide sufficient to direct transcription.

By “purified antibody” is meant an antibody that is at least 60%, by weight, free from polypeptides and naturally occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinant polypeptide of the invention and standard techniques.

By “refractory to RNAi” is meant a cell or gene that is resistant to the gene silencing effects of an inhibitory nucleic acid. Cells and genes that are refractory to RNAi fail to exhibit at least a 10%, 25%, 50%, or 75% decrease in the level of expression of a gene targeted for RNAi relative to the level of the target gene's expression present in an untreated control cell.

By “reporter gene” is meant a gene encoding a polypeptide whose expression may be detected; such polypeptides include, without limitation, glucuronidase (GUS), luciferase, chloramphenicol transacetylase (CAT), and beta-galactosidase.

By “specifically binds” is meant a compound or antibody which recognizes and binds a polypeptide of the invention but which does not substantially recognize and bind other molecules in a sample, for example, a biological sample, which naturally includes a polypeptide of the invention.

By “shRNA” is meant an RNA comprising a duplex region complementary to an mRNA. For example, a short hairpin RNA (shRNA) may comprise a duplex region containing nucleoside bases, where the duplex is between 17 and 29 bases in length, and the strands are separated by a single-stranded 4, 5, 6, 7, 8, 9, or 10 base linker region. Optimally, the linker region is 6 bases in length.

By “siRNA” is meant a double stranded RNA comprising a region of an mRNA. Optimally, an siRNA is 17, 18, 19, 20, 21, 22, 23, or 24 nucleotides in length and has a 2 base overhang at its 3′ end. siRNAs can be introduced to an individual cell, tissue, organ, or to a whole animals. For example, they may be introduced systemically via the bloodstream. Such siRNAs are used to downregulate mRNA levels or promoter activity. Desirably, the siRNA is capable of decreasing the expression or biological activity of a nucleic acid or amino acid sequence. In one embodiment, the decrease in expression or biological activity is at least 10%, relative to a control, more desirably 25%, and most desirably 50%, 60%, 70%, 80%, 90%, or more. The siRNA may contain a modified backbone, for example, phosphorothioate, phosphorodithioate, or other modified backbones known in the art, or may contain non-natural internucleoside linkages. Such siRNAs are used to downregulate mRNA levels or promoter activity.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and most preferably 90% or even 95% identical at the amino acid level or nucleic acid level to the sequence used for comparison. The comparison is over at least 25-50 nucleotides, more preferably 50-100 or 100-200 nucleotides, and most preferably 200-400, 400-600, 600-800, or even 800-1000 nucleotides.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “targets a gene” means specifically binds to and decreases the expression of the gene. For example, an inhibitory nucleic acid binds to and decreases the expression of a complementary target gene. Such a decrease is by at least 10%, 25%, 50%, 75%, or 100% relative to the expression of a corresponding control gene.

By “transformed cell” is meant a cell into which (or into an ancestor of which) has been introduced, by means of recombinant DNA techniques, a polynucleotide molecule encoding (as used herein) a polypeptide of the invention.

By “transgene” is meant any piece of DNA that is inserted by artifice into a cell and becomes part of the genome of the organism that develops from that cell or, in the case of a nematode transgene, becomes part of a heritable extrachromosomal array. Such a transgene may include a gene which is partly or entirely heterologous (i.e., foreign) to the transgenic organism, or may represent a gene homologous to an endogenous gene of the organism.

By “transgenic” is meant any cell which includes a DNA sequence which is inserted by artifice into a cell and becomes part of the genome of the organism which develops from that cell or part of a heritable extrachromasomal array. As used herein, the transgenic organisms are generally transgenic invertebrates, such as C. elegans, or vertebrates, such as, zebrafish, mice, and rats, and the DNA (transgene) is inserted by artifice into the nuclear genome or into a heritable extrachromasomal array.

The invention provides methods and compositions that are useful for enhancing RNAi. In addition, the methods of the invention provide a facile means to identify therapies that are safe for use in eukaryotic host organisms (i.e., compounds that do not adversely affect the normal development, physiology, or fertility of the organism). Other features and advantages of the invention will be apparent from the detailed description, and from the claims.

### BRIEF DESCRIPTION OF THE DRAWINGS

FIG. 1 shows a schematic diagram that depicts a molecular model of the RNAi mechanism.

FIG. 2 shows a schematic diagram that depicts a cellular model of the RNAi mechanism.

FIG. 3 shows a diagram that illustrates the construction of the GR1401 (same as Is41) RNAi sensor strain. GR1401 refers to the same strain as Is41. The C. elegans GR1401 (Is41) strain carries a transgene containing a snapback GFP construct under the control of the wrt-2 seam cell-specific promoter, which was introduced into JR672, a C. elegans strain expressing GFP under the control of a seam-cell specific promoter (Koh and Rothman, Development 128:2867 (2001)). In untreated GR1401 (Is41) nematodes, seam-cell GFP expression is suppressed by transgenic dsRNA expression, which targets GFP. When an essential component of the RNAi machinery is silenced, GFP expression is no longer suppressed by RNAi, resulting in restoration of normal levels of GFP, which can be visualized using UV microscopy.

FIG. 4 shows a flow chart illustrating the screening strategy. A library of 18,578 bacterial strains capable of silencing 94% of all C. elegans predicted genes by RNAi was assembled. To comprehensively screen the C. elegans genome for genes involved in RNAi, each of these clones was sequentially fed to the GR1401 (Is41) C. elegans strain. Feeding began at the L1 stage and continued until the animals were assayed for an RNAi-deficient phenotype in the L4-stage F1 progeny. Animals having decreased expression of an RNAi essential gene displayed increased expression of GFP in their seam cells. Those clones that were able to reproducibly generate high levels of GFP expression (≧2 on a scale from 0-4) during multiple experiments underwent further analysis.

FIG. 5 shows a schematic diagram showing a pseudo-interaction map of new genes implicated in RNAi. Each node represents a single predicted gene, and each line connecting two nodes represents a possible interaction based on the biology of known homologs in other organisms, as detailed in the text. Interactions reflected here include physical binding, association as part of a complex, signaling, or modification of one predicted polypeptide with another. These putative interactions may be substantiated by two-hybrid or mass spectrometry analyses to demonstrate a physical association or epistasis analysis to demonstrate involvement in a single pathway. The color scheme reflects the different classes of genes identified in Table 2.

FIG. 6 shows a table showing the gene classes that were identified in a screen for RNA essential genes that are also required for embryonic development.

FIGS. 7A and 7B show a scheme for a genome-wide screen to identify factors required for RNAi. FIG. 7A shows the RNAi sensor strain (Is41 or GR1401) is comprised of a GFP reporter expressed in the lateral seam cells along with a chromosomally-integrated plasmid expressing a dsRNA GFP hairpin in the seam cells which abrogates the reporter expression by RNAi. FIG. 7B shows inhibiting the RNAi pathway by feeding the RNAi sensor strain E. coli expressing dsRNA corresponding to the core RNAi genes dcr-1, rde-1, and rde-4 (but not empty control vector) restores the reporter GFP expression in the seam cells.

FIG. 8 shows gene silencing in the germline requires a subset of new RNAi factors. In the let-858::gfp reporter strain (PD7271), GFP is normally silenced in the germline (area between the two sets of dotted lines in each panel) but is expressed in nearly all somatic tissues (Kelly et al., Genetics 146:227 (1997)). GFP expression of let-858::gfp in the germline is restored when RNAi of mut-7 or mut-16 is sustained for two generations. However, rde-1, which is essential for RNAi in the soma, is dispensible for germline gene silencing (left panels). In total, five genes known to be required for RNAi (dcr-1, drh-1, mes-4, mut-16, and mut-7) were required for germline gene silencing. In addition, nine new factors were also required for gene silencing in the germline, including F22D6.6 (a Tudor domain protein), M03C11.3 (a chromatin associated factor) and R03D7.4 (homolog of RNA polymerase II transcription elongation factor Elongin) (right panels). Other new factors required for germline silencing are the chromatin factor HDA-3, the RNA binding/processing factors RNP-2 and F43G9.5, the transcription factors ZK1127.6 and ZK1127.9, and two factors of unknown function, T01C3.8 (RDE-5) and ZK1127.3. Control denotes PD7271 worms fed on control vector bacteria.

FIGS. 9A and 9B show transgene silencing in the soma requires many new RNAi genes. Transgene silencing is enhanced in the eri-1 (mg366) genetic background: both the seam-cell GFP reporter in eri-1 (GR1402) and the ubiquitously expressed sur-5::gfp reporter in eri-1 (mg366) (GR1403) show near-complete transgene silencing in non-neuronal somatic tissues (“control” panels in 9A and 9B, respectively). FIG. 9A shows that seam cell GFP expression is restored in GR1402 when subjected to RNAi of dcr-1 or mut-16. Of the 90 RNAi genes tested, RNAi inactivation of 87 restored GFP expression in GR1402. RNAi to hda-3 is shown as an example. FIG. 9B shows that expression of GFP in most somatic tissues is restored in GR1403 upon inactivation of known factors required for RNAi (dcr-1, mut-16, mes-4, or rsd-2), as well as the majority of the tested genes identified in the RNAi sensor screen. RNAi to ncl-1, T19B4.5 (unknown function), hda-3, and Y71G10AL.1 (RNA binding/processing) are shown as examples. See FIG. 11, FIG. 16, and FIG. 17 for details.

FIG. 10 is a table showing genes required for RNAi identified by the genome-wide RNAi sensor screen. Predicted homologs of the C. elegans genes were determined by BLASTP against the human (H.s.), mouse (M.m.), fly (D.m.), and fission yeast (S.p.) proteomes (see Methods and FIG. 15 for additional information). Dark grey boxes indicate best reciprocal BLASTP match with the C. elegans polypeptide, and light grey boxes indicate homology with a BLASTP e-value ≦10⁻⁶. “F1” designates that RNAi of the corresponding gene resulted in viable progeny whereas RNAi inactivation of “P₀” genes resulted in lethality in the progeny generation. “GFP” indicates average fluorescence scores, on a 0 to 4 scale (see Methods for details). GenBank accession numbers are listed for each gene; where several splice isoforms were detected the accession number for the largest isoform is listed. One clone targets two highly homologous predicted polypeptides, ZK1127.9 and ZK1127.6 (BLASTP e-value >10⁻²⁰¹ over <80% of total polypeptide length). The homology summary for ZK1127.6 is shown at the top of the cell, and the summary for ZK1127.9 is found at the bottom.

FIGS. 11A and 11B are a group of tables showing functions of genes identified in RNAi and related pathways. FIG. 11A shows a summary of assays performed with RNAi clones that result in viable progeny (“Viable Clones/F1”) and FIG. 11B shows the transgene silencing assay for the RNAi clones that cause embryonic lethality (“Lethal Clones/P0”). The dsRNA co-injection assay involves injecting dsRNA from each of the candidate RNAi genes with mom-2 dsRNA, a gene essential for embryonic viability. The survival of progeny indicates that inactivation of the candidate gene confers resistance to mom-2 RNAi. The assay was performed on the 36 RNAi clones that result in viable progeny. A “+” notation identifies genes whose dsRNA injection resulted in >45% viability with a p-value <0.05. Details are discussed in the text; see also FIG. 12 and Methods. Transgene silencing in somatic tissues, which is enhanced in the eri-1 background, was analyzed using either the seam-cell marker GFP (scm::gfp; GR1402) or the ubiquitously expressed sur-5::gfp reporter (sur-5::gfp; GR1403). Germline transgene silencing for the viable clones was assessed in the let-858::gfp reporter strain (PD7271). See text, FIGS. 16 and 17, and Methods for additional details.

FIG. 12 shows co-injection analysis with dsRNAs of genes identified in the RNAi sensor screen. Wild type (N2) young adult worms were co-injected with 300 ng/μl of each candidate dsRNA and 100 ng/μl mom-2 dsRNA. Protection from mom-2 lethality by RNAi was determined by scoring viability (eggs hatched/total eggs laid) of progeny in two consecutive egg-lays. The dsRNAs corresponding to the 36 genes that result in viable progeny were tested, including genes previously identified to be required for RNAi (“Known RNAi”) and new genes identified in the RNAi sensor screen (“RNAi Sensor Positive”). The red line indicates the cut-off for viability (>45%; p-value <0.05) used to define the positive genes reported in FIG. 11. Co-injection of dsRNA to all genes identified by the RNAi sensor screen that were previously implicated in RNAi show greater than 45% viability, indicating strong protection from mom-2 dsRNA induced lethality. dsRNA corresponding to ten genes produced an equivalent level of protection (>45%) upon coinjection, suggesting that these factors are required for robust RNAi. Notably, co-injection of dsRNA corresponding to T01C3.8, the gene mutated in rde-5 (C. Mello, personal communication), was not protective, indicating that not all genes necessary for RNAi will show robust protection in this assay. As controls for nonspecific protection by co-injected dsRNA, several additional genes were co-injected with mom-2. dsRNAs corresponding to lin-1, bli-4, and dpy-10 as a well as six genes (CD4.4, asp-5, F46E10.9, C14C11.5, F58A4.11, C36H8.1) that scored negative in the RNAi sensor screen were tested. These six genes were selected to represent a wide range of expression levels as indicated by SAGE data (http://tock.bcgsc.bc.ca/cgi-bin/sage), strength of phenotype upon knock-down (Kamath et al., Nature 421:231 (2003)), and molecular annotations (http://www.wormbase.org). None of the genes tested resulted in higher than 40% viability post-injection, with an average survival rate of 24% for all negative control dsRNAs injected.

FIG. 13 shows interactome analysis of factors required for RNAi and indicates integration of RNAi machinery into a wide range of RNA-mediated cellular processes. Polypeptide interaction data for 42 of the 92 factors identified in the GR1401 RNAi sensor screen were constructed by searching for direct interactors with these ORFs in the data sets of WI5 (L1 et al., Science 303:540 (2004)), new interactions identified for several ORFs in this study, and two published interactions not included in WI5 (see FIG. 18A). In total, the map consists of 161 direct interactions. RNAi factors identified in the present screen are indicated in dark grey and all other interactors are indicated in light grey and white. Highlighted regions: A) A region of the map that connects several factors required for RNAi, including factors known to function in RNAi (RSD-2, RSD-6), newly identified RNAi components (e.g., W05H7.4, F56A8.6), and NMD machinery (e.g., SMG-2, T25G3.3). B) A direct interaction between a novel RNAi factor, T 19B10.4, and RDE-4. C) Two new RNAi components (NPP-1 and ZK1127.3) are linked by a common interactor, MRG-1, which is required for somatic transgene silencing in the eri-1 background (data not shown).

FIG. 14 is a table showing RNAi clones from the Orfeome RNAi library corresponding to genes not targeted by the Ahringer RNAi library. These 1,821 clones were used to supplement the 16,757 clones from the Ahringer RNAi library for the genome-wide RNAi screen, bringing the total number of targeted loci in the screen to 18,578, or 94% of predicted C. elegans genes. WS100 gene names are listed for each predicted target. If more than one gene is predicted to be targeted by a particular RNAi clone, all potential targets are listed. Locations correspond to the plate and well addresses in the Orfeome RNAi library (Rual et al., Genome Res 14:2162 (2004)).

FIG. 15 is a table showing homologs of RNAi factors identified in C. elegans. BLAST searches identified homologs of the C. elegans RNAi factors in Homo sapiens (human), Mus musculus (mouse), Drosophila melanogaster (fly) and Schizosaccharomyces pombe (fission yeast). The highest scoring match from each organism is shown, together with the e-value and the ENSEMBL annotation when available. A BLAST e-value of ≦10⁻⁶ was used as the threshold for significant homology. Orthologs were classified based on best reciprocal BLAST hits and are indicated by an asterisk (*). In total 86% of the RNAi factors show significant homology to polypeptides in other species, and 73% have orthologs in at least one other species.

FIG. 16 is a table showing phenotypic analysis of viable/F1 RNAi clones of genes identified by the RNAi sensor screen. For the transgene silencing and germline silencing assays, the average scores are reported on a scale of 0 (no suppression of silencing) to 4 (strong suppression of silencing). P-values were calculated by t-test against L4440 vector control. Clones resulting in average fluorescence scores >1.0 were designated as positive in FIG. 11. At this cut-off, positive clones are significant to a p-value of <0.05 for eri-1; scm::gfp and <0.01 for all other assays. RNAi clones that resulted in significant defects in seam cell number (p-value <0.01) and let-7 (mg279) enhancement are highlighted in gray. See text for details. Brood sizes of the progeny were determined after feeding the parental generation starting from the L1 larval stage. SD, standard deviation; n, number of broods assayed.

FIG. 17 is a table showing phenotypic analysis of embryonic lethal/P₀ RNAi clones of genes identified by the RNAi sensor screen. For the transgene silencing assay, the average scores are reported on a scale of 0 (no suppression of silencing) to 4 (strong suppression of silencing). P-values are calculated by t-test against L4440 vector control. Clones resulting in average fluorescence scores >1.0 were designated as positive in FIG. 11. At this cut-off, positive clones are significant to a p-value of <0.01. RNAi clones that resulted in significant defects in seam cell number (p-value <0.01) and let-7 (mg279) enhancement are highlighted in gray. See text for details. SD, standard deviation.

FIGS. 18A-18C are a group of tables showing data related to protein-protein interaction factors. FIG. 18A is a table showing protein-protein interactions for factors identified in the RNAi sensor screen. WI5 datasets (L1 et al., Science 303:540 (2004)) from which the protein-protein interactions were identified are as follows: “CORE_{—}1”: (Yeast two-hybrid) Y2H interactions that were found at least three times independently; “CORE_{—}2”: Y2H interactions that were found fewer than three times but passed the retests; “INTERLOG”: in silico searches of orthologous pairs known to interact in one or more species; “LITERATURE”: known interactions identified from annotations in the Worm Proteome Database (http://www.proteome.com/) maintained by Biobase Biological Databases (Wolfenbüttel, Germany); “SCAFFOLD”: Previous interactome maps generated for specific biological processes. Interactions detected in both directions are noted with an asterisk (*). Interactions detected by Y2H or co-immunoprecipitation assays in published reports but not recorded in WI5 are indicated with ¶. FIG. 18B shows a summary of RNAi factors for which interactions were detected. Genes that were analyzed as baits in WI5 but that generated no interactions are indicated with #. FIG. 18C shows factors that interact with the RNAi factors and also required for somatic transgene silencing. Interactors not found to be positive in the RNAi sensor screen were tested for suppression of somatic transgene silencing in the GR1402 strain (eri-1 (mg366); [wIs54(scm:gfp)]) (see the legend of FIG. 13). 21 genes were found to be positive in the somatic transgene silencing assay, with the 0-4 GFP scores listed for each positive gene.

FIG. 19 is a table showing a summary of known RNAi factors detected in the RNAi sensor screen. Twenty six genes have been implicated in some aspect of RNAi function in C. elegans. The RNAi sensor screen identified nearly all of the core RNAi factors that have shown to be required in the soma (dcr-1, rde-1, rde-4, drh-1 (Tabara et al., Cell 109:861 (2002))). The somatic RNAi factors rde-3 and rrf-1 (Chen et al., Curr Biol published online Jan. 13, 2005; Tijsterman et al., Annu Rev Genet 36:489 (2002)) were not detected as positive in the screen, but rde-5 was discovered (this study; C. C. Mello, personal communications). rrf-1 was not detected because a feeding clone corresponding to the gene was not in the available RNAi libraries. We have subsequently obtained the feeding strain and shown that it is positive with the RNAi sensor strain (J. K. Kim, et al., data not shown) (indicated by ¶, clone provided by S. Fischer and R. Plasterk). Thus, the RNAi sensor screen detected 6 of 7 genes (86%) that were expected a priori to be positive in the screen. The remaining 19 genes function in RNAi persistence, spreading, or germline RNAi and therefore it was unclear whether they should have been identified in this RNAi screen (Tijsterman et al., Curr Biol 14:111 (2004), Vastenhouw and Plasterk, Trends Genet 20:314 (2004); Tijsterman et al., Curr Biol 12:1535 (2002); Tijsterman et al., Science 295:694 (2002); Caudy et al., Nature 425:411 (2003); Tops et al., Nucleic Acids Res 33:347 (2005); Dudley et al., Proc Natl Acad Sci USA 99:4191 (2002); Domeier et al., Science 289:1928 (2000)). The RNAi screen did identify 7 of these 19 genes including smg-2, a gene necessary for RNAi persistence (Domeier et al., Science 289:1928 (2000)); mut-7 and mut-16, genes previously characterized as required for germline RNAi (Tijsterman et al., Annu Rev Genet 36:489 (2002); Vastenhouw and Plasterk, Trends Genet 20:314 (2004)); and rsd-2, a gene required for RNAi spreading (Tijsterman et al., Curr Biol 14:111 (2004)). Detection of rsd-2 by the RNAi sensor strain suggests that it may have novel cellautonomous function in RNAi in addition to its role in RNAi spreading. Furthermore, the identification of smg-2 by the screen indicates that the sensor strain can identify factors that may be required in one aspect of silencing RNAi such as persistence, but that have not been identified by canonical Rde screens because of their subtle phenotypes. Overall, these results demonstrate that the RNAi sensor strain provides an effective reporter for detecting genes necessary for RNAi. *rsd-6 was not detected by the RNAi sensor screen but was subsequently identified as an RSD-2 interactor that functions in somatic transgene silencing (see FIG. 13 and FIG. 18C).

FIG. 20 is a table showing genes required for RNAi identified by the genome-wide RNAi sensor screen using the C. elegans GR1402 strain. Predicted homologs of the C. elegans genes were determined by BLASTP against the human (H.s.), mouse (M.m.), fly (D.m.), and fission yeast (S.p.).

FIG. 21 is a table showing the C. elegans genes identified in the GR1402 screen with human homologs that lack a known function.

### DESCRIPTION OF THE INVENTION

The present invention features methods and compositions useful for modulating RNAi in a wide variety of cell types.

As described below, 92 genes that are essential for RNAi were identified in a systematic screen of the C. elegans genome. Orthologs of these genes were also identified. Using a similar C. elegans strain including a mutation that enhances RNAi (eri-1), an additional 446 genes involved in the RNAi pathway were identified, along with their orthologs. Given the evolutionary conservation of the RNAi pathway, these orthologs are also likely to be required for RNAi. As described in more detail below, methods that increase the expression of an RNAi essential gene in a cell or organism enhance RNAi in that cell or organism, while methods that inhibit the expression of an RNAi essential gene reduce the efficacy of RNAi in a cell or organism.

RNAi Pathway

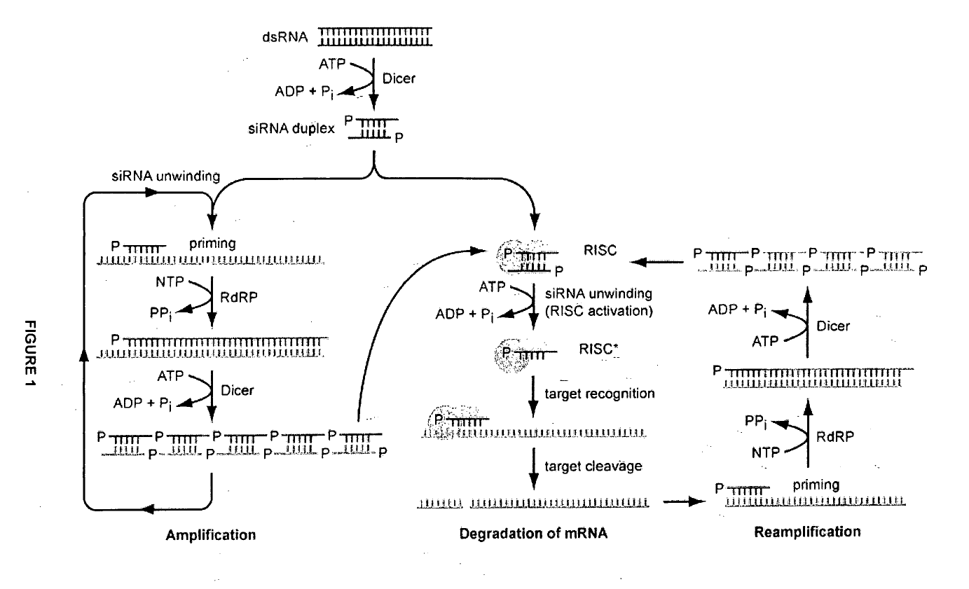

RNAi-like phenomena have been found in many highly-divergent species of eukaryotes, and it is likely that the RNAi machinery was developed before the evolutionary divergence of animals, plants, and fungi. The RNAi pathway includes a variety of conserved components that work together to degrade dsRNA as shown in FIG. 1. Post-transcriptional gene silencing by RNAi is a conserved process by which dsRNA triggers the destruction of homologous target mRNAs (Meister and Tuschl, Nature 431:343 (2004)). RNAi-related mechanisms also regulate transcriptional gene silencing by guiding heterochromatin formation at targeted promoters and regions of repetitive DNA elements such as centromeres (Lippman and Martienssen, Nature 431:364 (2004)). In various systems, RNAi has been shown to perform other important endogenous functions, including silencing of transposable elements and transgene arrays, destruction of aberrant mRNAs, triggering heterochromatin formation for proper chromosome segregation, and controlling genome rearrangements (Vastenhouw and Plasterk, Trends Genet 20:314 (2004); Grewal and Rice, Curr Opin Cell Biol 16:230 (2004); Mochizuki and Gorovsky, Curr Opin Genet Dev 14:181 (2004)). In animals, RNAi-related mechanisms play essential roles in development including maintenance of stem cell fates, patterning of the nervous system, hematopoiesis, cell death, and fat storage, while mis-regulation leads to unchecked polypeptide expression and has been implicated in cancerous cell proliferation (Bartel, D. P., Cell 116:281 (2004); Ambros, V., Nature 431:350 (2004); McManus, M. T., Semin Cancer Biol 13:253 (2003))

Studies in fungi, plants, worms, flies, and mammals have provided a basic framework for the mechanism of RNAi-mediated gene silencing. First, long dsRNAs are recognized and cleaved into 21-25 nt small interfering RNAs (siRNAs) by the RNase III nuclease Dicer (Bernstein et al., Nature 409:363 (2001); Knight and Bass, Science 293:2269 (2001); Zamore et al., Cell 101:25 (2000); Hannon, Nature 418:244 (2002)). Dicer contains a PAZ domain, RNase III domains, a dsRNA-binding domain, and a helicase domain that requires ATP for processive movement along the dsRNA substrate. As shown in FIG. 1, Dicer acts to generate siRNAs from input dsRNA. These siRNAs are then incorporated into an effector complex called the RNA Induced Silencing Complex (RISC), which guides siRNAs to base-pair with mRNA targets containing complementary sequences (Meister and Tuschl, Nature 431:343 (2004); Hammond et al., Nature 404:293 (2000)). The target mRNA is destroyed by complexing siRNAs with RISC. The subsequent cleavage of the mRNA by the RISC endonuclease potently inhibits expression of the target gene (Schwarz et al., Curr Biol 14:787; Martinez and Tuschl, Genes Dev 18:975 (2004); Meister et al., Mol Cell 15:185 (2004); Liu et al., Science 305:1437 (2004)). RISC is a nuclease that specifically degrades mRNA. It is activated when the siRNA is unwound. Unwinding targets RISC to the correct mRNAs. Interestingly, even small amounts of dsRNA are sufficient to result in systemic RNA interference. This suggests that RNAi involves a catalytic or amplification step, perhaps involving an RNA-dependent RNA polymerase (RdRP). In another embodiment, amplification may occur by copying mRNA using unwound siRNAs to prime RdRP or by also using partially degraded aberrant mRNAs as a template. In animals, endogenous small RNAs termed microRNAs can also block translation of their mRNA targets through an analogous RISC-like mechanism (Bartel, Cell 116:281 (2004)). Furthermore, recent findings in S. pombe indicate that siRNAs can also associate with a RISC-like RITS complex that targets siRNAs to the chromosome and leads to the formation of heterochromatin and subsequent transcriptional gene silencing (Verdel et al., Science 303:672 (2004); Noma et al., Nat Genet 36:1174 (2004))

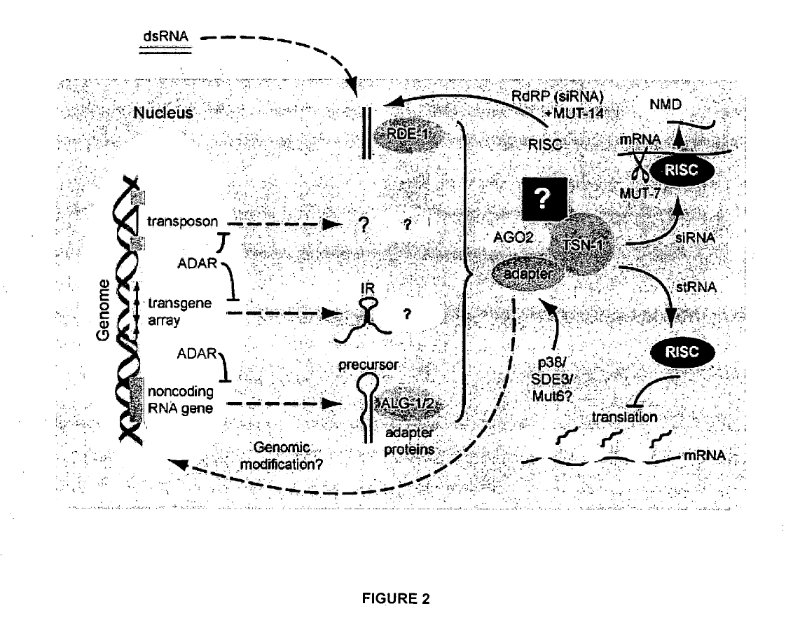

A cellular model for RNAi is shown in FIG. 2. An Argonaute-like adapter polypeptide is used to stabilize dsRNA and bring it into proximity with Dicer. For dsRNA the adapter is RDE-1, for single stranded precursors the adapter is ALG-1/2, and for transposons or transgenes, the adapter is unknown. Following Dicer-mediated cleavage, single-stranded (st) RNA products bind to mRNAs and inhibit translation. siRNAs are unwound by a helicase activity and are used to target RISC to the proper mRNA. mRNAs cleaved by RISC are recognized by the cell as aberrant and are degraded. siRNAs are also used in conjunction with an RdRP to amplify the number of siRNAs.

While genetic and biochemical studies have identified components of RNAi, biochemical analysis can miss components at steps in the pathway not being assayed or components that are in low abundance or transient in biochemical interactions. In addition, RNAi factors required for viability or fertility are likely to be under-represented in the forward genetics screens that have thus far selected for mutations with viable phenotypes. Because RNAi screens do not necessarily induce a null phenotype, a comprehensive genomic analysis by RNAi should in principle identify the complete pathway, including factors required for steps up-and downstream of the canonical dsRNA processing and mRNA cleavage events. In this study, we have employed a genome-wide approach to identify an extensive set of genes required for RNAi in C. elegans. Our findings provide a global view of how RNAi mechanisms are integrated into a wide range of RNA-mediated processes.

RNAi Essential Gene Screen

New genes required for RNAi in C. elegans were identified using high-throughput functional genomics. In order to enable genome-wide analyses of gene function by RNAi, a library of 16,757 bacterial strains was used. This library corresponded to 86% of the genome, with each strain capable of expressing a dsRNA that was designed to target a single predicted gene (Kamath et al., Nature 421:231 (2003); Kamath and Ahringer, Methods 30:313 (2003)). A similar library was obtained, containing 12,219 C. elegans ORFs, roughly corresponding to 63% of the genome, cloned into the same vector/bacteria system as part of the C. elegans ORFeome project (Reboul et al., Nat Genet 34:35 (2003)). Together, these libraries were assembled into a non-overlapping set estimated to be capable of targeting 94% of all C. elegans predicted genes, corresponding to 18,578 genes, by RNAi.

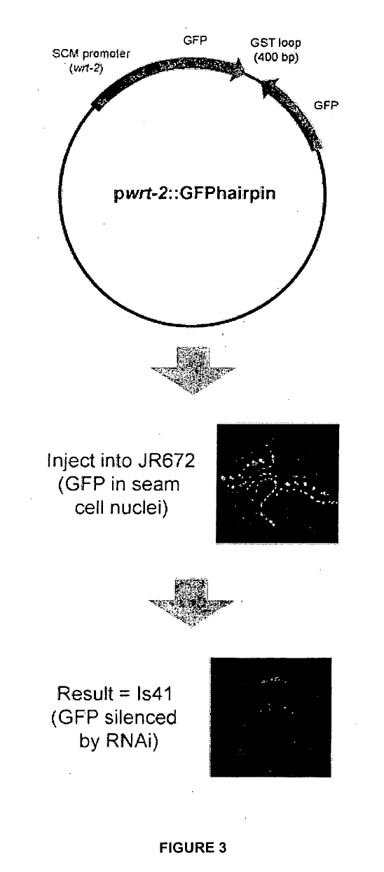

To screen for the RNAi-defective phenotype, a strain of C. elegans was developed that fluoresces when the action of RNAi is inhibited. The screen was carried out using this C. elegans strain, GR1401 (Is41), which expresses two transgenes. The first transgene encodes GFP under the control of a promoter that drives expression specifically in the seam cells. The second transgene expresses a dsRNA that interferes with this expression. The dsRNA in the GR1401 (Is41) strain is encoded by a snapback GFP construct described below, under the control of the wrt-2 seam cell-specific promoter. This transgene was introduced into the JR672 parental strain, which expresses GFP under the control of a seam-cell specific promoter (Koh et al., Development 128:2867 (2001)) (FIG. 3). In worms expressing both transgenes, GFP expression is suppressed. Each of the 18,578 non-overlapping bacterial strains from the two libraries were then fed sequentially to the C. elegans GR1401 (Is41) strain.

To carry out this screen, L1-stage larvae were fed each bacterial clone, and GFP fluorescence was monitored in their progeny at the late L4 or young-adult stage. For the 945 genes annotated as embryonic lethal (Kamath et al., Nature 421:231 (2003)), the screening procedure was modified so that L1-stage animals were fed and GFP fluorescence was monitored in the adult stage, thus bypassing the embryonic lethality caused by these clones. All experiments were scored on a GFP intensity and penetrance scale of 0 (no GFP expression) to 4 (highly penetrant, strong GFP expression) and those that scored an average of 2 or greater were designated as candidate RNAi genes (see Methods). All candidate clones were retested no fewer than five independent times in triplicate. The genes whose inactivation by RNAi restores GFP reporter expression were defined to be new RNAi factors. While it was expected that some of the genes identified to function in the canonical steps of RNAi (i.e., dsRNA processing into siRNAs and subsequent mRNA cleavage), it was anticipated that a genome-wide analysis of RNAi would reveal an expanded view of factors that modulate the pathway.

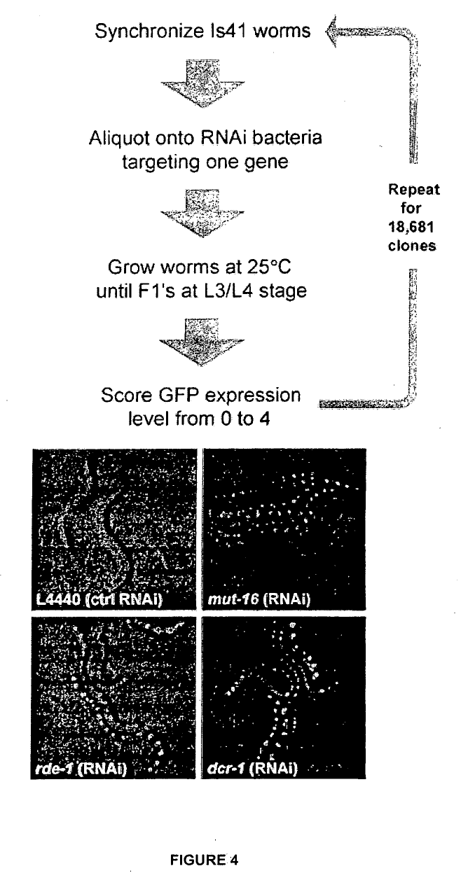

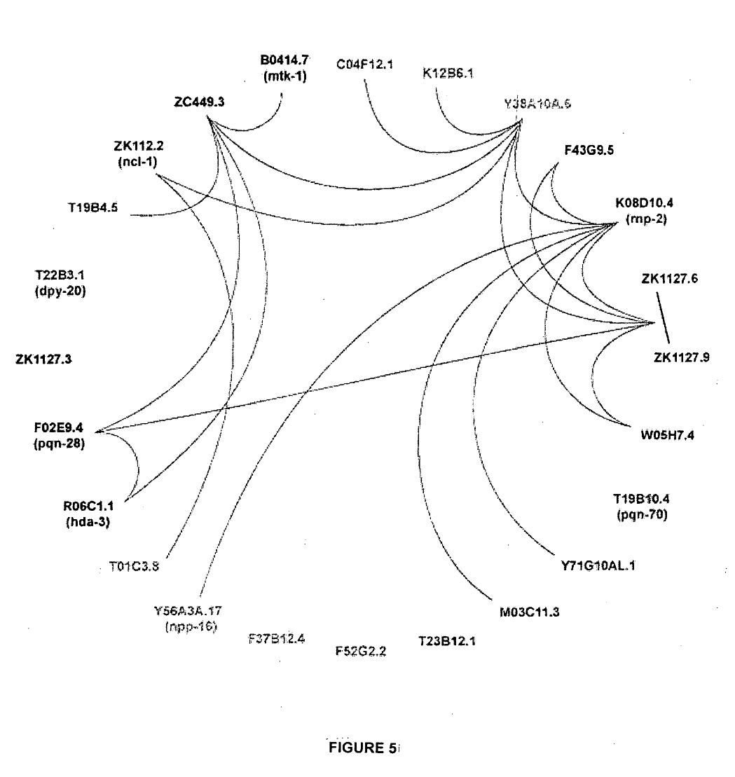

If an essential component of the RNAi machinery is silenced in GR1401 (Is41) worms, the expression of GFP is no longer suppressed, and normal levels of GFP expression can be visualized using fluorescent microscopy. The screen was performed by feeding bacterial strains expressing dsRNA corresponding to each C. elegans gene to GR1401 (Is41) worms and assaying for fluorescence in worms exposed to ultraviolet light (FIG. 4). Only those clones that were able to reproducibly generate high levels of GFP expression were considered for subsequent analyses, and all positive clones were sequenced twice to confirm their identities.

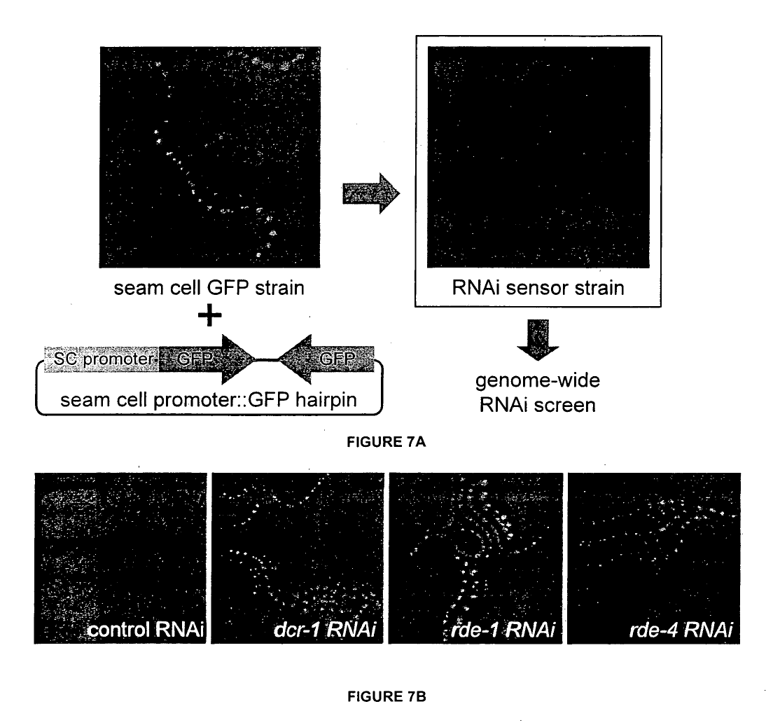

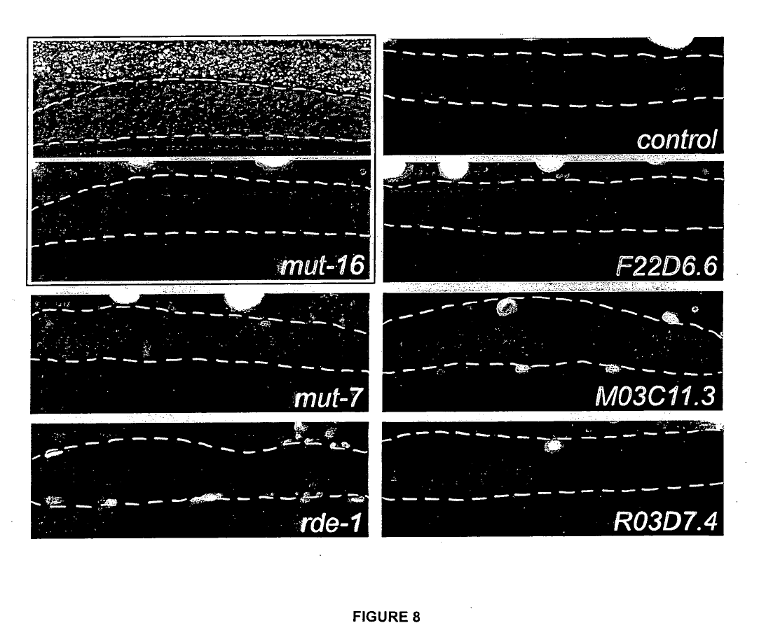

A feeding-based RNAi screen for dsRNAs that inactivate RNAi is more complicated than screens for other phenotypes: dsRNAs corresponding to any component of the RNAi pathway would be expected to be processed by the initially RNAi-proficient animal and would begin to inactivate that one component, but as the inactivation proceeds, RNAi becomes deficient in the animal with a decrement in that component, leading to less efficient silencing of that gene target. Thus depending on both the efficiency of the first phase of RNAi and polypeptide half-lives, a priori non-null phenotypes are expected from using RNAi to inactivate RNAi components. However, when the RNAi sensor strain was tested by feeding bacteria that express dsRNA corresponding to genes previously implicated in RNAi, such as rde-1, rde-4, or dcr-1 (Bernstein et al., Nature 409:363 (2001); Knight and Bass, Science 293:2269 (2001); Tabara et al., Cell 99:123 (1999); Tabara et al., Cell 109:861 (2002); Tijsterman et al., Annu Rev Genet 36:489 (2002)), expression of GFP was robustly restored, whereas feeding the strain bacteria expressing control dsRNA does not affect GFP fluorescence (FIG. 7B). Thus, RNAi of RNAi components can be used in such a screen, and the RNAi sensor strain provides a facile and sensitive assay for RNAi activity in vivo.

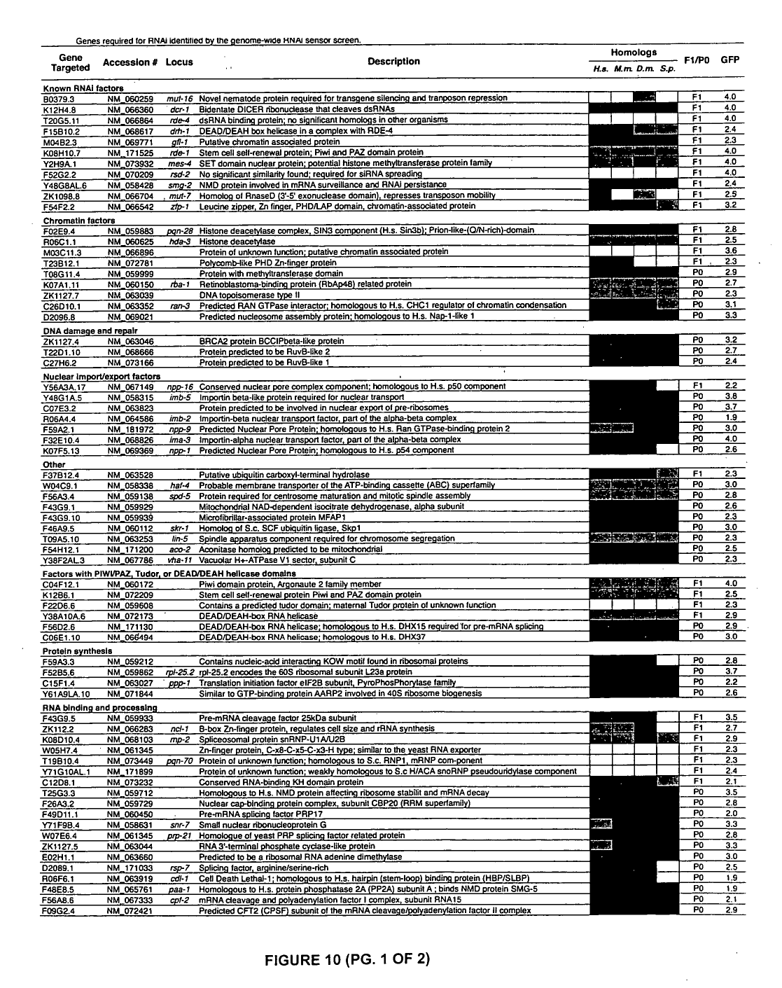

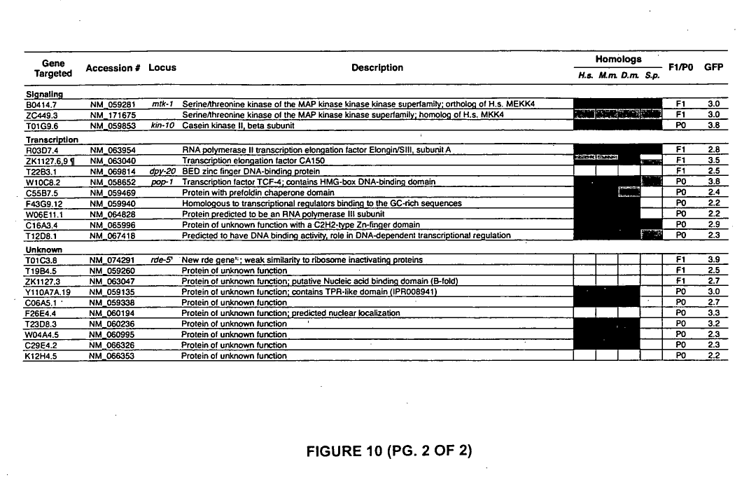

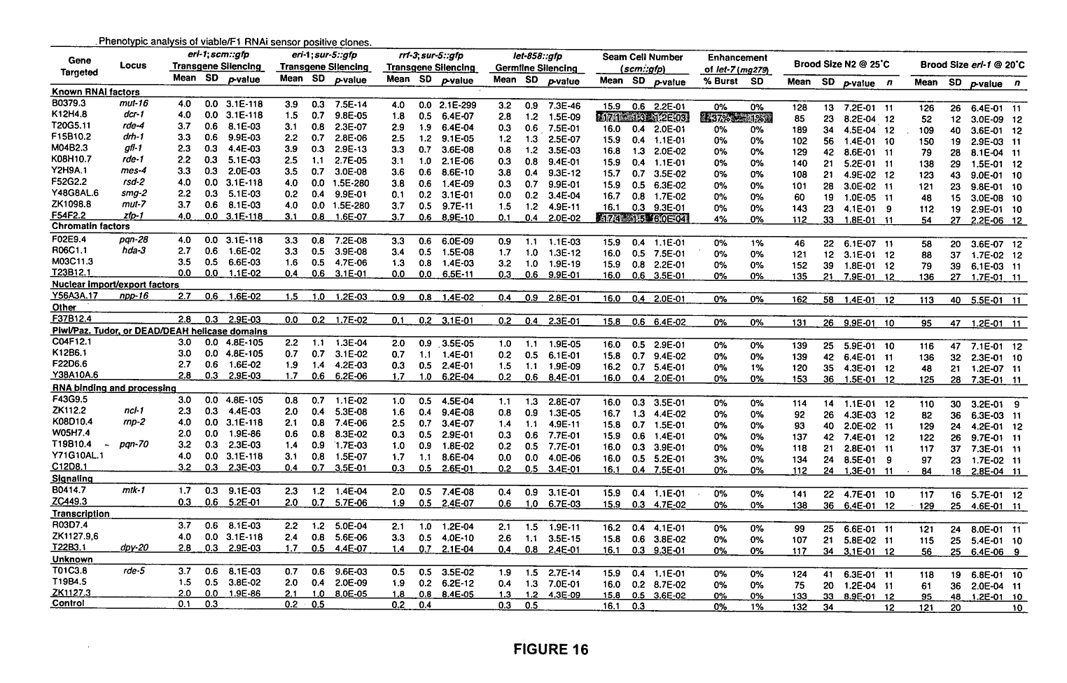

This unbiased approach allowed the identification of new factors that modulate RNAi and related phenomena in vivo. Using these tools, 92 clones were identified that reproducibly resulted in an RNAi-deficient phenotype upon silencing. Ten of these correspond to previously defined loci known to be required for RNAi, including the majority of those believed to comprise the core RNAi machinery (e.g., dcr-1, rde-1, rde-4, mut-7, mut-16, and drh-1 (Tabara et al., Cell 109:861 (2002); Tijsterman et al., Annu Rev Genet 36:489 (2002)); FIG. 10). Given that RNAi-related mechanisms operate in numerous essential processes, it was anticipated that many genes required for RNAi would also be required for viability. Indeed, 54 of the new genes are essential for viability and therefore may have evaded discovery in forward-genetic screens relying on the isolation of viable, RNAi-defective (Rde) mutants (Tabara et al., Cell 99:123 (1999)). Of the remaining 28 new genes whose inactivation resulted in viable progeny, one-third produced significantly reduced (p<0.01, t-test) brood sizes (FIG. 16). The observation that many genes required for RNAi are critical for viability or reproduction further highlights the importance of these factors in animal growth and development.

Other Assays of the New RNAi Factors

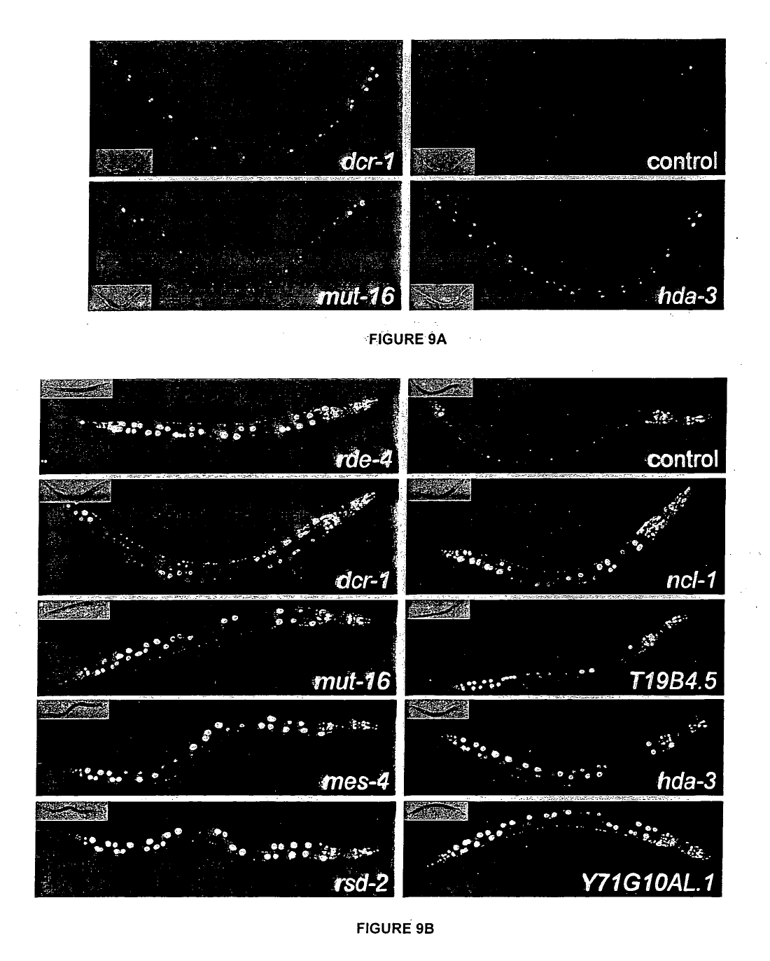

The initial screen for RNAi factors used multiple transgenes. Therefore, it was possible that some of the putative RNAi components might not be involved in RNAi per se and might instead regulate transgene expression or aberrant RNAs from transgenes. Two observations suggested that this was unlikely. First, silencing depends on expression of both the gfp dsRNA-expressing transgene and the gfp-expressing transgene, each of which uses a distinct epidermal promoter. For a clone to score in the screen for spurious reasons, it must selectively allow expression from the epidermal promoter (ajm-1) driving gfp expression but not from the other epidermal promoter (wrt-2) driving expression of gp dsRNA. Second, reactivation of transgene reporters expressed ubiquitously from promoters unrelated to those used in the primary screen (FIG. 9B) or in other tissues (data not shown) when the new factors are inactivated suggests that de-silencing is not a promoter-specific phenomenon.

Factors that affect RNAi only indirectly might also be expected to be identified in the present screen. For example, any of the RNA-binding proteins or helicases could affect RNA splicing or transcription indirectly to alter the expression of other RNAi components. However, a large number of factors involved in RNA regulation, splicing, or transcription were not identified. It is more likely that many of the new factors are particularly sensitive components of the RNAi pathway. Furthermore, a majority of the known RNAi components discovered by previous genetic and biochemical analyses were identified, supporting the view that this method was a highly sensitive in vivo assay for RNAi.

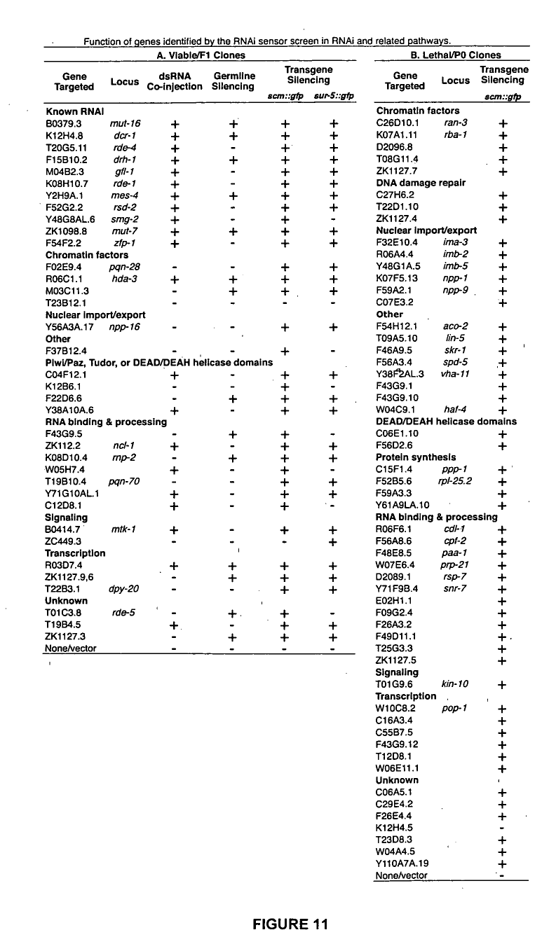

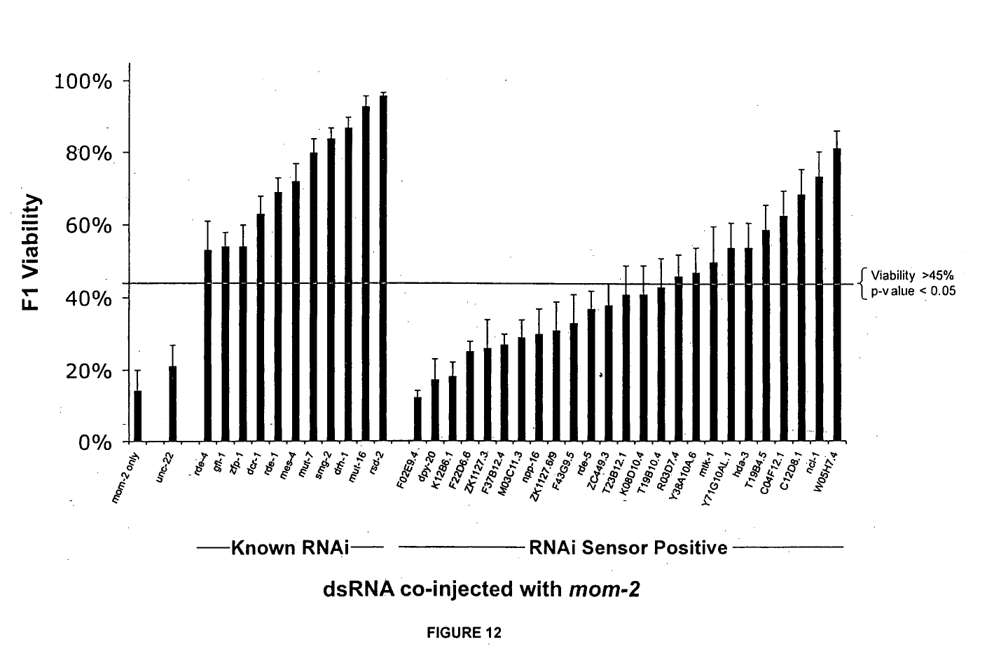

As one means to verify that genes uncovered in the present screen were required for RNAi of endogenous genes, animals were co-injected with dsRNA from each of the candidate RNAi genes together with mom-2 dsRNA, a gene essential for embryonic viability (FIG. 12). This co-injection assay has been previously employed to determine if a gene inactivation causes a canonically Rde response (Tabara et al., Cell 109:861 (2002); Dudley et al., Proc Natl Acad Sci USA 99:4191 (2002)). The survival of progeny from injected animals indicates that inactivation of the candidate gene renders animals resistant to the lethal effects of mom-2 RNAi. Because the co-injection assay relies on detecting a phenotype in the progeny of injected worms, only the 36 RNAi genes that are not essential for viability (summarized in FIG. 11 and FIG. 12) were examined. Inactivation of 11 newly-identified and 10 known RNAi genes rescued the lethality associated with injection of mom-2 dsRNA by a statistically significant margin (>45% viability, p<0.05), compared to injection of mom-2 dsRNA alone or co-injection with dsRNAs targeting genes dispensible for RNAi (e.g., unc-22; FIG. 12 and Methods).

While this assay was useful in validating RNAi factors, it could not be used to disqualify factors: injection of T01C3.8 dsRNA, recently identified as rde-5 by classical genetic analysis of RNAi (C. Mello, personal communication), failed to rescue the lethality of mom-2 dsRNAs, indicating that this assay can fail to detect known RNAi pathway genes. rde genes might fail to rescue mom-2 RNAi if introducing dsRNAs into the cytoplasm by injection bypasses the requirement for RNAi factors that act upstream of this step such as in transport of dsRNAs from the nucleus (e.g., nuclear export of the gfp dsRNA in the RNAi sensor strain). Furthermore, factors required for transcriptional gene silencing but dispensable for post-transcriptional gene silencing and factors that modulate but are not essential for RNAi would likely fail the co-injection assay.

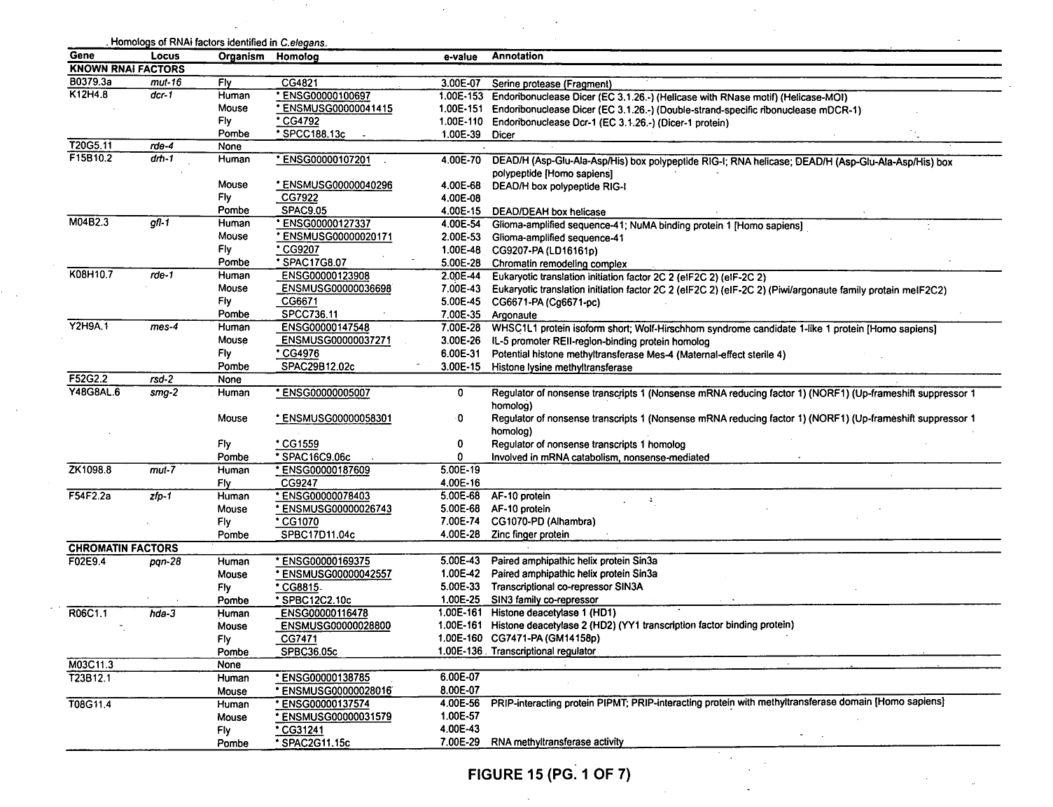

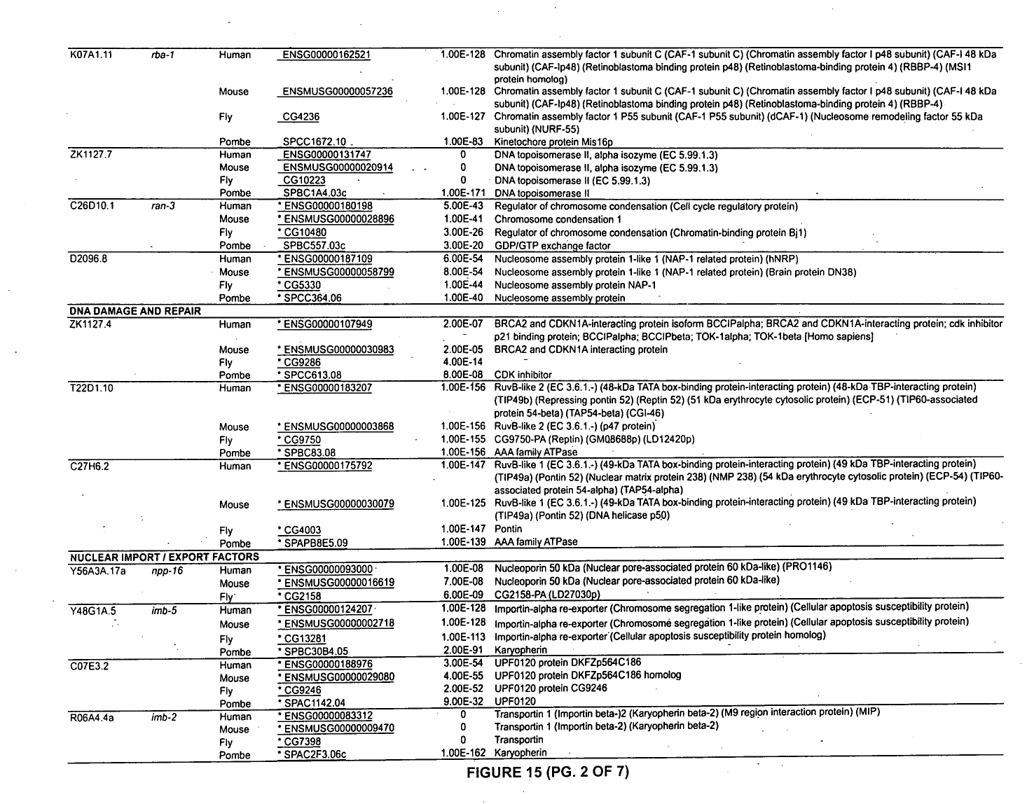

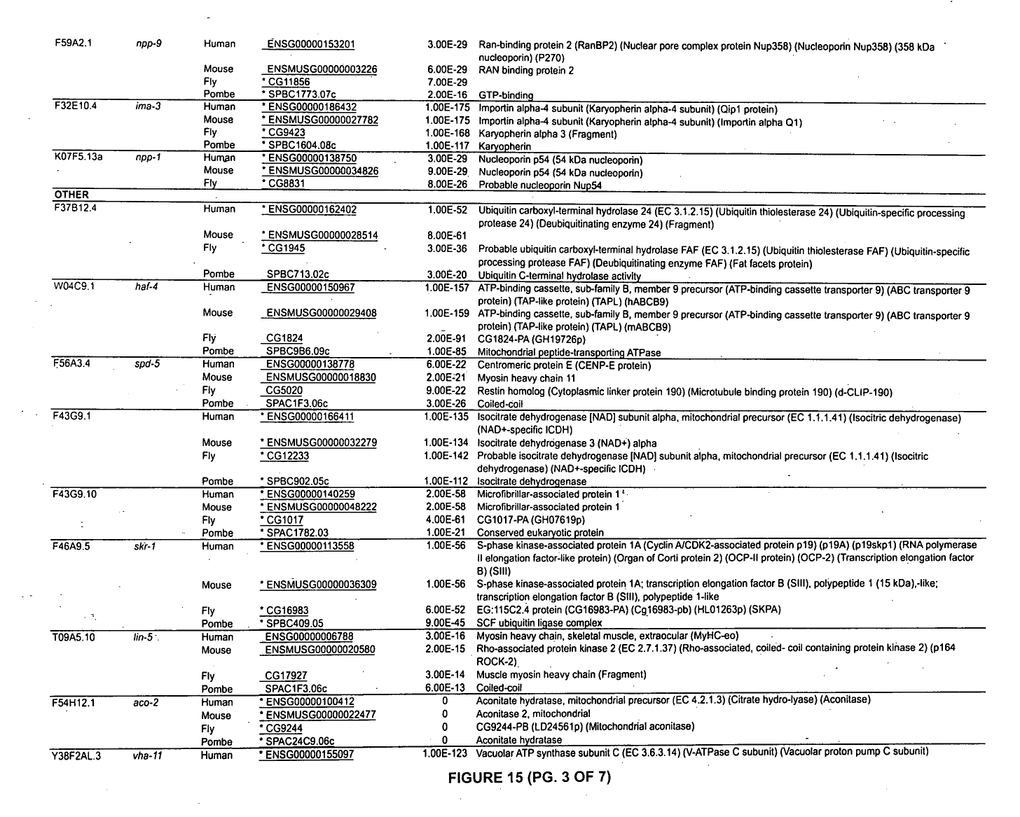

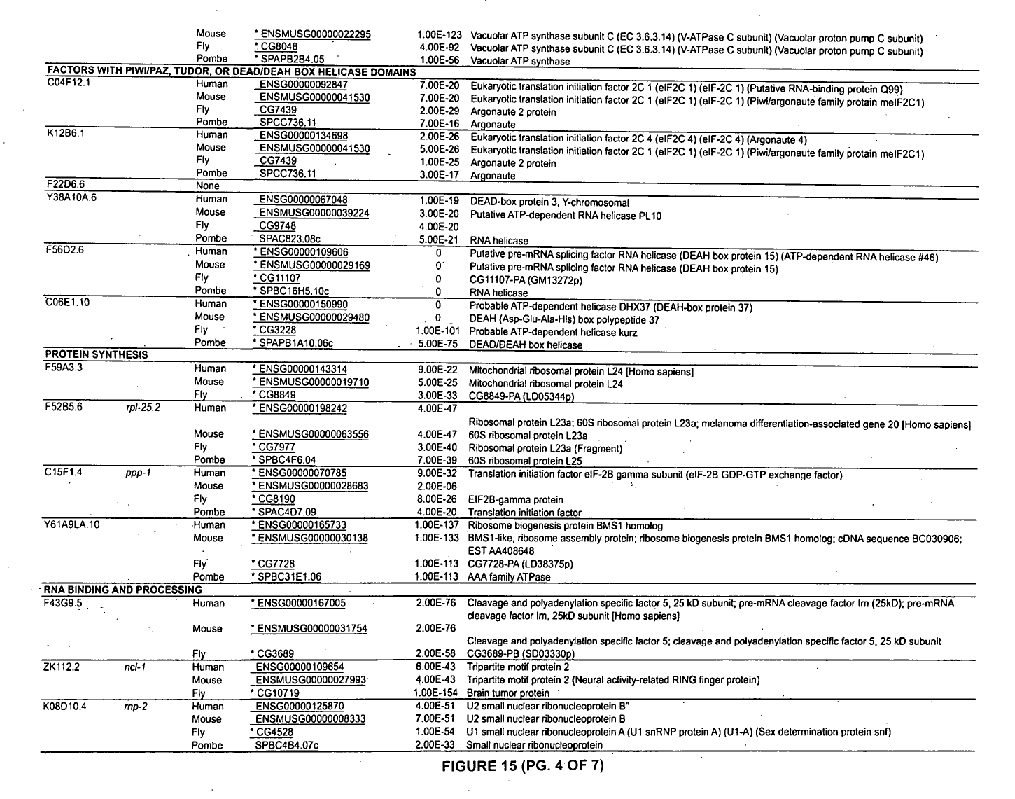

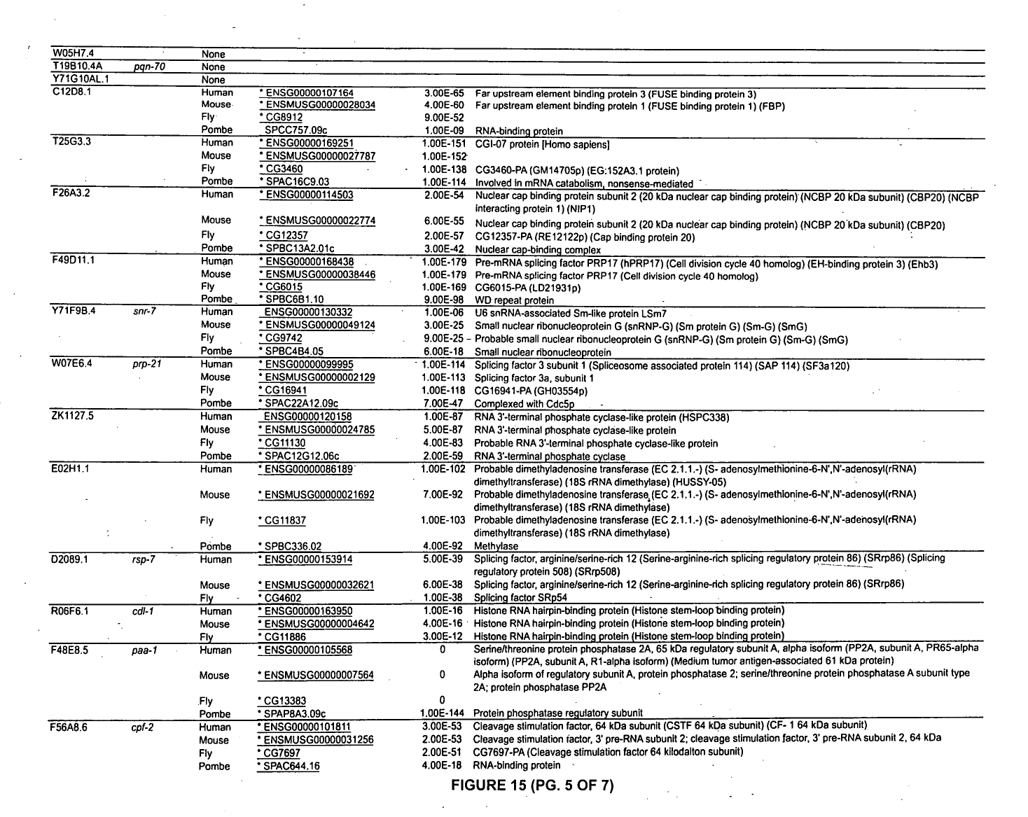

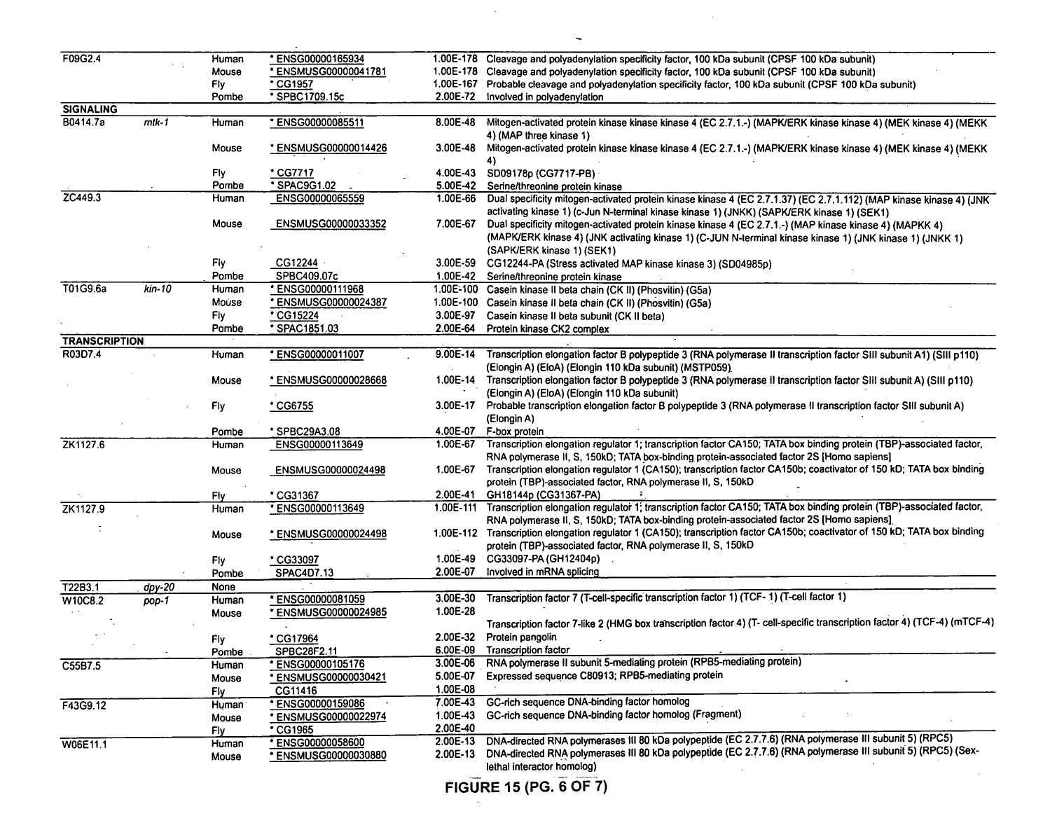

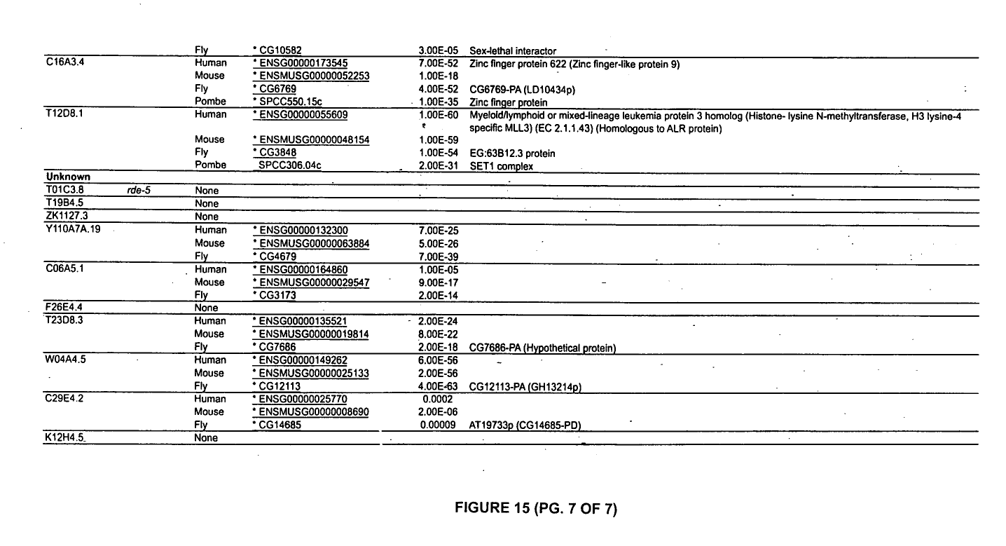

Among those genes identified were 10 of the 24 loci known to have RNAi-deficient loss-of-function phenotypes. Interestingly, known loci required for RNAi in both the germline and soma were identified more consistently than those genes known to be required in only one of the two tissues. Based on analysis of recognizable polypeptide domains, the new RNAi factors fall into distinct functional classes that delineate the scope of the cellular processes required for RNAi. The fact that 85% of the new genes have human homologs also suggests that their functions are likely conserved across metazoan phylogenies (FIGS. 10 and 15).

Among the new RNAi factors, six polypeptides with domains found in components of known RNAi complexes were identified: two polypeptides contain either a Piwi (C04F12.1) or both Piwi and PAZ (K12B6.1) domains found in the Argonaute polypeptides of the RISC and RITS complexes (Meister and Tuschl, Nature 431:343 (2004); Hammond et al., Nature 404:293 (2000); Verdel et al., Science 303:672 (2004)), one factor (F22D6.6) possesses a Tudor RNA-binding domain identified in the TSN micrococcal nuclease of RISC (Caudy et al., Nature 425:411 (2003)), and three helicases (Y38A10A.6, F56D2.6, and C06E1.10) have DEAD/DEAH-box motifs found in Dicer, MUT-14, and the DRH-1 and DRH-2 helicases (Bernstein et al., Nature 409:363 (2001); Tabara et al., Cell 109:861 (2002); Tijsterman et al., Science 295:694 (2002)). The new Piwi factors (C04F12.1 and K12B6.1) may function in the canonical RNAi complexes identified thus far, or they may act in analogous complexes that further process and present siRNAs to target mRNAs. The helicases and RNA binding factors could act at any step of the pathway, since such polypeptides mediate many steps in RNA processing.

Piwi/PAZ Polypeptides

C04F12.1 and K12B6.1

C. elegans gene C04F12.1 is predicted to encode a polypeptide 944 amino acids in length that contains a Piwi domain and is a homolog of Drosophila AGO2 (Argonaute2) (BLASTP E-value of 6.1 e-26 over 46.9% of the total polypeptide length) and human eukaryotic translation initiation factor 2C (eIF2C; BLASTP E-value of 1.1e-24 over 58.8% of the total polypeptide length) (ENSEMBL:ENSP00000257131).

The gene K12B6.1 is predicted to encode a polypeptide 881 amino acids in length that contains both a Piwi and a PAZ (Piwi, Argonaute, Zwille/Pinhead) domain. This polypeptide is also homologous to Drosophila AGO2 (BLASTP E-value of 2.9e-27 over 50.4% of the total polypeptide length) and human eIF2C (BLASTP E-value of 6.7e-27 over 62.7% of the total polypeptide length). Both genes are part of a large family of Piwi-containing polypeptides with at least 10 close homologs in the C. elegans genome (with a BLASTP E-value <1.0e-24). It is important to note that K12B6.1 contains both Piwi and PAZ domains, whereas C04F12.1 does not. The identification of these two genes as required for RNAi is particularly interesting, given that members of the Piwi/PAZ family have been implicated in RNAi-related phenomena in S. pombe, Arabidopsis, C. elegans, Drosophila, and humans (Cerutti et al., Trends Biochem Sci 25:481 (2000); Carmell et al., Genes Dev 16:2733 (2002); and Sasaki et al., Genomics 82:323 (2003)). No other cellular role for Piwi/PAZ polypeptides has been identified.

C. elegans having a mutation in ppw-1, which encodes a Piwi/PAZ polypeptide, are resistant to RNAi of germline-expressed genes. Another Piwi/PAZ encoding gene, ppw-2, is implicated in transgene-induced post transcriptional gene silencing of germline-expressed genes and may also function in transposon silencing in the germline. These finding suggest that PPW-1 or PPW-2 may substitute for RDE-1 in these processes (Tijsterman et al., Annu Rev Genet 36:489 (2002); and Vastenhouw et al., Curr Biol 13:1311 (2003)). The crystal structure of the PAZ domain of Argonaute2 was recently solved, revealing that this domain has RNA-binding activity that likely contributes to the incorporation of siRNAs into the RISC complex (Lingel et al., Nature 426:465 (2003); Yan et al., Nature 426:468 (2003); and Song et al., Nat Struct Biol 10:1026 (2003)). Recently, it has also been shown that a subregion of the Piwi domain, the Piwi-box, binds directly to the Dicer RNase III domain, thereby inhibiting the action of Dicer in vitro (Tahbaz et al., EMBO Rep 5:189 (2004)). Therefore, in contrast to these results, other Piwi/PAZ polypeptides may act as negative regulators of the RNAi pathway. Piwi/PAZ polypeptides and Dicer have also been shown to be present in both soluble and membrane-associated fractions in the cell, suggesting that interactions between these components may occur in multiple cellular compartments (Tahbaz et al., supra). Given these results, it is likely that C04F12.1 and K12B6.1 function in facilitating the interaction between a small interfering RNA and its substrate.

RNA Helicase

Y38A10A.6 The C. elegans gene Y38A10A.6, which was identified in the GR1401 (Is41) screen as essential for RNAi, is predicted to encode a polypeptide 520 amino acids in length that contains a DEAD/DEAH-box helicase domain. This gene encodes a polypeptide homologous to RNA helicases from numerous species, including E. coli (BLASTP E-value of 2.5e-17 over 55.6% of the total polypeptide length) (SW:SRMB_ECOLI), S. cerevisiae (BLASTP E-value of 3e-18 over 82.1% of the total polypeptide length) (SGD:YPL119C), Drosophila (BLASTP E-value of 4.3e-17 over 69.2% of the total polypeptide length) (GADFLY:CG9748-PA), and humans (BLASTP E-value of 7.4e-17 over 52.3% of the total polypeptide length) (ENSEMBL:ENSP00000277804). The identification of a novel DEAD/DEAH-box RNA helicase as a required component of the RNAi machinery is particularly interesting, since similar polypeptides are involved in RNAi-like phenomena in multiple species.

In C. elegans, three genes encoding putative DEAD/DEAH-box helicases were previously known to be involved in RNAi: drh-1, mut-14, and smg-2. The DRH-1 helicase is essential for RNAi and forms a complex with RDE-4, a dsRNA-binding polypeptide that also interacts with DCR-1 and that is thought to play a role in the recognition of foreign dsRNAs (Tabara et al., Science 282:430 (2002)). DRH-1 likely functions in changing the conformation of trigger dsRNAs, or in facilitating the transfer of dsRNAs from RDE-4 to Dicer. The MUT-14 helicase is specifically required for PTGS of germline-expressed genes (Tijsterman et al., Science 295:694 (2002)). MUT-14 is also important for gene silencing that is triggered by small antisense RNAs. Small antisense RNAs are thought to act as primers for dsRNA synthesis from mRNA, suggesting that MUT-14 facilitates RNA synthesis.

SMG-2 is an RNA helicase that is important for nonsense-mediated decay of mRNA, an evolutionarily-conserved surveillance mechanism that protects cells from the potentially deleterious effects of truncated polypeptides (Page et al., Mol Cell Biol 19:5943 (1999)). Three of seven C. elegans smg genes (smg-2, smg-5, and smg-6) are required for the persistence of phenotypes produced by RNAi, leading to the suggestion that these SMG proteins may facilitate the amplification step of RNAi (Domeier et al., Science 289:1928 (2000)).

RNA helicases have also been predicted to act at other stages of the silencing pathway. For example, they may potentially mediate the ATP-dependent unwinding of siRNAs associated with RISC, change the conformation of long dsRNAs that initiate PTGS, or bind RNAs to facilitate multistep reactions. Helicases governing these processes have been identified in other organisms, and some of these helicases are known to be required for RNAi. In Drosophila, for example, AGO2 and dFMR (the Drosophila Fragile X syndrome protein) are found in a complex with Dmp68, an ortholog of human p68, which has been shown to unwind short (but not long) dsRNAs in an ATP-dependent manner. dmp68 has also been shown to be essential for RNAi in Drosophila (Ishizuka et al., Genes Dev 16:2497 (2002)). Therefore, p68 may be the helicase required to activate RISC by unwinding the siRNA duplex. Spindle-E is a DEAH-box RNA helicase that is required for RNAi during oocyte maturation in Drosophila (Kennerdell et al., Genes Dev 16:1884 (2002)). This gene is also required for proper translational control, suggesting a connection between gene silencing and translation in oocytes. In Arabidopsis, SDE3 is an RNA helicase that is required for PTGS mediated by transgenes, but not by viruses (Dalmay et al., EMBO J. 20:2069 (2001)). Finally, the Chlamydomonas mut6 gene, which encodes an RNA helicase highly homologous to DEAH-family helicases, is required for PTGS mediated by transgenes (Wu-Scharf et al., Science 290:1159 (2000)). Increased levels of aberrant RNAs are found in Mut6 mutants, suggesting that this helicase, like SMG-2, is involved in the degradation of abnormal RNAs.

RNA Binding and Processing Factors

RNA binding and processing factors constitute the largest class of new RNAi factors identified and suggest new steps in the RNAi pathway as well as overlap with other RNA-mediated gene regulatory pathways (FIG. 10). Components of the multi-subunit pre-mRNA cleavage and polyadenylation complex known to function in the formation of mRNA 3′ ends were identified (Proudfoot, Curr Opin Cell Biol 16:272 (2004); Proudfoot and O'Sullivan, Curr Biol 12:R855 (2002)) including F09G2.4, cpf-2, and F43G9.5, key components of the cleavage and polyadenylation specificity factor (CPSF/F09G2.4), the cleavage stimulation factor (CstF/CPF-2), and the cleavage factor I (CF Iₘ/F43G9.5), respectively. In addition, others have shown that mutations in the predicted polyA polymerase component of this complex exhibit an Rde phenotype in C. elegans (C. Mello, personal communication). A direct connection between the polyA polymerase complex and RNAi was recently demonstrated in S. pombe by the association of the RITS complex with a polypeptide complex containing Cid12, a member of the polyA polymerase family (Motamedi et al., Cell 119:789 (2004)). Recent work has also shown that the 5′ fragment produced by microRNA-directed cleavage of a target mRNA is polyuridylated (with an occasional adenine as well) at the 3′ end, triggering degradation by the 5′-3′ exonuclease XRN-4 (Shen and Goodman, Science 306:997 (2004); Gazzani et al., Science 306:1046 (2004)). The mRNA cleavage and polyadenylation complex may modify the 3′ end of mRNAs cleaved by RISC, leading to degradation in a similar manner.

The nonsense-mediated decay (NMD) gene smg-2 as well as three genes predicted to function in NMD, T25G3.3, paa-1, and F26A3.2, were identified as modulators of RNAi, consistent with previous observations implicating smg-2, and, to a lesser degree, smg-5 and smg-6, as a subset of smg genes involved in RNAi (Domeier et al., Science 289:1928 (2000)). paa-1 encodes a subunit of protein phosphatase 2A, which dephosphorylates SMG-2 and physically interacts with SMG-5 (Anders et al., Embo J 22:641 (2003)). The kinetics of the processing of dsRNAs and initial degradation of the target mRNA are similar in smg (−) mutants and wild type animals, suggesting that the NMD factors act downstream of siRNA production and initial target cleavage (Domeier et al., Science 289:1928 (2000)). One possibility is that NMD factors facilitate the degradation of cleaved mRNA.

Inactivation of these RNA binding and processing factors may cause the accumulation of mRNAs cleaved by siRNA-guided RISC complexes. Given that the rate-limiting step of the multi-turnover RISC complex is the release of cleaved mRNA (Hutvagner and Zamore, Science 297:2056 (2002); Haley and Zamore, Nat Struct Mol Biol 11:599 (2004)), an abundance of cleaved mRNAs in the cytosol may prevent efficient dissociation of these products from RISC and impede the binding of new substrates to RISC for the next round of mRNA endonucleolysis. Such a function would also be consistent with the observation that in smg-2 (−) worms, the initial RNAi response is preserved but begins to deteriorate over time, suggesting that NMD is dispensable for RNAi initiation but is required for the persistence of RNAi (Domeier et al., Science 289:1928 (2000)). In another embodiment, defects in mRNA processing and degradation may lead to an accumulation of aberrant messages that could serve as templates for RdRP (RNA-dependent RNA polymerase) amplification of siRNAs, thereby saturating the RNAi machinery (Tijsterman et al., Annu Rev Genet 36:489 (2002)). F43G9.5

The C. elegans gene F43G9.5 is predicted to encode a polypeptide 227 amino acids in length that is highly homologous to the 25-kDa subunit of human pre-mRNA cleavage factor Iₘ (CF Iₘ) (BLASTP E-value of 6.9e-78 over 86.8% of the total polypeptide length) and that is homologus to related polypeptides in Drosophila and other organisms. CF Iₘ is a factor required for 3′ pre-mRNA cleavage and polyadenylation processing (Rüegsegger et al., J Biol Chem 271:6107 (1996)). Most primary eukaryotic mRNA transcripts are processed before being exported to the cytoplasm through capping of the 5′ end, removal of introns by splicing, and generation of a new 3′ end by endonucleolytic cleavage followed by polyadenylation of the major cleavage product. Splicing and 3′-end processing depend on cis-acting sequence in the pre-mRNA and also on a large number of trans-acting polypeptide factors, some of which are associated with small nuclear RNAs (snRNAs), that are thought to confer specificity (Zhao et al., Microbiol Mol Biol Rev 63:405 (1999)).

CPSF (cleavage and polyadenylation specificity factor) has been shown to bind to the conserved upstream polyadenylation signal on pre-mRNAs, and cleavage stimulation factor (CstF) has been shown to bind to a poorly-defined GU-rich sequence downstream of the polyadenylation site. CPSF, CstF, and the pre-mRNA form a stable ternary complex (Gilmartin and Nevins, Genes Dev 3:2180 (1989); Weiss et al., EMBO J. 10:215 (1991); and MacDonald et al., Mol Cell Biol 14:6647 (1994)). CF Iₘ, CF Iₘ, and poly(A) polymerase are additional factors required for cleavage and polyadenylation in vivo (Christofori and Keller, Cell 54:875 (1988); Takagaki et al., Cell 52:731 (1988); and Takagaki et al., Genes Dev 3:1711 (1989)). CF Iₘ and CF IIₘ are only involved in the first step of 3′ end processing, and CF Iₘ has been shown to stabilize the binding of CPSF to pre-mRNA; in addition, CF Iₘ been shown to be the endonuclease required for cleavage of the primary transcript (Rüegsegger et al., J Biol Chem 271:6107 (1996), and Ru egsegger et al., Mol Cell 1:243 (1998)).

CF Iₘ exists as mixed heterodimers containing one 25-kDa subunit and one of three larger subunits of 59, 68, or 72 kDa. The activity of the CF Iₘ can be reconstituted in vitro from the 25/68-kDa heterodimer, and this complex can stimulate both cleavage and poly(A) addition and can also suppress poly(A) site cleavage in a sequence-dependent manner, suggesting that it plays a central role in regulating pre-mRNA 3′ processing (Ru egsegger et al., Mol Cell 1: 243 (1998); and Brown and Gilmartin, Mol Cell 12:1467 (2003)). The 25-kDa subunit of CF Iₘ has no known polypeptide domains; however, the 68-kDa polypeptide has an RNP-type RNA binding domain and also an SR-like C-terminus which is associated with pre-mRNA splicing activity (Ruegsegger et al., J Biol Chem 271:6107 (1996); and Graveley, RNA 6:1197 (2000)). Therefore, the 68-kDa subunit is predicted to contain the activities required for both pre-mRNA recognition and cleavage, and the 25-kDa polypeptide is though to act more as a scaffold to facilitate the recruitment of other processing factors. Also, the 25-kDa subunit has recently been shown to bind with high specificity to the U1 small nuclear ribonucleoprotein (snRNP) (a component of the spliceosome) as part of a 25/68-kDa heterodimer, thereby supporting its role as a director of the activity of the larger subunits (Awasthi and Alwine, RNA 9:1400 (2003)). The presence of a splicing activity within the 68-kDa subunit and the association of CF Iₘ with the U1 snRNP makes it an attractive candidate for coupling splicing and 3′-end processing. This idea was recently supported by the detection of CF Iₘ during multiple large-scale proteomic analyses of the human spliceosome (Rappsilber et al., Genome Res 12:1231 (2002); and Zhou et al., Nature 419:182 (2002)).

A list of exemplary orthologs for F43G9.5 is provided in Table 1. The heading “Source Range” refers to the nucleotide sequence positions of F43G9.5 and the heading “Target Range” refers to the nucleotide sequence positions with which the target sequence shares identity.

TABLE 1
F43G9.5 Orthologs

| | | | | Source | Target |
|---|---|---|---|---|---|
| Hit | Species | Description | E-Value | Range | Range |
| C. briggsae | gene CBG12500 | | 3.10E − 119 | 1 . . . 227 | 1 . . . 227 |
| TR:Q8I712 | B. malayi | Putative pre-mRNA cleavage factor. | 4.80E − 82 | 5 . . . 223 | 9 . . . 228 |
| TR:Q7T3C6 | B. rerio | Hypothetical protein. | 4.00E − 78 | 27 . . . 224 | 30 . . . 228 |
| ENSEMBL: | H. sapiens | mRNA cleavage factor I 25 kDa | 8.40E − 78 | 27 . . . 224 | 29 . . . 227 |
| ENSP00000300291 | | subunit | | | |
| TR:Q9CQF3 | M. musculus | 3110048P04Rik protein (RIKEN | 8.40E − 78 | 27 . . . 224 | 29 . . . 227 |
| | | cDNA 3110048P04 gene). | | | |
| GADFLY: | D. melanogaster | Flybase gene name is CG3689 | 8.50E − 61 | 31 . . . 181 | 44 . . . 194 |
| CG3689-PB | | | | | |
| TR:Q9CZQ0 | M. musculus | 3110048P04Rik protein. | 1.80E − 57 | 27 . . . 176 | 29 . . . 179 |
| TR:Q8GXS3 | A. thaliana | Hypothetical protein. | 7.40E − 55 | 31 . . . 221 | 4 . . . 195 |
| TR:Q7XPV9 | O. sativa | OSJNBa0032F06.22 protein. | 1.90E − 54 | 34 . . . 221 | 9 . . . 197 |
| TR:Q94AF0 | A. thaliana | AT4g29820/F27B13_60 (mRNA | 1.00E − 42 | 34 . . . 221 | 29 . . . 217 |
| | | cleavage factor subunit-like | | | |
| | | protein). | | | |
| TR:Q9M0K5 | A. thaliana | Hypothetical protein (Fragment). | 1.80E − 33 | 31 . . . 166 | 3 . . . 154 |
| TR:O65606 | A. thaliana | Hypothetical protein. | 2.30E − 33 | 31 . . . 166 | 4 . . . 155 |
| TR:Q9SZQ4 | A. thaliana | mRNA cleavage factor subunit-like | 2.10E − 32 | 34 . . . 60 | 29 . . . 55 |
| | | protein. | | | |
| TR:Q9SZQ4 | A. thaliana | mRNA cleavage factor subunit-like | 2.10E − 32 | 77 . . . 221 | 47 . . . 180 |
| | | protein. | | | |

K08D10.4

Interestingly, other U1 snRNP-associated polypeptides were also identified as required for RNAi in vivo. The C. elegans gene K08D10.4, the rnp-2 locus, is predicted to encode a polypeptide 206 amino acids in length that contains two RNP RNA-binding domains and that is highly homologous to the U1 snRNP A polypeptide (U1A) from humans (BLASTP E-value of 4.1e-54 over 98.5% of the total polypeptide length), mice, Drosophila, Xenopus, and other species. Splicing of pre-mRNAs is a critical regulatory stage during which accurate recognition and removal of introns by the splicing machinery prepares the mRNA for polypeptide translation. The splicing reaction is carried-out by the spliceosome, a dynamic complex of five snRNPs (U1, U2, U4, U5, and U6) and many auxiliary polypeptides (reviewed in Jurica and Moore, Mol Cell 12:5 (2003)). According to current models of spliceosome assembly, the U1 snRNP initially recognizes the 5′ splice site through base-pairing interactions and non-snRNP splicing factors interact with the 3′ splice site to bring them into proximity with one another. In subsequent steps, the U1-5′ splice site. base pairing is weakened in an ATP-dependent step requiring the p68 RNA helicase, allowing the U2 snRNP to base pair with the branch site, and then the U4/U5/U6 tri-snRNP complex is added. As discussed above, p68 is found in a complex with AGO2 and FMRF. Ultimately, U1 is replaced by U5 and U6 at the 5′ splice site, and the other four factors undergo successive rearrangements to allow the constitutive catalytic step that generates a mature mRNA and liberates the intron (Malca et al., Mol Cell Biol 23:3442 (2003)).

The U1 snRNP is the most abundant of the spliceosomal snRNPs and is composed of 10 polypeptides and the 164-nucleotide U1 snRNA (Will and Luhrmann, Curr Opin Cell Biol 9:320 (1997)). U1A, which is a polypeptide component of U1, is thought to play an important role in 5′ and 3′ splice site communication. It is possible that U1A is not essential for the splicing reaction to occur, since in vitro splicing can still proceed in the absence of U1A (Tarn and Steitz, Proc Natl Acad Sci USA 92:2504 (1995)). U1A contains two conserved RNA recognition motifs (RRMs): one is thought to help fold or maintain U1 RNA in an active configuration, while the other interacts with sequences upstream of the polyadenylation signal to increase the efficiency of polyadenylation (Liao et al., Genes Dev 7:419 (1993); and Lutz et al., Genes Dev 8:576 (1994)). In addition, U1A physically interacts with CPSF, suggesting that this polypeptide may play a global role in RNA processing by linking splicing with polyadenylation.

Orthologs of K08D10.4 are provided in Table 2. The heading “Source Range” refers to the nucleotide sequence positions of K08D10.4 and the heading “Target Range” refers to the nucleotide sequence positions with which the target sequence shares identity.

TABLE 2
Orthologs of K08D10.4

| | | | | Source | Target |
|---|---|---|---|---|---|
| Hit | Species | Description | E value | Range | Range |
| BP:CBP03382 | C. briggsae | gene CBG13971 | 1.00E − 94 | 1 . . . 206 | 1 . . . 206 |
| WP:CE07355 | C. elegans | U1 small nuclear ribonucleoprotein A | 8.70E − 55 | 1 . . . 206 | 1 . . . 217 |
| ENSEMBL: | H. sapiens | U1 small nuclear ribonucleoprotein A | 4.10E − 54 | 3 . . . 129 | 5 . . . 132 |
| ENSP00000243563 | | | | | |
| ENSEMBL: | H. sapiens | U1 small nuclear ribonucleoprotein A | 4.10E − 54 | 129 . . . 206 | 205 . . . 282 |
| ENSP00000243563 | | | | | |
| BP:CBP03383 | C. briggsae | gene CBG13972 | 9.90E − 54 | 1 . . . 206 | 1 . . . 220 |
| TR:Q62189 | M. musculus | Small nuclear RNA (Small nuclear | 4.50E − 53 | 3 . . . 129 | 11 . . . 138 |
| | | ribonucleoprotein polypeptidesA). | | | |
| TR:Q62189 | M. musculus | Small nuclear RNA (Small nuclear | 4.50E − 53 | 129 . . . 206 | 210 . . . 287 |
| | | ribonucleoprotein polypeptidesA). | | | |
| TR:Q9CXX7 | M. musculus | Small nuclear ribonucleoprotein | 5.70E − 53 | 3 . . . 119 | 11 . . . 123 |
| | | polypeptide A. | | | |
| TR:Q9CXX7 | M. musculus | Small nuclear ribonucleoprotein | 5.70E − 53 | 129 . . . 206 | 210 . . . 287 |
| | | polypeptide A. | | | |
| GADFLY:CG4528- | D. melanogaster | Flybase gene name is snf | 1.30E − 51 | 3 . . . 206 | 2 . . . 216 |
| PA | | | | | |
| TR:Q41498 | S. tuberosum | U1snRNP-specific protein, U1A. | 1.90E − 51 | 3 . . . 129 | 18 . . . 145 |
| TR:Q41498 | S. tuberosum | U1snRNP-specific protein, U1A. | 1.90E − 51 | 125 . . . 206 | 172 . . . 253 |
| TR:Q9CQI7 | M. musculus | 2810052G09Rik protein (U2 small | 2.80E − 51 | 3 . . . 131 | 2 . . . 130 |
| | | nuclear ribonucleoprotein B). | | | |
| TR:Q9CQI7 | M. musculus | 2810052G09Rik protein (U2 small | 2.80E − 51 | 129 . . . 206 | 148 . . . 225 |
| | | nuclear ribonucleoprotein B). | | | |
| ENSEMBL: | | | 3.50E − 51 | 3 . . . 131 | 2 . . . 130 |
| ENSP00000246071 | | | | | |
| ENSEMBL: | | | 3.50E − 51 | 129 . . . 206 | 148 . . . 225 |
| ENSP00000246071 | | | | | |
| TR:Q9CZ66 | M. musculus | 2810052G09Rik protein. | 4.50E − 51 | 3 . . . 131 | 2 . . . 130 |
| TR:Q9CZ66 | M. musculus | 2810052G09Rik protein. | 4.50E − 51 | 129 . . . 206 | 148 . . . 225 |
| TR:Q39244 | A. thaliana | U1SNRNP-specific protein (Small | 3.60E − 48 | 2 . . . 125 | 12 . . . 133 |
| | | nuclear ribonucleoprotein | | | |
| | | U1A)s(AT2G47580/T30B22.12). | | | |
| TR:Q39244 | A. thaliana | U1SNRNP-specific protein (Small | 3.60E − 48 | 120 . . . 206 | 165 . . . 250 |
| | | nuclear ribonucleoprotein | | | |
| | | U1A)s(AT2G47580/T30B22.12). | | | |
| TR:Q41499 | S. tuberosum | Spliceosomal protein. | 2.70E − 47 | 3 . . . 127 | 6 . . . 130 |
| TR:Q41499 | S. tuberosum | Spliceosomal protein. | 2.70E − 47 | 117 . . . 206 | 142 . . . 231 |
| TR:Q94GW0 | O. sativa | Putative small nuclear ribonucleoprotein | 6.40E − 47 | 6 . . . 125 | 21 . . . 138 |
| | | U1A. | | | |
| TR:Q94GW0 | O. sativa | Putative small nuclear ribonucleoprotein | 6.40E − 47 | 130 . . . 206 | 177 . . . 253 |
| | | U1A. | | | |
| TR:O22922 | A. thaliana | Putative small nuclear ribonucleoprotein | 3.00E − 46 | 2 . . . 128 | 4 . . . 131 |
| | | U2B (At2g30260). | | | |
| TR:O22922 | A. thaliana | Putative small nuclear ribonucleoprotein | 3.00E − 46 | 124 . . . 206 | 150 . . . 232 |
| | | U2B (At2g30260). | | | |
| TR:Q8LB63 | A. thaliana | Putative small nuclear ribonucleoprotein | 2.10E − 45 | 2 . . . 128 | 4 . . . 131 |
| | | U2B. | | | |
| TR:Q8LB63 | A. thaliana | Putative small nuclear ribonucleoprotein | 2.10E − 45 | 124 . . . 206 | 150 . . . 232 |
| | | U2B. | | | |
| SW:RU1A_XENLA | X. laevis | U1 small nuclear ribonucleoprotein A | 2.70E − 35 | 3 . . . 171 | 5 . . . 176 |
| | | (U1 snRNP A protein). | | | |

ZK1127.6 and ZK1127.9

ZK1127.6 and ZK1127.9 are two highly homologous genes. ZK1127.6 is predicted to encode a polypeptide 424 amino acids in length, and ZK1127.9 is predicted to encode a polypeptide with five different splice forms, the longest of which is 914 amino acids in length. These two genes share extensive homology (BLASTP E-value of 1.5e-201 over 89.9% of the total polypeptide length of ZK1127.6). Since the bacterially expressed dsRNA was predicted to target this shared region, it is likely that it interferes with the expression of both of these genes. Both polypeptides contain an FF domain, which is associated with protein-protein interactions, and the longer isoforms of ZK1127.6 contain a WW/Rsp5 domain that binds to specific proline motifs as well as HMG-I and HMG-Y binding domains that are associated with dsDNA binding, nucleosome phasing, and 3′-end processing of mRNAs. Both polypeptides also share the same homologies to polypeptides from other species. They are most closely related to the human transcription elongation regulator 1 (CA150) (BLASTP E-value of 1.5e-135 over 82.3% of the total polypeptide length) and to the S. cerevisiae Prp40p protein, a splicing component that is associated with the U1 snRNP (BLASTP E-value of 3.6e-19 over 59.7% of the total polypeptide length).

CA150 is a nuclear factor implicated in transcriptional elongation. Transcriptional elongation is achieved through FF-domain binding of the hyperphosphorylated C-terminal repeat domain of RNA polymerase II, which is thought to be the primary means of coordinating mRNA processing events with transcription (Carty et al., Proc Natl Acad Sci USA 97:9015 (2000); Hirose and Manley, Genes Dev 14:1415 (2000)). CA150 has been shown to be involved in several disease processes, and is required for Tat-dependent HIV-1 transcriptional activation. Tat action is transduced via an RNA polymerase II (Pol II) holoenzyme containing CA150 (Suñé et al., Mol Cell Biol 17:6029 (1997)). ZK1127.9 directly interacts with the human Huntington's disease polypeptide, Huntingtin (htt), a ubiquitously-expressed polypeptide of unknown function (Holbert et al., Proc Natl Acad Sci USA 98: 1811 (2001)). C. elegans has no homolog of the human huntingtin gene. Therefore, by extension, CA150 is implicated in the pathogenesis of Huntington's disease, ostensibly through altered transcription mediated by interaction with polyglutamine-expanded htt.

Huntingtin has also been shown to interact with the SIN3 transcriptional repressor complex, thereby establishing a link between CA150 and SIN3. This is particularly interesting since SIN3 was also found to be required for RNAi. In another embodiment, CA150 may be part of a holoenzyme required for an RNA synthesis step within the actual RNAi mechanism, such as the synthesis of dsRNA from mRNA by RdRP during the amplification step. Interestingly, the yeast homolog of ZK1127.9, Prp40p, has a completely different function. It is a U1 snRNP protein involved in splicing. Prp40p has been shown to form a complex with Msl5p (the branchpoint bridging protein) and Mud2p; together, these polypeptides help to bring together the two ends of the intron prior to the actual splicing reaction (Abovich and Rosbash (1997)). The possibility of another U1 snRNP component being required for RNAi is compelling, but it is worth noting that the homology of ZK1127.9 with Prp40p is much worse than with CA150.

W05H7.4

The C. elegans gene W05H7.4, which was identified as required for RNAi in the GR1401 (Is41) screen, is predicted to encode a polypeptide with four different splice forms ranging from 38 to 663 amino acids in length. The two larger splice forms, isoforms A and B, contain a Zn finger domain. W05H7.4 is weakly homologous to the O. sativa U2 snRNP auxiliary factor small subunit (BLASTP E-value of 4.5e-06 over 22.2% of the total polypeptide length), S. cerevisiae NPL3, an RNA-binding protein that carries poly(A)+ mRNA from the nucleus into the cytoplasm (BLASTP E-value of 1.3e-04 over 31.8% of the total polypeptide length), and Drosophila CG6043, which has also been associated with intracellular transport and regulation of transcription from the Pol II promoter (BLASTP E-value of 1.9e-06 over 35.4% of the total polypeptide length). Npl3p is a shuttling heterogeneous nuclear RNP (hnRNP) and RNA-binding protein that is thought to package pre-mRNA into an export-competent RNP and to escort it through the nuclear pore complex (NPC) (Lee et al., Genes Dev 10:1233 (1996); and Shen et al., Genes Dev 12:679 (1998)).

In eukaryotes, mrRNAs must be transported from the site of transcription in the nucleus to the cytoplasm for translation to occur, a process that requires processing, packaging by RNA-binding proteins into ribonucleoprotein particles (RNPs), recognition by export factors, and translocation through the nuclear pore complex (NPC) into the cytoplasm (Lei et al., Genes Dev 15:1771 (2001)). On arrival in the cytoplasm, Npl3p is phosphorylated by Skylp, dissociates from mRNA, and is transported back into the nucleus by the importin Mtr10p (Pemberton et al., J Cell Biol 139:1645 (1997); Senger et al., EMBO J. 17:2196 (1998); Gilbert et al., RNA 7: 302 (2001)). Npl3p is found in a complex with RNA Pol II and is associated with genes in a transcription-dependent manner independent of RNA sequence, suggesting that the co-transcriptional recruitment of export factors may be critical for proper mRNA export (Lei et al., Genes Dev 15:1771 (2001); Lei and Silver, Genes Dev 16:2761 (2002); and Lei and Silver, Dev Cell 2:261 (2002)). This association is particularly interesting given that another Pol II-associated protein is required for RNAi, as described above. The function of Npl3p is coupled with an nonsense-mediated decay-like pathway in yeast that proceeds through a 3′-5′ exonuclease (Burkard and Butler, Mol Cell Biol 20:604 (2000)). Interestingly, Npl3p co-purifies with the U1 snRNP, though this association was not salt-resistant and no functional link has been established. Nevertheless, this interaction is intriguing in light of the results presented above, which connect U1-associated proteins with RNAi (Gottschalk et al., RNA 4:374 (1998)).

T19B10.4

The C. elegans gene T19B10.4, which was identified in the GR1401 (1s41) screen as required for RNAi, is predicted to encode a polypeptide with two splice forms: the longer A isoform is 243 amino acids in length, and the B isoform is 125 amino acids in length. Neither splice form contains a known polypeptide domain. T19B10.4 was given the locus designation pqn-70, since it is one of a family of polypeptides predicted to contain a prion-like glutamine/arginine (Q/N)-rich domain. The T19B10.4 polypeptide was found to be homologous to S. cerevisiae PUB1 (BLASTP E-value 4.3e-08 over 49.8% of the total polypeptide length), which is thought to play a role in the nonsense-mediated decay pathway in yeast. The degradation of mRNAs containing nonsense mutations within the polypeptide coding region is a paradigm for the relationship between mRNA turnover and translation. The nonsense-mediated decay pathway also degrades transcripts with frameshifts, splicing errors, extended 3′-UTRs, or other errors or unusual features (Jacobson and Peltz, Annu Rev Biochem 65:693 (1997); Czaplinski et al., Bioessays 21:685 (1999); and Hilleren and Parker, Annu Rev Genet 33:229 (1999)). The overall stability of a given mRNA is determined by the interplay between the stability determinants present in the mRNA and the factors with which they interact, such as RNA-binding proteins that affect transcript stability (Burd and Dreyfuss, Science 265:615 (1994); and Weighardt et al., Bioessays 18:747 (1997)).

Some yeast mRNAs, particularly those with unusual structures such as upstream ORFs, are known to contain ‘stabilizer elements’ (STEs) that block the activity of the nonsense-mediated decay pathway. Publp is an abundant polypeptide that is a cellular poly(A)-mRNA binding protein (Anderson et al., Mol Cell Biol 13:6102 (1993); and Matunis et al., Mol Cell Biol 13:6114 (1993)). Pub1p has also been identified as a factor that specifically interacts with STEs to prevent nonsense-mediated decay of certain substrates (Ruiz-Echevarria and Peltz, Cell 101:741 (2000)). Similarly, mammalian homologs of Pub1p play important roles in cellular differentiation and proliferation (Antic and Keene, Am J Hum Genet 61:273 (1997); Fan and Steitz, EMBO J. 17:3448 (1998); Peng et al., EMBO J. 17:3461 (1998)). It is interesting that loss of a polypeptide preventing mRNAs from entering the nonsense-mediated decay (NMD) pathway (i.e., resulting in a net increase of transcripts subjected to nonsense-mediated decay is required for RNAi, since components of the nonsense-mediated decay pathway are also required for RNAi. Without being tied to any particular theory, it is possible that loss of Pub1p affects a normally ‘protected’ transcript and causes it to be degraded. T19B10.4 likely interacts with known components of the NMD pathway in C. elegans. Interestingly, like other polypeptides identified, Publp interacts with the p38/Hog1 MAP kinase pathway. Inhibition of this pathway leads to the instability of transcripts that are subsequently stabilized by the interaction of Pub1p with AU-rich elements (AREs) in their sequences (Vasudevan and Peltz, Mol Cell 7:1191 (2001)).

Chromatin Factors

A prominent set of new RNAi genes encodes predicted chromatin factors that may mediate transcriptional gene silencing in response to dsRNAs. Consistent with this view, dsRNAs introduced via transgenesis or transcribed by an endogenous locus are known to recruit heterochromatin factors and drive heterochromatin formation at the homologous genomic locus in a manner dependent on the RNAi machinery, including the RITS complex, in various organisms (Lippman and Martienssen, Nature 431:364 (2004); Verdel et al., Science 303:672 (2004); Pal-Bhadra et al., Mol Cell 9:315 (2002)). For example, the production of particular endogenous siRNAs mediates transcriptional silencing of repetitive DNA elements, such as at centromeres (Grewal and Rice, Curr Opin Cell Biol 16:230 (2004)). Given that transgenes in C. elegans form tandem arrays resembling repetitive elements, gfp dsRNAs derived from both the transgene and the gfp hairpin are thus likely to activate TGS, in addition to PTGS, of the GFP reporter.

Two Polycomb-related components, MES-4 and T23B12.1 were identified as required for RNAi, consistent with the discovery that transcriptional gene silencing requires Polycomb in Drosophila and that transgene silencing requires the Polycomb associated component, MES-4, in the C. elegans germline (Pal-Bhadra et al., Mol Cell 9:315 (2002); Pal-Bhadra et al., Cell 90:479 (1997); Pirrotta, V., Cell 110:661 (2002); FIG. 2). In addition, Sin3 and the core components of the histone deacetylase complex (HDAC) were identified, including hda-3 (histone deacetylase 1), pqn-28 (a SIN3 component), and rba-1 (related to RbAp48 and chromatin assembly factor 1, CAF-1), as essential for RNAi. Interestingly, repression by Polycomb is expected to require deacetylation and subsequent histone methylation, suggesting coordinated function between HDAC and Polycomb in transcriptional repression (Levine et al., Trends Biochem Sci 29:478 (2004)). Furthermore, components of Polycomb and HDAC, in addition to the RNAi machinery and the RITS complex, are all required for heterochromatin formation (Lippman and Martienssen, Nature 431:364 (2004); Pal-Bhadra et al., Science 303:669 (2004); Grewal and Moazed, Science 301:798 (2003)). Consistent with these findings, the worm orthologs of HP1, HPL-1 and HPL-2, are required for germline transgene silencing as observed for MES-4 (Dudley et al., Proc Natl Acad Sci USA 99:4191 (2002); Couteau et al., EMBO Rep 3:235 (2002)). Taken together, these findings suggest a model in which Polycomb and HDAC complexes may function to recruit HP1 and other factors to specific sites on DNA that are targeted by siRNAs of the RITS complex, thus leading to heterochromatin formation. The discovery of chromatin factors essential for RNAi supports the view that TGS is part of how dsRNAs silence transgenes in C. elegans. In addition, our findings for the first time identify members of the Polycomb and Sin3/HDAC complexes as regulators of the RNAi silencing machinery.

Six genes predicted to encode chromatin-related factors required for RNAi were also identified in the GR1401 (Is41) screen. Three of these genes, T23B12.1, M03C11.3, Y71G10AL.1, were not previously known to be associated with RNAi. This is consistent with the fact that RNAi is an epigenetic phenomenon that can result in heritable changes in gene expression. A mechanism to propagate either the signal or the silenced state independent of the initial trigger is required for heritability. RNAi may maintain gene silencing through direct genomic modification, such as through methylation (reviewed in Wolffe and Matzke, Science 286:481 (1999); Wassenegger, Plant Mol Biol 43:203 (2000); Grewal and Elgin, Curr Opin Gen Dev 12:178 (2002); Grewal and Moazed, Science 301:798 (2003)). In plants dsRNA can act as a trigger for silencing at both the transcriptional (TGS) and post-transcriptional (PTGS) levels. Current models suggest that dsRNA is capable of inducing PTGS and, independently, RNA-dependent DNA methylation (RdDM) of the genome (Ingelbrecht et al., Proc Natl Acad Sci USA 91:10502 (1994); Wassenegger et al., Cell 76:567 (1994); Furner et al., Genetics 149:651 (1998); Fagard et al., Proc Natl Acad Sci USA 97:11650 (2000); and Bender, Cell 106:129 (2001)).

Evidence correlating TGS and PTGS in animals also exists. For example, in Drosophila, endogenous loci silenced by cosuppression are bound by the Polycomb complex. In C. elegans the silencing of tandem arrays requires the mes (maternal-effect sterile) genes, which are also members of the Polycomb group (Pal-Bhadra et al., Cell 90:479 (1997), Pal-Bhadra et al., Cell 99:35 (1999), Pal-Bhadra et al., Mol Cell 9:315 (2002); Kelly and Fire, Development 125:2451 (1998); and Korf et al., Development 125:2469 (1998)). Mutations in piwi, aubergine, or spindle-E result in loss of silencing through heterochromatin. Since these genes are also involved in RNAi, this suggests that the RNAi machinery targets heterchromatin formation in Drosophila (Pal-Bhadra et al., Science 303:669 (2004)).

In C. elegans, RNAi-based screens to identify genes required for RNAi have identified several genes that are predicted to encode chromatin-associated polypeptides (Dudley et al., Proc Natl Acad Sci USA 99:4191(2002)). This suggests a connection between RNAi and transcriptional gene silencing in vivo.

T23B12.1

The C. elegans gene T23B12.1, which was identified in the GR1401 (Is41) screen, is predicted to encode a polypeptide 234 amino acids in length that contains a (plant homeodomain) PHD finger domain and that has homology to genes in mice (BLASTP E-value of 7.3e-06 over 41.9% of the total polypeptide length) and humans (BLASTP E-value of 1.3e-06 over 41.9% of the total polypeptide length). No function has been ascribed to either of these mammalian genes.

PHD finger domains are found in nuclear proteins and are involved in chromatin-mediated transcriptional regulation. It is likely that the T23B12.1 functions in chromatin regulation. A plant PHD finger domain was recently shown to act as a nuclear phosphoinositide receptor, though it is unclear whether this is a general purpose for this domain (Gozani et al., Cell 114:99 (2003)). Interestingly, two other PHD finger-containing polypeptides were identified in the screen as required for RNAi. These polypeptides, ZFP-1 and MES-4, were previously suggested to be involved in RNAi and in chromatin remodeling: ZFP-1 (which also contains leucine zipper and Zn finger domains) is a homolog of the putative human transcription factor AF10, and MES-4 (which also contains SET and RING finger domains) has previously been shown to regulate chromatin in the germline (Dudley et al., supra); and Fong et al., Science 296:2235 (2002)).

M03C11.3

The C. elegans gene M03C11.3, which was also identified in the GR1401 (Is41) screen, is predicted to encode a polypeptide 832 amino acids in length. In BLAST searches, M03C11.3 was homologous to the mouse gene acinus (apoptotic chromatin condensation inducer in the nucleus) (BLASTP E-value of 6.0e⁻⁰⁴ over 56.4% of the total polypeptide length). Chromatin condensation and nuclear fragmentation are unique cellular morphological changes that occur during apoptosis, and Acinus is a caspase-3-activated protein that is required for apoptotic chromatin condensation (Sahara et al., Nature 401:168 (1999)). Interestingly, among apoptotic pathways downstream of caspase-3, the one regulated by Acinus is the only pathway that induces chromatin condensation without DNA fragmentation. Because Acinus was found to be ubiquitously expressed, this polypeptide may function in chromatin condensation or nuclear structure changes that occur in normal cells. (Sahara et al., supra). M03C11.3 is also likely to be involved in chromatin remodeling. In addition, multiple recent studies have identified the human Acinus protein as part of the spliceosome. This is particularly interesting, since several putative spliceosomal factors were identified as being essential for RNAi.

Y71G10AL.1

The C. elegans gene Y71G10AL.1, which was identified using the GR1401 (Is41) screen, is predicted to encode a polypeptide with two splice forms: the A isoform is 472 amino acids in length, and the long B isoform is 474 amino acids in length. Y71G10AL.1 is homologous to human nucleosomal binding protein 1 (NSBP1) (BLASTP E-value of 3.1 e-06 over 35.9% of the total polypeptide length) and also to known nucleolar factors from other species, including yeast FPR3 (BLASTP E-value of 5.3e-06 over 20.0% of the total polypeptide length). NSBP1 contains an N-terminus with a nuclear localization signal and nucleosomal binding domain, and the C-terminus is thought to function in the remodeling of higher-order chromatin structure to facilitate transcription (Bustin and Reeves, Prog Nucleic Acid Res Mol Biol 54:35 (1997); Ding et al., Mol Cell Biol 17:5843 (1997); Trieschmann et al., Mol Cell Biol 15:6663 (1995)). Based on these features and also based on functional studies of its mouse homolog, NSBP1 is predicted to function as a nucleosomal binding and transcriptional activating element (King and Francomano, Genomics 71:163 (2001)). The exact role of NSBP1 in vivo remains unclear, but its homology with Y71G10AL.1 suggests that the latter polypeptide may also be involved in transcriptional regulation through chromatin remodeling. Interestingly, the yeast homolog of Y71G10AL.1, Fpr3p, encodes a peptidyl-prolyl cis-trans isomerase that is localized to the nucleolus. While this particular polypeptide was not detected as part of the spliceosome, many other cis-trans prolyl isomerases were, suggesting that there may be a general role for such enzymatic activities during splicing (Benton et al., J Cell Biol 127:623 (1994); Rappsilber et al., Genome Res 12:1231 (2002); Zhou et al., Nature 419:182 (2002); and Jurica et al., RNA 8:426 (2002)). Again, this is compelling due to the fact that several other putative spliceosomal factors were also identified as required for RNAi.

Histone Modifying Enzymes

Many of the genes identified herein as factors regulating chromatin dynamics in C. elegans have homology to counterparts in other organisms that function in chromatin modulation. The large number of such factors suggests that chromatin regulation plays an important role in RNAi. In addition, factors involved in the modification of histones by acetylation were identified: F02E9.4 and R06C1.1. This process is directly associated with gene silencing at the transcriptional level.

F02E9.4 and R06C1.1

The C. elegans gene F02E9.4 is predicted to encode a polypeptide 1505 amino acids in length with a paired amphipathic helix repeat. F02E9.4 is orthologous to the S. cerevisiae gene SIN3 (sensitivity in nystatin) (BLASTP E-value of 5.6e-47 over 45.3% of the total polypeptide length) (SGD:YOL004W) and to polypeptides in Drosophila (GADFLY:CG8815-PA), Xenopus (TR:Q9W6S7), mouse (SW:SN3B_MOUSE), and humans (ENSEMBL:ENSP00000248054). F02E9.4 was given the locus designation pqn-28, since it is one of a family of polypeptides predicted to contain a prion-like glutamine/arginine (Q/N)-rich domain. The C. elegans gene R06C1.1 is predicted to encode a polypeptide 465 amino acids in length with a histone deacetylase domain and is highly homologous to both S. cerevisiae RPD3 (reduced potassium dependency) (BLASTP E-value of 3.2e-136 over 90.1% of the total polypeptide length) and human histone deacetylase 1 (BLASTP E-value of 1.2e-160 over 96.8% of the total polypeptide length). R06C1.1 was given the locus name hda-3 due to its predicted function as a histone deacetylase.

Interestingly, the homologs of these two polypeptides, Sin3p and Rpd3p, respectively, are associated in yeast as part of a large multiprotein complex (Kasten et al., Mol Cell Biol 17:4852 (1997)). Sin3p is known to function broadly in eukaryotes as a transcriptional repressor of polypeptide-coding genes, primarily by acting as a scaffold for the assembly of a complex of other transcriptional regulatory polypeptides such as histone deacetylases and histone acetyltranferases (Ayer, Trends Cell Biol 9:193 (1999); Knoepfler and Eisenman, Cell 99:447 (1999); Ahringer, Trends Genet 16:351 (2000); and Jenuwein and Allis, Science 293:1074 (2001)). For example, the mammalian SIN3 complex contains two histone deacetylases (HDAC1 and HDAC2, homologs of Rpd3p), two histone-binding proteins (RbAp46 and RbAp48), and two polypeptides of unknown function (the SIN3-associated proteins SAP18 and SAP30) (Hassig et al., Cell 89:341 (1997); Laherty et al., Cell 89: 349 (1997); and Zhang et al., Cell 89:357 (1997)).

Neither SIN3 nor other components of the SIN3 complex can bind directly to DNA. Instead, targeting of the complex occurs through protein-protein interactions, typically involving SIN3 and DNA-binding repressors or co-repressors (Ayer, supra (1999); Knoepfler and Eisenmann, supra (1999); and Ahringer, supra (2000)). For example, the SIN3 complex can either activate or repress transcription in conjunction with nuclear hormone receptors, such as the retinoic acid and thyroid hormone receptors, nuclear hormone receptor-specific corepressors, such as N-CoR and SMRT, or sequence-specific DNA-binding proteins, such as Mad, p53, and UME6, (Chen and Evans, Nature 377:454 (1995); Horlein et al., Nature 377:397 (1995); Heinzel et al., Nature 387:434 (1997); Nagy et al., Cell 89:373 (1997); Rundlett et al., Nature 392:831 (1998); and Murphy et al., Genes Dev 13:2490 (1999)). SIN3 may also play a role in transcriptional repression by DNA methylation, since the methyl CpG-binding protein MeCP2 has been shown to recruit the SIN3 complex (Jones et al. (1998)).

SIN3 and RPD3 can also function independently. Mutations in Sin3p that prohibit Rpd3p binding do not abolish transcriptional repression, suggesting that SIN3 has intrinsic repressor activity or may associate with other deacetylases (Laherty et al., Cell 89:349 (1997); Wong and Privalsky, Mol Cell Biol 18:5500 (1998)). And RPD3 is a known component of NuRD (nucleosome remodeling and histone deacetylation), another transcriptional repressor complex. Recent studies have shown that in Drosophila and C. elegans, Polycomb group repression occurs through histone deacetylation via the NuRD complex. This is particularly interesting given that several C. elegans Polycomb group polypeptides are known to be required for RNAi as described herein (Kehle et al., Science 282:1897 (1998); and Unhavaithaya et al., Cell 111:991 (2002)). Also, the SIN3-RPD3 complex may not function exclusively in transcriptional repression, since studies in yeast have shown that the SIN3-RPD3 complex is required for both activation and inactivation of induced genes and also for the stability of silencing at telomeres and HM mating loci, both processes that require transcriptional activation (Vidal and Gaber, Mol Cell Biol 11:6317 (1991); Vidal et al., Mol Cell Biol 11:6306 (1991); and Sun and Hampsey, Genetics 152:921 (1999)). Nevertheless, the independent identification of both SIN3 and RPD3 as genes required for RNAi provides further links between RNAi and transcriptional gene silencing in C. elegans.

A list of orthologs for R06C1.1 is provided in Table 3. The heading “Source Range” refers to the nucleotide sequence positions of R06C1.1 and the heading “Target Range” refers to the nucleotide sequence positions with which the target sequence shares identity.

TABLE 3
R06C1.1 Orthologs

| | | | | Source | Target |
|---|---|---|---|---|---|
| Hit | Species | Description | E-Value | Range | Range |
| BP:CBP04404 | C. briggsae | gene CBG18689 | 2.90E − 212 | 3 . . . 462 | 1 . . . 458 |
| SW:HDA1_STRPU | S. purpuratus | Histone deacetylase 1 | 1.30E − 161 | 1 . . . 434 | 1 . . . 430 |
| | | (HD1). | | | |
| SW:HD12_XENLA | X. laevis | Probable histone deacetylase | 2.60E − 161 | 1 . . . 441 | 1 . . . 439 |
| | | 1-2 (HD1) (RPD3 homolog). | | | |
| SW:HDA1_MOUSE | M. musculus | Histone deacetylase 1 | 2.60E − 161 | 4 . . . 454 | 5 . . . 451 |
| | | (HD1). | | | |
| TR:Q8QGJ8 | F. rubripes | Histone deacetylase. | 3.30E − 161 | 1 . . . 464 | 3 . . . 466 |
| TR:Q7ZYT5 | X. laevis | Similar to histone | 4.20E − 161 | 1 . . . 441 | 1 . . . 439 |
| | | deacetylase 1. | | | |
| SW:HDA1_CHICK | G. gallus | Histone deacetylase 1 | 6.90E − 161 | 1 . . . 462 | 1 . . . 459 |
| | | (HD1). | | | |
| ENSEMBL | H. sapiens | Histone deacetylase 1 | 6.90E − 161 | 4 . . . 454 | 5 . . . 451 |
| ENSP00000271095 | | | | | |
| SW:HD11_XENLA | X. laevis | Probable histone deacetylase | 8.81E − 161 | 6 . . . 441 | 7 . . . 439 |
| | | 1-1 (HD1) (Maternally- | | | |
| | | expressedshistone | | | |
| | | deacetylase) (HDM) (AB21). | | | |
| TR:Q8JIY7 | B. rerio | Histone deaceytlase 1. | 3.00E − 160 | 1 . . . 454 | 3 . . . 449 |
| GADFLY:CG7471-PA | D. melanogaster | Flybase gene name is Rpd3 | 5.70E − 160 | 5 . . . 430 | 4 . . . 423 |
| ENSEMBL | H. sapiens | Histone deacetylase 2 | 8.49E − 160 | 7 . . . 459 | 94 . . . 541 |
| ENSP00000275182 | | | | | |
| SW:HDA2_MOUSE | M. musculus | Histone deacetylase 2 (HD2) | 1.60E − 159 | 7 . . . 459 | 9 . . . 456 |
| | | (YY1 transcription factor | | | |
| | | bindingsprotein). | | | |
| TR:Q7SYZ5 | X. laevis | Hypothetical protein | 1.40E − 157 | 6 . . . 430 | 8 . . . 426 |
| | | (Fragment). | | | |
| WP:CE08952 | C. elegans | Yeast RPD3 protein like | 1.10E − 155 | 3 . . . 443 | 8 . . . 447 |
| SW:HDA2_CHICK | G. gallus | Histone deacetylase 2 | 3.20E − 154 | 7 . . . 459 | 9 . . . 456 |
| | | (HD2). | | | |
| BP:CBP06924 | C. briggsae | gene CBG04588 | 5.11E − 153 | 4 . . . 443 | 9 . . . 447 |
| BP:CBP08173 | C. briggsae | gene CBG09063 | 2.10E − 140 | 6 . . . 429 | 11 . . . 433 |
| TR:Q96VP0 | K. lactis | Reduced potassium | 1.20E − 139 | 5 . . . 425 | 16 . . . 432 |
| | | dependency 3 Rpd3p. | | | |
| TR:Q7Y0Y8 | O. sativa | Histone deacetylase | 2.10E − 139 | 6 . . . 463 | 20 . . . 465 |
| | | HDAC1. | | | |
| TR:Q9P4F5 | E. nidulans | Histone deacetylase RpdA. | 2.60E − 139 | 1 . . . 459 | 20 . . . 478 |
| TR:Q94F82 | Z. mays | Histone deacetylase | 8.99E − 139 | 7 . . . 421 | 22 . . . 427 |
| | | HDA101. | | | |
| TR:Q8T7M1 | P. polycephalum | Putative histone deacetylase. | 3.10E − 137 | 6 . . . 429 | 4 . . . 425 |
| SW:HDAC_ARATH | A. thaliana | Histone deacetylase (HD). | 7.50E − 137 | 7 . . . 440 | 16 . . . 447 |
| TR:Q8H0W2 | A. thaliana | Hypothetical protein | 7.91E − 136 | 6 . . . 428 | 3 . . . 419 |
| | | (At3g44680). | | | |
| SGD:YNL330C | S. cerevisiae | Histone deacetylase; | 1.30E − 135 | 7 . . . 426 | 18 . . . 433 |
| | | regulates transcription and | | | |
| | | silencing | | | |
| SW:HDA3_CHICK | G. gallus | Histone deacetylase 3 | 2.40E − 134 | 10 . . . 434 | 5 . . . 422 |
| | | (HD3). | | | |
| SW:CLR6_SCHPO | S. pombe | Histone deacetylase clr6 | 3.20E − 134 | 7 . . . 410 | 5 . . . 405 |
| | | (Cryptic loci regulator 6). | | | |
| ENSEMBL:ENSP00000302967 | H. sapiens | Splice isoform 1 of O15379 | 4.50E − 133 | 10 . . . 434 | 5 . . . 422 |
| | | Histone deacetylase 3 | | | |
| GADFLY:CG2128-PA | D. melanogaster | Flybase gene name is | 5.60E − 133 | 9 . . . 434 | 5 . . . 432 |
| | | HDAC3 | | | |
| TR:Q9H368 | H. sapiens | Splice isoform 2 of O15379 | 7.80E − 129 | 17 . . . 434 | 13 . . . 423 |
| | | Histone deacetylase 3 | | | |
| BP:CBP18408 | C. briggsae | gene CBG15416 | 1.90E − 112 | 4 . . . 427 | 7 . . . 428 |
| BP:CBP09368 | C. briggsae | gene CBG13352 | 5.50E − 109 | 7 . . . 431 | 26 . . . 461 |
| SGD:YGL194C | S. cerevisiae | Protein with similarity to | 1.10E − 104 | 9 . . . 422 | 27 . . . 446 |
| | | Hda1p, Rpd3p, Hos1p, and | | | |
| | | Hos3p | | | |
| WP:CE01472 | C. elegans | transcriptional regulatory | 1.40E − 103 | 7 . . . 431 | 28 . . . 463 |
| | | protein (RPD3) | | | |
| SW:Q9BY41 | H. sapiens | Splice isoform 1 of Q9BY41 | 6.10E − 82 | 31 . . . 373 | 35 . . . 376 |
| | | Histone deacetylase 8 | | | |
| ENSEMBL:ENSP00000316586 | H. sapiens | Splice isoform 2 of Q9BY41 | 1.20E − 52 | 31 . . . 260 | 35 . . . 264 |
| | | Histone deacetylase 8 | | | |
| SGD:YPR068C | S. cerevisiae | Protein with similarity to | 1.70E − 47 | 28 . . . 83 | 20 . . . 77 |
| | | Hda1p, Rpd3p, Hos2p, and | | | |
| | | Hos3p | | | |
| SGD:YPR068C | S. cerevisiae | Protein with similarity to | 1.70E − 47 | 77 . . . 315 | 148 . . . 387 |
| | | Hda1p, Rpd3p, Hos2p, and | | | |
| | | Hos3p | | | |
| SGD:YNL021W | S. cerevisiae | Putative catalytic subunit of | 1.30E − 30 | 21 . . . 328 | 77 . . . 403 |
| | | a class II histone deacetylase | | | |
| | | complex that also contains | | | |
| | | Hda2p and Hda3p; Hda1p | | | |
| | | interacts with the Hda2p- | | | |
| | | Hda3p subcomplex to form | | | |
| | | an active tetramer; deletion | | | |
| | | increases histone H2B, H3 | | | |
| | | and H4 acetylation | | | |
| GADFLY:CG6170-PA | D. melanogaster | Flybase gene name is | 8.00E − 30 | 26 . . . 308 | 552 . . . 840 |
| | | HDAC6 | | | |

Transcription Factors

RNAi may also act in vivo by effecting changes in transcription that target individual genes or groups of genes. Three genes predicted to encode specific transcription factors were identified in the GR1401 (Is41) screen: ZK112.2, T 19B4.5, and T22B3.1. These genes may be required for the transcription of other factors required for RNAi. In another embodiment, these genes may modulate the activity of ‘master’ genes that act as switches controlling the transcription of a wide variety of downstream genes involved in RNAi. Regardless of their mechanism of action, the identification of multiple transcription factors required for RNAi suggests that transcription regulation and RNAi are more closely linked than had previously been shown.

ZK112.2

The gene ZK112.2 is predicted to encode a polypeptide 851 amino acids in length that contains a B-box Zn finger domain. B-box Zn finger domains are typically found in transcription factors, ribonucleoproteins, and proto-oncoproteins. ZK112.2 corresponds to the locus ncl-1 (abnormal nucleoli) and is orthologous to the Drosophila gene brat (brain tumor) (BLASTP E-value of 7.91 e-163 over 89.4% of the total polypeptide length). In C. elegans, ncl-1 mutants are larger than wild-type worms and have enlarged nucleoli that contain twice as much ribosomal RNA (rRNA) as nucleoli of wild-type worms. NCL-1 is predicted to be a repressor of RNA polymerase I and III-mediated transcription and an inhibitor of cell growth (Frank and Roth, J Cell Biol 140:1321 (1998)). The B-box Zn-finger domain and coiled-coil motifs present in NCL-1 are also common to several human oncogenes, including PML, T18, and RFP. Because tumor cells also often have enlarged nucleoli, it is conceivable that these genes and ncl-1 may have overlapping functions (Frank and Roth, supra (1998)). NCL-1 also shares some features with the tumor suppressor Rb, since loss of Rb function may de-repress transcription by RNA polymerase I and III and result in increases in cell size. This suggests that tumor cells may be able to achieve increased cell growth via mutations in Rb (Williams et al., EMBO J. 13:4251 (1994); Cavanaugh et al., Nature 374:177 (1995); White et al., Nature 382:88 (1996); and White, Trends Biochem Sci Soc 22:77 (1997)).

Interestingly, mutations in the Drosophila homolog of ncl-1, brat (brain tumor), result in the formation of tumors in the larval brain; therefore, Brat is also a tumor suppressor (Arama et al., Oncogene 19:3706 (2000)). Like ncl-1 mutants, brat mutants have larger cells, larger nucleoli, and more rRNA than wild-type cells, and cells overexpressing brat have less rRNA than normal; therefore, like NCL-1, Brat is also an inhibitor of cell growth (Frank et al., Development 129:399 (2002)). Moreover, the brat gene is able to rescue the large nucleolus phenotype of a ncl-1 mutant. Brat has been shown to represses the translation of hunchback, a gene important for axis formation, by binding to the 3′ UTR through interactions with two other polypeptides, Nanos and Pumilio (Sonoda and Wharton, Genes Dev 15:762 (2001)). hunchback is ultimately repressed by this complex either by removing the poly(A) tail or by another unknown mechanism that is poly(A)-independent (Chagnovich and Lehmann, Proc Natl Acad Sci USA 98:11359 (2001)). Essentially, then, Brat is a polypeptide required for the silencing of a specific mRNA; in this sense, the roles of this polypeptide (and, by extension, NCL-1) and RNAi are complementary, and therefore it is unclear why the loss of NCL-1-mediated silencing would result in deficient RNAi. One possible role for NCL-1 in RNAi may be through regulation of ribosomal synthesis, since siRNAs have been shown to be associated with ribosomes (Djikeng et al., RNA 7:1522 (2003)). Given that the mechanism underlying the growth defects of ncl-1 mutants remains unknown, it is likely that NCL-1 has other cellular roles that have yet to be discovered.

T19B4.5

The C. elegans gene T19B4.5, which was also identified in the GR1401 (Is41) screen, is predicted to encode a polypeptide 400 amino acids in length. T19B4.5 is known to be an essential gene in C. elegans, since RNAi of this gene results in embryonic lethality or sterility (Kamath et al., Nature 421:231 (2003)). This gene was detectable in the screen since the lethality and sterility phenotypes were only partially penetrant. T19B4.5 is homologous to the Kaposi's sarcoma-associated herpesvirus (KSHV) latent nuclear antigen (LANA) (BLASTP E-value of 1.8e-08 over 94.8% of the total polypeptide length). LANA is a KSHV protein that is important for maintenance of viral episomal DNA during latent infection and that modulates viral and cellular gene expression (Verma and Robertson (2003)). Specifically, LANA tethers viral episomes to host chromosomes by binding to specific sites within the terminal repeats and also binds to histone H1, but not to core histones, to tether the viral episomes to host chromatin (Ballestas et al., Science 284:641 (1999); and Cotter and Robertson, Virology 264:254 (1999)).

LANA has been shown to regulate transcription by binding to a number of cellular polypeptides involved in transcriptional regulation, including SIN3; this is particularly interesting, since SIN3 was identified as a factor required for RNAi as described herein. Evidence exists suggesting that LANA is involved in transcriptional repression through interaction with p53 and downregulation of p53-mediated activation of responsive promoters (Friborg et al., Nature 402:889 (1999)). Therefore, LANA is a multifunctional polypeptide capable of modulating the transcription of a wide variety of genes (Renne et al., J Virol 75:458 (2001)). LANA has also recently been shown to be a co-activator of c-Jun, thereby also implicating it in the JNK MAP kinase pathway; this is particularly interesting since other activators of the JNK pathway were also identified as required for RNAi (An et al., Oncogene 22:3371 (2004)). Although its exact role remains unclear, based on homology, T19B4.5 is predicted to be involved in transcriptional regulation. Further work is required to identify other possible targets.

T22B3.1

The C. elegans gene T22B3.1, which was identified in the GR1401 (Is41) screen, is predicted to encode a polypeptide 359 amino acids in length that contains a BED Zn finger domain and that has no known homologs outside of nematodes. T22B3.1 has been given the locus name dpy-20, since dpy-20 null mutants have an abnormal, dumpy body morphology (Hosono et al., J Exp Zool 224:135 (1982); and Clark et al., Mol Gen Genet 247:367 (1995)). Based on its Zn finger domain, DPY-20 is predicted to function as a transcription factor.

MAP Kinases

Other classes of factors that regulate RNAi include the MAP kinase kinase, ZC449.3 and the MAP kinase kinase kinase, MTK-1. The established role of the ancient p38/MAP kinase pathway in the innate immune response to pathogens suggests that RNAi mechanisms may also be coupled to these stress and pathogen sensing pathways (Young and Dillin, Proc Natl Acad Sci USA 101:12781 (2004); Kim et al., Science 297:623 (2002)). In the GR1401 (Is41) screen, putative components of the mitogen-activated protein (MAP) kinase (MAPK) signaling pathway were identified that are required for RNAi: B0414.7 and ZC449.3. The MAP kinase signaling pathways control eukaryotic genes expression programs in response to extracellular signals. The MAPK signal transduction cascade forms a well-conserved cellular stress response system that governs cell survival and adaptation through changes in gene expression, cell proliferation, cell survival and death, and cell motility. In mammals, distinctly regulated groups of MAPKs—extracellular signal-regulated kinases (ERK), c-Jun N-terminal kinases (JNK)/stress-activated protein kinases (SAPK), and p38 proteins—are activated by multiple stimuli including UV or ionizing radiation, hyperosmolarity, oxidative stress, translation inhibitors, other signaling factors (such as the Ras/Raf pathway) and also cytokines such as interleukin 1, tumor necrosis factor α, and transforming growth factor β (Chang and Karin, Nature 410:37 (2001); and Chen et al., Mol Cell 7:227 (2001)). MAPK pathways are regulated through a conserved cascade of three protein kinases, with each pathway typically including two to four different MAPKs, two different MAPK kinases (MAPKK, MKK, or MEK), and a variable number of MAPKK/MEK kinases (MAPKKK or MEKK) (Marshall, Curr Opin Genet Dev 4:82 (1994); and Garrington and Johnson, Curr Opin Cell Biol 11:211 (1999)).

Each MAPKK can be activated by more than one MAPKKK through phosphorylation of conserved serine and/or threonine residues; this, combined with the highly pleiotropic output of these signaling cascades, reflects the ultimate complexity of MAPK signaling. Each MAPKKK phoshorylates and activates a specific MAPKK, which then activates a specific MAPK, and therefore each MAPKKK is presumed to confer responsiveness to distinct stimuli, though the molecular mechanism of this selectivity remains unclear (Chang and Karin, Nature 410:37 (2001)). MAPKs can also target co-activators and co-repressors and can affect nucleosomal structure by inducing histone modifications. Furthermore, multiple inputs into individual promoters can be elicited by MAPKs by targeting different components of the same co-regulatory complex or by triggering different events on the same transcription factor. Therefore, the MAPK signaling pathways represent a vast and complex network for integrating input from multiple external stimuli in order to effect balanced changes in gene expression.

B0414.7 and ZC449.3

The C. elegans gene B0414.7, which was identified in the GR1401 (Is41) screen, is predicted to have two different splice forms; the A isoform is 1418 amino acids in length, and the longer B isoform is 1420. Both isoforms contain a canonical eukaryotic serine/threonine kinase domain. B0414.7 was given the locus name mtk-1 because it encodes a close homolog of the human MAPKKK known as MTK1 (MAP three kinase) or MEKK4 (MAP or ERK kinase kinase) (BLASTP E-value of 1.6e-64 over 73.2% of the total polypeptide length). It is also homologous to the S. cerevisiae MAPKKK SSK22 (suppressor of sensor kinase) (BLASTP E-value of 5.8e-35 over 35.1% of the total polypeptide length).

The gene ZC449.3 is also predicted to have two different splice forms; the A isoform is 371 amino acids in length, and the longer B isoform is 411. And both isoforms of ZC449.3 also contain a eukaryotic serine/threonine kinase domain. This gene is predicted to encode a MAPKK that is a close homolog of the human dual-specificity MAPKK 4, also known as MKK4, MEK4, JNK kinase, or SAPK/ERK kinase 1 (SEK1) (BLASTP E-value of 1.9e-64 over 87.6% of the total polypeptide length), human MAPKK 3 (MKK3) (BLASTP E-value of 7.5e-56 over 81.2% of the total polypeptide length), and the S. cerevisiae MAPKK PBS2 (polymyxin B-sensitive), also known as SSK4 or HOG4 (high osmolarity glycerol) (BLASTP E-value of 1.8e-46 over 78.6% of the total polypeptide length). Both of these polypeptides are predicted to function in the MAP kinase intracellular signal transduction pathway.

Human MTK1 (and its mouse homolog MEKK4) is one of the MAPKKKs that act upstream of the JNK and p38 signaling pathways (Gerwins et al., J Biol Chem 272:8288 (1997)). Similarly, the yeast mtk-1 homolog SSK22 is a MAPKKK that has been shown to activate the HOG/p38 MAPK signaling pathway (Takekawa et al., EMBO J. 16:4973 (1997); and Posas and Saito, EMBO J. 17:1385 (1998)). Human SEK1 (MKK4), a human homolog of ZC449.3, is a MAPKK that also acts upstream of both the JNK and p38 signaling pathways (Sanchez et al., Nature 372: 794 (1994); Yan et al., Nature 372:798 (1994); and Guan et al., J Biol Chem 273:12901 (1998)). The other human homolog of ZC449.3 is MKK3, which specifically activates the p38 signaling pathway (Derijard et al., Science 267:682 (1995)). Its yeast homolog, PBS2, is also a MAPKK that specifically activates the HOG/p38 signaling pathway (Brewster et al., Science 259:1760 (1993); and Maeda et al., Nature 369:242 (1994)). Interestingly, in S. cerevisiae, the SSK22 MAPKKK and its close homolog SSK2 specifically activate the PBS2 MAPKK, which then activates the HOG/p38 signaling pathway (Maeda et al., Science 269:554 (1995); Posas et al., Cell 86:865 (1996)). There is also further evidence that the MTK1-MKK3/6-p38 pathway in humans is both structurally and functionally very similar to the yeast SSK2/SSK22-PBS2-HOG1 pathway. MTK1 complements the ssk2A ssk22 defect in yeast, and p38 can complement the osmosensitivity of the yeast hog1 mutation (Han et al., Science 265:808 (1994); and Takekawa et al., EMBO J. 16:4973 (1997)). Also, although the specific substrate of the HOG1 MAPK has not been defined, the substrate of it S. pombe homolog Spc1 was identified as Atf1, a transcription factor containing a bZIP domain which is homologous to ATF-2, a mammalian transcription factor that has been shown to be the substrate of p38 (Raingeaud et al. (1996); Shiozaki and Russell (1996); Wilkinson et al. (1996)).

This evidence strongly suggests that MTK-1 signals through ZC449.3 to activate the p38 signaling pathway in C. elegans. Furthermore, the independent identification of both genes as essential for RNAi strongly confirms their important role in the RNAi mechanism. Unfortunately, no published studies have specifically addressed the functions of C. elegans MTK-1 or ZC449.3. MAP kinase signaling pathways in C. elegans has been implicated in a wide variety of biological phenomena, including vulval cell differentiation (Wu and Han, Genes Dev 8:147 (1994); and Lackner et al., Genes Dev 8:160 (1994)), meiotic cell-cycle progression (Church et al., Development 121:2525 (1995)), embryonic polarity (Rocheleau et al., Cell 97:717 (1999); Meneghini et al., Nature 399:793 (1999); Ishitani et al., Nature 399:798 (1999); and Timm et al., EMBO J22:5090 (2003)), neuronal cell fare (Hirotsu et al., Nature 404:289 (2000); Sagasti et al., Cell 105:221 (2001); and Takeda and Ichijo, Genes Cells 7:1099 (2002)), environmental stress response (Koga et al., EMBO J. 19:5148 (2000); and Villanueva et al., EMBO J 20:5114 (2001)), and innate immunity (Kim et al. (2002); Aballay et al. (2003)), thereby underscoring the wide-range of its possible outputs in vivo. The mechanism by which MAP kinases affect RNAi is suggested by a recent study demonstrating a link between the HOG1 MAPK signaling pathway and RPD3-mediated histone deacetylation (De Nadal et al., Nature 427:370 (2004)). Where HOG1 was shown to physically interact with RPD3 both in vitro and in vivo. In addition, in response to stress HOG1 targets RPD3 to specific osmostress-responsive genes, leading to histone deacetylation, entry of RNA polymerase II, and induces gene expression. Also, recruitment of SIN3 is enhanced in response to activation of the ERK MAPK pathway in vivo (Yang et al., Mol Cell Biol 21:2802 (2001)). These connections are extremely tantalizing, given that C. elegans homologs of RPD3, SIN3, and other histone modifying enzymes were identified as required for RNAi. Without being tied to any particular theory, the fact that these two genes encode signaling molecules suggests that they are involved in modulating the output of the RNAi pathway, rather than being components directly involved in the core RNAi machinery.

Nuclear Import/Export Factors

The itinerary of endogenous dsRNAs, from transcription in the nucleus to processing by Dicer in the cytoplasm and subsequent target recognition, necessitates transport between subcellular compartments. For example, the nuclear export factor Exportin-5 is required for the transport of microRNA precursors from the nucleus to the cytoplasm (Yi et al., Genes Dev 17:3011 (2003); Lund et al., Science 303:95 (2004); Kim, V. N., Trends Cell Biol 14:156 (2004)). RNAi factors which by annotation are required for both nuclear export as well as import were identified, including the Ran GTPase exchange factor RCC1 (ran-3), the Ran GTPase Binding Protein 1 (npp-9), and two nucleoporins (npp-1 and npp-16)(Quimby and Dasso, Curr Opin Cell Biol 15:338 (2003)). In addition, the identification of nuclear import receptors of the importin-α and -β families (imb-2, imb-5, and ima-3) (Harel and Forbes, Mol Cell 16:319 (2004)) may represent a step in the silencing pathway whereby siRNAs generated in the cytoplasm are subsequently re-imported into the nucleus for transcriptional gene silencing. The recent discovery in S. pombe that transcriptional gene silencing at the centromere requires the Ran GTPase activating protein, RanGAP, further suggests an essential role for nucleocytoplasmic transport factors in RNAi (Kusano et al., Mol Biol Cell 15:4960 (2004)).

Y56A3A.17

The C. elegans gene Y56A3A.17 is predicted to encode a polypeptide with two different splice forms: the A isoform is 512 amino acids in length, and the shorter B isoform is 497 amino acids. Y56A3A.17 contains a Ran binding protein 1 domain and has been given the locus designation npp-16 because it is predicted to encode a member of the nuclear pore complex protein family. The Y56A3A.17 polypeptide is most homologous to a predicted nuclear pore protein from Drosophila (BLASTP E-value of 8.3e-14 over 63.7% of the total polypeptide length) and human nucleoporin (NUP50 or NPAP60L) (BLASTP E-value of 7.3e-12 over 48.0% of the total polypeptide length). Eukaryotic cells are defined by the presence of a nuclear envelope that separates transcription from translation; therefore, a system of macromolecular transport has developed that moves cargo between the cytoplasmic and nuclear compartments through nuclear pore complexes (NPCs), which are composed of proteins called nucleoporins (Ryan and Wente, Curr Opin Cell Biol 12:361 (2000)). Potential cargo must possess a nuclear localization signal (NLS) or nuclear export signal (NES) that can be recognized by soluble transport receptors, karyopherins, that are functionally divided into importins and exportins (Mattaj and Englmeier, Annu Rev Biochem 67:265 (1998); Pemberton et al., Curr Opin Cell Biol 10:392 (1998); and Görlich and Kutay, Annu Rev Cell Dev Biol 15:607 (1999)).

Classically, nuclear import is mediated by the importin-α:β transport receptor. And until recently, the nucleoporin Nup50/Npap60 was believed to be a structural component of the NPC that primarily regulated nuclear export (Smitherman et al., Mol Cell Biol 20:5631 (2000); and Guan et al., Mol Cell Biol 20:5619 (2000)). It has recently been shown that Npap60 is a soluble cofactor that functions as another subunit of the importin-α:β complex (Lindsay et al., Cell 110:349 (2002)). It also enhances the nuclear import of cargo possessing a basic NLS by associating directly with the import cargo-carrier complex and bringing it through the NPC. Based on its strong sequence similarity, C. elegans Y56A3A.17 would also be expected to act as a cofactor for nuclear import or export, but the exact nature of its cargo remains undetermined. One possibility is that Npap60 is required for the export of ribosomal RNAs, thereby connecting it to NCL-1 (as described above) or for the import of siRNAs or mRNAs from ribosomes, since it has also been demonstrated that these small RNAs are associated with ribosomes (Ishizuka et al., Genes Dev 16:2497 (2002); and Djikeng et al., RNA 7:1522 (2003)). Also, it was recently shown in Xenopus that Exportin-5 is required for the efficient nuclear export of mRNA precursors; therefore, another possibility is that Y56A3A.17 may be involved in the same process (Lund et al., Science 303:95 (2004)). In another embodiment, Npap60 may be required for the import of U snRNPs, since an RNAi-deficient loss-of-function phenotype for the spliceosome component U1A and for other snRNP-associated proteins was identified. With the exception of U6, which does not leave the nucleus, the biogenesis of U snRNPs requires the bi-directional transport of snRNA across the nuclear membrane; newly synthesized snRNAs exit to the cytoplasm after transcription, where they undergo processing and assembly with other snRNP core proteins prior to returning to the nucleus (Luhrnann et al., Biochim Biophys Acta 1087:265 (1990)). Studies have shown that the U1 snRNP proteins U1A and U1C are transported back to the nucleus separately from the rest of the snRNP and also that the central region of U1A contains an NLS responsible for the nuclear import of U1A in an ATP-dependent manner (Feeney and Zieve, J Cell Biol 110:871 (1990); and Kambach and Mattaj, J Cell Biol 118:11(1992)). A recent study has also shown specifically that U1A undergoes nuclear import via an importin-α:β and Ran-dependent pathway (Hieda et al., J Biol Chem 276:16824 (2001)). Therefore, RNAi of Npap60 would result in deficient nuclear import and resultant cytoplasmic accumulation of U1A, which would be expected to produce the same phenotype as loss-of-function U1A.

Other Polypeptides

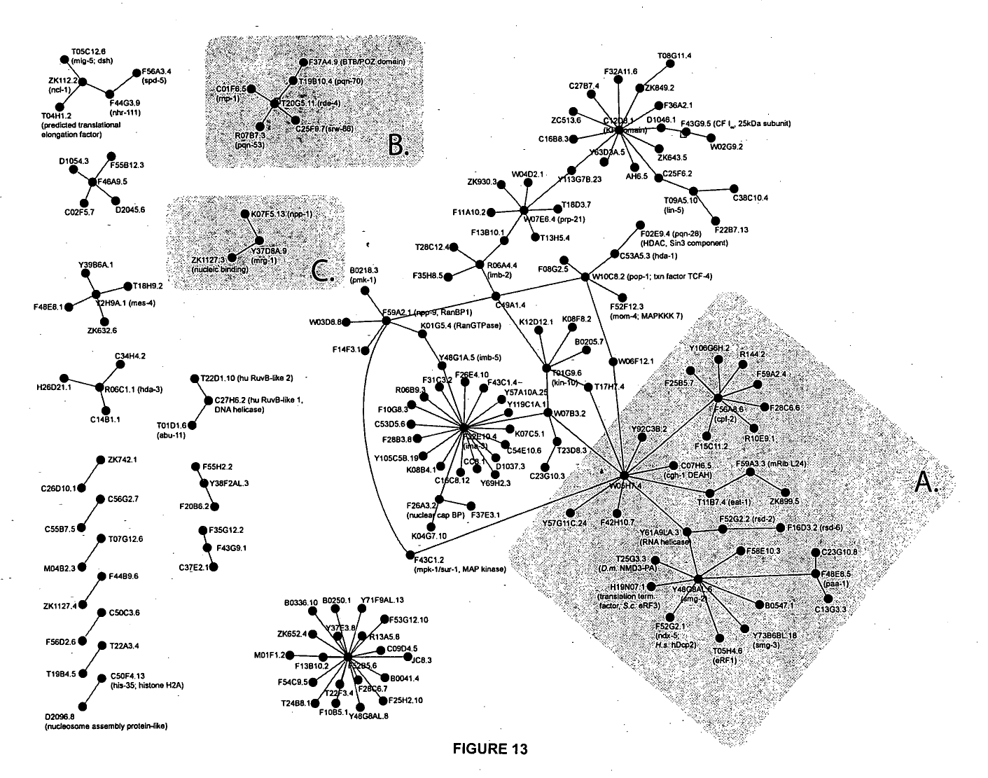

A number of other genes required for RNAi were identified in the GR1401 (Is41) screen that do not fall into any of the above functional classes: ZK1127.3, T01 C3.8, F37B112.4, and F52G2.2. A group of genes with no known function includes the new rde-5 gene identified by classical genetics (C. Mello, personal communication). Five members of this group have orthologs in humans, suggesting that their functions in RNAi are likely conserved (FIG. 10 and FIG. 13). Further study of these genes should provide insights into how RNAi and related pathways regulate gene expression in higher eukaryotes.

ZK1127.3

The C. elegans gene ZK1127.3 is predicted to encode a polypeptide 206 amino acids in length. It contains a nucleic acid-binding domain, and is homologous to S. cerevisiae EAF7 (Esa1-associated factor) (BLASTP E-value of 7.7e-05 over 40.3% of the total polypeptide length). Eaf7p is a putative subunit of the NuA4 histone acetyltransferase complex that specifically acetylates nucleosomal histone H4 (Allard et al., EMBO J. 18:5108 (1999)). N-terminal acetylation of lysines on histones is associated with transcriptional activity, which is presumed to occur due to reduced interactions of transcriptional repressors with nucleosomes and also due to enhanced binding of transcriptional activators or basal transcription factors (Imbalzano et al., Nature 370:481 (1994); Hecht et al., Cell 80:583 (1995); Vettese-Dadey et al., EMBO J. 15:2508 (1996)). H4 acetylation is the modification most frequently implicated in proposed mechanisms of chromatin modulation, which underscores the importance of H4-specific HATs in vivo.

Esa1p functions as a HAT that preferentially modifies histone H4 and is the catalytic subunit of NuA4 (Smith et al., Proc Natl Acad Sci USA 95:3561 (1998); and Clarke et al., Mol Cell Biol 19: 2515 (1999)). Eaf7p, the Esa1-interacting protein, is predicted to be another component of NuA4. Without being tied to any particular theory, given this homology, ZK1127.3 may be involved in acetylation of histones or may serve as link between a target DNA and a transcriptional activator complex, given its nucleic acid-binding domain. The former idea is particularly compelling given recent evidence demonstrating that the regulation of heterochromatic silencing via modification of histone H3 is dependent on components of the RNAi machinery (Volpe et al., Science 297:1833 (2002); and Hall et al., Science 297:2232 (2002)).

T01C3.8

The C. elegans gene T01C3.8, which was identified in the GR1401 (Is41) screen as required for RNAi, is predicted to encode a polypeptide 541 amino acids in length that contains a ribosome-inactivating protein (RIP) domain and is therefore predicted to be a negative regulator of polypeptide synthesis. T01C3.8 is one of 4 RIP-family proteins in the C. elegans genome. In contrast to NCL-1, which is predicted to encode a gene that affects polypeptide synthesis at the transcriptional level, T01C3.8 is predicted to act as a polypeptide synthesis inhibitor at the ribosome. It will be interesting to discover whether T01C3.8 and NCL-1 are able to effect complementary actions at different steps of the polypeptide synthesis pathway. The identification of two genes that regulate polypeptide synthesis as required for RNAi underscores the importance of polypeptide synthesis regulation in gene silencing phenomena.

F37B12.4

The C. elegans gene F37B12.4 is predicted to encode a polypeptide 1430 amino acids in length that contains a ubiquitin carboxyl-terminal hydrolases (UCHs) family 2 domain. F37B12.4 is homologous to UCHs from other organisms, including humans (BLASTP E-value of 1.6e-59 over 46.8% of the total polypeptide length), mice, Drosophila, and S. cerevisiae. In eukaryotes, ubiquitin (Ub) is activated by ATP and is then transferred to one of the 20-40 different Ub-carrier proteins (E2s). E2s work with Ub ligases (E3s) to catalyze the formation of a Ub chain on a polypeptide substrate, triggering its rapid degradation by the 26S proteasome. The exquisite specificity of this pathway lies in the E3s; mammalian cells contain hundreds of different E3s, each of which is specific for different polypeptide substrates (Goldberg, Nature 426:895 (2003)).

UCHs are essentially de-ubiquitinating enzymes that are thought to cleave polymeric Ub into monomers and to hydrolyze bonds between Ub and small adducts such as glutathione and cellular amines (Larsen et al., Biochemistry 37:3358 (1998)). Therefore, UCHs prevent polypeptides from being degraded by proteasomes. C. elegans has over 30 such polypeptides, and therefore they would be expected to have a certain degree of specificity for their targets. In C. elegans, most deficiencies in Ub-related polypeptides result in early lethality (Jones et al., Genome Biol 3:research 0002 (2002); and Kamath et al., Nature 421:231 (2003)). In humans, mutations causing partial loss of the catalytic activity of UCH have been associated with an abnormal build-up of polypeptides, as occurs in Alzheimer's and Parkinson's diseases, and oxidative damage to UCH has been shown to cause some sporadic cases (Leroy et al., Nature 395:451 (1998); and Choi et al., J Biol Chem 279:13256 (2004). Epub 2004 Jan. 13). Given the specificity of the Ub pathway for degrading individual polypeptides, the requirement of a particular UCH for RNAi is presumably the result of a defect in the degradation of that polypeptide; since the loss of a UCH would lead to increased ubiquitination of its target polypeptide, this would be expected to cause increased degradation of that polypeptide. However, the nature of such a polypeptide that may be targeted by this particular UCH remains unclear. F52G2.2

The C. elegans gene F52G2.2 is predicted to encode a polypeptide 1265 amino acids in length that does not have any known polypeptide domains and also does not have any known homologs outside of nematodes. Therefore, it role in RNAi remains a complete mystery, and further experiments are needed to suggest a putative molecular of cellular role for this gene.

The GR1401 (Is41) strain is capable of acting as a sensor of RNAi activity in vivo, an ability that should prove useful in future genetic screens. It is important to note that saturation of the RNAi mechanism does not appear to undermine the ability of this strain to identify genes required for RNAi, since genes that are known to be highly expressed, such as collagens, were not identified as essential for RNAi.

dsRNA corresponding to each RNAi essential gene was co-injected with pos-1, which has a highly-penetrant embryonic-lethal phenotype. Interestingly, levels of survival were directly proportional to levels of GFP expression, suggesting that the RNAi sensor activity of GR1401 (Is41) may be somewhat quantitative. In total, thirty-four genes were identified that reproducibly resulted in an RNAi-deficient phenotype upon silencing. Only 10 of these genes had previously been implicated in RNAi (Table 4).

TABLE 4

| | | | |
|---|---|---|---|
| Gene | Locus | Score | Predicted molecular function |

Known proteins

| | | | |
|---|---|---|---|
| K08H10.7 | rde-1 | 4.0 | PiWi/PAZ protein |
| T20G51.1 | rde-4 | 4.0 | dsRNA binding protein |
| K12H4.8 | dcr-1 | 4.0 | Bidentate ribonuclease that cleaves dsRNAs |
| ZK1098.8 | mut-7 | 3.0 | 3′-5′ exonuclease |
| B0379.3 | mut-16 | 4.0 | Novel protein |
| F15B10.2 | drh-1 | 2.4 | DEAD/DEAH box helicase |
| Y48G8AL.6 | smg-2 | 2.2 | RNA helicase required for nonsense-mediated decay of mRNA |
| F54F2.2 | zfp-1 | 3.2 | Leucine zipper, Zn-finger, PHD/LAP domain, chromatin-assoc |
| M04B2.3 | gfi-1 | 2.4 | Txn factor, chromatin-associated |
| Y2H9A.1 | mes-4 | 4.0 | SET RING finger, PHD finger, regulates chromatin germline |

PIWI/PAZ proteins

| | | |
|---|---|---|
| C04F12.1 | 4.0 | Piwi domain protein, homolog of AGO2 |
| K12B6.1 | 2.4 | Piwi/PAZ protein, homolog of AGO2 |
| RNA helicases | | |
| Y38A10A.6 | 2.8 | DEAD/DEAH-box helicase, homolog of human RHII/Gu |

RNA processing factors

| | | | |
|---|---|---|---|
| F43G9.5 | | 3.2 | Pre-mRNA cleavage factor |
| K08D10.4 | mp-2 | 3.1 | RNA-binding protein, homolog of human U1 snRNP protein A |
| (ZK1127.9/6) | | 3.2 | Txn elongation factor, similar to yeast U1 snRNP protein |
| W05H7.4 | | 2.4 | CCCH Zn-finger protein, similar to yeast mRNA nuclear exporter |
| T19B10.4 | pqn-70 | 2.0 | Similar to RNA-binding proteins in yeast and humans |

General chromatin factors

| | | |
|---|---|---|
| T23B12.1 | 2.3 | PHD-finger protein |
| M03C11.3 | 3.1 | Similar to mouse Acinus (apoptotic chromatin condenser) |
| Y71G10AL.1 | 2.6 | Similar to human nucleosomal binding protein 1 |

Histone modifying enzymes

| | | | |
|---|---|---|---|
| ZK1127.3 | | 2.4 | Similar to yeast EAF7, a component of the NuA4 HAT complex |
| F02E9.4 | pqn-28 | 2.9 | Histone deacetylase, ortholog of yeast SIN3 |
| R06C1.1 | hda-3 | 2.3 | Histone deacetylase, homolog of yeast RPD3 and human HP1 |

Transcription factors

| | | | |
|---|---|---|---|
| ZK112.2 | ncl-1 | 2.2 | B-box Zn-finger protein, regulates cell size and rRNA synthesis |
| T19B4.5 | | 3.0 | Protein similar to KSHV LANA (also Emb/Ste) |
| T22B3.1 | dpy-20 | 2.7 | BED Zn-finger protein required for normal body morphology |
| MAP kinases | | | |
| B0414.7 | mtk-1 | 3.0 | MAPKKK, homolog of human MTK1 and yeast SSK22 |
| ZC449.3 | | 2.8 | MAPKK, homolog of human SEK1 and yeast PBS2 |
| Other proteins | | | |
| T01C3.8 | | 4.0 | Ribosome-inactivating protein |
| Y56A3A.17 | npp-16 | 2.0 | Nuclear pore protein, Ran-binding protein |
| F37B12.4 | | 2.3 | Ubiquitin carboxyl-terminal hydrolase |
| F52G2.2 | | 4.0 | Novel protein |

Table 4 provides a list of 34 genes identified in the initial GR1401 (Is41) screen for genes required for RNAi. The genes are listed in classes based on their putative functions, which were identified by homology. Listed for each gene is the gene name, the locus name if available, the GFP score (an average score over all experiments), and the predicted molecular function of the genes based on homology. Polypeptides previously identified as functioning in RNAi have been grouped into a separate class. As shown above, 24 new genes were identified that are required for RNAi in C. elegans. Previously, only 29 genes had been identified as associated with RNAi in a combination of large-scale forward-genetic screens, biochemical assays, and small-scale RNAi-based screens (Table 5) (Tijsterman et al., Annu Rev Genet 36:489 (2002); Denli and Hannon, Trends Biochem Sci 28:196 (2003)).

| Mutant Phonotype | Gene | Domains | Putative function | Detected |
|---|---|---|---|---|
| RNAi defective | rde-1 | PiWi/PAZ | RNAi initiation | + |
| | rde-4 | dsRNA binding | RNAi initiation | + |
| | dcr-1 | Helicase, PAZ, RNA binding, RNaseIII | siRNA production | + |
| | mut-16 | | ? | + |
| | drh-1 | RNA helicase | ? | + |
| PTGS resistant in GL | ppw-2 | Piwi/PAZ | ? | |
| RNAi resistant in GL | ppw-1 | PiWi/PAZ | | |
| | mut-7 | 3′-5′ exonuclease | ? | + |
| | rde-2/mut-8 | | ? | |
| | mut-14 | RNA heilcase | ? | |
| | ego-1 | RdRP | RNAi amplification | |
| | mes-3 | | TGS | |
| | mes-4 | SET | TGS | + |
| | mes-6 | WD40 repeats | TGS | |
| | gfi-1 | YEATS | TGS | + |
| | zfp-1 | PHD finger | TGS | + |
| RNAi resistant in soma | nf-1 | RdRP | RNAi amplification | |
| Rapid recovery from RNAi | smg-2 | RNA helicase | RNAi maintenance | + |
| | smg-5 | PINc nuclectide binding | RNAi maintenance | |
| RNAi spreading defective | sid-f/rsd-8 | Transmembrane × 11 | RNAi spreading | |
| RNAi feeding resistant | rsd-2 | ENTH | RNAi spreading | |
| | rsd-3 | | RNAi spreading | |
| | rsd-6 | Tudor | RNAi spreading | |
| Hypersensitive to RNAi | nf-3 | RdRP | Negative regulator | |
| Hypersensitive to PTGS in soma | adr-1/adr-2 | Adenosine deaminase | Negative regulator | |
| Unknown | vig-1 | DNA binding, mRNA binding | RISC component | |
| | tsn-1 | Tudor, nuclease | RISC nuclease | |

Table 5 provides a list of the cloned genes that were identified as involved in the mechanism of RNAi in C. elegans, together with their mutant phenoypes, polypeptide domains, and putative functions. The final column of the table reflects whether a given locus was detected during the high-throughput screen.

In the screen, 10 of 24 known loci with an RNAi-deficient loss-of-function phenotype were identified (mut-16, mut-7, smg-2, rde-4, dcr-1, rde-1, drh-1, zfp-1, gfl-1, and mes-4) (Tijsterman et al., Annu Rev Genet 36:489 (2002); Tabara et al., Cell 109:861 (2002); Dudley et al., Proc Natl Acad Sci USA 99:4191 (2002); Denli and Hannon, Trends Biochem Sci 28:196 (2003)). The GR1401 (Is41) screen successfully detected 5 loci that are generally required for RNAi in both the germline and soma, but only 5 of 17 loci that are required for RNAi in specific tissues or under specific conditions. This suggests that the genes identified in the GR1401 (Is41) screen are those that are more generally required for RNAi. These genes are likely to be required for RNAi in other organisms, as well. Of the twenty-four new genes identified as P₀ viable, twenty-one encode polypeptides with significant homologies to polypeptide from other non-nematode organisms, and most have homologs in multiple organisms, including humans. This high level of conservation underscores the functional importance of these genes in vivo.

Two new Piwi/PAZ family genes were identified as required for RNA1, C04F12.1 and K12B6.1. These polypeptides are likely to be components of the RNAi core machinery, since several other members of the Piwi/PAZ family have been implicated in RNAi-related phenomena in organisms, including S. pombe, Arabidopsis, C. elegans, Drosophila, and humans, and since no other cellular roles have been ascribed to these polypeptides (Cerutti et al., Trends Biochem Sci 25:481 (2000); Carmell et al., Genes Dev 16:2733 (2002); and Sasaki et al., Genomics 82:323 (2003)). The recent identification of the Piwi domain as a Dicer-recognition motif and the PAZ domain as an siRNA binding motif supports the notion that these are modular polypeptides that serve as general adapters in the RISC complex (Lingel et al., Nature 426:465 (2003); Yan et al., Nature 426:468 (2003); Song et al., Nat Struct Biol 10:1026 (2003); and Tahbaz et al., supra). By this model, it is possible that Piwi/PAZ polypeptides transiently associate with Dicer via the Piwi domain in order to facilitate loading of siRNAs from Dicer onto the PAZ domain. The ‘loaded’ Piwi/PAZ polypeptide may then use the specificity of the siRNA to direct the action of RISC.

Y38A10A.6, a new DEAD/DEAH-box helicase was identified required for RNAi. Similar molecules have previously been implicated in the RNAi machinery, although some are predicted to act as part of the core mechanism (DRH-1 and MUT-14), whereas others are thought to act more peripherally (SMG-2) (Domeier et al., Science 289:1928 (2000); Tijsterman et al., Science 295:694 (2002); and Tabara et al., Cell 109:861 (2002)). In principle, RNA helicases may act at many different steps of the silencing pathway, including by mediating the ATP-dependent unwinding of siRNAs associated with RISC, by changing the conformation of the long dsRNAs that initiate the PTGS process, or by binding RNAs to facilitate proximity in multistep reactions. DEAD/DEAH-box helicases have also recently been implicated in other processes tangentially related to RNAi, including pre-mRNA splicing, nuclear export of mRNAs, and ribosome biogenesis, and they have also been shown to act as ‘RNPases’ that use chemical energy to remodel the interactions of RNA and polypeptides. Y38A10A.6 may theoretically perform any of these functions (Chen et al., Chem Rev 101:2449 (2001); Jankowsky et al., Science 291:121 (2001); Schwer, Nat Struct Biol 8:113 (2001); and Reed and Hurt, Cell 108:523 (2002)). The human homolog of Y38A10A.6 transduces signals through the JNK MAP kinase pathway (Westermarck et al., EMBO J21:451(2001)). The functional diversity of DEAD/DEAH-box helicases paints a confusing picture for the possible roles of this polypeptide in vivo. However, the possibility remains that it may be part of the core RNAi machinery

The GR1401 (Is41) screen identified six RNA processing factors that are required for RNAi (F43G9.5, K08D10.4, ZK1127.6/ZK1127.9, W05H7.4, and T19B10.4). RNA metabolism has previously been associated with RNAi at the level of nonsense-mediated decay and RNA-editing by ADARs (adenosine deaminase that acts on RNA) (Bass, Cell 101:235 (2000); Domeier et al., Science 289:1928 (2000); and Scadden and Smith, EMBO Rep 2:1107 (2001)). However, the six factors identified herein are likely to be part of the the core RNA processing machinery implicated in the splicing, polyadenylation, stabilization, and nuclear export steps of mRNA synthesis. The identification of polypeptides required at successive steps in this process highlights its importance in RNAi. It is likely that the RNAi pathway interacts with the RNA processing machinery more directly than was previously realized. In this regard, RNAi may be involved in other forms of post-transcriptional regulation in which it affects the processing of nascent transcripts in the nucleus in addition to its presumed cytoplasmic degradation of fully-processed mRNAs.

Five putative chromatin remodeling factors that are required for RNAi were identified. A number of other chromatin factors have previously been implicated in RNAi in C. elegans, and much recent work has begun to establish connections between these two processes, although the mechanistic details are still unclear (Dudley et al., Proc Natl Acad Sci USA 99:4191 (2002); and Grewal and Moazed, Science 301:798 (2003)). T23B12.1 is likely to be involved in chromatin remodeling given that it contains a PHD finger domain, which is present in other genes implicated in chromatin remodeling. M03C11.3 and Y71G10AL.1 are most homologous to mammalian polypeptides known to be chromatin remodeling factors, Acinus and NBSP1, respectively. In addition, the discovery of the histone deacetylases, F02E9.4 and R06C 1.1, as required for RNAi, is particularly compelling, since good homologs for these polypeptides exist in yeast that have been shown to interact as part of the SIN3 transcriptional repressor complex (Kasten et al., Mol Cell Biol 17:4852 (1997); reviewed in Jenuwein and Allis, Science 293:1074 (2001)). Interestingly, the other factor identified, ZK1127.3, is predicted to be part of a histone acetyltransferase complex, which in principle has an opposite biochemical activity to the HDACs described above. It remains to be determined whether these factors are involved in establishing a baseline balance of transcriptional activation at particular loci. The identification of these genes as essential components of the RNAi mechanism further substantiates the connections between transcriptional and post-transcriptional gene silencing.

GR1401 (Is41) screen also identified three specific transcription factors required for RNAi. Interestingly, two of these, NCL-1 and T19B4.5, are predicted to act as global regulators of transcription. NCL-1 appears to control a genetic program responsible for regulating cell size and rRNA synthesis (Frank and Roth, J Cell Biol 140:1321 (1998)). While NCL-1 is predicted to be a DNA-binding transcription factor, due to the presence of a Zn finger, NCL-1 might also act as RNA-binding polypeptide as its Drosophila ortholog Brat does. Brat regulates specific genes at the post-transcriptional level by binding to their 3′-UTRs and repressing their translation. Therefore, NCL-1 may be an example of a polypeptide that is regulated by the RNAi machinery to increase or decrease post-transcriptional silencing of particular mRNAs by a mechanism roughly analogous to that employed by mRNAs. T19B4.5 is homologous to a highly promiscuous viral antigen that is known to modulate cellular gene expression through a variety of intermediaries (Verma and Robertson (2003)). Therefore, this polypeptide may act as a general co-factor for transcription, and its regulation may be important for gene regulation at the pre-transcriptional level as a complement to post-transcriptional silencing by RNAi.

The GR1401 (Is41) screen also identified two members of the MAP kinase signaling pathway, B0414.7 and ZC449.3, which have homologs in yeast and humans that are known to interact via the p38 signaling pathway (Han et al., Science 265:808 (1994); Posas et al., Cell 86:865 (1996); and Takekawa et al., EMBO J. 16:4973 (1997)). Although this pathway has pleiotropic outputs, two of its putative functions are particularly interesting in this context. First, the yeast p38, Hog1, has been shown to target the Rpd3p deacetylase to specific genes, leading to histone deacetylation and activation of gene expression (De Nadal et al., supra, 2004). Also, recruitment of mSin3A is enhanced by activation in response to the ERK MAP kinase pathway (Yang et al., Mol Cell Biol 21:2802 (2001)). Since RPD3 and SIN3 are known to associate, and since it was also discovered that both of these polypeptides are required for RNAi, it may be that they are the relevant downstream effectors of this signaling cascade. Second, the p38 pathway has been shown to regulate innate immunity in C. elegans (Kim et al., Science 297:623 (2002); Aballay et al., Curr Biol 13:47 (2003)). This finding is particularly interesting, since RNAi is thought to act as an innate antiviral defense mechanism, and therefore p38 may act as a master regulatory pathway that governs both cellular and ‘genetic’ immunity (Vaucheret et al., Plant J 16:651 (1998); and Tijsternan et al., supra (2002)).

Among the other factors discovered that are essential for RNAi, one of the most interesting is NPP-16, a nuclear pore protein with a human homolog, Npap 60, that acts as a soluble co-factor that associates with the importin α:β complex and shuttles polypeptides with an appropriate nuclear localization signal through the nuclear pore complex (Lindsay et al., Cell 110:349 (2002)). Without being tied to any particular theory, it is possible that Npap60 is required for the import of siRNAs or mRNAs from ribosomes when they are associated with certain co-factors (Ishizuka et al., supra (2002); Djikeng et al. (2003)). In another embodiment, Npap60 may be involved in the nuclear export of mRNA precursors, as was recently shown for Xenopus Exportin-5 (Lund et al., Science 303:95 (2004)). Perhaps the most likely role for Npap60 is in the import of U snRNPs; U1A, which was also identified as required for RNAi, undergoes nuclear import via an importin α:β and Ran-dependent pathway (Hieda et al., J Biol Chem 276:16824 (2001)). Therefore, RNAi of Npap60 may result in deficient nuclear import and the cytoplasmic accumulation of U1A, which would be expected to produce the same RNAi-deficient phenotype as loss of U1A.

As described above, the new factors identified herein are required for RNAi and are predicted to functionally interact with each other based on their homologies.

Certain polypeptides, such as RNP-2, are predicted to act as nexuses of functional interaction (FIG. 5). Based on homology, seven putative functional interactions are indicated for RNP-2, which is a protein component of the spliceosomal U1 snRNP. Other putative spliceosomal proteins also have a large number of interactions. This highlights the importance of the spliceosome in RNAi. The MAP kinase signaling components, MTK-1 and ZC449.3, also share many interactions. Finally, the DEAH/DEAD-box helicase, Y38A10A.6, also acts as a nexus of interactions.

It is likely that many of the genes identified herein act as modulators of the RNAi pathway, rather than as core components. Indeed, only 3 of the polypeptides fall into classes of genes known to function in the core machinery of RNAi, the two Piwi/PAZ polypeptides and the DEAH/DEAD-box helicase.

Steady-state levels of siRNAs produced in response to injection of dsRNAs are assayed by feeding each of the clones to worms that are subsequently injected with dsRNA corresponding to an endogenous gene. Northern blot analysis of siRNAs is then performed to determine levels of siRNAs generated against the targeted endogenous gene.

GFP translational fusions under the control of the endogenous promoters of RNAi essential genes are constructed to assay for the temporal and spatial localization of polypeptides encoded by RNAi essential genes. Co-localization of candidate polypeptides and known RNAi machinery proteins may be performed using double labeling strategies with GFP and RFP. These strains may also be used in RNAi-based screens to identify other genes that modulate their activity. Genetic mutants of genes identified herein are generated to identify their organismal, cellular, and molecular phenotypes. Northern blots for siRNAs and mRNAs or transgenic rescue experiments to characterize the domain structure and activities of the corresponding genes are also carried out.

Genes identified herein may be required for the processing of mRNAs. lin-4 and let-7 are mRNAs that temporally regulate seam-cell number and differentiation during larval development via the heterochronic pathway (Lee et al. (1993); and Reinhart et al., Science 297:1831 (2000)). The RNAi and mRNA pathways are known to share some common machinery, so it is possible that some of the genes identified as required for RNAi also function in heterochronic and/or developmental processes controlled by mRNAs (Grishok et al., Cell 106:23 (2001)). Since suppression of RNAi in GR1401 (Is41) results in seam-cell GFP expression, the roles of genes identified herein can be assayed for their effect on the heterochronic pathway by counting seam cell number. Those genes whose downregulation causes an alteration in the number of seam cells are identified as functioning in a heterochronic pathway.

Northern blots have been carried out to assay production of known mRNAs in worms following RNAi by feeding to silence the candidate genes. Candidate genes have been tested for genetic interactions with known mutations in the mRNA processing machinery; for example, RNAi against putative heterochronic genes in MG279, a weak let-7 allele, would allow for detection of enhancement or suppression of heterochronic phenotypes.

Screen to Identify RNAi Essential Genes that are Also Required for Embryonic Development

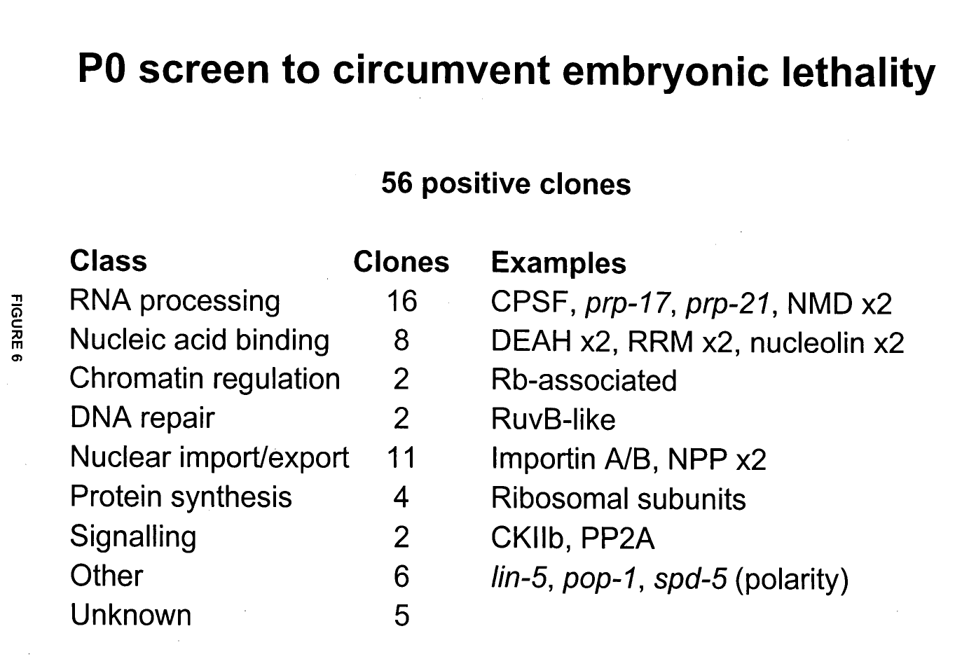

In the screen described above, approximately 1000 putative RNAi essential genes were also embryonic lethal. Because this screen was carried out in the F1 generation, these genes could not be identified. To allow for the identification of such genes, a second screen was carried out which modified the screen described above by evaluating the REG phenotype in the Po worms. During the L1 stage, GR1401 (Is41) worms (Pos) were transferred to bacteria expressing dsRNA. The worms were allowed to develop to the L4 stage, and then were scored for the REG phenotype. This screen identified 56 new genes that fall into the following eight classes: RNA processing, nucleic acid binding, chromatin regulation, DNA repair, trafficking polypeptide synthesis, signalling, unknown, and other (FIG. 6). The 56 genes identified in this screen are listed in Table 6 by C. elegans cosmid and open reading frame number.

TABLE 6

| | |
|---|---|
| GR1401 | |
| (Is41) P0 | |
| Functional | |
| Classes | Gene Function and Homology |

RNA processing (16)

| | |
|---|---|
| C31H1.8 | Weak: S. cerevisiae SGD:YGR159C. Nucleolar protein that binds nuclear localization |
| | sequences, required for pre-rRNA processing and ribosome biogenesis. 0.00058, 13.4% |
| C55B7.5 | hu: RNA polymerase II subunit 5-mediating protein. 1.1e-10, 92.4% |
| E02H1.1 | RNA methyltransferase |
| F09G2.4 | Cleavage and polyadenylation specificity factor, 100 kDa subunit (CPSFs 100 kDa subunit). |
| F26A3.2 | RNA cap binding protein |
| F43G9.1 | contains similarity to Saccharomyces cerevisiae Nucleolar protein that binds nuclear |
| | localization sequences, required for pre-rRNA processing and ribosome biogenesis; |
| | SGD:YGR159C |
| F43G9.12 | Contains similarity to Saccharomyces cerevisiae Nucleolar protein that binds nuclear |
| | localization sequences, required for pre-rRNA processing and ribosome biogenesis |
| F46A9.5 | skr-1. The skr-1 gene encodes a homolog of Skp1 in S. cerevisiae that is required for the |
| | restraint of cell proliferation, progression through the pachytene stage of meiosis, and the |
| | formation of bivalent chromosomes at diakinesis. Similar to transcriptional elongation factor |
| F49D11.1 | Hu: ENSEMBL:ENSP00000304370; Pre-mRNA splicing factor PRP17; 1.8e-166, 99.5% |
| R06F6.1 | cdl-1 encodes a homolog of human hairpin (stem-loop) binding proteins (HBP/SLBP) that |
| | bind to the hairpin (stem-loop) structure in the 3′ untranslated region (3′ UTR) of histone |
| | mRNAs, and thus promote histone pre-mRNA processing and translation of mature histone |
| | mRNA; CDL-1 is required for normally high levels of histone gene expression, normal cell |
| | division during late larval development, embryonic viability, normal vulval morphogenesis, |
| | normally rapid apoptosis, and fertility; CDL-1 binds to the stem-loop structure in the 3′ UTR |
| | of core-histone mRNA; the cdl-1 promoter is most active in dividing cells during |
| | embryogenesis and postembryonic development; both CDL-1 and human HBP contain a |
| | minimal RNA-binding domain (RBD) of roughly 73 amino acids that has no similarity with |
| | other known RNA-binding motifs |
| T25G3.3 | Yeast nonsense-mediated mRNA decay protein like; Flybase gene name is Nmd3-PA; hu is |
| | CGI-07 protein. Highly conserved. |
| W06E11.1 | hu: Splice isoform 1 of Q9NVU0 DNA-directed RNA polymerases III 80 kDa polypeptide |
| | 6.8 e-19, 93.4% |
| W07E6.4 | prp-21. Member of the yeast PRP (splicing factor) related gene class |
| Y71F9B.4 | snr-7. Member of the Small Nuclear Ribonucleoprotein gene class |
| ZK1127.5 | contains similarity to Pfam domains PF05189 (RNA 3′-terminal phosphate cyclase (RTC), |
| | insert domain), PF01137 (RNA 3′-terminal phosphate cyclase) |
| ZK1127.7 | hu: Splice isoform 1 of P11388 DNA topoisomerase II, alpha isozyme. 8.69e-229, 94% |

Nucleic acid binding (8)

| | |
|---|---|
| C06E1.10 | The C06E1.10 gene encodes a DEAH helicase orthologous to the Drosophila KURZ, the |
| | human KIAA1517, and the S. cerevisiae ECM16 proteins. |
| C16A3.4 | contains similarity to Pfam domain PF00096 (Zinc finger, C2H2 type); weak similarity to rat |
| | nucleolin |
| D2089.1 | C. elegans RSP-7 protein; contains similarity to Pfam domain PF00076 (RNA recognition |
| | motif. (aka RRM, RBD, or RNP domain)) |
| F11A10.3 | Ring finger protein |
| F56A8.6 | C. elegans CPF-2 protein; contains similarity to Pfam domain PF00076 (RNA recognition |
| | motif. (aka RRM, RBD, or RNP domain)) |
| F56D2.6 | The F56D2.6 gene encodes a DEAH helicase orthologous to the Drosophila CG11107, the |
| | human DDX15, and the S. cerevisiae PRP43 proteins. |
| T08G11.4 | contains similarity to Interpro domain IPR000051 (SAM (and some other nucleotide) binding |
| | motif) |
| T23D8.3 | RNA binding protein. Similar to nucleolin (hu 2.3 e-32, 96.6%) |

Chromatin regulation (2)

| | |
|---|---|
| K07A1.11 | rba-1. Chromatin assembly factor 1 subunit C (CAF-1 subunit C) (Chromatinsassembly |
| | factor I p48 subunit) (CAF-I 48 kDa subunit) (CAF-Ip48)s(Retinoblastoma binding protein |
| | p48) (Retinoblastoma-binding proteins4) (RBBP-4). |
| T12D8.1 | Mm: Myeloid/lymphoid or mixed-lineage leukemia protein 3 homolog (Histone-slysine N- |
| | methyltransferase, H3 lysine-4 specific MLL3) (EC 2.1.1.43). 2.9 e-156, 57.1%. |

DNA repair (2)

| | |
|---|---|
| T22D1.10 | RuvB-like |
| C27H6.2 | Hu: RuvB-like 1. 6.5 e-137, 88.9% |

Trafficking (11)

| | |
|---|---|
| C07E3.2 | Yeast ortholog (also has hu): Protein that forms a nucleolar complex with Mak21p that binds |
| | to 90S and 66S pre-ribosomes, as well as a nuclear complex with Noc3p that binds to 66S |
| | pre-ribosomes; both complexes mediate intranuclear transport of ribosomal precursors 5.7 e- |
| | 44, 94%. |
| C26D10.1 | ran-3. Member of the associated with RAN (nuclear import/export) function gene class |
| D2096.8 | contains similarity to Pfam domain PF00956 (Nucleosome assembly protein (NAP)) |
| F59A2.1 | npp-9. Member of the Nuclear Pore complex Protein gene class |
| F32E10.4 | Member of the IMportin Alpha family gene class |
| K07F5.13a | C. elegans NPP-1 protein; contains similarity to Rattus norvegicus Nuclear pore complex |
| | protein Nup 153 (Nucleoporin Nup153) (153 kDasnucleoporin).; SW:N153_RAT |
| R06A4.4a | C. elegans IMB-2 protein; contains similarity to Pfam domain PF03810 (Importin-beta N- |
| | terminal domain) |
| W04C9.1 | C. elegans HAF-4 protein; contains similarity to Pfam domains PF00664 (ABC transporter |
| | transmembrane region), PF00005 (ABC transporters) |
| Y38F2AL.3 | vhla-11. Member of the Vacuolar H ATPase gene class |
| Y48G1A.5 | imb-5 encodes an importin-beta-like protein orthologous to mammalian CAS proteins |
| | (cellular apoptosis susceptibility) and Saccharomyces cerevisiae CSE1 (chromosome |
| | segregation 1); IMB-5 is predicted to function in nuclear transport of proteins required for |
| | mitotic progression or apoptosis as well as in re-export of importin-alpha, a nuclear import |
| | protein; in C. elegans, IMB-5 is essential for embryogenesis and required for normal |
| | pronuclear envelope dynamics, and may also play a role in vulval morphogenesis. |
| ZK1127.4 | contains similarity to Saccharomyces cerevisiae vacuole import and degradation (VID); TOR |
| | inhibitor (TIN); SGD:YGL227W |

Protein synthesis (4)

| | |
|---|---|
| C15F1.4 | C. elegans PPP-1 protein, contains similarity to Homo sapiens GDP-mannose |
| | pyrophosphorylase A and translation initiation factor; ENSEMBL:ENSP00000315925 |
| F52B5.6 | rpl-25.2 encodes a large ribosomal subunit L23a protein |
| F59A3.3 | contains similarity to Pfam domain PF00467 (Ribosomal protein L24) |
| Y61A9LA.10 | Hu: Ribosome biogensis BMS1 homoog. 1.5e-142, 64.6% |

Signalling (2)

| | |
|---|---|
| F48E8.5 | paa-1 encodes a homolog of PR65, the structural subunit of protein phosphatase 2A (PP2A); |
| | PAA-1 protein is bound by SMG-5; PAA-1 is required in mass RNAi assays for embryonic |
| | viability, fertility, and cuticular integrity |
| T01G9.6a | kin-10. Ortholog of Human Casein kinase II beta chain 2.1e-94, 83.4% |

Unknown (5)

| | |
|---|---|
| C06A5.1 | Contains similarity to Pan troglodytes Breast cancer 2 (Fragment).; TR:Q8HZQ1 |
| C29E4.2 | Unknown function |
| K12H4.5 | Unknown function |
| W04A4.5 | Unknown function |
| Y110A7A.19 | contains similarity to Interpro domain IPR008941 (TPR-like) |

Other (6)

| | |
|---|---|
| F26E4.4 | contains similarity to Gallus gallus Collagen alpha 2(I) chain precursor (Fragments).; |
| | SW:CA21_CHICK |
| 43G9.10 | Microfibrillar-associated protein |
| F54H12.1 | C. elegans ACO-2 protein; contains similarity to Pfam domains PF00694 (Aconitase C- |
| | terminal domain), PF00330 (Aconitase family (aconitate hydratase)) |
| F56A3.4 | C. elegans SPD-5 protein; contains similarity to Saccharomyces cerevisiae Synaptonemal |
| | complex (SC) protein that connects homologous chromosomes partially during zygotene and |
| | entirely during pachytene; potential Cdc28p substrate; SGD:YDR285W |
| T09A5.10 | C. elegans LIN-5 protein; contains similarity to Saccharomyces cerevisiae EH domain protein |
| | involved in endocytosis; SGD:YBL047C |
| W10C8.2 | pop-1. The pop-1 gene encodes a homolog of Drosophila PANGOLIN; POP-1 is required for |
| | proper polarity in both early embryos and postembryonic development. |

These experiments were carried out using methods described below.
Strains and Clones

All C. elegans experiments were performed in a standard wild-type (N2) background unless otherwise indicated. Standard methods were used for culturing C. elegans on NGM (nematode growth medium) and NGM-derivative media, except where specified (Brenner, Genetics 77:711974; Sulston and Horvitz, Dev Biol 56:110 (1977); and Lewis and Fleming, Methods Cell Biol 48:4 (1995)).

To create the GR1401 (Is41) strain, a plasmid expressing a hairpin GFP under the wrt-2 seam cell promoter, pwrt-2::GFP hairpin, was constructed. The loop of the GFP hairpin was first cloned by amplifying a 499-bp segment of GST from pGEX-2TK (Amersham) using the following oligonucleotides: 5′ GSTloop-Xma I (5′-GGGCCTTgtgcaacccGGGcgacttc-3′) and 3′ GSTloop-Nhe I (5′-ctaattttgggCTAgcatccAGGCAC-3′). The GST PCR product was digested and inserted into the Xma I and Nhe I sites in the pPD49.26 vector (a gift from A. Fire), resulting in the pPD49.26:GSTloop plasmid. The 952-bp forward version of GFP was then PCR-amplified using the 5′ GFP Forward hp-Xba I (5′-CTGCTCCAAtctAGAAGCGTAAGGTA-3) and 3′ GFP Forward hp-Xma I (5′-GGTAATGGTAGCGcCCGGgGCTCAG-3′) oligonucleotides, while the 952-bp reverse-complement version of GFP for the second half of the GFP hairpin was PCR-amplified using the 5′ GFP Rev hp-Sac I (5′-CTGCTCCAAAGAAGgAGCtcAAGGTA-3′) and 3′ GFP Rev hp-Nhe I (5′-GGTAATGGTAGCtAgCGGCGCTCAG-3) oligonucleotides. The front and back halves of the GFP hairpin were inserted to flank the GST loop in the pPD49.26:GSTloop plasmid using the Xba I/Xma I and Sac I/Nhe I sites, respectively, resulting in the p49.26:GFPhairpin plasmid. Finally, a 1.8-kb upstream region of the wrt-2 gene was amplified using the 5′ wrt-2 HinD III (5′-CAAAAATCTCCCAAAgCTtTCGATATG-3′) and 3′ wrt-2 Pst I (5′-CGGCGTATGCTGCAGAGAAACAATTGG-3) oligonucleotides and was introduced into the p49.26:GFPhairpin plasmid by a HinD III/Pst I ligation, thus completing the pwrt-2::GFPhairpin construct.

The starting C. elegans strain for GR1401 (Is41) was JR672, which expresses a nuclear-localized GFP marker in the seam cells. This reagent was provided by Joel Rothman (Koh and Rothman, supra (2001)). Both pwrt-2::GFPhairpin (20 ng/μl) and a co-injection marker (ttx-3::RFP at 10 ng/μl) were injected into JR672. Subsequent transgenic worms, as scored by the ttx-3::RFP in the extrachromosomal array, showed a substantial reduction in the expression of GFP in the nuclei of seam cells, suggesting that the GFP hairpin in the seam cells was silencing GFP expression. The transgenic worms carrying the Ex[pwrt-2::GFPhairpin; ttx-3::RFP] array were isolated and were subjected to UV-irradiation to integrate the array. The resulting integrated strain, GR1401 (Is41) [Is (pwrt-2::GFPhairpin; ttx-3::RFP) in the JR672 background], was backcrossed into the N2 wild-type strain by following the ttx-3::RFP marker four times and then into the JR672 once.

RNAi Libraries

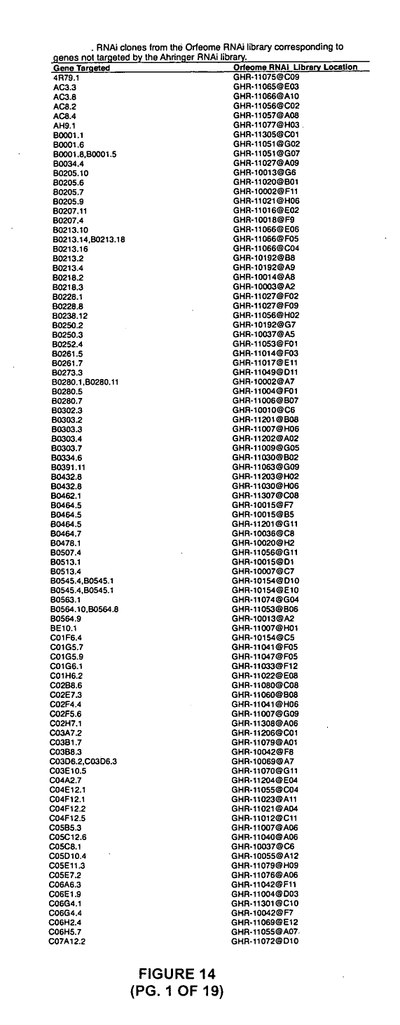

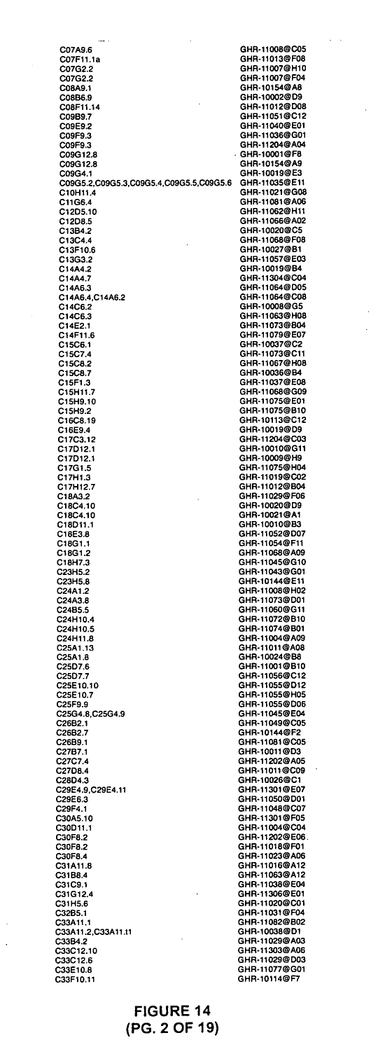

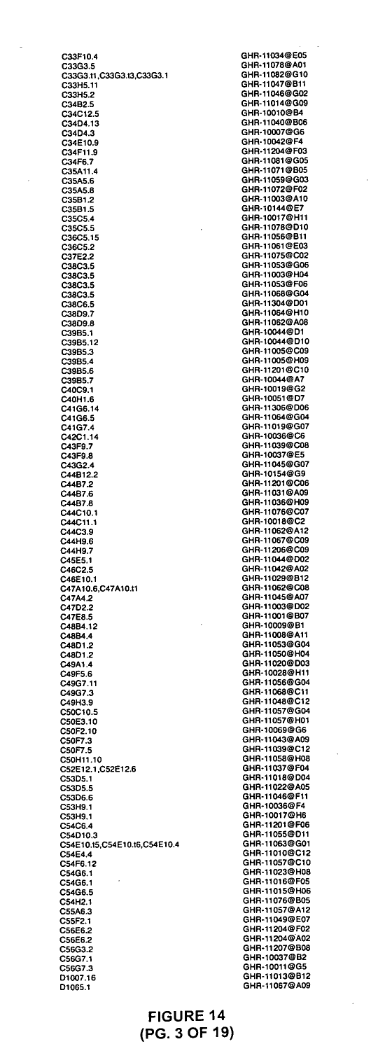

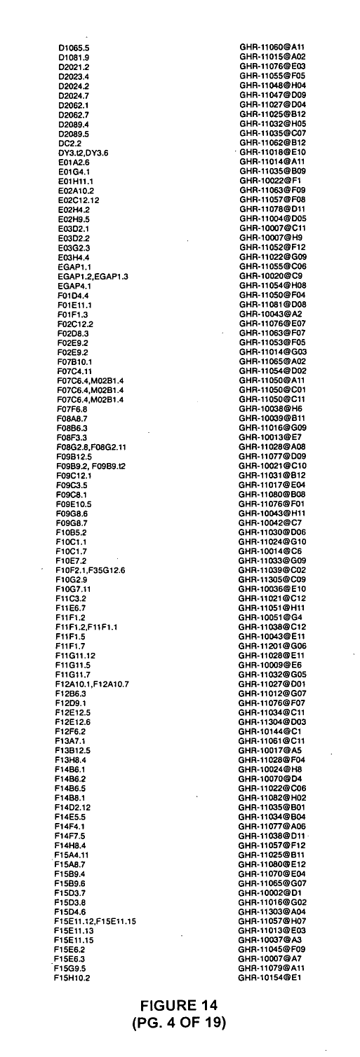

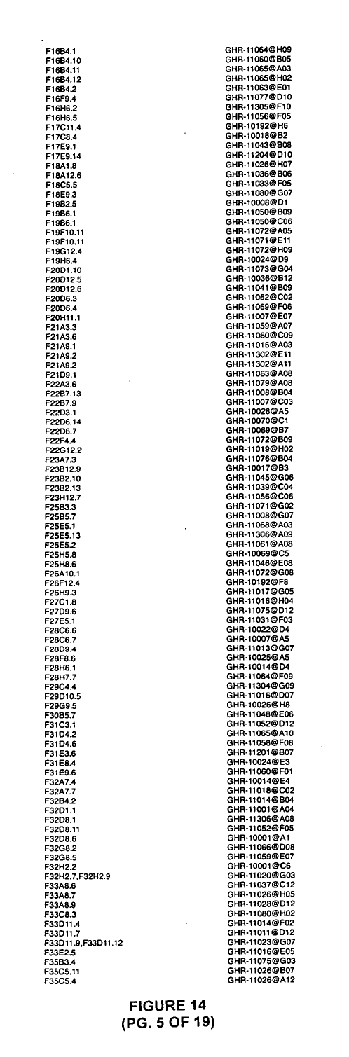

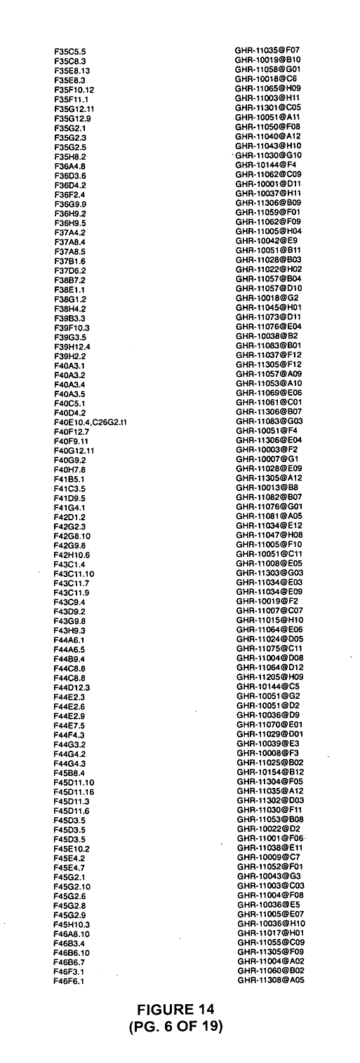

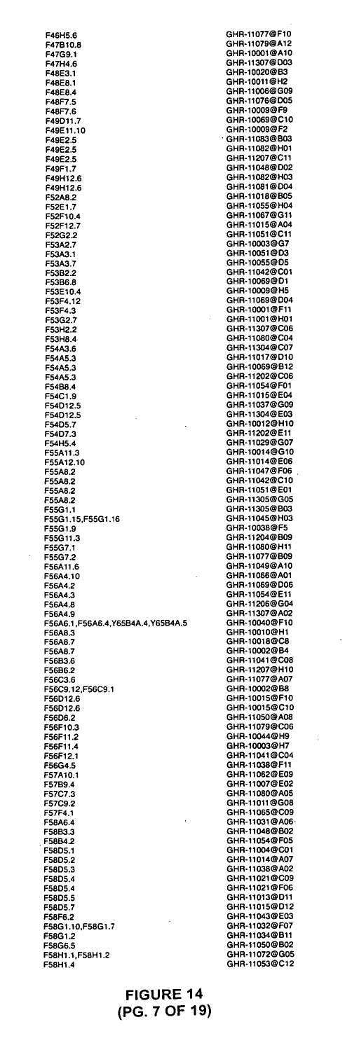

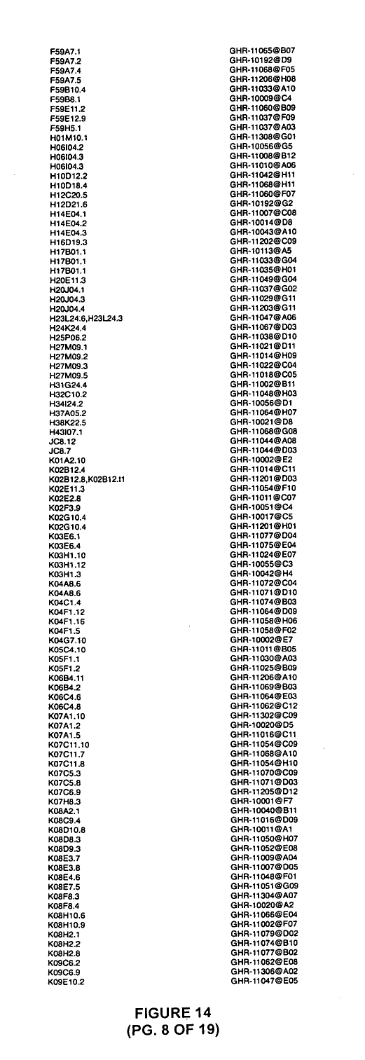

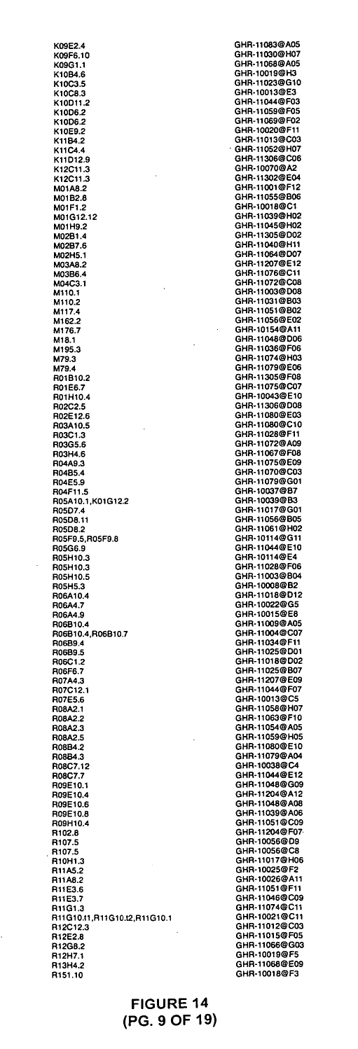

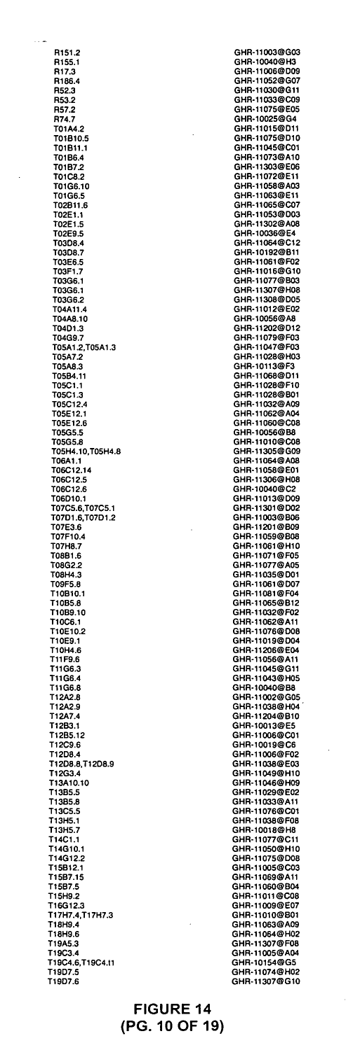

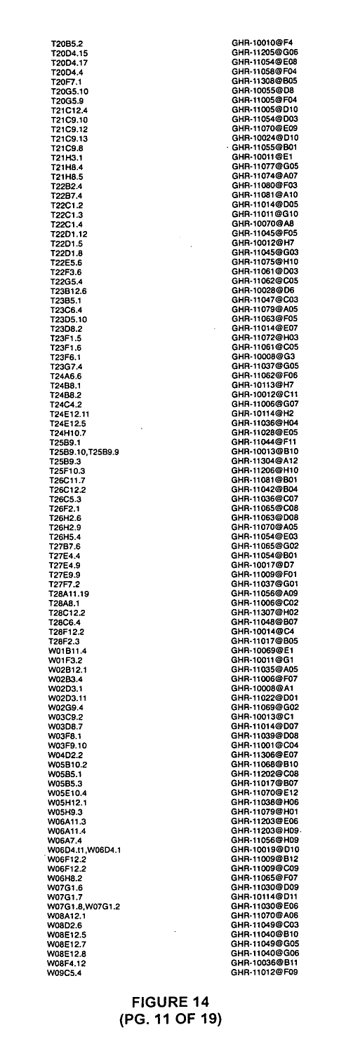

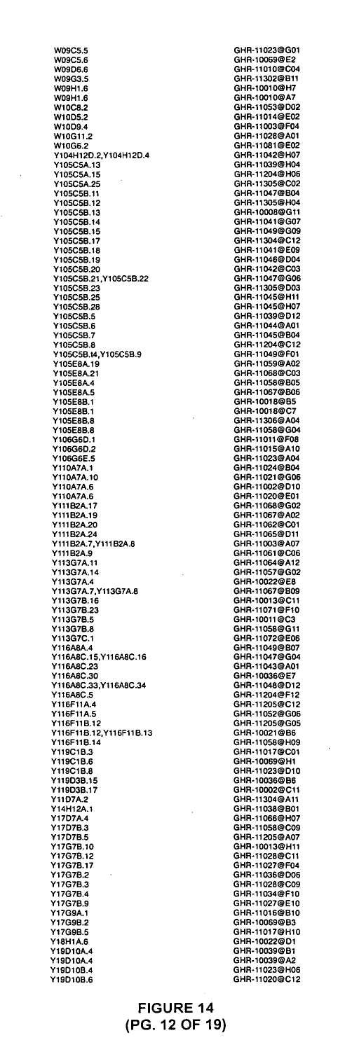

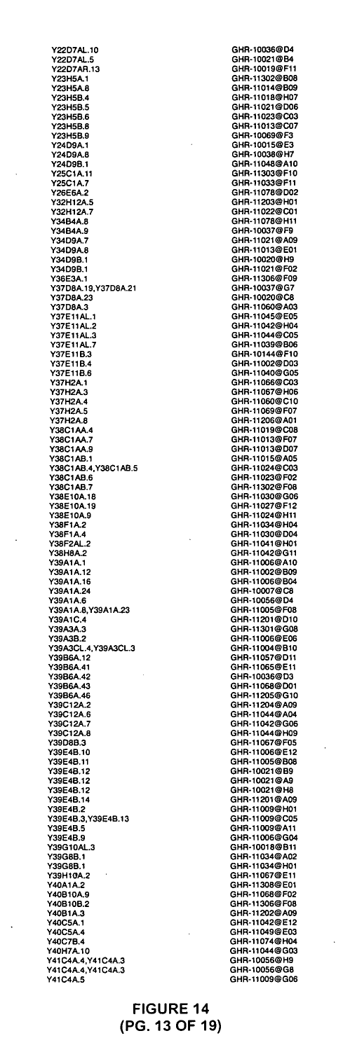

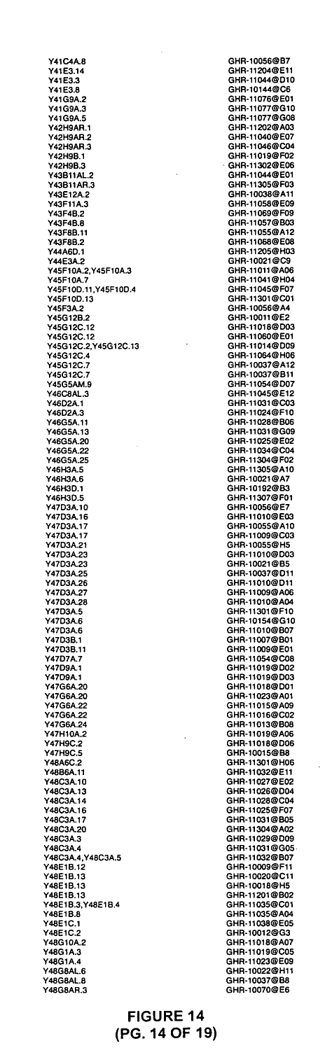

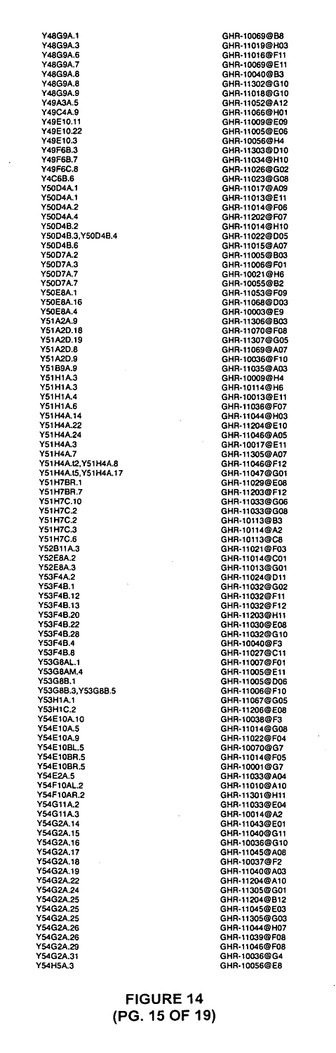

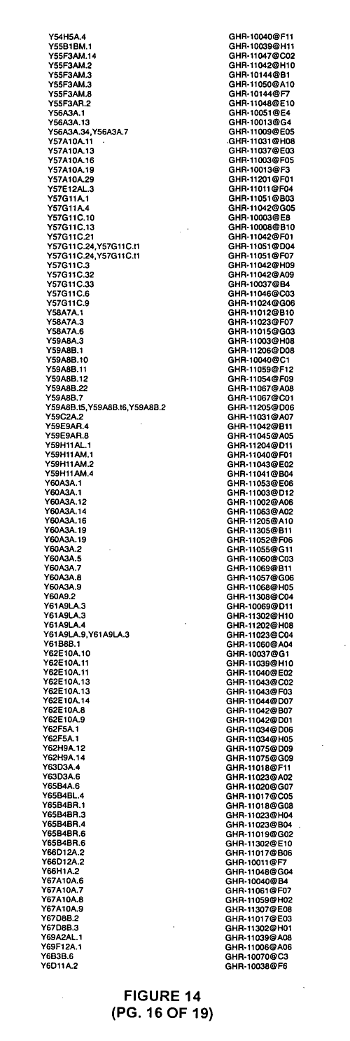

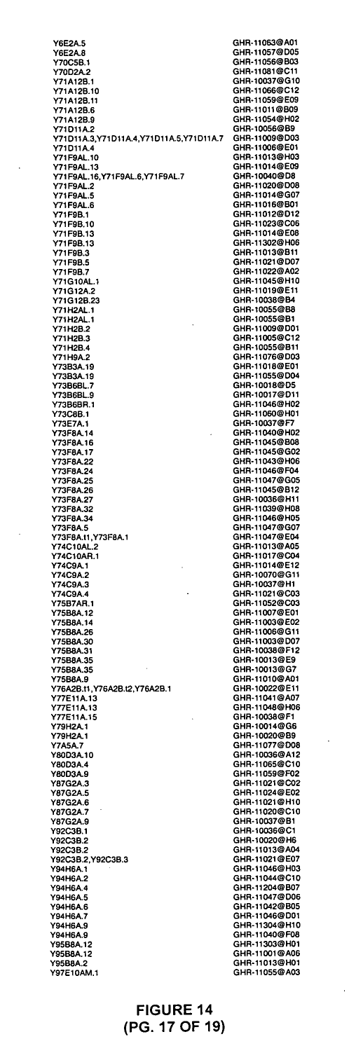

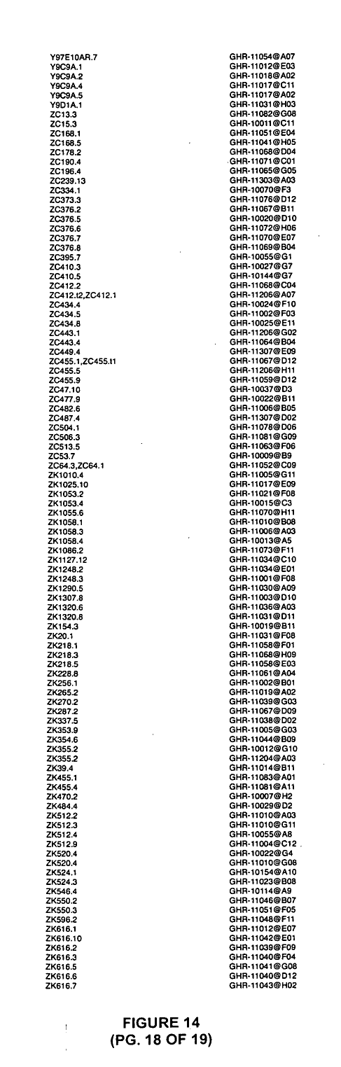

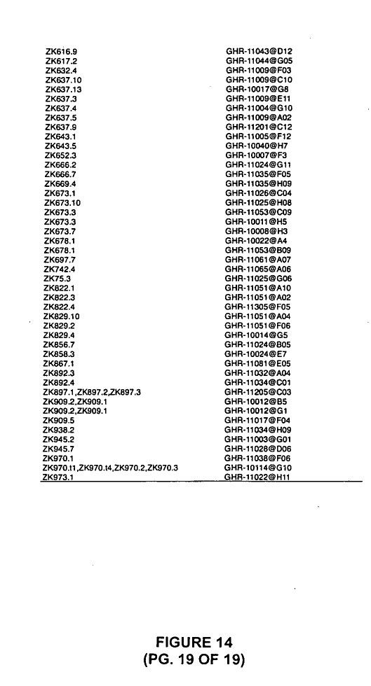

The Ahringer RNAi library was previously created using the methods described by Kamath and Ahringer, supra (2003). The 16,757 clones contained in this library are those previously described in the literature; in total, these clones correspond to roughly 86% of the C. elegans genome (Kamath et al., supra (2003)). More information about each clone can also be obtained through Wormbase (http://www.wormbase.org; maintained by the Wellcome Trust Sanger Institute, Cambridge, UK) (Stein et al. (2001)). The Vidal RNAi library was obtained from Marc Vidal and was generated by using the Gateway cloning system (Promega) to transfer ORFs from the C. elegans ORFeome project into the L4440 RNAi feeding vector and transforming these constructs into the RNAi feeding bacterial strain (Reboul et al., supra (2003)). Information about the 12,219 clones contained in this library can be obtained through WorfDB (http://worfdb.dfci.harvard.edu; Dana-Farber Cancer Institute, Boston, USA); in total, these clones represent roughly 63% of the C. elegans genome (Vaglio et al., Nucleic Acids Res 31:237 (2003)). For both libraries, plasmids were transformed into the HT115(DE3) RNase III-deficient E. coli strain for use in RNAi by feeding (Timmons et al., supra (2001)); this strain is publicly available through the Caenorhabditis Genetics Center at http://www.cbs.umn.edu/CGC/CGChomepage.htm). In HT115, the RNase III gene is disrupted by a Tn10 transposon carrying a tetracycline resistance marker; the genotype is as follows: [F—, mcrA, mcrB, IN(rrnD-rrnE)1, lambda-, rncl4::Tn10(DE3 lysogen:lacUV5 promoter-T7 polymerase) (IPTG-inducible T7 polymerase) (RNase III minus)]. A total of 1,821 RNAi clones were identified in the Vidal library (FIG. 14) that were not targeted by the Ahringer library or that were targeted but, for technical reasons, were not represented in the final Ahringer library. In total, the combined RNAi library contains a set of 18,578 non-overlapping bacterial strains covering 94% of all C. elegans genes predicted in the genome.

RNAi Feeding

Special methods were developed in adapting the optimized protocol for RNAi by feeding as described previously for high-throughput phenotypic screening in conjunction with chromosomal feeding libraries. Following are detailed protocols of these methods.

1. Bacterial Preparation and Induction

For feeding plates, special NGM agar was prepared including 25 μg/ml carbenicillin (Carb) and 1 mM IPTG; 1.5 ml of agar was dispensed into each well of 24-well TC plates (Nunc™, Rochester, N.Y.). After the agar set, plates were allowed to dry overnight before being stored at 4° C. for a maximum of 10 days before use. Bacteria from glycerol stocks were streaked or spotted onto LB-agar plates including 50 μg/ml Amp and 15 μg/ml Tet; for large-scale analyses, this was done in Omnitray flat plates (Nunc) using a 96-pin replicator (Invitrogen, San Diego, Calif.). Bacterial strains were inoculated into LB with 50 μg/ml Amp and were grown for 8-10 hours shaking at 37° C.; they were then seeded into the above NGM-derivative plates, one well per gene, in duplicate (i.e., two separate wells for each gene), and were allowed to dry thoroughly before being incubated overnight (˜12-16 hours) at room temperature (RT) to allow the bacteria to grow and to begin induction.

2. Worm Synchronization, Aliquoting, and Feeding

Worms were cultured on standard NGM plates with OP50 E. coli prior to use in feeding experiments. Plates with large numbers of eggs (and, optimally, small numbers of surviving worms) were washed with hypochlorite solution (1.5 ml 4N NaOH, 750 μl Aldrich sodium hypochlorite solution, 7.5 ml dH₂O). The solution was transferred to tubes, which were shaken vigorously by hand for 3-5 minutes to homogenize large worm particles. Eggs were pelleted by gentle centrifugation (<7000 rpm) and were washed 3× in M9 buffer (Sulston and Hodgkin, Methods. In The Nematode Caenorhabditis elegans. (Wood W B, ed.), pp. 587-606. Cold Spring Harbor Laboratory Press, 1988); 3 g KH₂PO₄, 6 g Na₂HPO₄, 5 g NaCl, 1 ml 1M MgSO₄, in H₂O to 1 L). Eggs were incubated in M9 and were allowed to hatch overnight (˜12 hours) at RT. Roughly 5-7 of the resulting synchronized L1-stage worms were then aliquoted onto feeding plates. The plates were allowed to dry, and the worms were fed at 25° C. until they reached early adulthood (5 days).

3. Phenotypic Scoring

After the feeding period, GFP expression was scored one a scale of 0 to 4 under UV at four time points over four successive 6-hour intervals (roughly corresponding to 102, 108, 114, and 120 hours) using either a LEICA MZ12 (Leica, Solms, Del.) or ZEISS Stemi SV6 microscope (Zeiss, Oberkachen, Del.). The scores were roughly scaled as follows: 0=GFP expression is baseline, i.e., roughly equivalent to worms fed non-specific dsRNA (i.e., empty L4440 vector); 1=GFP expression faintly brighter than baseline in few worms; 2=GFP expression significantly brighter than baseline in few worms or slightly brighter in many worms; 3=GFP expression very bright in few worms or bright in many worms; 4=GFP expression very bright in almost all worms. All clones that resulted in a positive score following RNAi by feeding (to be defined below for each separate pass of the screen) were subsequently refed and reanalyzed, and those clones failing to recapitulate previously observed phenotypes were repeated a final time. Only those clones capable of reproducing significant levels of a consistent, specific phenotype were considered in subsequent analyses.

High-Throughput Screening

The protocols described above are general methods that were employed for large-scale experiments using RNAi by feeding in conjunction with the GR1401 (Is41) or GR1402 strain. These methods were adapted as follows for each step of the screening process based upon the number of genes being screened.

1. Ahringer RNAi Library Screens

During first-pass screening of the genome using the Ahringer RNAi library, experiments were performed in duplicate in 24-well plates. Each clone was scored on the 0-4 scale described above at two time points by two independent observers, for a maximum score of 16 per clone. For each set of experiments (one per duplicate set), 6 wells were seeded with bacteria containing the feeding vector (L4440) and the following inserts for comparison while scoring: none (empty feeding vector), GFP, dcr-1, and mut-16. Of the 16,757 clones screened, 420 were considered ‘putative positives’ with a first-pass score ≧3. Those clones were then subjected to a second-pass screening performed in triplicate in 6-well plates to increase the number of scorable worms and to ensure that sufficient bacteria were present to prevent starvation. These experiments were scored as above by three independent observers, for a maximum score of 72 observations per clone. As described above, for each set of experiments, 3 wells each of the L4440, GFP, dcr-1, and mut-16 clones were included for comparison while scoring. In total, of the 420 clones re-screened, 120 were still considered ‘putative positives,’ either because they had a first-pass score ≧6 or a second-pass score ≧16. Those clones were then subjected to a third round of screening with two sets done in triplicate in 6-well plates and scored as above by two independent observers using controls as described for the previous pass. At this point, clones that were not at least weakly positive in at least two rounds (≧6/16 in 24-well format or ≧16/72 in 6-well format) were eliminated, leaving a total of 36 clones. Those clones were then subjected to a fourth and final round of screening in 6-well plates as described for the previous pass; however, this time 23 samples of L4440 bacteria and 3 samples each of GFP, dcr-1, and mut-16 were randomly inserted among the other clones as controls. In total, 28 clones (20 novel, 8 known) were strongly positive with a score ≧36/72, and these were included in the final set.

2. Vidal RNAi Library Screens

Computational methods were used to identify clones present in the Vidal RNAi library that are not predicted to be targeted by the Ahringer RNAi library; in total, 1,821 such clones were identified. These clones were regridded into a smaller sublibrary that was subsequently screened as described above for the Ahringer RNAi library. The first-pass screen was performed in 24-well plates in duplicate with 6 wells each of L4440, GFP, dcr-1, and mut-16 including for scoring comparison. Each clone was scored on the same 0-4 scale at two time points by two independent observers, for a maximum score of 16 per clone. Of the 1,924 clones screened, 227 were considered ‘putative positives’ with a first-pass score ≧3. Those clones were then subjected to a second-pass screening again performed in 24-well plates in duplicate and using the same controls and scoring system. A total of 30 clones were positive in both screens. Those clones were then subjected to a third and final round of screening in triplicate in 6-well plates using the same controls as described above with 3 wells each of the L4440, GFP, dcr-1, and mut-16 clones included for comparison while scoring. These experiments were again scored on the same scale at two time points by two independent observers, for a maximum score of 72 per clone. Of these, 11 were reproducibly positive, but only 3 clones (1 novel, 2 known) were strongly positive with a score ≧36/72, and these were included in the final set.

3. Screening the 945 Embryonic Lethal RNAi Clones

The 945 RNAi clones annotated as causing an embryonic lethal phenotype (Kamath et al., Nature 421:231 (2003)) were assayed in 24-well plates (first-pass) and in 6-well plates (second-pass), both in duplicate. Each clone was scored on the O-to-4 GFP scale by two independent observers at two time points. Of the 944 clones screened, 146 were considered ‘putative positives’ if the RNAi clones scored an average of ≧2 in either the first or second-pass tests. For the third-pass and the fourth-pass tests, the 146 putative positives were then tested, in duplicate, with 46 copies of control vector bacteria randomly inserted into the testing matrix. A total of 54 RNAi clones were consistently positive with an overall average score of ≧2. These 54 RNAi clones were confirmed by a final test that was performed in triplicate with an equivalent number of control vector bacteria randomly inserted in the testing matrix.

Sequencing of Clones

Plasmids for all putative positive RNAi clones were isolated and submitted for sequencing analysis at the DNA Core Facility of the Department of Molecular Biology, Massachusetts General Hospital (https://dnacore.mgh.harvard.edu/index.shtml). The plasmids were sequenced using a primer upstream of the multi-cloning site in the pL4440 vector (5′ L4440 Forward primer: 5′-GCAACCTGGCTTATCGAAAT-3′).

dsRNA Coinjection Experiments

The procedure for injecting dsRNAs to two genes was described previously (Dudley et al., Proc Natl Acad Sci USA 99:4191 (2002)). In brief, DNA templates, ranging in size from 0.5 kb to 1.6 kb, were PCR-amplified from plasmids corresponding to each RNAi clone. The resulting PCR products contained flanking T7 promoters (5′-CGTAATACGACTCACTATAG-3′) that were incorporated into the primers used for amplification. Sense and antisense RNAs were synthesized in a single reaction in vitro using a T7 polymerase-based transcription kit (Ambion Megascript Kit, Ambion Inc.). After phenol/chloroform extraction and ethanol precipitation, the dsRNAs were resuspended in H₂O, denatured for 10 minutes at 70° C., and re-annealed at room temperature. Concentrations of dsRNAs were determined by spectrophotometry (Thermos) and the quality and size of the dsRNAs were assessed by gel electrophoresis. Wild type young adults were injected with mixes containing 300 ng/ml dsRNA of the candidate gene and 100 ng/ml of mom-2 dsRNA. Worms were allowed to recover for 14-16 hours before being singled for two consecutive 24-hour egg lays. The viability of the progenty from each egg lay was assessed after 24 hours of growth and expressed as the (number of hatched larvae)/(total number of larvae+unhatched eggs). All worms were incubated at 20° C. throughout the experiment

RNAi Injection

RNAi by direct microinjection was performed as previously described by Fire et al. (Gene 93:189 (1998)). Templates for dsRNA synthesis were made by PCR on L4440-based feeding constructs using T7 primer (5′-CGTAATACGACTCACTATAG-3′). Sense and antisense RNAs were synthesized in a single reaction in vitro using a T7 polymerase-based kit (Promega). Double-stranding was achieved by incubation at 72° C. for 10 min, and the sizes and integrity of dsRNA products were verified by electrophoresis. dsRNA was injected at a concentration of 0.5-1.0 mg/ml. Injected worms were allowed to recover at 15° C. for 10-14 hours post-injection, then were replica plated and were allowed to lay eggs for 15-18 hours before being removed. The progeny of injected worms were incubated for another 18-21 hours before being scored for embryonic lethality. Another 24 hours later, the worms were re-scored for either molting defects (nhr-23) or fluorescent embryos (pos-1).

Computational Methods

Information about C. elegans genes, including sequences, was obtained from Wormbase (http://www.wormbase.org) (Stein et al., Nucleic Acids Res 29:82 (2001)). Homologies were established using the basic local alignment search tool (BLAST) (http://www.ncbi.nlm.nih.gov/blast; mainted by the National Institutes for Health, USA) (Altschul et al., J Mol Biol 215:403 (1990)).

Other Assays

Assays for somatic transgene silencing, seam cell counting, and enhancement of weak let-7 were performed as follows unless otherwise indicated: for RNAi factors whose inactivation did not cause lethality, L1 larvae were fed the corresponding RNAi bacterial clone, and late L4 larvae or adults of the subsequent progeny generation were assayed. For the 54 RNAi clones that resulted in embryonic lethality, L1 larvae were fed the corresponding RNAi bacterial clone and were assayed in the late L4 larvae to adult stage in the same generation.

1. Somatic Transgene Silencing

The restoration of GFP fluorescence of eri-1 (mg366) in JR672 (Koh and Rothman, Development 128:2867 (2001)) (GR1402) was assayed employing the same GFP scale used in the genome-wide screen of the RNAi sensor strain (GR1401). The feeding of the RNAi bacteria was performed at 22° to 23° C. For the transgene silencing assays with eri-1 (mg366); sur-5::gfp (GR1403) and rrf-3 (pk1426); sur-5::gfp (GR1404) strains, the worms were grown at 20° C. and scored on a 0 to 4 scale at the L2 to L3 stage in the progeny.

2. Germline Transgene Silencing

To assay transgene silencing in the germline, L1 larvae of the let-858::gfp reporter strain (PD7271, provided by W. Kelly (Kelly et al., Genetics 146:227 (1997)) were fed the 36 RNAi bacteria clones that result in non-lethal phenotypes. In the progeny generation, 5-10 L2 worms were transferred to new RNAi feeding plates and the restoration of GFP expression in the germline was assayed on a 0 to 4 GFP scalse in adults of the following generation.

3. Counting Seam Cell Number

To determine if inactivation of any of the RNAi factors causes an aberrant number of seam cells, the JR672 strain was observed, which expresses nuclear-localized GFP in the seam cells, after inactivation of each of the genes identified in the screen. For RNAi clones that do not result in lethality, L1 larvae were fed the RNAi bacteria and seam cell numbers were counted at the young adult stage in the next generation. For the 56 RNAi clones that cause embryonic lethality, the young adults of JR672 were scored in the same generation. All experiments were performed at 20° C. and seam cells were counted on an average of 20-30 worms from 2 independent experiments.

4. Enhancement of Weak let-7 (mg-279)

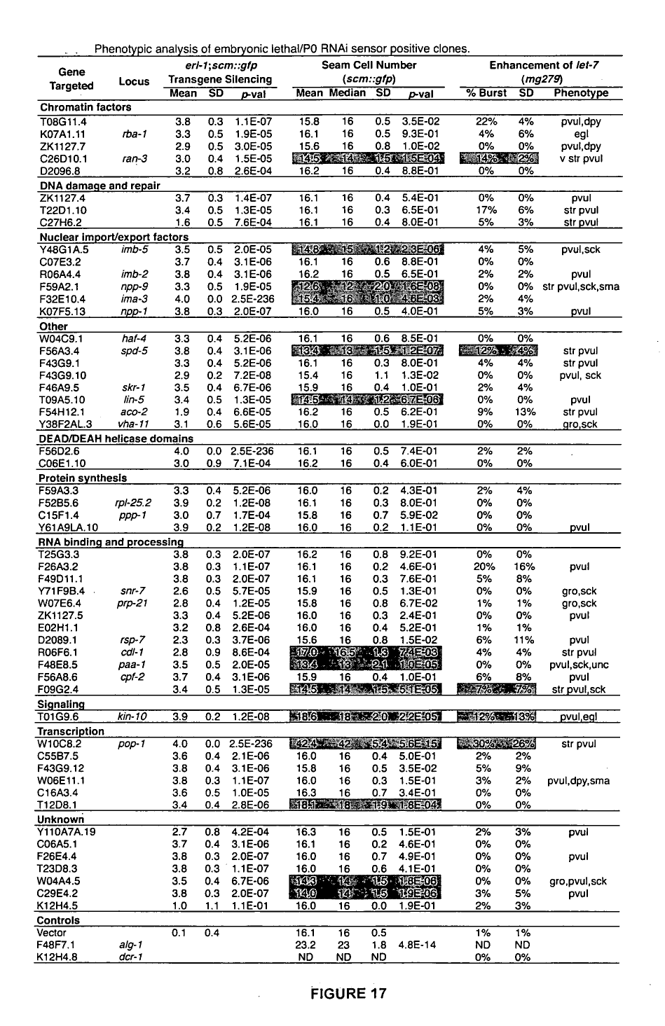

Enhancing the protruding vulva (pvul) phenotype of the weak let-7 (mg279) mutation results in the more severe bursting vulva phenotype. The bursting vulva phenotype was scored in the gravid adults of the parental generation for the animals fed on the bacteria expressing dsRNA to essential genes and in the gravid adults of the progeny generation for animals fed on bacteria expressing dsRNA of genes not required for viability (FIG. 16 and FIG. 17). All experiments were performed at 20° C. Percentage burst vulva was reported as the average of 2-3 experiments.

Homolog Detection

The RNAi factors identified in the present screen were compared to the proteomes from the following organisms (assembly): Homo sapiens (NCBI 35), Mus musculus (NCBIM 33), Drosophila melanogaster (DROM 3) and Schizosaccharomyces pombe (Version 19) using BLASTP (Altschul et al., J Mol Biol 215:403 (1990)). For the highest scoring BLAST hit from each organism, a reciprocal BLAST search was then conducted against the C. elegans proteome (Wormpep 94). Orthologs were defined as polypeptides that were reciprocal best BLAST hits. Homologs were defined as genes having an e-value of <10⁻⁶. Roughly 89% (64/72) of genes characterized as homologs had an e-value <10⁻¹⁰.

Methods for Additional Y2H Screens

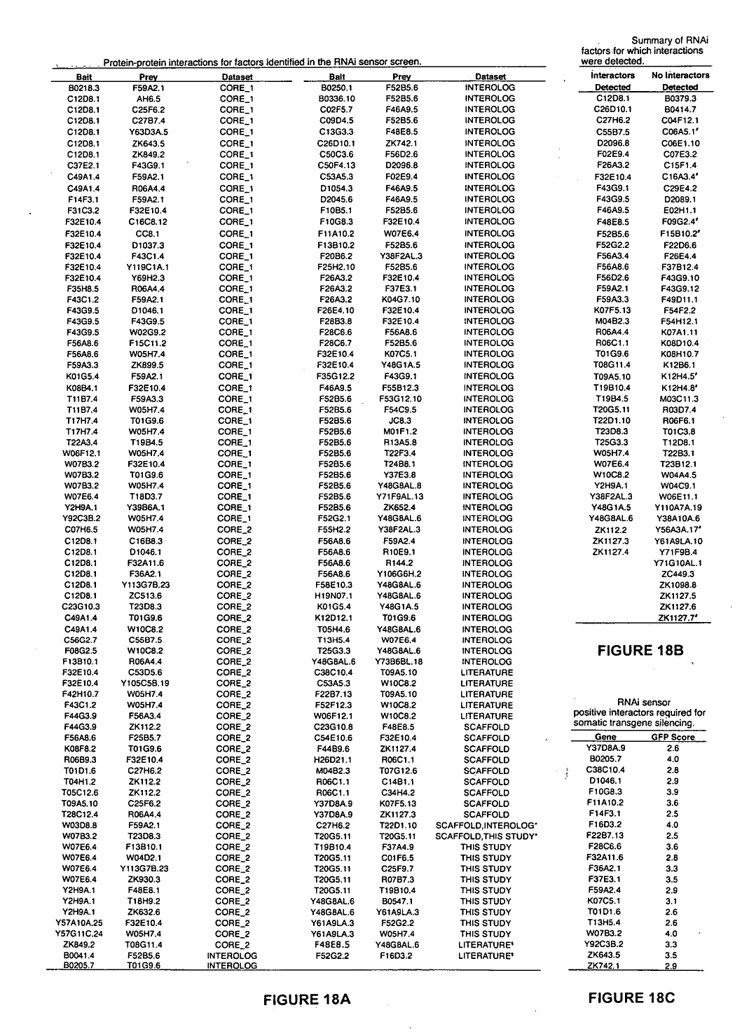

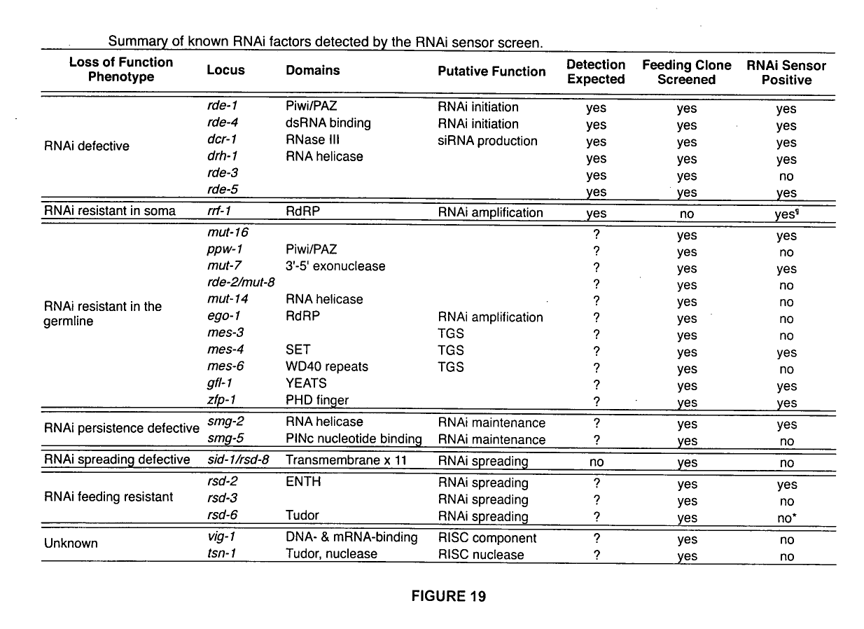

The protein interaction network was delineated by searching the WI5 interactome dataset (L1 et al., Science 303:540 (2004)) with the genes identified in the present screen. Independent of WI5, four additional yeast two-hybrid (Y2H) screens of a C. elegans AD-cDNA library were performed using methods described previously (Tewari et al., Mol Cell 13:469 (2004)) using the following ORFs as baits: T19B10.4, Y48G8AL.6, T20G5.11 (rde-4), and Y61A9LA.3. Ten of the interactions represented in the interactome network (FIG. 13 and FIG. 18) are derived from these additional screens.

Inhibitory Nucleobase Oligomers

Inhibitory nucleobase oligomers (e.g., double stranded RNA (dsRNA), short interfering RNA (siRNA), antisense RNA, short hairpin RNA (shRNA), and mimetics thereof) decrease the expression of target genes. In some cases, RNAi can virtually eliminate the expression of the target gene. For some applications, such a dramatic decrease in gene expression is undesirable, or even deleterious. Decreasing the expression of RNAi essential genes (REGs) in a subject, by targeting such genes for RNAi, decreases the efficacy of RNAi in that subject, and when co-administered with a second RNAi therapy, preserves limited expression of a target gene. Thus, REG RNAi allows the modulation of RNAi to a desired level.

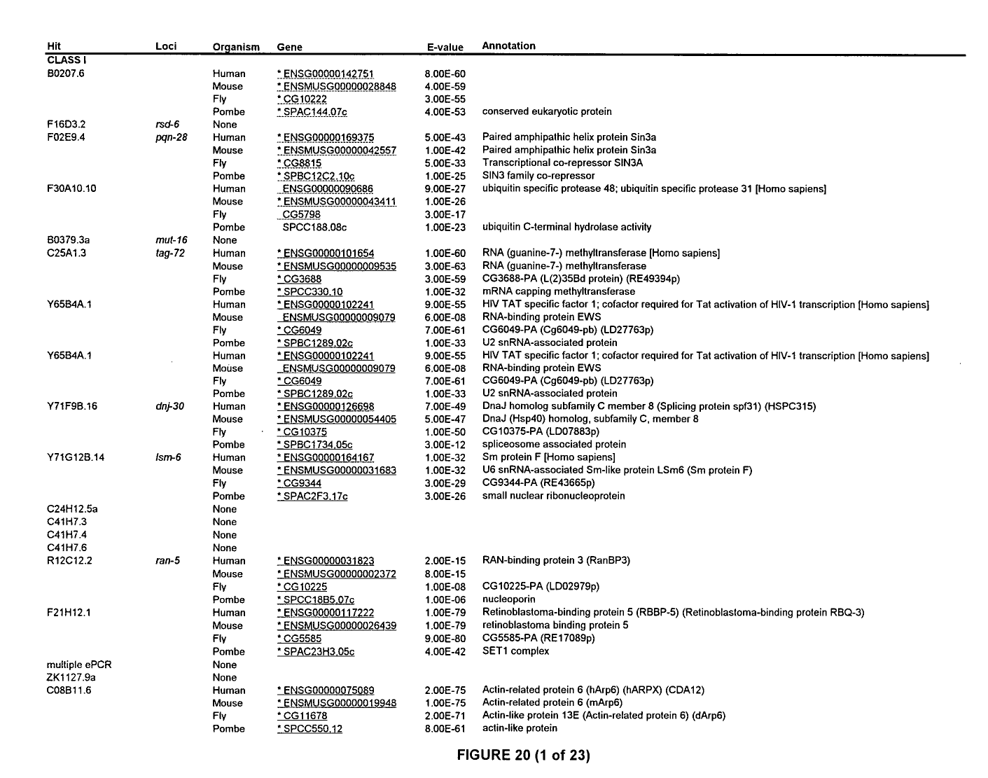

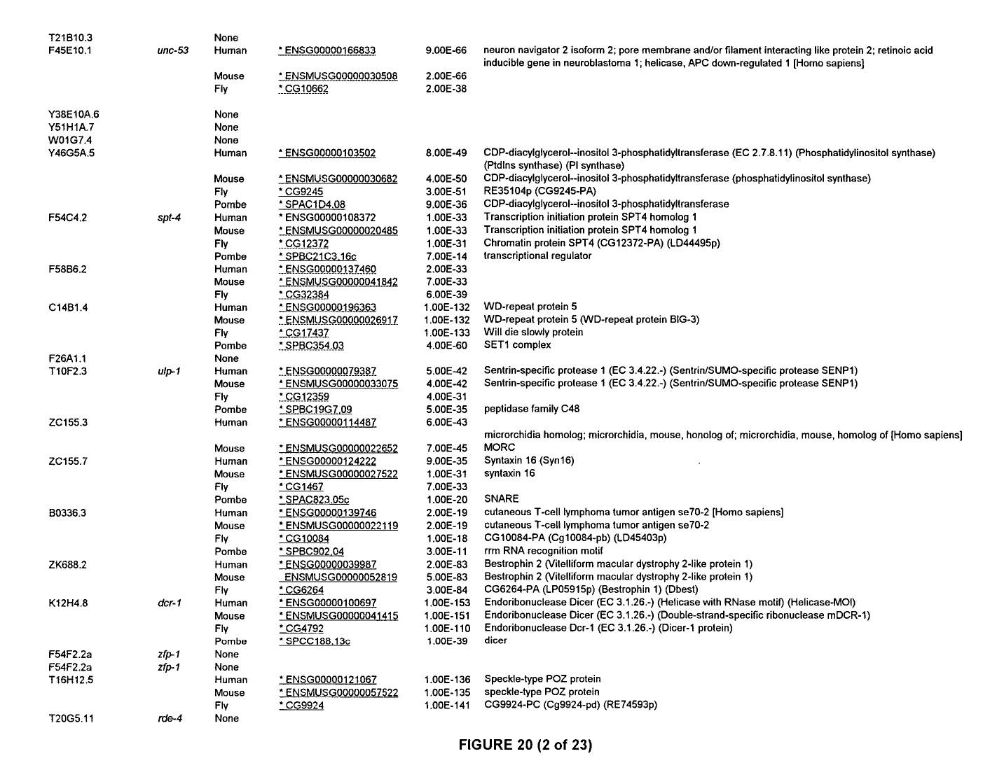

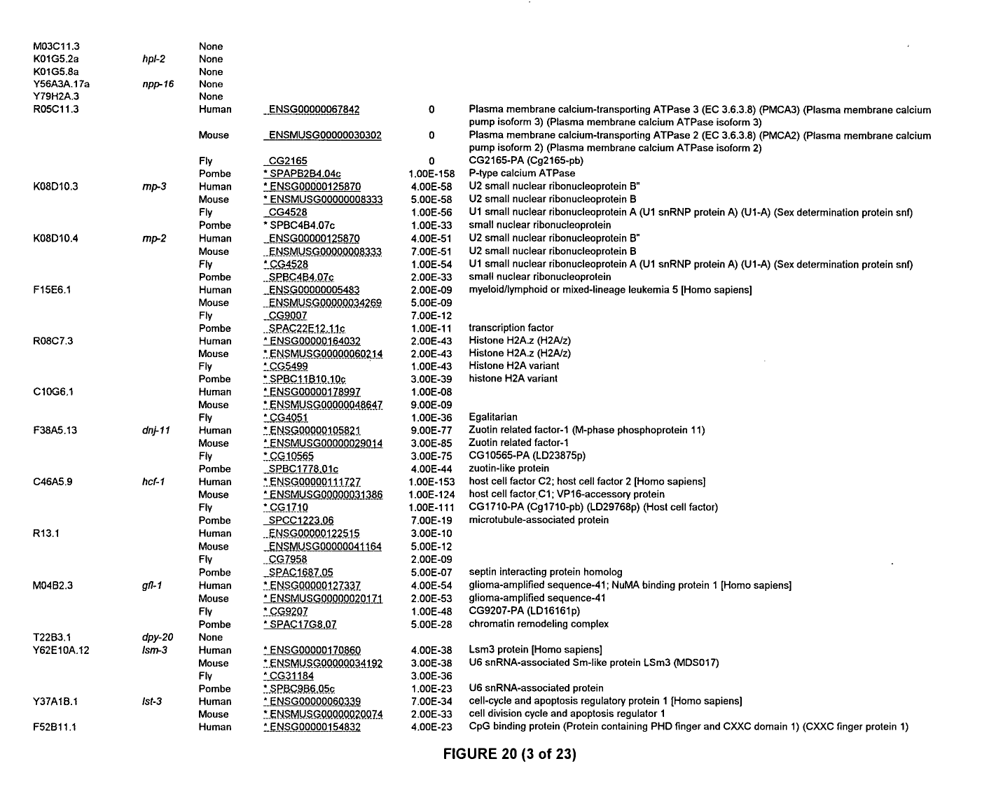

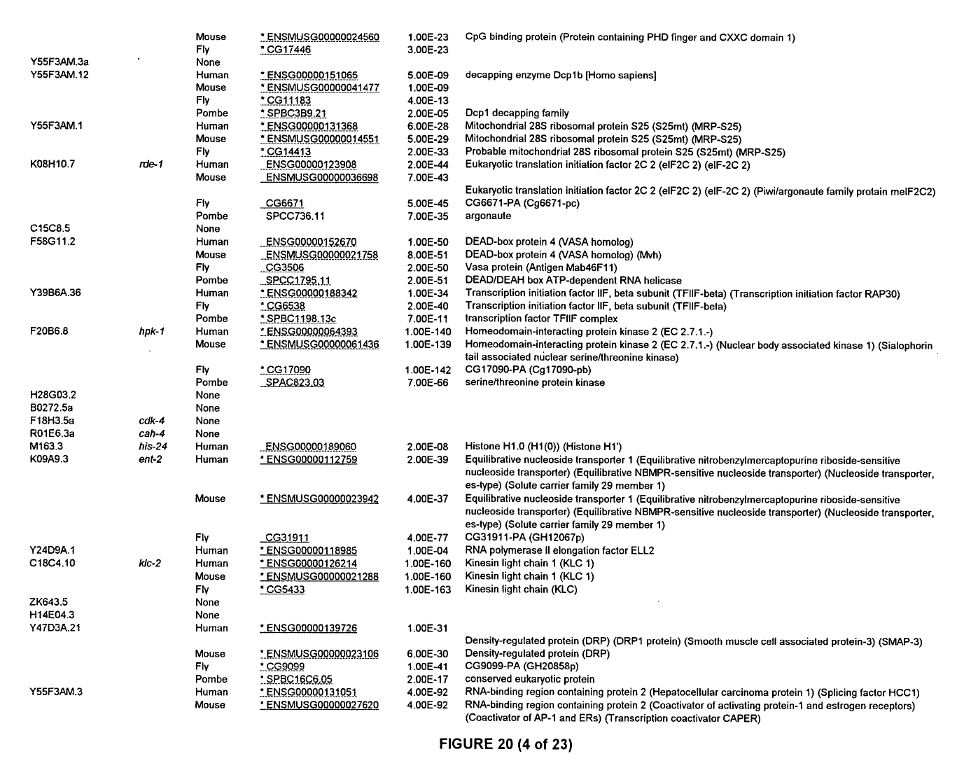

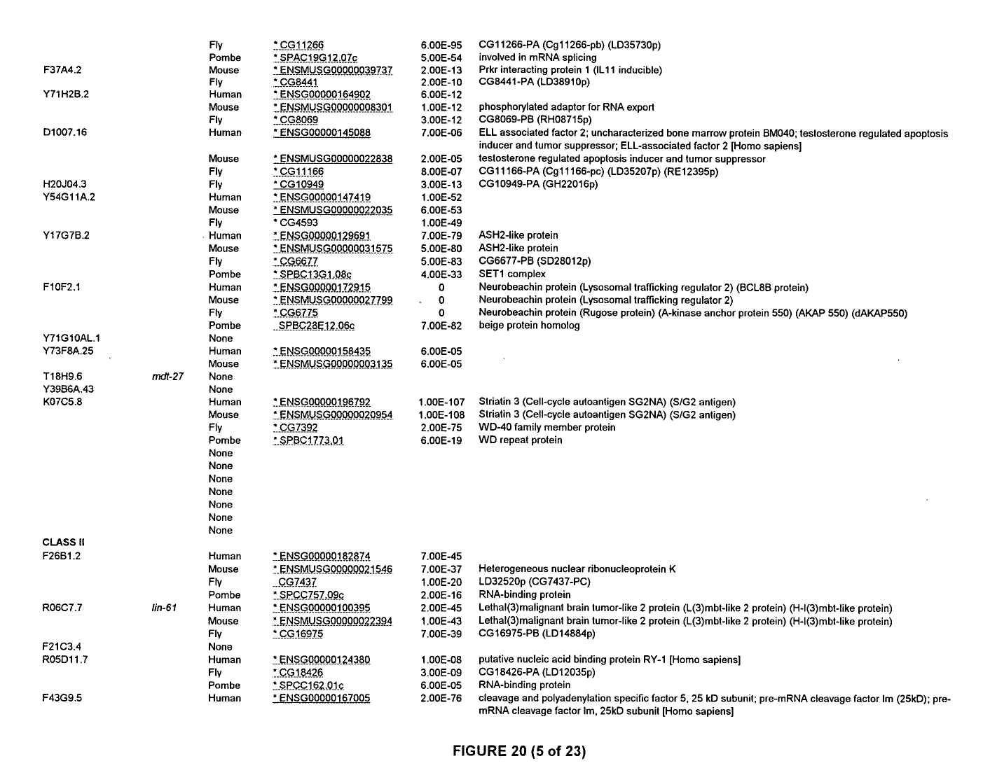

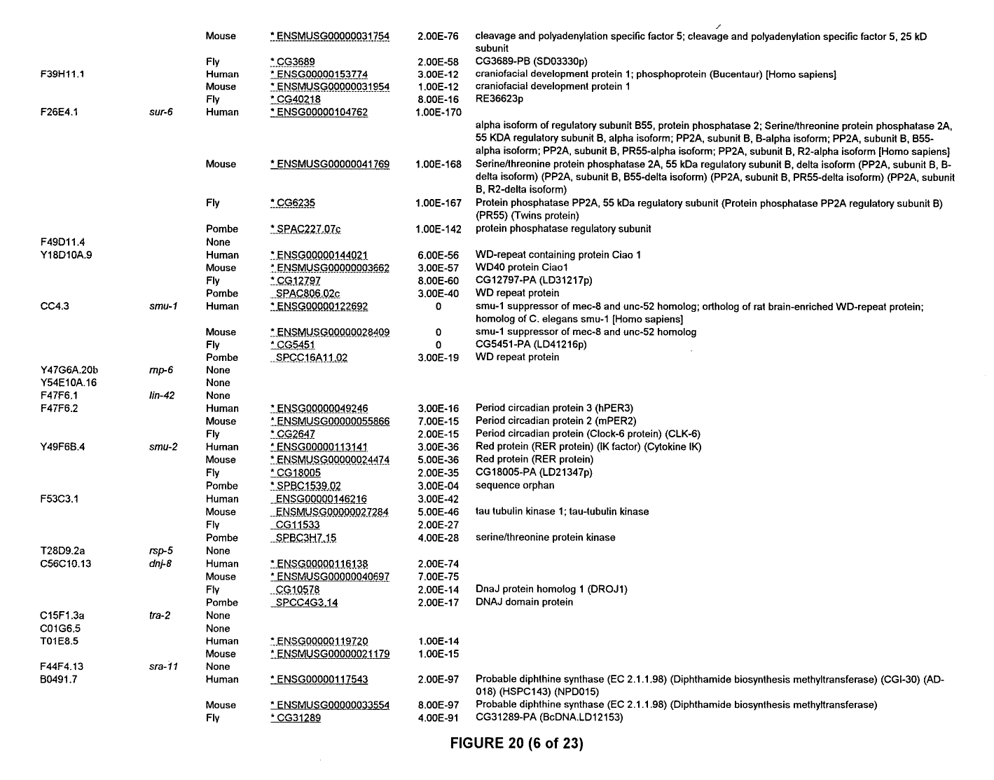

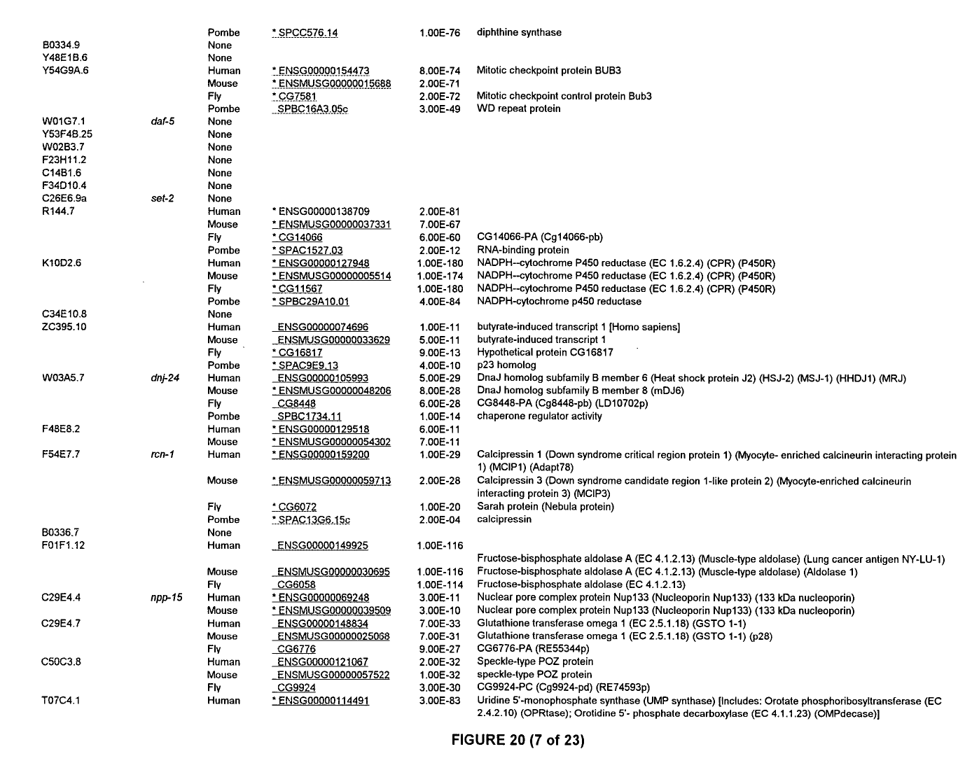

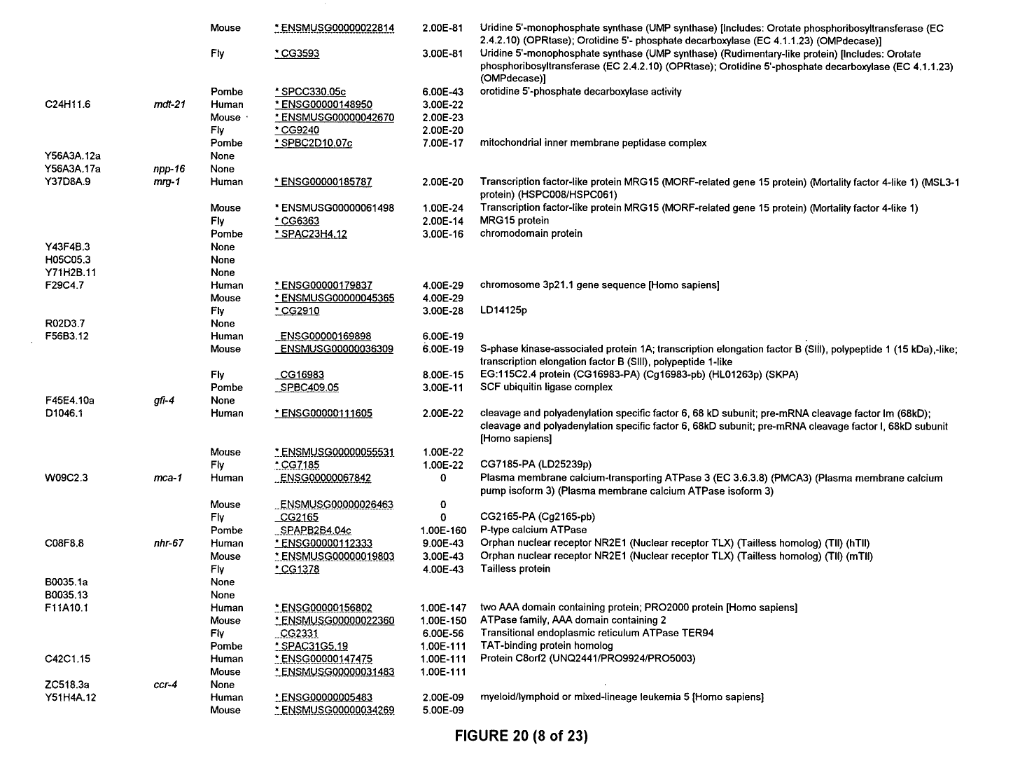

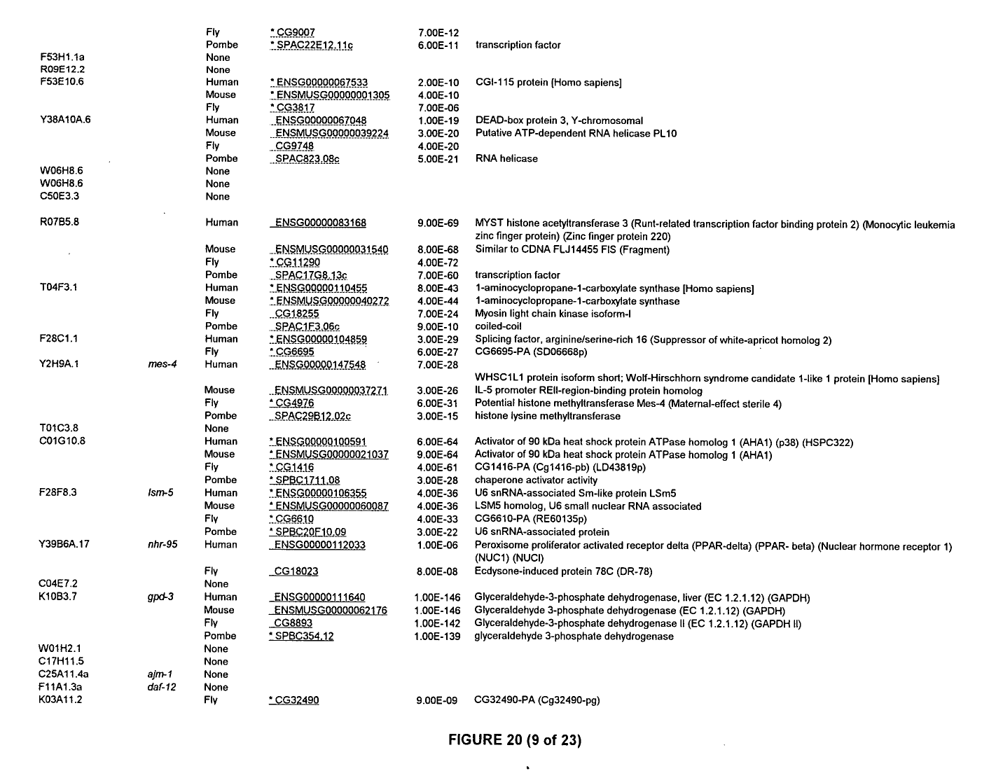

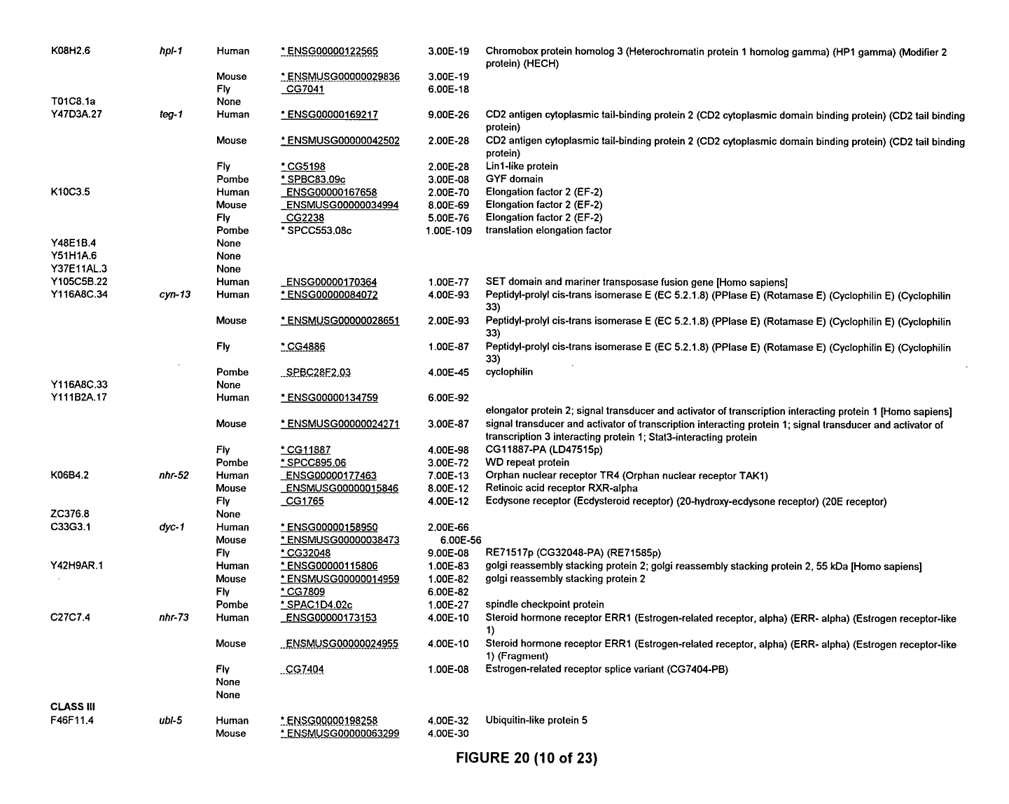

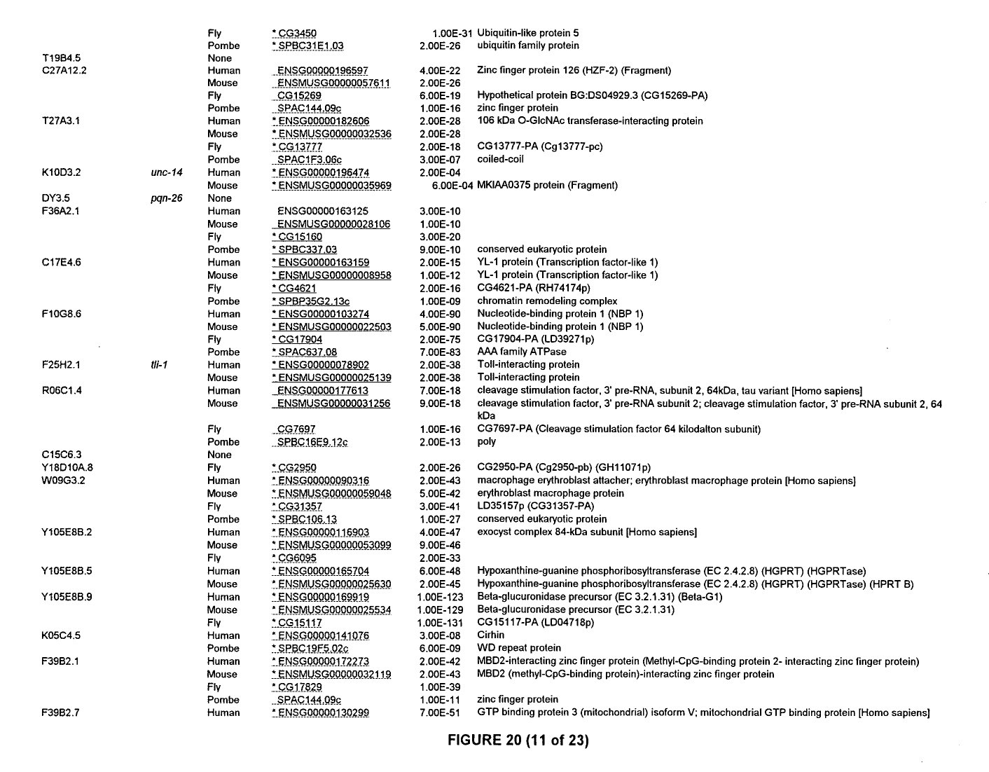

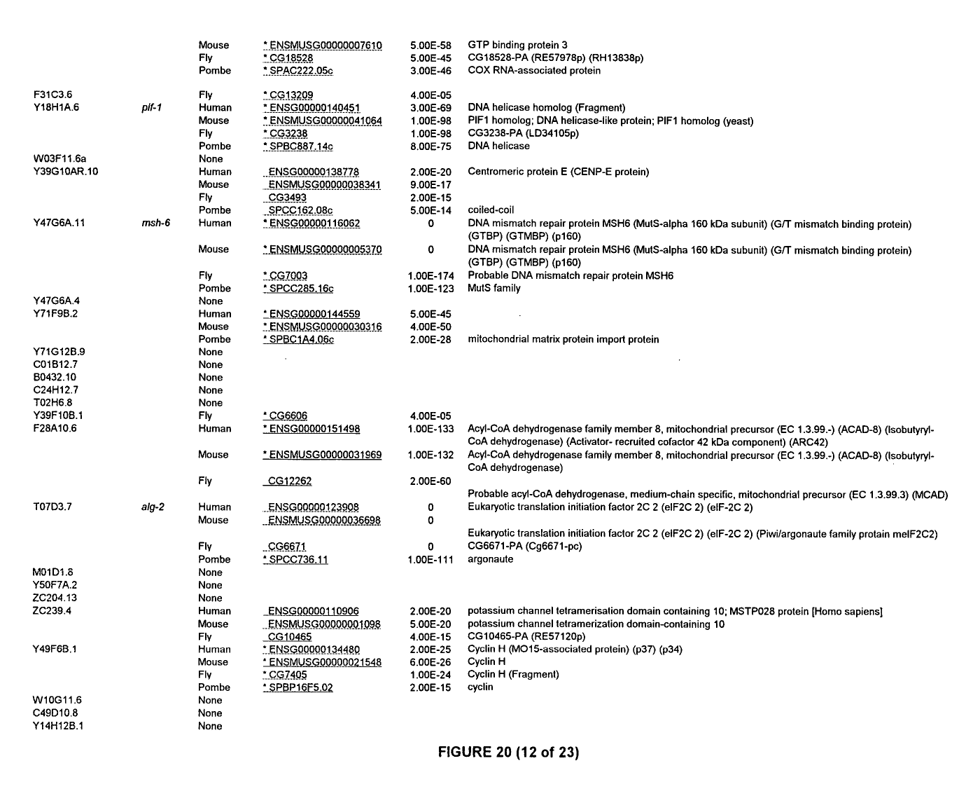

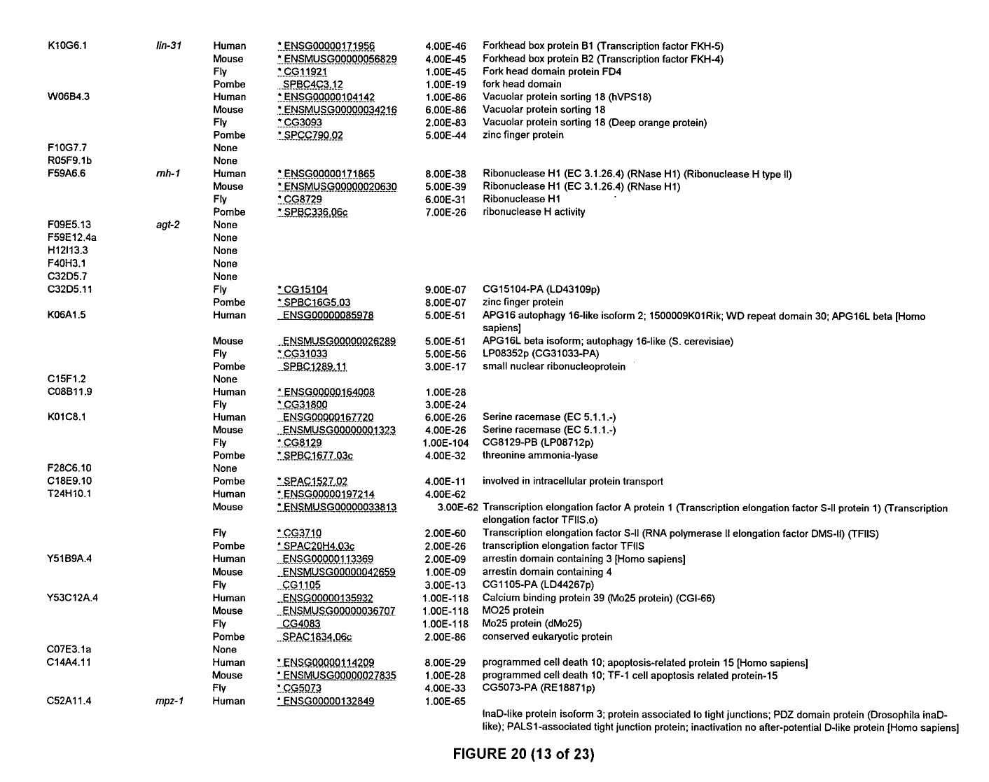

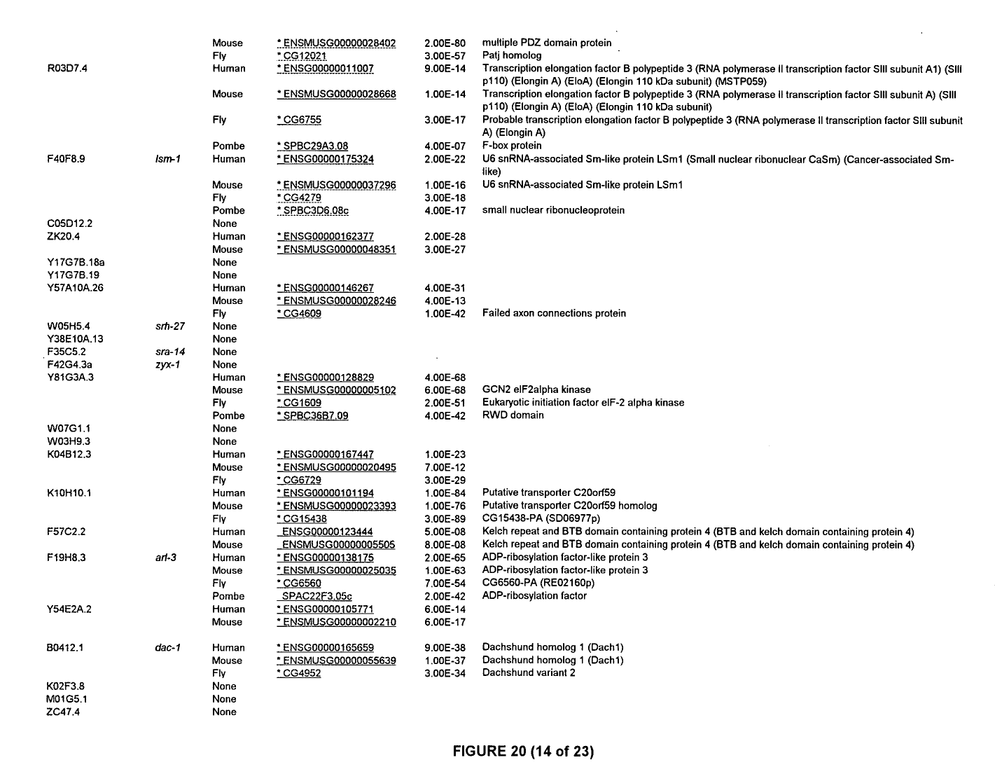

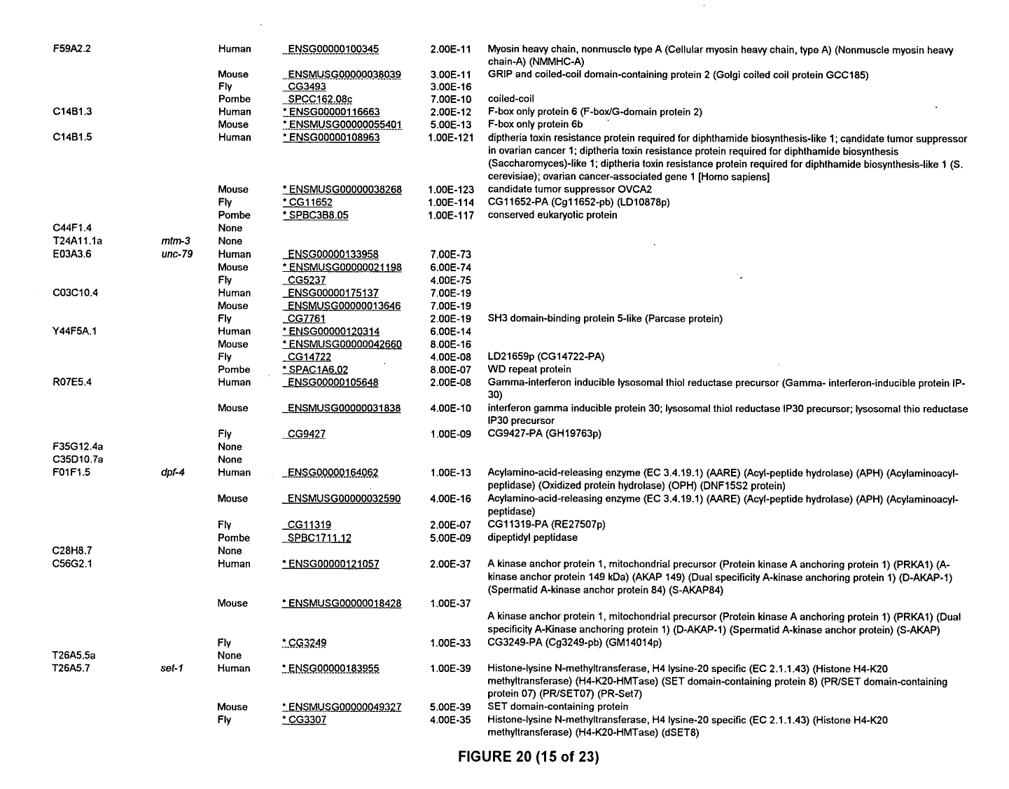

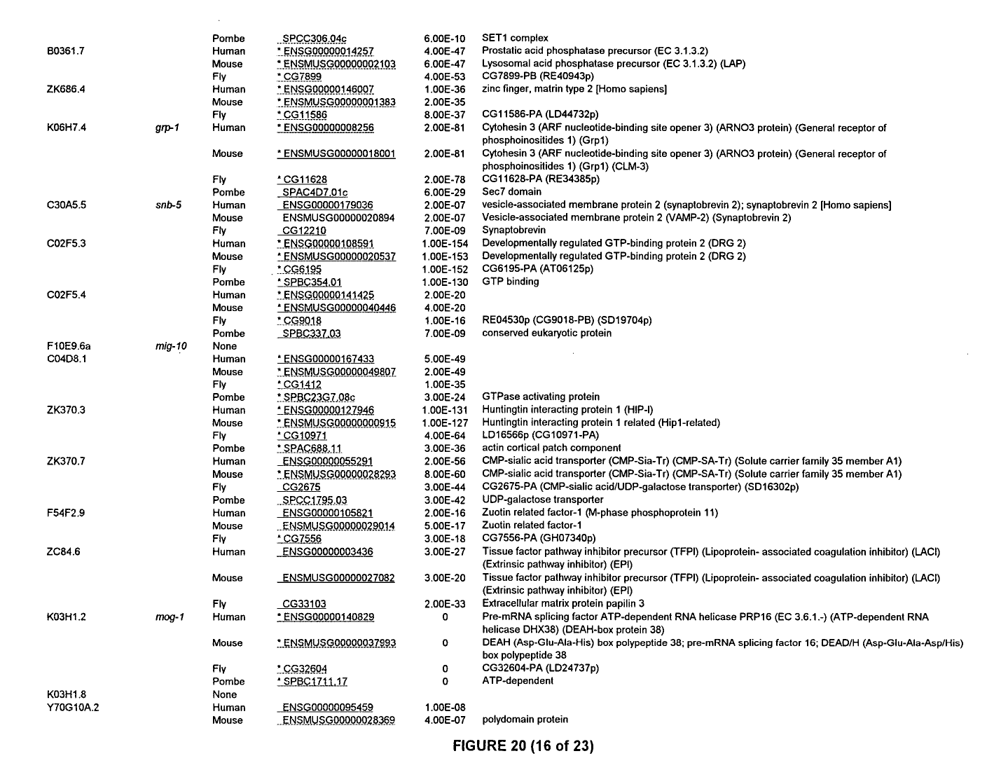

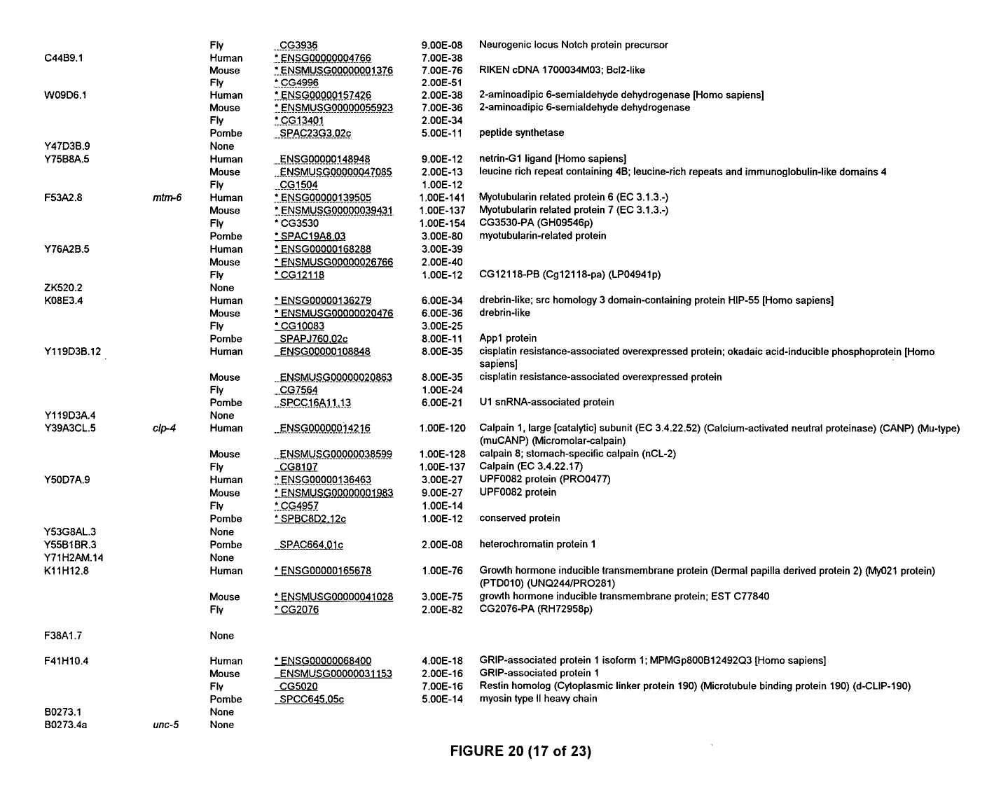

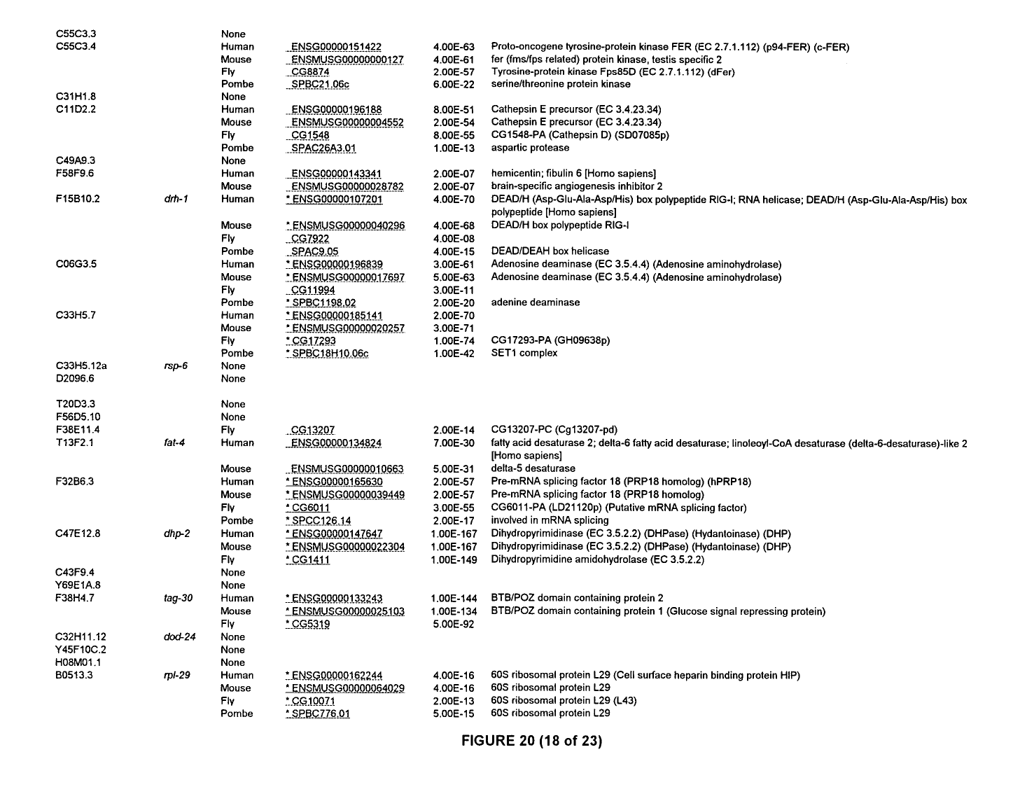

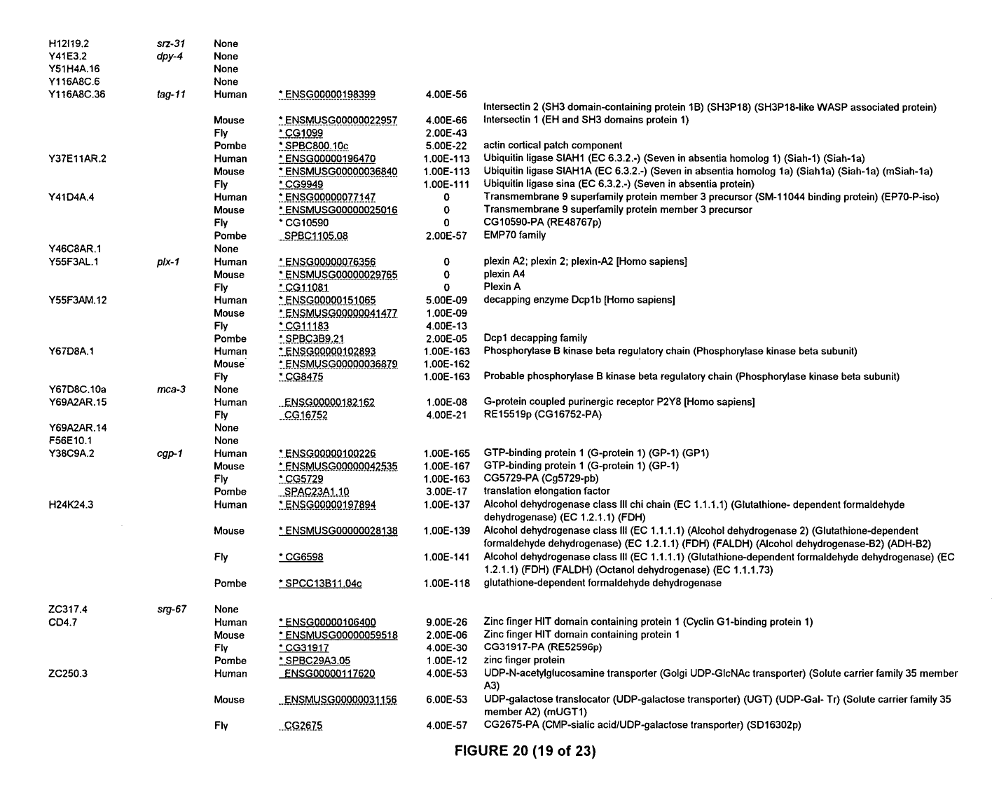

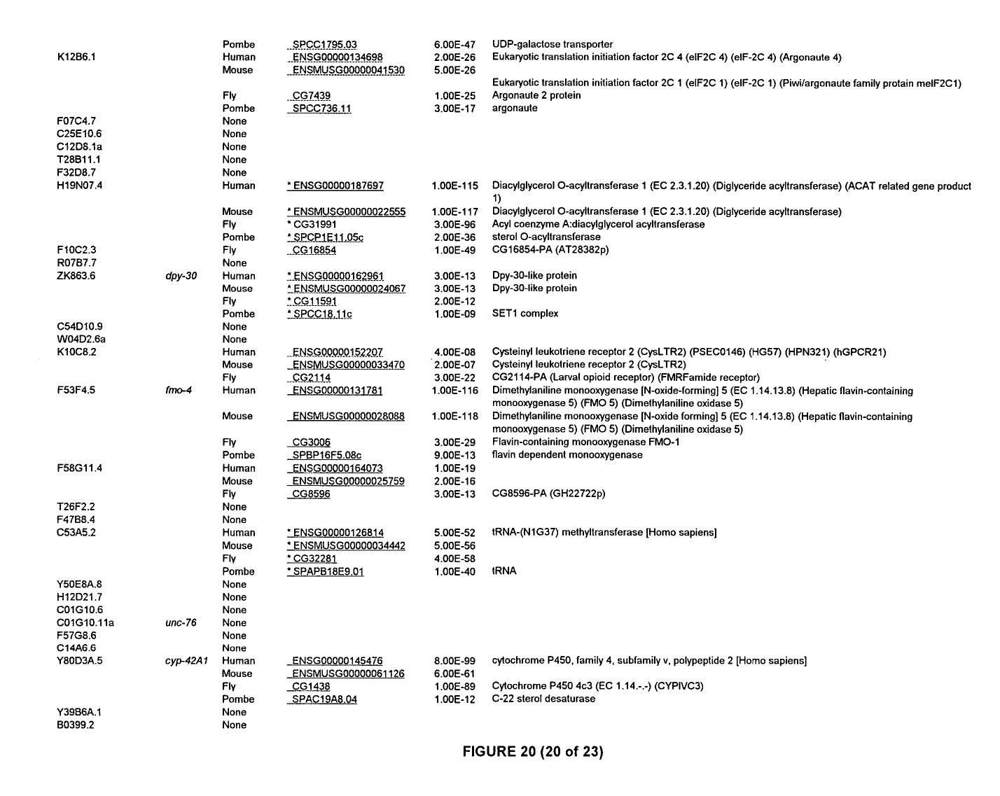

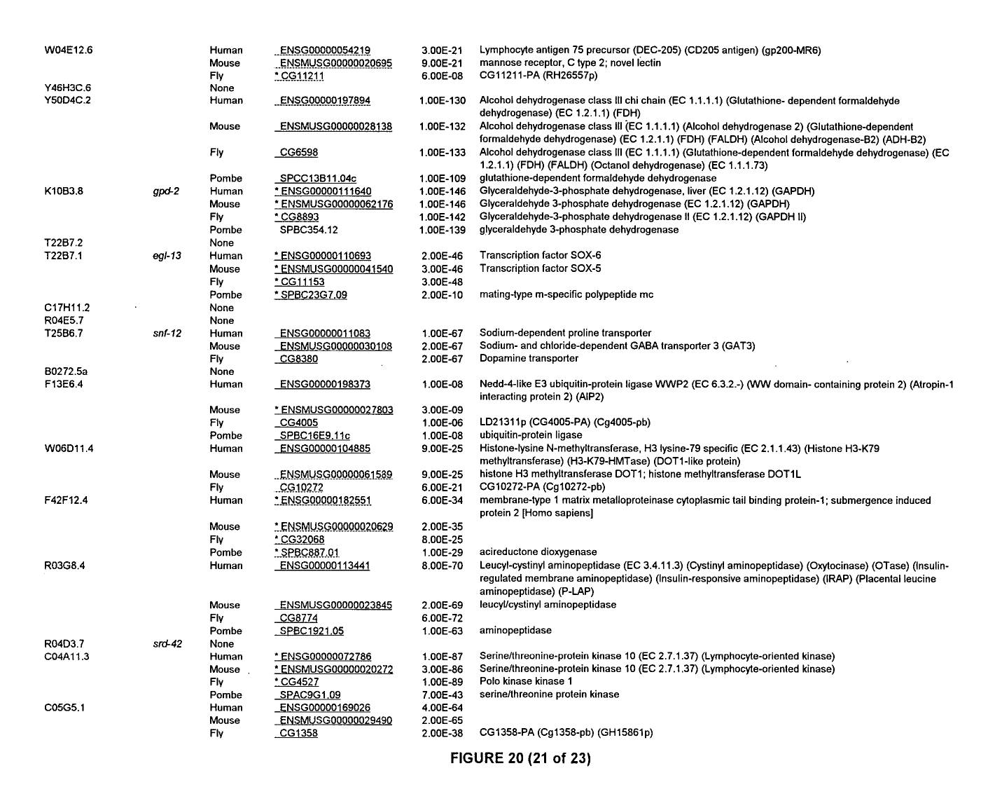

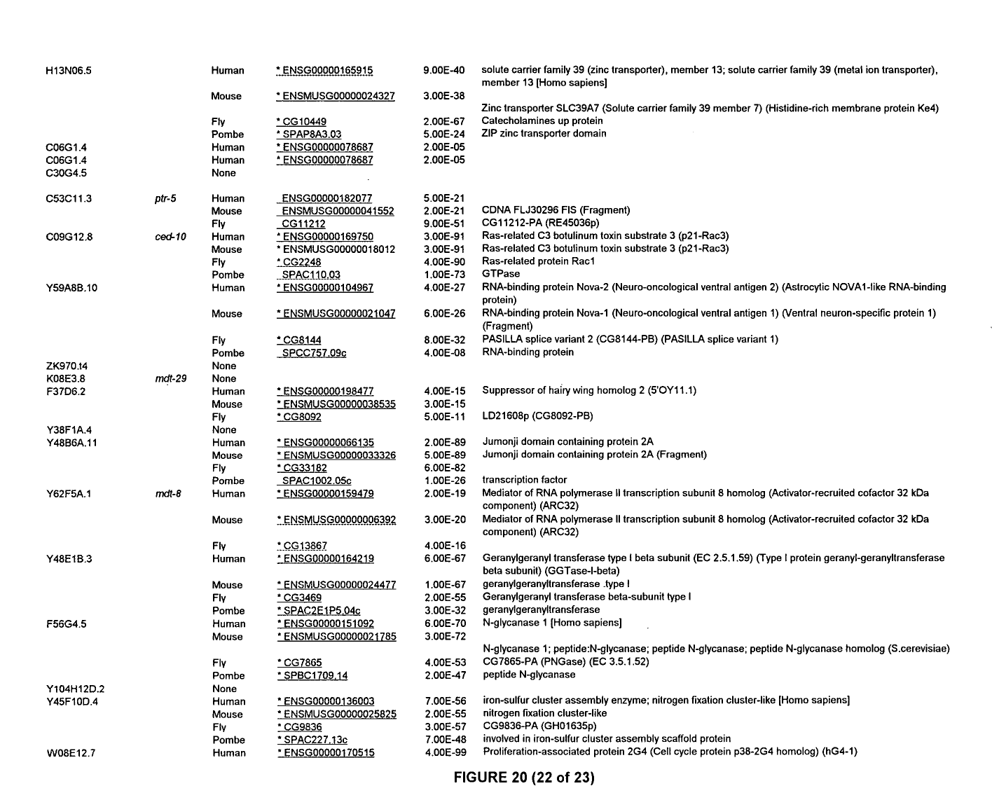

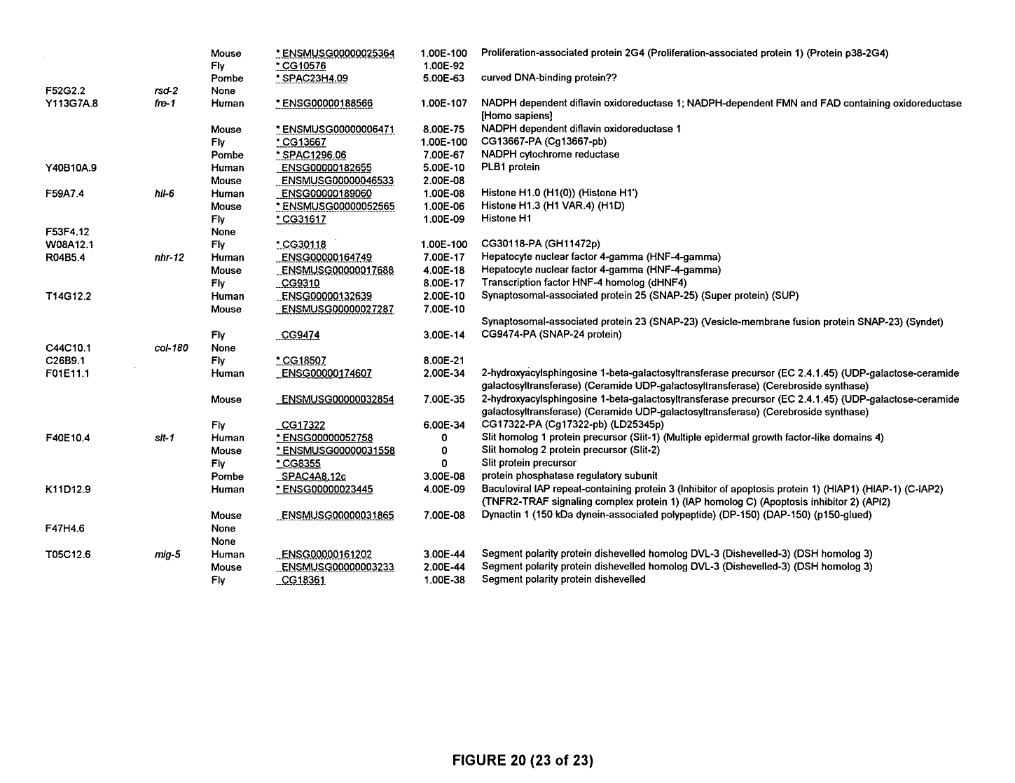

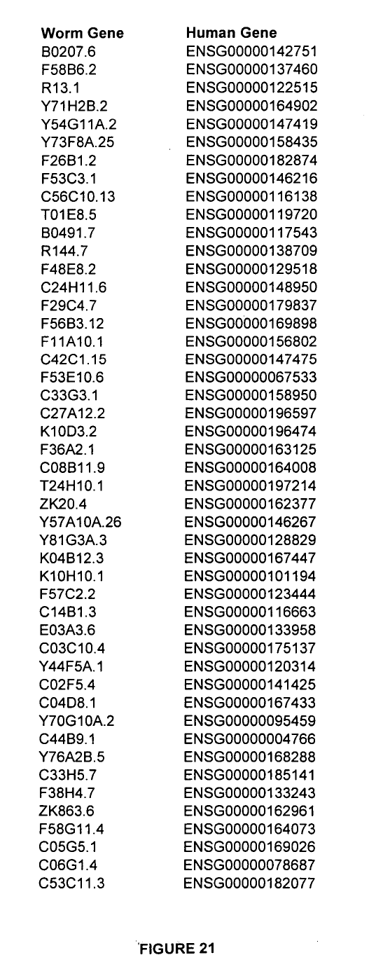

For some applications, it may be desirable to restrict RNAi to a particular cell type, tissue, or organ. Administering an REG RNAi (e.g., derived from the nucleic acids in FIGS. 15 and 20) to such cells, tissues, or organs provides localized protection to those targets from the effects of a second RNAi therapy. This allows the expression of an RNAi target gene to be preserved in desired locations where a decrease in gene expression would be undesirable. For example, an REG RNAi may be administered or targeted using a tissue specific promoter to a healthy tissue to protect that tissue from the toxic affect of a second RNAi therapeutic, where the second RNAi targets genes essential for the proliferation of a tumor cell.

Vectors encoding an inhibitory nucleic acid that targets an REG may be used to transform a cell or an organism, such as a plant, nematode, Drosophila, or mammal. Cells or organisms expressing such vectors are refractory to the effects of RNAi and are valuable model systems in which additional RNAi pathway components may be identified or characterized.

Using the nucleic acid sequence of genes identified herein, or plant or mammalian orthologs thereof, inhibitory oligonucleotides (e.g., nucleic acids or nucleobase oligomers) targeting REGs may be identified. Inhibitory oligonucleotides targeting REGs are useful for a variety of applications, including RNAi therapies

Therapeutic Uses of REG Polypeptides or Nucleic Acids

As described above REG nucleic acids (e.g., nucleic acids in FIGS. 15 and 20) encode polypeptides that are required for RNAi. In some organisms, certain cell types, such as neurons, are refractory to RNAi. It may be that these cells fail to express or express an inadequate amount of an RNAi essential gene. To enable RNAi in such cells, it may be useful to provide an RNAi therapeutic in conjunction with a polypeptide cocktail containing at least one REG polypeptide that is lacking in a cell, tissue, or organ that is refractory to RNAi.

In another embodiment, it may be desirable to enhance RNAi in a cell, tissue, or organ having a functional RNAi pathway. When an inhibitory nucleic acid is administered to such cells in conjunction with a polypeptide cocktail containing at least one REG polypeptide, the efficacy of RNAi is enhanced.

REG cocktails may contain one or more REG polylpeptides (e.g., polypeptides coded for by the nucleic acids in FIGS. 15 and 20) and are useful for enhancing RNAi for the treatment of a variety of pathological conditions, including but not limited to the treatment of pathogen infections (bacterial, viral, parasitic), hyperproliferative disorders (e.g., neoplasms, such as cancer), and genetic disorders resulting from the expression or overexpression of a gene or mutant allele.

For some applications, an REG cocktail is administered in combination with an inhibitory nucleic acid that targets a portion of a pathogen genome, where inactivation of a portion of a pathogen genome is sufficient to prevent, ameliorate, or eliminate infection by the pathogen. By enhancing RNAi of a pathogen genome, an REG cocktail of the invention facilitates both the treatment and prevention of pathogen infections in a subject. Methods for the use of RNAi in the treatment of pathogen infections are described, for example, in U.S. Patent Publications 20030219407, 20030203868, 20030206887, 2003020386, and 2003020386.

In other embodiments, an REG cocktail is administered in combination with an inhibitory nucleic acid that targets an endogenous gene of interest whose expression or overexpression induces a disease or disorder, such as a neoplasm. In one example, an REG cocktail is administered in combination with an inhibitory nucleic acid that targets a gene whose expression contributes to cancer. In other examples, an REG cocktail enhances RNAi of a targeted gene that promotes abnormal angiogenesis. In still other examples, an REG cocktail enhances RNAi used to treat a genetic disorder (e.g., familial hypercholesteremia, dominant forms of retinal degeneration, Parkinson's disease, spinobulbar muscular atrophy, Huntington's disease, myotonic dystrophy, or other trinucleotide repeat disorders). Therapeutic RNAi methods that target endogenous genes are known to the skilled artisan. See, for example, U.S. Patent Publications 20030148519, 20030148519, 20030143204. In still other examples, an REG cocktail enhances allele specific RNAi, which allows allele-specific silencing of a mutant target allele, while not interfering with the expression of a wild-type allele. Such methods are described, for example, in Xia et al. (Nucleic Acids Res 31:e100 (2003)), Abdelgany et al. (Hum Mol Genet 12:2637 (2003)), and Caplen et al. (Hum Mol Genet 11:175 (2002));

Dosage

With respect to the therapeutic methods of the invention, it is not intended that the administration of a polypeptide, nucleic acid, or compound of the invention to a patient be limited to a particular mode of administration, dosage, or frequency of dosing; the present invention contemplates all modes of administration, including intramuscular, intravenous, intraperitoneal, intravesicular, intraarticular, intralesional, subcutaneous, or any other route sufficient to provide a dose adequate to inhibit or enhance RNAi. The therapeutics(s) may be administered to the patient in a single dose or in multiple doses. When multiple doses are administered, the doses may be separated from one another by, for example, one day, two days, one week, two weeks, or one month. For example, treatments such as an REG polypeptide, a REG nucleobase oligomer, or an RNAi therapeutic compound, (e.g., included in an REG cocktail described herein), or a vector including a nucleic acid molecule that encodes an REG polypeptide or a nucleobase oligomer, may be administered once a week for, e.g., 2, 3, 4, 5, 6, 7, 8, 10, 15, 20, or more weeks. It is to be understood that, for any particular subject, specific dosage regimes should be adjusted over time according to the individual need and the professional judgment of the person administering or supervising the administration of the compositions. The precise dose will vary dependent on the treatment used, the concentration of the target of the treatment, and the rate of clearance of the treatment. For example, the dosage of an REG cocktail described herein can be increased if the lower dose does not provide sufficient RNA inhibition or enhancement. Conversely, the dosage of an REG cocktail described herein can be decreased if the need for RNAi inhibition or enhancement is no longer needed in the patient.

While the attending physician ultimately will decide the appropriate amount and dosage regimen, a therapeutically effective amount of a polypeptide, nucleic acid, or compound, may be, for example, in the range of about 0.1 mg to 50 mg/kg body weight/day or 0.70 mg to 350 mg/kg body weight/week. Desirably a therapeutically effective amount is in the range of about 0.50 mg to 20.0 mg/kg, and more desirably in the range of about 0.50 mg to 15.0 mg/kg, for example, about 0.2, 0.3, 0.5, 1.0, 1.5, 2.0, 2.5, 3.0, 3.5, 4.0, 4.5, 5.0, 5.5, 6.0, 7.0, 8.0, 8.5, 9.0, 10.0, 11.0, 12.0, 13.0, 14.0, or 15.0 mg/kg body weight administered daily, every other day, or twice a week.

For instance, a suitable dose is an amount of the treatment that, when administered as described above, is capable of inhibiting or enhancing RNAi, and is at least 20% from the basal (i.e., untreated) level. In general, an appropriate dosage and treatment regimen provides the active compound(s) in an amount sufficient to provide therapeutic and/or prophylactic benefit. Such a response can be monitored by establishing an improved clinical outcome in treated patients as compared to non-treated patients. According to this invention, the administration of the therapeutic can enhance or inhibit RNAi by at least 20%, 40%, 50%, or 75% as compared to an untreated control as measured by any standard assay known in the art. In another embodiment, RNAi may be enhanced or inhibited by 80%, 90%, 95%, or even 100% as compared to an untreated control. Such responses can be monitored by any standard technique known in the art, including those described herein. Suitable dose sizes will vary with the size of the patient, but will typically range from about 0.1 mL to about 5 mL.

Formulation of Pharmaceutical Compositions

A treatment described herein may be administered by any suitable means that results in a concentration having RNAi-modulating properties upon reaching the target region. The treatment may be contained in any appropriate amount in any suitable carrier substance, and is generally present in an amount of 1-95% by weight of the total weight of the composition. The composition may be provided in a dosage form that is suitable for parenteral (e.g., subcutaneous, intravenous, intramuscular, or intraperitoneal) administration route. The pharmaceutical compositions may be formulated according to conventional pharmaceutical practice (see, e.g., Remington: The Science and Practice of Pharmacy (20th ed.), ed. A. R. Gennaro, Lippincott, Williams & Wilkins, 2000 and Encyclopedia of Pharmaceutical Technology, eds. J. Swarbrick and J. C. Boylan, 1988-1999, Marcel Dekker, New York).

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. If the cells in need of therapy are in direct contact with the blood, or if the targeted cells are only accessible by the bloodstream then the intravenous (I.V.) route may be used. In cases in which the targeted cells are in confined spaces such as the pleural cavity or the peritoneal cavity, the therapeutic may be directly administered into the cavity rather than into the blood stream. The formulation and preparation of such compositions are well known to those skilled in the art of pharmaceutical formulation. Formulations can be found, for example, in Remington (The Science and Practice of Pharmacy (20th ed.), ed. A. R. Gennaro, Lippincott, Williams & Wilkins, 2000 and Encyclopedia of Pharmaceutical Technology, eds. J. Swarbrick and J. C. Boylan, 1988-1999, Marcel Dekker, New York).

REG Polypeptide Expression

In general, REG polypeptides (e.g., polypeptides coded by the nucleic acids of FIGS. 15 and 20) of the invention may be produced by transformation of a suitable host cell with all or part of an REG nucleic acid molecule described herein or a fragment thereof in a suitable expression vehicle.

The REG polypeptide may be produced in a prokaryotic host, for example, E. coli, or in a eukaryotic host, for example, Saccharomyces cerevisiae, mammalian cells (for example, COS 1 or NIH 3T3 cells), or any of a number of plant cells. Such cells are available from a wide range of sources including the American Type Culture Collection (Rockland, Md.); or from any of a number seed companies, for example, W. Atlee Burpee Seed Co. (Warminster, Pa.), Park Seed Co. (Greenwood, S.C.), Johnny Seed Co. (Albion, Me.), or Northrup King Seeds (Harstville, S.C.).

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The polypeptide of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, polypeptides expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant polypeptide can, if desired, be further purified, e.g., by high performance liquid chromatography (see, e.g., Fisher, Laboratory Techniques in Biochemistry and Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). Also included in the invention are polypeptides which are modified in ways which do not abolish their biological activity (assayed, for example as described herein). Such changes may include certain mutations, deletions, insertions, or post-translational modifications, or may involve the inclusion of any of the polypeptides of the invention as one component of a larger fusion protein.

The invention further includes analogs of any naturally occurring polypeptide of the invention. Analogs can differ from the naturally occurring the polypeptide of the invention by amino acid sequence differences, by post-translational modifications, or by both. Analogs of the invention will generally exhibit at least 85%, more preferably 90%, and most preferably 95% or even 99% identity with all or part of a naturally occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

In addition to full-length polypeptides, the invention also includes fragments of any one of the REG polypeptides of the invention. As used herein, the term “fragment,” means at least 5, preferably at least 20 contiguous amino acids, preferably at least 30 contiguous amino acids, more preferably at least 50 contiguous amino acids, and most preferably at least 60 to 80 or more contiguous amino acids. Fragments of the invention can be generated by methods known to those skilled in the art or may result from normal polypeptide processing (e.g., removal of amino acids from the nascent polypeptide that are not required for biological activity or removal of amino acids by alternative mRNA splicing or alternative polypeptide processing events). The aforementioned general techniques of polypeptide expression and purification can also be used to produce and isolate useful peptide fragments or analogs (described herein).

For eukaryotic expression, the method of transformation or transfection and the choice of vehicle for expression of the REG polypeptide will depend on the host system selected. Transformation and transfection methods are described, e.g., in Ausubel et al. (supra); Weissbach and Weissbach, Methods for Plant Molecular Biology, Academic Press, 1989; Gelvin et al., Plant Molecular Biology Manual, Kluwer Academic Publishers, 1990; Kindle, Proc Natl Acad Sci USA 87:1228 (1990); Potrykus, Ann Rev Plant Physio Plant Mol Biol 42:205 (1991); and BioRad (Hercules, Calif.) Technical Bulletin #1687 (Biolistic Particle Delivery Systems). Expression vehicles may be chosen from those provided, e.g., in Cloning Vectors: A Laboratory Manual (Pouwels et al., 1985, Supp. 1987); Gasser and Fraley (supra); Clontech Molecular Biology Catalog (Catalog 1992/93 Tools for the Molecular Biologist, Palo Alto, Calif.); and the references cited above. Other expression constructs are described by Fraley et al. (U.S. Pat. No. 5,352,605).

siRNA

Short twenty-one to twenty-five nucleotide double stranded RNAs effectively down-regulate gene expression in vitro, for example, in mammalian tissue culture cell lines (Elbashir et al., Nature 411:494 (2001)) and in vivo (McCaffrey et al., Nature 418:38 (2002)).

Provided with the sequence of a human homolog of an REG identified herein (e.g., the nucleic acids in FIGS. 15 and 20), siRNAs may be designed that decrease the efficacy of RNAi in a particular cell type, tissue, or organs. Methods for designing siRNAs are known to the skilled artisan. (See, for example, Dykxhoom, Nature Rev Mol Cell Biol 4:457 (2003); Paddison et al., Genes Dev 16:948 (2002); Paddison et al., Proc Natl Acad Sci USA 99:1443 (2002); Sohail et al., Nucleic Acids Res 31:e38 (2003); Yu et al., Proc Natl Acad Sci USA 99:6047 (2002)). While various parameters are used to identify promising RNAi targets, the most effective siRNA and shRNA candidate sequences are identified by empirical testing.

In one example, human siRNAs are identified as follows. An siRNA targeting a human homolog of an REG and an siRNA targeting a gene of interest are transferred into mammalian cells in culture. The administration of the REG siRNA may be prior to, co-incident with, or shortly after the administration of an siRNA targeting a gene of interest. The expression of the gene of interest is compared in cells contacted with REG siRNA and in corresponding control cells not contacted with REG siRNA. siRNAs that limit the downregulation of a gene of interest in an REG contacted cell relative to a control cell are useful in the methods of the invention.

Specific REG siRNAs that limit RNAi in vitro can be used in vivo as therapeutics and are especially useful in limiting the downregulation of an RNAi target genes.

Methods for Producing siRNAs and Other Oligonucleotides

Methods for producing REG siRNAs and other REG inhibitory nucleobase oligomers are standard in the art. For example, an REG siRNA can be chemically synthesized or recombinantly produced using methods known in the art. For example, short sense and antisense RNA oligomers can be synthesized and annealed to form double-stranded RNA structures with 2-nucleotide overhangs at each end (Caplen et al., Proc Natl Acad Sci USA 98:9742 (2001); Elbashir et al., EMBO J 20:6877 (2001)).

21-23 nucleotide REG dsRNAs can be chemically synthesized by any method known to one of skill in the art, for example, using Expedite RNA phosphoramidites and thymidine phosphoramidite (PROLIGO, Boulder, Colo.). Synthetic oligonucleotides can be deprotected and gel-purified. dsRNA annealing can be carried out by any method known in the art, for example: a phenol-chloroform extraction, followed by mixing equimolar concentrations of sense and antisense RNA (50 nM to 10 mM, depending on the length and amount available) and incubating in an appropriate buffer (such as 0.3 M NaOAc, pH 6) at 90° C. for 30 seconds and then extracting with phenol/chloroform and chloroform. The resulting dsRNA can be precipitated with ethanol and dissolved in an appropriate buffer depending on the intended use of the dsRNA. These double-stranded siRNA structures can then be directly introduced to cells, either by passive uptake or a delivery system of choice.

In some embodiments, the REG siRNA constructs can be generated by processing longer double-stranded RNAs, for example, in the presence of the enzyme dicer under conditions in which the dsRNA is processed to RNA molecules of about 21 to about 23 nucleotides.

In other embodiments, an REG RNA can be transcribed from PCR products, followed by gel purification. Standard procedures known in the art for in vitro transcription of RNA from PCR templates carrying, for example, T7 or SP6 promoter sequences can be used. The dsRNAs can be synthesized by using a PCR template and the AMBION (Austin, Tex.) T7 MEGASCRIPT kit, following the Manufacturer's recommendations and the RNA can then be precipitated with LiCl and resuspended in buffer. The specific dsRNAs produced can be tested for resistance to digestion by RNases A and T1. The dsRNAs can be produced with 3′ overhangs at both termini or one terminus of preferably 1-10 nucleotides, more preferably 1-3 nucleotides or with blunt ends at one or both termini. In one embodiment, thymidine nucleotide overhangs are useful for enhancing nuclease resistance of siRNAs.

Other standard methods for the preparation of siRNAs and other nucleobase oligomers are described, for example, in Ausubel et al., Current Protocols in Molecular Biology (Supplement 56), John Wiley & Sons, New York (2001); Sambrook and Russel, Molecular Cloning: A Laboratory Manual, 3rd ed., Cold Spring Harbor Press, Cold Spring Harbor (2001); and Dieffenbach and Dveksler, PCR Primer: A Laboratory Manual, Cold Spring Harbor Press (1995), all of which are incorporated herein by reference in their entirety.

REG siRNA molecules can be purified using a number of techniques known to those of skill in the art. For example, gel electrophoresis can be used to purify REG siRNAs.

Non-denaturing methods, such as non-denaturing column chromatography, can be used to purify the REG siRNA. In addition, chromatography (e.g., size exclusion chromatography), glycerol gradient centrifugation, affinity purification with antibody can be used to purify siRNAs.

In preferred embodiments, at least one strand of the REG siRNA molecules has a 3′ overhang from about 1 to about 6 nucleotides in length, though the overhang may be from 2 to 4 nucleotides in length. More preferably, the 3′ overhangs are 1-3 nucleotides in length. In other embodiments, one strand has a 3′ overhang and the other strand is blunt-ended or also has an overhang. The length of the overhangs may be the same or different for each strand. In order to further enhance the stability of the siRNA, the 3′ overhangs can be stabilized against degradation. In one embodiment, the REG RNA is stabilized by including purine nucleotides, such as adenosine or guanosine nucleotides or by substituting pyrimidine nucleotides by modified analogs, e.g., substitution of uridine nucleotide 3′ overhangs by 2′-deoxythyinidine. In other embodiments, the absence of a 2′ hydroxyl can significantly enhance nuclease resistance of the overhang.

Also useful in the methods of the invention are REG shRNAs. Such RNAs can be synthesized exogenously or can be formed by transcribing from a promoter in vivo. For expression of REG shRNAs within cells, plasmid or viral vectors may contain, for example, a promoter, including, but not limited to the polymerase I, II, and III H1, U6, BL, SMK, 7SK, tRNA polIII, tRNA(met)-derived, and T7 promoters, a cloning site for the stem-looped RNA coding insert, and a 4-5-thymidine transcription termination signal. The Polymerase III promoters generally have well-defined initiation and stop sites and their transcripts lack poly(A) tails. Examples of making and using shRNAs for gene silencing in mammalian cells are described in, for example, Paddison et al. (Genes Dev 16:948 (2002)); McCaffrey et al. (Nature 418:38 (2002)); McManus et al. (RNA 8:842 (2002)); and Yu et al. (Proc Natl Acad Sci USA 99:6047 (2002)). Preferably, such shRNAs are engineered in cells or in an animal to ensure continuous and stable suppression of a target gene. It is known that siRNAs can be produced by processing a hairpin RNA in a cell.

siRNA Delivery

For some applications, a plasmid is used to deliver an REG inhibitory nucleobase oligomer (e.g., nucleobase oligomers with sequence from the nucleic acids in FIGS. 15 and 20), such as a double stranded RNA, siRNA, or shRNA, as a transcriptional product. In such embodiments, the plasmid is designed to include a coding sequence for each of the sense and antisense strands of an REG RNAi construct. The coding sequences can be the same sequence, e.g., flanked by inverted promoters, or can be two separate sequences each under the transcriptional control of separate promoters. After the coding sequence is transcribed, the complementary REG RNA transcripts base pair to form a double-stranded RNA. PCT application WO01/77350 describes an exemplary vector for bi-directional transcription of a transgene to yield both sense and antisense RNA transcripts of the same transgene in a eukaryotic cell.

Methods for the production and therapeutic administration of siRNAs for in vivo therapies are described in U.S. Patent Application Publications: 20030180756, 20030157030, and 20030170891. Methods describing the successful in vivo use of siRNA are described by Song et al. (Nature Med 9: 347 (2003).

Administration to cells of REG inhibitory nucleic acids, or vectors encoding such nucleic acids, can be carried out by any standard method. For example, an REG inhibitory nucleic acid or a vector encoding an REG inhibitory nucleic acid can be introduced in vivo by lipofection. Liposomes for encapsulation and transfection of nucleic acids in vitro may be used. For some applications, synthetic cationic lipids can be used to prepare liposomes for in vivo transfection (Felgner et al., Proc Natl Acad Sci USA 84:7413 (1987); see also, Mackey, et al., Proc Natl Acad Sci USA 85:8027 (1988); Ulmer et al., Science 259:1745 (1993)). The use of cationic lipids may promote encapsulation of negatively charged nucleic acids, and also promote fusion with negatively charged cell membranes (Felgner and Ringold, Science 337:387 (1989)). Particularly useful lipid compounds and compositions for transfer of nucleic acids are described in WO95/18863, WO96/17823, and in U.S. Pat. No. 5,459,127. Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce an REG inhibitory nucleic acid or an expression vector encoding such a nucleic acid in vivo as a naked DNA. Methods for formulating and administering naked DNA to mammalian tissue are disclosed in U.S. Pat. Nos. 5,580,859 and 5,589,466.

Because inhibitory nucleic acids may be substrates for nuclease degradation, modified or substituted inhibitory nucleic acids are often preferred because of properties such as, for example, enhanced cellular uptake and increased stability in the presence of nucleases.

Modified Nucleobase Oligomers

An REG inhibitory nucleic acid or nucleobase oligomer may include modifications that increase nuclease resistance or that enhance the activity, cellular distribution or cellular uptake of the oligonucleotide. In various embodiments, an REG oligomeric mimetic contains novel groups in place of the sugar, the backbone, or both. The base units are maintained to allow hybridization with an appropriate nucleic acid target compound.

Specific examples of some preferred REG nucleic acids envisioned for this invention may contain phosphorothioates, phosphotriesters, methyl phosphonates, short chain alkyl or cycloalkyl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—P—O—CH₂). Also preferred are oligonucleotides having morpholino backbone structures (Summerton and Weller, U.S. Pat. No. 5,034,506). In other preferred embodiments, such as the protein-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al., Science 254:1497 (1991)). Other preferred REG oligonucleotides may contain alkyl and halogen-substituted sugar moieties comprising one of the following at the 2′ position: OH, SH, SCH₃, F, OCN, O(CH₂)ₙNH₂ or O(CH₂)ₙ CH₃, where n is from 1 to about 10; C₁ to C₁₀ lower alkyl, substituted lower alkyl, alkaryl or aralkyl; Cl; Br; CN; CF₃; OCF₃; O-, S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; an RNA cleaving group; a conjugate; a reporter group; an intercalator; a group for improving the pharmacokinetic properties of an oligonucleotide; or a group for improving the pharmacodynamic properties of an oligonucleotide and other substituents having similar properties. REG oligonucleotides may also have sugar mimetics such as cyclobutyls in place of the pentofuranosyl group.

In other preferred embodiments, an REG oligomer may include at least one modified base form. Some specific examples of such modified bases include 2-(amino)adenine, 2-(methylamino)adenine, 2-(imidazolylalkyl)adenine, 2-(aminoalklyamino)adenine, or other heterosubstituted alkyladenines. In one embodiment, an REG oligomer includes one or more G-clamp nucleotides. A G-clamp nucleotide is a modified cytosine analog having a modification that confers the ability to hydrogen bond both Watson-Crick and Hoogsteen faces of a complementary guanine within a duplex, see for example Lin and Matteucci (J Am Chem Soc 120, 8531 (1998)). A single G-clamp analog substitution within an oligomer can result in substantially enhanced helical thermal stability and mismatch discrimination when hybridized to complementary oligonucleotides. In another embodiment, REG nucleic acid molecules of the invention include one or more LNA “locked nucleic acid” nucleotides such as a 2′,4′-C mythylene bicyclo nucleotide (see for example Wengel et al., International PCT Publication No. WO 00/66604 and WO 99/14226).

In other embodiments, an REG oligomer contains one or more moieties or conjugates which enhance the activity, cellular distribution or cellular uptake of the oligonucleotide.

The compounds of the invention can include conjugate groups covalently bound to functional groups such as primary or secondary hydroxyl groups. Conjugate groups include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the properties of an oligonucleotide include groups that improve oligomer uptake, enhance oligomer resistance to degradation, and/or strengthen sequence-specific hybridization with RNA, improve oligomer uptake, distribution, metabolism, or excretion. Conjugate moieties include, but are not limited to, lipid moieties such as a cholesterol moiety, cholic, a thioether, e.g., hexyl-S-tritylthiol, athiocholesterol, analiphatic chain, a phospholipid, e.g., di-hexadecyl-rac-glycerol or triethyl-ammonium1,2-di-O-hexadecyl-rac-glyc-ero-3-H-phosphonate, a polyamine or a polyethylene glycol chain, or adamantane acetic acid, a palmityl moiety, or an octadecylamine orhexylamino-carbonyl-oxycholesterol moiety. Methods for the preparation of such oligonucleotide conjugates are standard in the art, and include, but are not limited to exonuclease resistant terminally substituted oligonucleotides, which are described in 5,245,022; oligonucleotide-enzyme conjugates, which are described in 5,254,469; boronated phosphoramidate conjugates, which are described in 5,272,250; detectably tagged oligomers, which are described in 5,317,098; oligomer polypeptide conjugates, which are described in 5,391,723; and steroid modified oligomers, which are described in 5,416,203. Other oligonucleotide conjugates are described in, for example, in U.S. Pat. Nos. 5,258,506; 5,262,536; 5,292,873; 5,371,241, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

REG oligomers may also include nucleobase (often referred to in the art simply as “base”) modifications or substitutions. “Unmodified” or “natural” nucleotides include the purine bases adenine (A) and guanine (G), and the pyrimidine bases thymine (T), cytosine (C) and uracil (U). Modified nucleotides are known in the art, and are described in U.S. Pat. No. 3,687,808, The Concise Encyclopedia Of Polymer Science And Engineering, pages 858-859, Kroschwitz, J. I., ed. John Wiley & Sons, 1990, Englisch et al., Angewandte Chemie, International Edition (1991), 30, 613, and those disclosed by Sanghvi, Y. S., Chapter 15, Antisense Research and Applications, pages 289-302, Crooke, S. T. and Lebleu, B. ed., CRC Press, 1993. Modified nucleobases are particularly useful for increasing the binding affinity of the oligomeric compounds of the invention. These modified nucleobases include, but are not limited to, 5-substituted pyrimidines, 6-azapyrimidines and N-2, N-6 and 0-6 substituted purines, including 2-aminopropyladenine, 5-propynyluracil and S-propynylcytosine. 5-methylcytosine substitutions.

Oligonucleotide Backbones

At least two types of oligonucleotides induce the cleavage of RNA by Rnase H: oligodeoxynucleotides with phosphodiester (PO) or phosphorothioate (PS) linkages. Although 2′-OMe-RNA sequences exhibit a high affinity for RNA targets, these sequences are not substrates for RNase H. A desirable oligonucleotide is one based on 2′-modified oligonucleotides containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅₀. This modification also increases the nuclease resistance of the modified oligonucleotide. Peptide Nucleic Acids (PNA) may also be employed.

Locked Nucleic Acids

Locked nucleic acids (LNA) are nucleotide analogs that can be employed in the present invention. LNA contain a 2′O, 4′-C methylene bridge that restrict the flexibility of the ribofuranose ring of the nucleotide analog and locks it into the rigid bicyclic N-type conformation. LNA show improved resistance to certain exo- and endonucleases and activate RNAse H, making them suitable for use in methods described herein. LNA can be incorporated into almost any oligonucleotide. Moreover, LNA-containing oligonucleotides can be prepared using standard phosphoramidite synthesis protocols. Additional details regarding LNA can be found in PCT publication WO99/14226, hereby incorporated by reference.

Arabinonucleic Acids

Arabinonucleic acids (ANA) can also be employed in the methods and reagents of the present invention. ANA are based on D-arabinose sugars instead of the natural D-2′-deoxyribose sugars. Underivatized ANA analogs have similar binding affinity for RNA as phosphorothioates. When the arabinose sugar is derivatized with fluorine (2′ F-ANA), an enhancement in binding affinity results, and selective hydrolysis of bound RNA occurs efficiently in the resulting ANA/RNA and F-ANA/RNA duplexes. These analogs can be made stable in cellular media by a derivatization at their termini with simple L sugars.

Isolation of Additional REG Genes

Based on the REG nucleotide and amino acid sequences described herein, the isolation and identification of additional coding sequences of orthologous REG genes is made possible using standard strategies and techniques that are well known in the art.

In one example, the REG polypeptides disclosed herein are used to search a database to identify orthologs, as described herein.

In another example, any one of the REG nucleotide sequences described herein may be used in conventional methods of nucleic acid hybridization screening. Such hybridization techniques and screening procedures are well known to those skilled in the art and are described, for example, in Benton and Davis (Science 196:180 (1977)); Grunstein and Hogness (Proc Natl Acad Sci USA 72:3961 (1975)); Ausubel et al. (Current Protocols in Molecular Biology, Wiley Interscience, New York (2001)); Berger and Kimmel (Guide to Molecular Cloning Techniques, 1987, Academic Press, New York); and Sambrook et al. (Molecular Cloning: A Laboratory Manual, Cold Spring Harbor Laboratory Press, New York). In one particular example, all or part of an REG nucleic acid sequence may be used as a probe to screen a recombinant DNA library for genes having sequence identity to an REG gene. Hybridizing sequences are detected by plaque or colony hybridization according to standard methods.

In another embodiment, using all or a portion of an REG nucleic acid sequence one may readily design gene- or nucleic acid sequence-specific oligonucleotide probes, including degenerate oligonucleotide probes (i.e., a mixture of all possible coding sequences for a given amino acid sequence). These oligonucleotides may be based upon the sequence of either DNA strand or any appropriate portion of the nucleic acid sequence. General methods for designing and preparing such probes are provided, for example, in Ausubel et al. (supra), and Berger and Kimmel (supra). These oligonucleotides are useful for REG gene isolation, either through their use as probes capable of hybridizing to an REG gene, or as complementary sequences or as primers for various amplification techniques, for example, polymerase chain reaction (PCR) cloning strategies. If desired, a combination of different, detectably-labelled oligonucleotide probes may be used for the screening of a recombinant DNA library. Such libraries are prepared according to methods well known in the art, for example, as described in Ausubel et al. (supra), or they may be obtained from commercial sources.

As discussed above, REG sequence-specific oligonucleotides may also be used as primers in amplification cloning strategies, for example, using PCR. PCR methods are well known in the art and are described, for example, in PCR Technology, Erlich, ed., Stockton Press, London, 1989; PCR Protocols: A Guide to Methods and Applications, Innis et al., eds., Academic Press, Inc., New York, 1990; and Ausubel et al. (supra). Primers are optionally designed to allow cloning of the amplified product into a suitable vector, for example, by including appropriate restriction sites at the 5′ and 3′ ends of the amplified fragment (as described herein). If desired, nucleotide sequences may be isolated using the PCR “RACE” technique, or Rapid Amplification of cDNA Ends (see, e.g., Innis et al. (supra)). By this method, oligonucleotide primers based on a desired sequence are oriented in the 3′ and 5′ directions and are used to generate overlapping PCR fragments. These overlapping 3′- and 5′-end RACE products are combined to produce an intact full-length cDNA. This method is described in Innis et al. (supra); and Frohman et al. (Proc Natl Acad Sci USA 85:8998 (1988)).

Partial sequences, e.g., sequence tags, are also useful as hybridization probes for identifying full-length sequences, as well as for screening databases for identifying previously unidentified related virulence genes.

In general, the invention includes any nucleic acid sequence that may be isolated as described herein or which is readily isolated by homology screening or PCR amplification using any of the nucleic acid sequences disclosed herein.

It will be appreciated by those skilled in the art that, as a result of the degeneracy of the genetic code, a multitude of polynucleotide sequences encoding REG genes, some bearing minimal similarity to the polynucleotide sequences of any known and naturally occurring gene, may be produced. Thus, the invention contemplates each and every possible variation of polynucleotide sequence that may be made by selecting combinations based on possible codon choices. These combinations are made in accordance with the standard triplet genetic code as applied to the polynucleotide sequence of naturally occurring REG genes, and all such variations are to be considered as being specifically disclosed.

Although nucleotide sequences of REG genes or their variants are preferably capable of hybridizing to the nucleotide sequence of a naturally occurring REG genes under appropriately selected conditions of stringency, it may be advantageous to produce nucleotide sequences encoding REG genes, or their derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding REG genes and their derivatives without altering the encoded amino acid sequences include the production of RNA transcripts having more desirable properties, such as a greater half-life, than transcripts produced from the naturally occurring sequence.

The invention also encompasses production of DNA sequences that encode REG genes, or fragments thereof generated entirely by synthetic chemistry. After production, the synthetic sequence may be inserted into any of the many available expression vectors and cell systems using reagents well known in the art. Moreover, synthetic chemistry may be used to introduce mutations into a sequence encoding any REG gene, or any fragment thereof.

Also encompassed by the invention are polynucleotide sequences that are capable of hybridizing to any REG polynucleotide sequences, and fragments thereof under various conditions of stringency. (See, e.g., Wahl and Berger, Methods Enzymol 152:399 (1987); and Kimmel, Methods Enzymol 152:507 (1987)) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The washing steps which follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

Methods for DNA sequencing are well known in the art and may be used to practice any of the embodiments of the invention. The resulting sequences are analyzed using a variety of algorithms which are well known in the art. (See, e.g., Ausubel, Short Protocols in Molecular Biology, John Wiley & Sons, New York N.Y., (1997) unit 7.7).

Screening Assays

As discussed above, the identified REG polypeptides are essential for RNAi. Based on this discovery, screening assays to identify compounds that increase the activity of an REG polypeptide or that increase the expression of an REG nucleic acid sequence of the invention were developed. The method of screening may involve high-throughput techniques. In addition, these screening techniques may be carried out in cultured cells or in animals (such as nematodes).

Any number of methods are available for carrying out such screening assays. In one example, candidate compounds are added at varying concentrations to the culture medium of cultured cells or nematodes expressing one of the REG nucleic acid sequences of the invention. REG gene expression is then measured, for example, by standard Northern blot analysis (Ausubel et al., supra) or RT-PCR, using any appropriate fragment prepared from the nucleic acid molecule as a hybridization probe. The level of REG gene expression in the presence of the candidate compound is compared to the level measured in a control culture medium lacking the candidate molecule. Such cultured cells include nematode cells (for example, C. elegans cells), mammalian, insect, or plant cells. A compound that increase REG expression is considered useful in the invention; such a molecule may be used, for example, as a therapeutic to enhance RNAi.

In another example, the effect of candidate compounds is measured at the level of REG polypeptide production using the same general approach and standard immunological techniques, such as Western blotting or immunoprecipitation with an antibody specific for an REG polypeptide. For example, immunoassays may be used to detect or monitor the expression of at least one of the polypeptides of the invention in an organism. Polyclonal or monoclonal antibodies (produced as described above) that are capable of binding to such a polypeptide may be used in any standard immunoassay format (e.g., ELISA, Western blot, or RIA assay) to measure the level of the polypeptide. In another example, REG polypeptide expression is detected by fusing the REG polypeptide to a detectable reporter. A compound that increase the expression of the polypeptide is considered particularly useful. Again, such a molecule may be used, for example, as a therapeutic to enhance RNAi.

In yet another working example, candidate compounds are screened for those that specifically bind to and agonize an REG polypeptide. Particularly useful are those polypeptides that increase the biological activity of an REG polypeptide. The efficacy of such a candidate compound is dependent upon its ability to interact with REG or a functional equivalent thereof. Such an interaction can be readily assayed using any number of standard binding techniques and functional assays (e.g., those described in Ausubel et al., supra). For example, a candidate compound may be tested in vitro for interaction and binding with a polypeptide of the invention and its ability to enhance RNAi may be assayed by any standard assay (e.g., those described herein).

In one particular working example, a candidate compound that binds to an REG polypeptide, may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the REG polypeptide is identified on the basis of its ability to bind to an REG polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to enhance RNAi (e.g., as described herein). Compounds isolated by this approach may also be used, for example, as therapeutics to delay or ameliorate human diseases associated with the expression or overexpression of a gene. Compounds that are identified as binding to an REG polypeptide with an affinity constant less than or equal to 10 mM are considered particularly useful in the invention.

Potential agonists include organic molecules, peptides, peptide mimetics, polypeptides, nucleic acids, and antibodies that bind to an REG nucleic acid sequence or polypeptide of the invention and thereby increase its biological activity.

Each of the REG DNA sequences provided herein may also be used in the discovery and development of RNAi enhancing compounds. The encoded REG polypeptide, upon expression, can be used as a target for the screening of RNAi enhancing drugs that increase REG polypeptide activity.

The agonists of the invention may be employed, for instance, to prevent, delay or ameliorate human or plant diseases associated with the expression or overexpression of a gene or to treat or prevent a pathogen infection.

Optionally, compounds identified in any of the above-described assays may be confirmed as useful in delaying or ameliorating human diseases associated in either standard tissue culture methods or animal models and, if successful, may be used as therapeutics for enhancing RNAi in a subject in need of gene silencing.

Small molecules of the invention preferably have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of enhancing REG activity are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). In another embodiment, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). In another embodiment, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity in inhibiting nuclease activity should be employed whenever possible.

When a crude extract is found to have an REG polypeptide enhancing activity, or an REG binding activity, further fractionation of the positive lead extract is necessary to isolate chemical constituents responsible for the observed effect. Thus, the goal of the extraction, fractionation, and purification process is the careful characterization and identification of a chemical entity within the crude extract having an REG polypeptide enhancing activity. Methods of fractionation and purification of such heterogenous extracts are known in the art. If desired, compounds shown to be useful agents for enhancing an REG polypeptide activity are chemically modified according to methods known in the art.

REG Polypeptides in Plants

As described above, an inhibitory nucleic acid that targets a gene of interest can be administered to a plant cell in a cocktail of one or more REG polypeptides. Such co-administration enhances RNAi in a plant cell or tissue, particularly in cells or tissues that are refractory to RNAi because they fail to express an essential component of the RNAi pathway.

For some applications, an attenuated strain of a microorganism is engineered to express REG nucleic acids in addition to inhibitory nucleic acids that target a pathogen gene of interest, such as an essential pathogen gene. Exposure of the pathogen to the host plant results in ingestion of the RNAi microorganisms leading to the REG enhanced silencing of the target pathogen gene. By enhancing the silencing of, for example, an essential pathogen gene, an expressed REG polypeptide prevents, reduces, or eliminates infection or infestation of the host plant by the pathogen.

For other applications, the inhibitory nucleic acid molecules are encapsulated in a synthetic matrix, such as a polymer, that includes REG polypeptides and the matrix is applied to the surface of a host plant. Ingestion of host cells by a pathogen delivers the inhibitory molecules and REG polypeptides to the pathogen and results in the enhanced down-regulation of a target gene in the pathogen. Examples of plant pathogens include, but are not limited to viruses, bacteria, parasites, or insects in contact with the plant cell. Methods for using inhibitory nucleic acids in plants are known to the skilled artisan (see, for example, U.S. Pat. Nos. 6,452,067, 6,500,670, 6,395,962, 6,369,296, 6,002,071; or U.S. Patent Publication No. 20030150017).

REG RNAi in Plants

As described herein, REG nucleic acid molecules and polypeptides are also expressed in plants. As in other eukaryotic cells, inhibitory REG nucleic acids or nucleobase oligomers are useful in limiting RNAi in a plant cell. RNAi provides a convenient mechanism for altering the phenotype of a plant by reducing or eliminating the expression of a particular endogenous target gene. Inhibitory nucleic acids that target an REG are useful in limiting RNAi in a plant cell. For example, in limiting RNAi to a particular location or cell type or in limiting the efficacy of RNAi. While it may be desirable to decrease the expression of a target gene using RNAi, co-administration of an inhibitory nucleic acid that targets an REG can be used to limit this down regulation. Such limits are useful when it would be undesirable to eliminate the expression of a gene.

In one example, an inhibitory REG nucleic acid molecule is administered to or expressed in a plant cell in conjunction with an inhibitory nucleic acid molecule that targets an endogenous gene of interest to limit the silencing of that gene.

Construction of Plant Transgenes

Transgenic plants expressing an REG transgene encoding an REG inhibitory nucleic acid, including, but not limited to, dsRNA, siRNA, shRNA, or antisense RNA, are useful for limiting the efficacy of RNAi in a plant. In another embodiment, transgenic plants expressing an REG transgene encoding at least one or more REG polypeptides exhibits enhanced RNAi. This is particularly useful in plants that lack an essential component of the RNAi machinery. A transgenic plant, or population of such plants, expressing at least one transgene encoding an REG inhibitory nucleic acid would be expected to show a limited response to RNAi. For some applications, an REG inhibitory nucleic acid molecule is co-expressed with a transgene encoding an inhibitory nucleic acid molecule that targets a gene of interest. In another embodiment, a transgenic plant expressing at least one REG inhibitory nucleic acid is entirely refractory to RNAi. Such plants are useful for the identification or characterization of essential components of the RNAi pathway.

In one preferred embodiment, an REG nucleic acid (is expressed by a stably-transfected plant cell line, a transiently-transfected plant cell line, or by a transgenic plant. A number of vectors suitable for stable or extrachromosomal transfection of plant cells or for the establishment of transgenic plants are available to the public; such vectors are described in Pouwels et al. (supra), Weissbach and Weissbach (supra), and Gelvin et al. (supra). Methods for constructing such cell lines are described in, e.g., Weissbach and Weissbach (supra), and Gelvin et al. (supra).

Vectors useful in the methods of the invention are described, for example, in U.S. Pat. No. 5,922,602; WO 99/36516; MacFarlane and Popovich. Virology 267:29 (2000); and U.S. Patent Publication No. 20020165370.

Plant expression constructs having an REG gene that encodes an REG inhibitory nucleic acid or an REG polypeptide may be employed with a wide variety of plant life, particularly plant life involved in the production of storage reserves (for example, those involving carbon and nitrogen metabolism). Such genetically-engineered plants are useful for a variety of industrial and agricultural applications. Importantly, this invention is applicable to dicotyledons and monocotyledons, and will be readily applicable to any new or improved transformation or regeneration method.

The expression constructs include at least one promoter operably linked to at least one REG gene. Examples of plant expression constructs are found in Fraley et al., U.S. Pat. No. 5,352,605. In most tissues of transgenic plants, the CaMV 35S promoter is a strong promoter (see, e.g., Odell et al., Nature 313:810 (1985)). Other useful plant promoters include, without limitation, the nopaline synthase (NOS) promoter (An et al., Plant Physiol 88:547 (1988) and Rodgers and Fraley, U.S. Pat. No. 5,034,322), the octopine synthase promoter (Fromm et al., Plant Cell 1:977 (1989)), figwort mosiac virus (FMV) promoter (Rodgers, U.S. Pat. No. 5,378,619), and the rice actin promoter (Wu and McElroy, WO91/09948).

Exemplary monocot promoters include, without limitation, commelina yellow mottle virus promoter, sugar cane badna virus promoter, rice tungro bacilliform virus promoter, maize streak virus element, and wheat dwarf virus promoter.

For certain applications, it may be desirable to produce REG inhibitory nucleic acid or polypeptide in an appropriate tissue, at an appropriate level, or at an appropriate developmental time. For this purpose, there are an assortment of gene promoters, each with its own distinct characteristics embodied in its regulatory sequences, shown to be regulated in response to inducible signals such as the environment, hormones, and/or developmental cues. These include, without limitation, gene promoters that are responsible for heat-regulated gene expression (see, e.g., Callis et al., Plant Physiol 88:965 (1988); Takahashi and Komeda, Mol Gen Genet 219:365 (1989); and Takahashi et al., Plant J 2:751 (1992)), light-regulated gene expression (e.g., the pea rbcS-3A described by Kuhlemeier et al., Plant Cell 1:471 (1989); the maize rbcS promoter described by Schaffner and Sheen, Plant Cell 3:997 (1991); the chlorophyll a/b-binding protein gene found in pea described by Simpson et al., EMBO J. 4:2723 (1985); the Arabssu promoter; or the rice rbs promoter), hormone-regulated gene expression (for example, the abscisic acid (ABA) responsive sequences from the Em gene of wheat described by Marcotte et al., Plant Cell 1:969 (1989); the ABA-inducible HVA1 and HVA22, and rd29A promoters described for barley and Arabidopsis by Straub et al., Plant Cell 6:617 (1994) and Shen et al., Plant Cell 7:295 (1995); and wound-induced gene expression (for example, of wuni described by Siebertz et al., Plant Cell 1:961 (1989)), organ-specific gene expression (for example, of the tuber-specific storage protein gene described by Roshal et al., EMBO J 6:1155 (1987); the 23-kDa zein gene from maize described by Schemthaner et al., EMBO J. 7:1249 (1988); or the French bean β-phaseolin gene described by Bustos et al., Plant Cell 1:839 (1989)), or pathogen-inducible promoters (for example, PR-1, prp-1, or -1,3 glucanase promoters, the fungal-inducible wirla promoter of wheat, and the nematode-inducible promoters, TobRB7-5A and Hmg-1, of tobacco and parsley, respectively).

Plant expression vectors may also optionally include RNA processing signals, e.g., introns, which have been shown to be important for efficient RNA synthesis and accumulation (Callis et al., Genes and Dev 1:1183 (1987)). The location of the RNA splice sequences can dramatically influence the level of transgene expression in plants. In view of this fact, an intron may be positioned upstream or downstream of an REG inhibitory nucleic acid or polypeptide-encoding sequence in the transgene to modulate levels of gene expression.

In addition to the aforementioned 5′ regulatory control sequences, the expression vectors may also include regulatory control regions which are generally present in the 3′ regions of plant genes (Thornburg et al., Proc Natl Acad Sci USA 84:744 (1987); An et al., Plant Cell 1:115 (1989)). For example, the 3′ terminator region may be included in the expression vector to increase stability of the mRNA. One such terminator region may be derived from the PI-II terminator region of potato. In addition, other commonly used terminators are derived from the octopine or nopaline synthase signals.

The plant expression vector also typically contains a dominant selectable marker gene used to identify those cells that have become transformed. Useful selectable genes for plant systems include genes encoding antibiotic resistance genes, for example, those encoding resistance to hygromycin, kanamycin, bleomycin, G418, streptomycin, or spectinomycin. Genes required for photosynthesis may also be used as selectable markers in photosynthetic-deficient strains. Finally, genes encoding herbicide resistance may be used as selectable markers; useful herbicide resistance genes include the bar gene encoding the enzyme phosphinothricin acetyltransferase and conferring resistance to the broad spectrum herbicide Basta® (Frankfurt, Germany).

Efficient use of selectable markers is facilitated by a determination of the susceptibility of a plant cell to a particular selectable agent and a determination of the concentration of this agent which effectively kills most, if not all, of the transformed cells. Some useful concentrations of antibiotics for tobacco transformation include, e.g., 75-100 μg/mL (kanamycin), 20-50 μg/mL (hygromycin), or 5-10 μg/mL (bleomycin). A useful strategy for selection of transformants for herbicide resistance is described, e.g., by Vasil et al., supra.

In addition, if desired, the plant expression construct may contain a modified or fully-synthetic structural REG inhibitory nucleic acid or polypeptide coding sequence that has been changed to enhance the performance of the gene in plants. Methods for constructing such a modified or synthetic gene are described in Fischoff and Perlak, U.S. Pat. No. 5,500,365.

It should be readily apparent to one skilled in the art of molecular biology, especially in the field of plant molecular biology, that the level of gene expression is dependent, not only on the combination of promoters, RNA processing signals, and terminator elements, but also on how these elements are used to increase the levels of selectable marker gene expression.

Plant Transformation

Upon construction of the plant expression vector, several standard methods are available for introduction of the vector into a plant host, thereby generating a transgenic plant. These methods include (1) Agrobacterium-mediated transformation (A. tumefaciens or A. rhizogenes) (see, e.g., Lichtenstein and Fuller In: Genetic Engineering, vol 6, P W J Rigby, ed, London, Academic Press, 1987; and Lichtenstein, C. P., and Draper, J., In: DNA Cloning, Vol II, D. M. Glover, ed, Oxford, IRI Press, 1985); (2) the particle delivery system (see, e.g., Gordon-Kamm et al., Plant Cell 2:603 (1990)); or BioRad Technical Bulletin 1687, supra); (3) microinjection protocols; (4) polyethylene glycol (PEG) procedures (see, e.g., Draper et al., Plant Cell Physiol 23:451 (1982); or e.g., Zhang and Wu, Theor Appl Genet 76:835 (1988)); (5) liposome-mediated DNA uptake (see, e.g., Freeman et al., Plant Cell Physiol 25:1353 (1984)); (6) electroporation protocols (see, e.g., Fromm et al., Nature 319:791 (1986); Sheen Plant Cell 2:1027 (1990); or Jang and Sheen Plant Cell 6:1665 (1994)); and (7) the vortexing method (see, e.g., Kindle, supra). The method of transformation is not critical to the invention. Any method that provides for efficient transformation may be employed. As newer methods are available to transform crops or other host cells, they may be directly applied. Suitable plants for use in the practice of the invention include, but are not limited to, sugar cane, wheat, rice, maize, sugar beet, potato, barley, manioc, sweet potato, soybean, sorghum, cassaya, banana, grape, oats, tomato, millet, coconut, orange, rye, cabbage, apple, watermelon, canola, cotton, carrot, garlic, onion, pepper, strawberry, yam, peanut, onion, bean, pea, mango, citrus plants, walnuts, and sunflower.

The following is an example outlining one particular technique, an Agrobacterium-mediated plant transformation. By this technique, the general process for manipulating genes to be transferred into the genome of plant cells is carried out in two phases. First, cloning and DNA modification steps are carried out in E. coli, and the plasmid containing the gene construct of interest is transferred by conjugation or electroporation into Agrobacterium. Second, the resulting Agrobacterium strain is used to transform plant cells. Thus, for the generalized plant expression vector, the plasmid contains an origin of replication that allows it to replicate in Agrobacterium and a high copy number origin of replication functional in E. coli. This permits facile production and testing of transgenes in E. coli prior to transfer to Agrobacterium for subsequent introduction into plants. Resistance genes can be carried on the vector, one for selection in bacteria, for example, streptomycin, and another that will function in plants, for example, a gene encoding kanamycin resistance or herbicide resistance. Also present on the vector are restriction endonuclease sites for the addition of one or more transgenes and directional T-DNA border sequences which, when recognized by the transfer functions of Agrobacterium, delimit the DNA region that will be transferred to the plant.

In another example, plant cells may be transformed by shooting into the cell tungsten microprojectiles on which cloned DNA is precipitated. In the Biolistic Apparatus (Bio-Rad) used for the shooting, a gunpowder charge (22 caliber Power Piston Tool Charge) or an air-driven blast drives a plastic macroprojectile through a gun barrel. An aliquot of a suspension of tungsten particles on which DNA has been precipitated is placed on the front of the plastic macroprojectile. The latter is fired at an acrylic stopping plate that has a hole through it that is too small for the macroprojectile to pass through. As a result, the plastic macroprojectile smashes against the stopping plate, and the tungsten microprojectiles continue toward their target through the hole in the plate. For the instant invention the target can be any plant cell, tissue, seed, or embryo. The DNA introduced into the cell on the microprojectiles becomes integrated into either the nucleus or the chloroplast.

In general, the transfer and expression of transgenes in plant cells is now routine for one skilled in the art, and have become major tools to carry out gene expression studies in plants and to produce improved plant varieties of agricultural or commercial interest.

Transgenic Plant Regeneration

Plant cells transformed with a plant expression vector can be regenerated, for example, from single cells, callus tissue, or leaf discs according to standard plant tissue culture techniques. It is well known in the art that various cells, tissues, and organs from almost any plant can be successfully cultured to regenerate an entire plant.

In one particular example, a cloned REG inhibitory nucleic acid or polypeptide expression construct under the control of the 35S CaMV promoter and the nopaline synthase terminator and carrying a selectable marker (for example, kanamycin resistance) is transformed into Agrobacterium. Transformation of leaf discs, with vector-containing Agrobacterium is carried out as described by Horsch et al. (Science 227:1229 (1985)). Putative transformants are selected after a few weeks (for example, 3 to 5 weeks) on plant tissue culture media containing kanamycin (e.g., 100 μg/mL). Kanamycin-resistant shoots are then placed on plant tissue culture media without hormones for root initiation. Kanamycin-resistant plants are then selected for greenhouse growth. If desired, seeds from self-fertilized transgenic plants can then be sowed in a soil-less medium and grown in a greenhouse. Kanamycin-resistant progeny are selected by sowing surfaced sterilized seeds on hormone-free kanamycin-containing media. Analysis for the integration of the transgene is accomplished by standard techniques (see, for example, Ausubel et al., supra; Gelvin et al., supra).

Transgenic plants expressing the selectable marker are then screened for transmission of the transgene DNA by standard immunoblot and DNA detection techniques. Each positive transgenic plant and its transgenic progeny are unique in comparison to other transgenic plants established with the same transgene. Integration of the transgene DNA into the plant genomic DNA is in most cases random, and the site of integration can profoundly affect the levels and the tissue and developmental patterns of transgene expression. Consequently, a number of transgenic lines are usually screened for each transgene to identify and select plants with the most appropriate expression profiles.

Transgenic lines are evaluated for levels of transgene expression. Expression at the RNA level is determined initially to identify and quantitate expression-positive plants. Standard techniques for RNA analysis are employed for transgenic plants expressing REG inhibitory nucleic acids or polypeptides. Such techniques include PCR amplification assays using oligonucleotide primers designed to amplify only transgene RNA templates and solution hybridization assays using transgene-specific probes (see, e.g., Ausubel et al., supra). Those RNA-positive plants that encode an REG inhibitory nucleic acid or REG polypeptide are then analyzed for polypeptide expression by Western immunoblot analysis using specific antibodies (see, e.g., Ausubel et al., supra) to detect a decrease or increase in the level of expression of a gene of interest. In addition, immunocytochemistry according to standard protocols can be done using specific antibodies to detect a decrease in the level of expression of a target gene within transgenic tissue.

Ectopic expression of one or more REG inhibitory nucleic acids is useful for the production of transgenic plants that exhibit limited RNAi, while ectopic expression of REG polypeptides is useful for the production of transgenic plants that exhibit enhanced RNAi.

Use of Transgenic and Knockout Animals in Diagnosis or Drug Screening

The present invention also includes transgenic animals expressing REG inhibitory nucleic acids, or REG knock-out animals that fail to exhibit RNAi or that exhibit RNAi of decreased efficacy. Such animals are useful to determine genetic and physiological features of RNAi or to study the biological activity of polypeptides required for RNAi.

Transgenic animals include animals expressing a dsRNA that targets an endogenous REG nucleic acid sequence. Because such transgenic animals and REG knock-out animals likely express decreased levels of an REG, relative to a wild-type control animal, they are likely to exhibit no RNAi or limited RNAi, and are useful for the analysis of RNAi pathway components.

An REG knockout organism may be a conditional, i.e., somatic, knockout. For example, FRT sequences may be introduced into the organism so that they flank the gene of interest. Transient or continuous expression of the FLP protein may then be used to induce site-directed recombination, resulting in the excision of an REG gene. The use of the FLP/FRT system is well established in the art and is described in, for example, U.S. Pat. No. 5,527,695, and in Lyznik et al. (Nucleic Acid Res 24:3784 (1996)).

Conditional, i.e., somatic knockout organisms may also be produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a nucleic acid sequence described herein, flanked by loxP sites is excised, generating a knockout. This system is described, for example, in Kilby et al. (Trends Genet 9:413 (1993)).

Particularly desirable is a mouse model where a dsRNA targeting a gene of interest, such as an REG is expressed in specific cells of the transgenic mouse such that those cells fail to exhibit RNAi or exhibit limited RNAi. In addition, cell lines from these mice may be established by methods standard in the art.

The present invention also includes transgenic animals expressing an REG nucleic acid that encodes an REG polypeptide. Such transgenic animals exhibit RNAi of increased efficacy.

Construction of transgenes can be accomplished using any suitable genetic engineering technique, such as those described in Ausubel et al. (Current Protocols in Molecular Biology, John Wiley & Sons, New York (2000)). Many techniques of transgene construction and of expression constructs for transfection or transformation in general are known and may be used for the disclosed constructs.

One skilled in the art will appreciate that a promoter is chosen that directs expression of an REG nucleic acid in a desired tissue. For example, as noted above, any promoter that regulates expression of a nucleic acid sequence described herein can be used in the expression constructs of the present invention. One skilled in the art would be aware that the modular nature of transcriptional regulatory elements and the absence of position-dependence of the function of some regulatory elements, such as enhancers, make modifications such as, for example, rearrangements, deletions of some elements or extraneous sequences, and insertion of heterologous elements possible. Numerous techniques are available for dissecting the regulatory elements of genes to determine their location and function. Such information can be used to direct modification of the elements, if desired. It is desirable, however, that an intact region of the transcriptional regulatory elements of a gene is used. Once a suitable transgene construct has been made, any suitable technique for introducing this construct into embryonic cells can be used.

Animals suitable for transgenic experiments can be obtained from standard commercial sources such as Taconic (Germantown, N.Y.). Many strains are suitable, but Swiss Webster (Taconic) female mice are desirable for embryo retrieval and transfer. B6D2F (Taconic) males can be used for mating and vasectomized Swiss Webster studs can be used to stimulate pseudopregnancy. Vasectomized mice and rats are publicly available from the above-mentioned suppliers. However, one skilled in the art would also know how to make a transgenic mouse or rat. An example of a protocol that can be used to produce a transgenic animal is provided below.

Production of Transgenic Mice And Rats

The following is but one desirable means of producing transgenic mice. This general protocol may be modified by those skilled in the art.

Female mice six weeks of age are induced to superovulate with a 5 IU injection (0.1 cc, IP) of pregnant mare serum gonadotropin (PMSG; Sigma) followed 48 hours later by a 5 IU injection (0.1 cc, IP) of human chorionic gonadotropin (hCG, Sigma). Females are placed together with males immediately after hCG injection. Twenty-one hours after hCG injection, the mated females are sacrificed by CO₂ asphyxiation or cervical dislocation and embryos are recovered from excised oviducts and placed in Dulbecco's phosphate buffered saline with 0.5% bovine serum albumin (BSA, Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with humidified atmosphere at 5% CO₂, 95% air until the time of injection. Embryos can be implanted at the two-cell stage.

Randomly cycling adult female mice are paired with vasectomized males. Swiss Webster or other comparable strains can be used for this purpose. Recipient females are mated at the same time as donor females. At the time of embryo transfer, the recipient females are anesthetized with an intraperitoneal injection of 0.015 ml of 2.5% avertin per gram of body weight. The oviducts are exposed by a single midline dorsal incision. An incision is then made through the body wall directly over the oviduct. The ovarian bursa is then torn with watchmaker's forceps. Embryos to be transferred are placed in DPBS (Dulbecco's phosphate buffered saline) and in the tip of a transfer pipet (about 10 to 12 embryos). The pipet tip is inserted into the infundibulum and the embryos are transferred. After the transferring the embryos, the incision is closed by two sutures.

A desirable procedure for generating transgenic rats is similar to that described above for mice (Hammer et al., Cell 63:1099 (1990)). For example, thirty-day old female rats are given a subcutaneous injection of 20 IU of PMSG (0.1 cc) and 48 hours later each female placed with a proven, fertile male. At the same time, 40-80 day old females are placed in cages with vasectomized males. These will provide the foster mothers for embryo transfer. The next morning females are checked for vaginal plugs. Females who have mated with vasectomized males are held aside until the time of transfer. Donor females that have mated are sacrificed (CO₂ asphyxiation) and their oviducts removed, placed in DPBA (Dulbecco's phosphate buffered saline) with 0.5% BSA and the embryos collected. Cumulus cells surrounding the embryos are removed with hyaluronidase (1 mg/ml). The embryos are then washed and placed in EBSs (Earle's balanced salt solution) containing 0.5% BSA in a 37.5° C. incubator until the time of microinjection.

Once the embryos are injected, the live embryos are moved to DPBS for transfer into foster mothers. The foster mothers are anesthetized with ketamine (40 mg/kg, IP) and xulazine (5 mg/kg, IP). A dorsal midline incision is made through the skin and the ovary and oviduct are exposed by an incision through the muscle layer directly over the ovary. The ovarian bursa is torn, the embryos are picked up into the transfer pipet, and the tip of the transfer pipet is inserted into the infundibulum. Approximately 10 to 12 embryos are transferred into each rat oviduct through the infundibulum. The incision is then closed with sutures, and the foster mothers are housed singly.

Generation of Knockout Mice

The following is but one example for the generation of a knockout mouse and the protocol may be readily adapted or modified by those skilled in the art.

Embryonic stem cells (ES), for example, 10⁷ AB1 cells, may be electroporated with 25 μg targeting construct in 0.9 ml PBS using a Bio-Rad Gene Pulser (500 μF, 230 V). The cells may then be plated on one or two 10-cm plates containing a monolayer of irradiated STO feeder cells. Twenty-four hours later, they may be subjected to G418 selection (350 μg/ml, Gibco) for 9 days. Resistant clones may then be analyzed by Southern blotting after Hind III digestion, using a probe specific to the targeting construct. Positive clones are expanded and injected into C57BL/6 blastocysts. Male chimeras may be back-crossed to C57BL/6 females. Heterozygotes may be identified by Southern blotting and intercrossed to generate homozygotes.

The targeting construct may result in the disruption of the gene of interest, e.g., by insertion of a heterologous sequence containing stop codons, or the construct may be used to replace the wild-type gene with a mutant form of the same gene, e.g., a “knock-in.” Furthermore, the targeting construct may contain a sequence that allows for conditional expression of the gene of interest. For example, a sequence may be inserted into the gene of interest that results in the polypeptide not being expressed in the presence of tetracycline. Such conditional expression of a gene is described in, for example, Yamamoto et al. (Cell 101:57 (2000)).

Microarrays

The global analysis of gene expression using gene chips can provide insights into the expression of genes regulated by REGs. For example, nucleic acids that function in RNAi are also important in stem cell development, maintenance, or differentiation. This is not suprising given that many REGs function in both RNAi and in chromatin modulation and gene silencing. Microarrays are used to identify sets of genes that are differentially regulated in stem cells versus differentiated cells. In one example, microarrays are used to compare the expression of a genes in a cell or organism that lacks one or more REGs relative to a wild-type cell or organism. This comparison identifies sets of genes that are differentially regulated in response to REG expression. In another example, microarrays are used to compare the expression of REGs in a stem cell, or other pluripotent cell type, with the expression of REGs in a differentiated cell derived from that stem cell. This comparison identifies differentially regulated REGs that may contribute to the pluripotency displayed by stem cells. Genes that are upregulated in stem cells may be required for stem cell generation or maintenance. Such genes may be useful in generating genetically engineered stem cells that over express factors that are required for pluripotency.

Genes that are upregulated in differentiated cells may be required for cell fate determination or for the maintenance of a differentiated phenotype. Such genes might also be upregulated in cancer cells, which typically display an undifferentiated phenotype. Such genes or their gene products present attractive therapeutic candidates for cancer treatment.

Microarrays containing nucleic acids that are derived from a cell or organism that lacks at least one REG may be prepared, used, and analyzed using methods known in the art. In another embodiment, a microarray may contain sets of REG nucleic acids. (See, e.g., Brennan et al., U.S. Pat. No. 5,474,796; Schena et al., Proc Natl Acad Sci USA 93:10614 (1996); Baldeschweiler et al., PCT application WO95/251116 (1995); Shalon et al., PCT application WO95/35505; Heller et al., Proc Natl Acad Sci USA 94:2150 (1997); and Heller et al., U.S. Pat. No. 5,605,662).

In general, hybridizable array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or polypeptides. Methods for making nucleic acid microarrays are known to the skilled artisan and are described, for example, in U.S. Pat. No. 5,837,832, Lockhart, et al. (Nat Biotechnol 14:1675 (1996)), and Schena, et al. (Proc Natl Acad Sci USA 93:10614 (1996)), herein incorporated by reference. Methods for making polypeptide microarrays are described, for example, by Ge (Nucleic Acids Res 28:e3.1 (2000)), MacBeath et al. (Science 289:1760 (2000)), Zhu et al. (Nat Genet 26:283 (2000)), and in U.S. Pat. No. 6,436,665, hereby incorporated by reference.

Nucleic Acid Microarrays

To produce a nucleic acid microarray oligonucleotides may be synthesized or bound to the surface of a substrate using a chemical coupling procedure and an ink jet application apparatus, as described in Baldeschweiler et al., PCT application WO95/251116, incorporated herein by reference. In another embodiment, a gridded array may be used to arrange and link cDNA fragments or oligonucleotides to the surface of a substrate using a vacuum system, thermal, UV, mechanical or chemical bonding procedure.

A nucleic acid molecule (e.g., RNA or DNA) derived from a biological sample, such as a cultured cell, a tissue specimen, or other source, may be used to produce a hybridization probe as described herein. The mRNA is isolated according to standard methods, and cDNA is produced and used as a template to make complementary RNA suitable for hybridization using standard methods. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

A detection system may be used to measure the absence, presence, and amount of hybridization for all of the distinct sequences simultaneously (e.g., Heller et al., Proc Natl Acad Sci USA 94:2150 (1997)). Preferably, a scanner is used to determine the levels and patterns of fluorescence.

Protein Microarrays

REG polypeptides, such as those described herein, may also be analyzed using protein microarrays. Such arrays are useful in high-throughput low-cost screens to identify peptide or candidate compounds that bind an REG polypeptide of the invention, or fragment thereof. Typically, protein microarrays feature a polypeptide, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., polypeptides of interest or antibodies against such polypeptides) are spotted on a substrate using any convenient method known to the skilled artisan (e.g., by hand or by inkjet printer). Preferably, such methods retain the biological activity or function of the polypeptide bound to the substrate (Ge et al., supra; Zhu et al., supra).

The protein microarray is hybridized with a detectable probe. Such probes can be polypeptide, nucleic acid, or small molecules. For some applications, polypeptide and nucleic acid probes are derived from a biological sample taken from a patient, such as a bodily fluid (such as blood, urine, saliva, or phlegm); a homogenized tissue sample (e.g., a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, polypeptide concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked colorimetric assay), direct immunoassay, radiometric assay, or any other suitable detectable method known to the skilled artisan.

### Other Embodiments

From the foregoing description, it will be apparent that variations and modifications may be made to the invention described herein to adapt it to various usages and conditions. Such embodiments are also within the scope of the following claims.

All publications mentioned in this specification are herein incorporated by reference to the same extent as if each independent publication was specifically and individually indicated to be incorporated by reference.

1. A method for identifying a gene encoding a polypeptide that is required for RNA interference (RNAi), said method comprising:
(a) providing a nematode comprising a dsRNA;
(b) contacting said nematode with an inhibitory nucleobase oligomer that targets a candidate gene; and
(c) detecting a decrease in RNAi mediated by said dsRNA in said nematode relative to a control nematode not contacted with said inhibitory nucleobase oligomer, wherein said decrease indicates that said candidate gene encodes a polypeptide that is required for RNAi.

2. A method for identifying a gene encoding a polypeptide that enhances RNAi, said method comprising:
(a) providing a nematode comprising a dsRNA;
(b) contacting said nematode with an inhibitory nucleobase oligomer that targets a candidate gene; and
(c) detecting an increase in RNAi mediated by said dsRNA in said nematode relative to a control nematode not contacted with said inhibitory nucleobase oligomer, wherein said increase indicates that said candidate gene encodes a polypeptide that enhances RNAi.

3. The method of claim 1 or 2, wherein said decrease or said increase is detected by monitoring the expression of a reporter gene whose expression is modulated by said dsRNA.

4. The method of claim 1 or 2, wherein said inhibitory nucleobase oligomer is a dsRNA, siRNA, or dsRNA mimetic.

5. The method of claim 1 or 2, wherein said nematode comprises a mutation that enhances RNAi.

6. The method of claim 1 or 2, wherein said nematode comprises a mutation selected from the group consisting of eri-1 and rrf-3.

7. A method for identifying a candidate compound that modulates RNAi, said method comprising:
(a) providing a cell expressing an REG nucleic acid molecule;
(b) contacting said cell with a candidate compound; and
(c) comparing the expression or biological activity of said REG nucleic acid molecule in said cell contacted with said candidate compound with the expression or biological activity of said REG nucleic acid molecule in a control cell, wherein an alteration in said expression or biological activity identifies said candidate compound as a candidate compound that modulates RNAi.

8. The method of claim 7, wherein said REG nucleic acid molecule is selected from the group consisting of C04F12.1, K12B6.1, Y38A10A.6, F43G9.5, K08D10.4, ZK1127.6, ZK1127.9, W05H7.4, T19B10.4, T23B12.1, M03C11.3, Y71G10AL.1, ZK1127.3, F02E9.4, R06C1.1, ZK112.2, T19B4.5, T22B3.1, T01C3.8, B0414.7, ZC449.3, Y56A3A.17, F37B12.4, F52G2.2, C31H1.8, C55B7.5, E02H1.1, F09G2.4, F26A3.2, F43G9.1, F43G9.12, F46A9.5, F49D11.1, R06F6.1, T25G3.3, W06E11.1, W07E6.4, Y71F9B.4, ZK1127.5, ZK1127.7, C06E1.10, C16A3.4, D2089.1, F11A10.3, F56A8.6, F56D2.6, T08G11.4, T23D8.3, K07A1.11, T12D8.1, T22D1.10, C27H6.2, C07E3.2, C26D10.1, D2096.8, F59A2.1, F32E10.4, K07F5.13a, R06A4.4a, W04C9.1, Y38F2AL.3, Y48G1A.5, ZK1127.4, C15F1.4, F52B5.6, F59A3.3, Y61A9LA.10, F48E8.5, T01G9.6a, C06A5.1, C29E4.2, K12H4.5, W04A4.5, Y110A7A.19, F26E4.4, F43G9.10, F54H12.1, F56A3.4, T09A5.10, W10C8.2, B0035.13, B0035.1a, B0207.6, B0272.5a, B0273.1, B0273.4a, B0334.9, B0336.3, B0336.7, B0361.7, B0379.3a, B0399.2, B0412.1, B0432.10, B0491.7, B0513.3, C01B12.7, C01G10.11a, C01G10.6, C01G10.8, C01G6.5, C02F5.3, C02F5.4, C03C10.4, C04A11.3, C04D8.1, C04E7.2, C05D12.2, C05G5.1, C06G1.4, C06G1.4, C06G3.5, C07E3.1a, C08B11.6, C08B11.9, C08F8.8, C09G12.8, C10G6.1, C11D2.2, C12D8.1a, C14A4.11, C14A6.6, C14B1.3, C14B1.4, C14B1.5, C14B1.6, C15C6.3, C15C8.5, C15F1.2, C15F1.3a, C17E4.6, C17H11.2, C17H11.5, C18C4.10, C18E9.10, C24H11.6, C24H12.5a, C24H12.7, C25A1.3, C25A11.4a, C25E10.6, C26B9.1, C26E6.9a, C27A12.2, C27C7.4, C28H8.7, C29E4.4, C29E4.7, C30A5.5, C30G4.5, C31H1.8, C32D5.11, C32D5.7, C32H11.12, C33G3.1, C33H5.12a, C33H5.7, C34E10.8, C35D10.7a, C41H7.3, C41H7.4, C41H7.6, C42C1.15, C43F9.4, C44B9.1, C44C10.1, C44F1.4, C46A5.9, C47E12.8, C49A9.3, C49D10.8, C50C3.8, C50E3.3, C52A11.4, C53A5.2, C53C11.3, C54D10.9, C55C3.3, C55C3.4, C56C10.13, C56G2.1, CC4.3, CD4.7, D1007.16, D1046.1, D2096.6, DY3.5, E03A3.6, F01E11.1, F01F1.12, F01F1.5, F02E9.4, F07C4.7, F09E5.13, F10C2.3, F10E9.6a, F10F2.1, F10G7.7, F10G8.6, F11A1.3a, F11A10.1, F13E6.4, F15B10.2, F15E6.1, F16D3.2, F18H3.5a, F19H8.3, F20B6.8, F21C3.4, F21H12.1, F23H11.2, F25H2.1, F26A.1, F26B1.2, F26E4.1, F28A10.6, F28C1.1, F28C6.10, F28F8.3, F29C4.7, F30A10.10, F31C3.6, F32B6.3, F32D8.7, F34D10.4, F35C5.2, F35G12.4a, F36A2.1, F37A4.2, F37D6.2, F38A1.7, F38A5.13, F38E 1.4, F38H4.7, F39B2.1, F39B2.7, F39H1.1, F40E10.4, F40F8.9, F40H3.1, F41H10.4, F42F12.4, F42G4.3a, F43G9.5, F44F4.13, F45E10.1, F45E4.10a, F46F11.4, F47B8.4, F47F6.1, F47F6.2, F47H4.6, F48E8.2, F49D11.4, F52B1.1, F52G2.2, F53A2.8, F53C3.1, F53E10.6, F53F4.12, F53F4.5, F53H1.1a, F54C4.2, F54E7.7, F54F2.2a, F54F2.2a, F54F2.9, F56B3.12, F56D5.10, F56E10.1, F56G4.5, F57C2.2, F57G8.6, F58B6.2, F58F9.6, F58G11.2, F58G11.4, F59A2.2, F59A6.6, F59A7.4, F59E12.4a, H05C05.3, H08M01.1, H12D21.7, H12113.3, H12I19.2, H13N06.5, H14E04.3, H19N07.4, H20J04.3, H24K24.3, H28G03.2, K01C8.1, K01G5.2a, K01G5.8a, K02F3.8, K03A11.2, K03H1.2, K03H1.8, K04B12.3, K05C4.5, K06A1.5, K06B4.2, K06H7.4, K07C5.8, K08D10.3, K08D10.4, K08E3.4, K08E3.8, K08H10.7, K08H2.6, K09A9.3, K10B3.7, K10B3.8, K10C3.5, K10C8.2, K10D2.6, K10D3.2, K10G6.1, K10H10.1, K11D12.9, K11H12.8, K12B6.1, K12H4.8, M01D1.8, M01G5.1, M03C11.3, M04B2.3, M163.3, R01E6.3a, R02D3.7, R03D7.4, R03G8.4, R04B5.4, R04D3.7, R04E5.7, R05C11.3, R05D11.7, R05F9.1b, R06C1.4, R06C7.7, R07B5.8, R07B7.7, R07E5.4, R08C7.3, R09E12.2, R12C12.2, R13.1, R144.7, T01C3.8, T01C8.1a, T01E8.5, T02H6.8, T04F3.1, T05C12.6, T07C4.1, T07D3.7, T10F2.3, T13F2.1, T14G12.2, T16H12.5, T18H9.6, T19B4.5, T20D3.3, T20G5.11, T21B10.3, T22B3.1, T22B7.1, T22B7.2, T24A11.1a, T24H10.1, T25B6.7, T26A5.5a, T26A5.7, T26F2.2, T27A3.1, T28B11.1, T28D9.2a, W01G7.1, W01G7.4, W01H2.1, W02B3.7, W03A5.7, W03F11.6a, W03H9.3, W04D2.6a, W04E12.6, W05H5.4, W06B4.3, W06D11.4, W06H8.6, W07G.1, W08A12.1, W08E12.7, W09C2.3, W09D6.1, W09G3.2, W10G1.6, Y104H12D.2, Y1O₅C5B.22, Y105E8B.2, Y105E8B.5, Y105E8B.9, Y111B2A.17, Y113G7A.8, Y116A8C.33, Y116A8C.34, Y116A8C.36, Y116A8C.6, Y119D3A.4, Y119D3B.12, Y14H12B.1, Y17G7B.18a, Y17G7B.19, Y17G7B.2, Y18D10A.8, Y18D10A.9, Y18H1A.6, Y24D9A.1, Y2H9A.1, Y37A1B.1, Y37D8A.9, Y37E11AL.3, Y37E11AR.2, Y38A10A.6, Y38C9A.2, Y38E10A.13, Y38E10A.6, Y38F1A.4, Y39A3CL.5, Y39B6A.1, Y39B6A.17, Y39B6A.36, Y39B6A.43, Y39F10B.1, Y39G10AR.10, Y40B10A.9, Y41D4A.4, Y41E3.2, Y42H9AR.1, Y43F4B.3, Y44F5A.1, Y45F10C.2, Y45F10D.4, Y46C8AR.1, Y46G5A.5, Y46H3C.6, Y47D3A.21, Y47D3A.27, Y47D3B.9, Y47G6A.11, Y47G6A.20b, Y47G6A.4, Y48B6A.11, Y48E1B.3, Y48E1B.4, Y48E1B.6, Y49F6B.1, Y49F6B.4, Y50D4C.2, Y50D7A.9, Y50E8A.8, Y50F7A.2, Y51B9A.4, Y51H1A.6, Y51H1A.7, Y51H4A.12, Y51H4A.16, Y53C12A.4, Y53F4B.25, Y53G8AL.3, Y54E10A.16, Y54E2A.2, Y54G11A.2, Y54G9A.6, Y55B1BR.3, Y55F3AL.1, Y55F3AM.1, Y55F3AM.12, Y55F3AM.12, Y55F3AM.3, Y55F3AM.3a, Y56A3A.12a, Y56A3A.17a, Y56A3A.17a, Y57A10A.26, Y59A8B.10, Y62E10A.12, Y62F5A.1, Y65B4A.1, Y65B4A.1, Y67D8A.1, Y67D8C.10a, Y69A2AR.14, Y69A2AR.15, Y69E1A.8, Y70G10A.2, Y71F9B.16, Y71F9B.2, Y71G10AL.1, Y71G12B.14, Y71G12B.9, Y71H2AM.14, Y71H2B.11, Y71H2B.2, Y73F8A.25, Y75B8A.5, Y76A2B.5, Y79H2A.3, Y80D3A.5, Y81G3A.3, ZC155.3, ZC155.7, ZC204.13, ZC239.4, ZC250.3, ZC317.4, ZC376.8, ZC395.10, ZC47.4, ZC518.3a, ZC84.6, ZK1127.9a, ZK20.4, ZK370.3, ZK370.7, ZK520.2, ZK643.5, ZK686.4, ZK688.2, ZK863.6, ZK970.t4, ENSG0000085511, ENSG00000092847, ENSG00000164860, ENSG00000150990, ENSG00000188976, ENSG00000017164, ENSG00000070785, ENSG00000173545, ENSG00000180198, ENSG00000175792, ENSG00000025770, ENSG00000025770, ENSG00000105176, ENSG00000153914, ENSG00000187109, ENSG00000086189, ENSG00000169375, ENSG00000165934, ENSG00000185619, ENSG00000114503, ENSG00000186432, ENSG00000162402, ENSG00000166411, ENSG00000140259, ENSG00000159086, ENSG00000167005, ENSG00000113558, ENSG00000105568, ENSG00000168438, ENSG00000198242, ENSG00000001412, ENSG00000138778, ENSG00000011811, ENSG00000019606, ENSG00000153201, ENSG00000143314, ENSG00000162521, ENSG00000138750, ENSG00000125870, ENSG00000134698, ENSG000000111007, ENSG00000083312, ENSG00000116478, ENSG00000163950, ENSG00000111968, ENSG00000137574, ENSG00000006788, ENSG00000055609, ENSG00000183207, ENSG00000138785, ENSG00000135521, ENSG00000169251, ENSG00000149262, ENSG00000150967, ENSG00000058600, ENSG00000099995, ENSG00000081059, ENSG00000132300, ENSG00000067048, ENSG00000155097, ENSG00000124207, ENSG00000093000, ENSG00000165733, ENSG00000130332, ENSG00000065559, ENSG00000109654, ENSG00000107949, ENSG00000120158, ENSG00000113649, ENSG00000131747, ENSG00000113649, ENSG00000142751, ENSG00000169375, ENSG00000090686, ENSG00000011654, ENSG00000012241, ENSG00000012241, ENSG00000126698, ENSG00000164167, ENSG00000031823, ENSG00000117222, ENSG00000075089, ENSG00000166833, ENSG00000013502, ENSG00000108372, ENSG00000137460, ENSG00000196363, ENSG00000079387, ENSG00000114487, ENSG00000124222, ENSG00000139746, ENSG00000039987, ENSG00000100697, ENSG00000121067, ENSG00000067842, ENSG00000125870, ENSG00000125870, ENSG00000005483, ENSG00000164032, ENSG00000178997, ENSG00000015821, ENSG00000111727, ENSG00000122515, ENSG00000127337, ENSG00000170860, ENSG00000060339, ENSG00000154832, ENSG00000151065, ENSG00000131368, ENSG00000123908, ENSG00000152670, ENSG00000188342, ENSG00000064393, ENSG00000189060, ENSG00000112759, ENSG00000118985, ENSG00000126214, ENSG00000139726, ENSG00000131051, ENSG00000164902, ENSG00000145088, ENSG00000147419, ENSG00000129691, ENSG00000172915, ENSG00000158435, ENSG00000196792, ENSG00000182874, ENSG00000001395, ENSG00000124380, ENSG00000167005, ENSG00000153774, ENSG00000104762, ENSG00000144021, ENSG00000122692, ENSG00000049246, ENSG00000113141, ENSG00000146216, ENSG00000116138, ENSG00000119720, ENSG000000117543, ENSG00000154473, ENSG00000138709, ENSG00000127948, ENSG00000074696, ENSG00000105993, ENSG00000129518, ENSG00000159200, ENSG00000149925, ENSG00000069248, ENSG00000148834, ENSG00000121067, ENSG00000114491, ENSG00000148950, ENSG00000185787, ENSG00000179837, ENSG00000169898, ENSG00000111605, ENSG00000067842, ENSG00000112333, ENSG00000156802, ENSG00000147475, ENSG00000005483, ENSG00000067533, ENSG00000067048, ENSG00000083168, ENSG00000110455, ENSG00000104859, ENSG00000147548, ENSG00000100591, ENSG00000106355, ENSG00000112033, ENSG00000111640, ENSG00000122565, ENSG00000169217, ENSG00000167658, ENSG00000170364, ENSG00000084072, ENSG00000134759, ENSG00000177463, ENSG00000158950, ENSG00000115806, ENSG00000173153, ENSG00000198258, ENSG00000196597, ENSG00000182606, ENSG00000196474, ENSG00000163125, ENSG00000163159, ENSG00000103274, ENSG00000078902, ENSG00000177613, ENSG00000090316, ENSG00000116903, ENSG00000165704, ENSG00000169919, ENSG00000141076, ENSG00000172273, ENSG00000130299, ENSG00000140451, ENSG00000138778, ENSG00000116062, ENSG00000144559, ENSG00000151498, ENSG00000123908, ENSG00000110906, ENSG00000134480, ENSG00000171956, ENSG00000104142, ENSG00000171865, ENSG00000085978, ENSG00000164008, ENSG00000167720, ENSG00000197214, ENSG00000113369, ENSG00000135932, ENSG00000114209, ENSG00000132849, ENSG00000011007, ENSG00000175324, ENSG00000162377, ENSG00000146267, ENSG00000128829, ENSG00000167447, ENSG00000101194, ENSG00000123444, ENSG00000138175, ENSG00000015771, ENSG00000165659, ENSG00000001345, ENSG00000116663, ENSG00000108963, ENSG00000133958, ENSG00000175137, ENSG00000120314, ENSG00000105648, ENSG00000164062, ENSG00000121057, ENSG00000183955, ENSG00000014257, ENSG00000146007, ENSG00000008256, ENSG00000179036, ENSG00000018591, ENSG00000141425, ENSG00000167433, ENSG00000127946, ENSG00000055291, ENSG00000015821, ENSG00000003436, ENSG00000140829, ENSG00000095459, ENSG00000004766, ENSG00000157426, ENSG00000148948, ENSG00000139505, ENSG00000168288, ENSG00000136279, ENSG00000108848, ENSG00000014216, ENSG00000136463, ENSG00000165678, ENSG00000068400, ENSG00000151422, ENSG00000196188, ENSG00000143341, ENSG00000017201, ENSG00000196839, ENSG00000185141, ENSG00000134824, ENSG00000165630, ENSG00000147647, ENSG00000133243, ENSG00000162244, ENSG00000198399, ENSG00000196470, ENSG00000077147, ENSG00000076356, ENSG00000151065, ENSG00000102893, ENSG00000182162, ENSG00000001226, ENSG00000197894, ENSG00000106400, ENSG00000117620, ENSG00000134698, ENSG00000187697, ENSG00000162961, ENSG00000152207, ENSG00000131781, ENSG00000164073, ENSG00000126814, ENSG00000145476, ENSG00000054219, ENSG00000197894, ENSG00000111640, ENSG000000110693, ENSG0000011083, ENSG00000198373, ENSG00000104885, ENSG00000182551, ENSG00000113441, ENSG00000072786, ENSG00000169026, ENSG00000165915, ENSG00000078687, ENSG00000078687, ENSG00000182077, ENSG00000169750, ENSG00000104967, ENSG00000198477, ENSG00000066135, ENSG00000159479, ENSG00000164219, ENSG00000151092, ENSG00000136003, ENSG00000170515, ENSG00000188566, ENSG00000182655, ENSG00000189060, ENSG00000164749, ENSG00000132639, ENSG00000174607, ENSG00000052758, ENSG00000023445, and ENSG00000161202, or an ortholog thereof.

9. The method of claim 7, wherein said REG nucleic acid encodes a polypeptide selected from the group consisting of B0414.7b, B0414.7a, C04F12.1, C06A5.1, C06E1.10, C07E3.2, C12D8.1b.3, C12D8.1b.1, C12D8.1a, C12D8.1b.2, C15F1.4, C16A3.4, C26D10.1, C27H6.2, C29E4.2, C31H1.8, C55B7.5, D2089.1b.1, D2089.1b.2, D2089.1a, D2089.1b.3, D2096.8, E02H1.1, F02E9.4.1, F02E9.4.2, F09G2.4, F11A10.3, F22D6.6, F26A3.2, F26E4.4, F32E10.4, F37B12.4, F43G9.1, F43G9.10, F43G9.12, F43G9.5, F46A9.5.1, F46A9.5.2, F48E8.5.2, F48E8.5.3, F48E8.5.1, F49D11.1, F52B5.6, F52G2.2, F54H12.1a, F54H12.1c, F54H12.1b, F56A3.4, F56A8.6, F56D2.6a, F56D2.6b, F59A2.1b.2, F59A2.1b.1, F59A2.1a, F59A3.3, K07A1.11, K07F5.13c, K07F5.13b, K07F5.13a, K08D10.4, K12B6.1, K12H4.5.3, K12H4.5.1, K12H4.5.4, K12H4.5.5, K12H4.5.6, K12H4.5.2, M03C11.3, R03D7.4, R06A4.4a, R06A4.4b, R06C1.1, R06F6.1, T01C3.8, T01G9.6a.2, T01G9.6b, T01G9.6a.1, T08G11.4, T09A5.10, T12D8.1.1, T19B10.4b, T19B10.4a, T19B4.5, T22B3.1, T22D1.10, T23B12.1, T23D8.3, T25G3.3, W04A4.5, W04C9.1, W05H7.4c, W05H7.4d, W05H7.4a, W05H7.4b, W06E11.1, W07E6.4, W10C8.2, Y110A7A.19, Y38A10A.6, Y38F2AL.3b, Y38F2AL.3a, Y48G1A.5, Y56A3A.17b, Y56A3A.17a, Y61A9LA.10, Y71F9B.4, Y71G10AL.1a, Y71G10AL.1b, ZC449.3b, ZC449.3a.2, ZC449.3a.1, ZK112.2, ZK1127.3, ZK1127.4, ZK1127.5, ZK1127.6.1, ZK1127.6.2, ZK1127.7, ZK1127.9e.2, ZK1127.9d, ZK1127.9c, ZK1127.9e.3, ZK1127.9b, ZK1127.9a, ZK1127.9e.1, ENSP00000257131, ENSP00000207451, ENSP00000277804, ENSP00000300291, ENSP00000243563, ENSP00000296702, ENSP00000251819, ENSP00000331699, ENSP00000265155, ENSP00000289371, TR:Q7Z4R6, ENSP00000248054, ENSP00000271095, ENSP00000227588, ENSP00000297332, ENSP00000262445, ENSP00000330758, ENSP00000294383, ENSP00000252172, ENSP00000234697, ENSP00000347325, ENSP00000353218, ENSP00000262189, ENSP00000299853, ENSP00000352140, ENSP00000354285, ENSP00000353284, ENSP00000336725, ENSP00000234553, ENSP00000353575, ENSP00000335644, ENSP00000340347, ENSP00000354863, ENSP00000326540, ENSP00000326654, ENSP00000261412, ENSP00000336712, ENSP00000297332, ENSP00000265125, ENSP00000199320, ENSP00000345895, ENSP00000347396, ENSP00000215793, ENSP00000216254, ENSP00000263028, ENSP00000353817, ENSP00000312530, ENSP00000355399, ENSP00000324804, ENSP00000354405, ENSP00000318177, ENSP00000351562, ENSP00000263214, ENSP00000278100, ENSP00000299130, ENSP00000336741, ENSP00000295561, ENSP00000348370, ENSP00000339659, ENSP00000229695, ENSP00000231487, ENSP00000331708, ENSP00000326806, ENSP00000239262, ENSP00000262982, ENSP00000343253, ENSP00000246071, ENSP00000252622, ENSP00000269577, ENSP00000350217, ENSP00000342944, ENSP00000254630, ENSP00000354379, ENSP00000304233, ENSP00000260129, ENSP00000264883, ENSP00000344052, ENSP00000265148, ENSP00000340737, ENSP00000267812, ENSP00000354525, ENSP00000271551, ENSP00000346913, ENSP00000278560, ENSP00000280560, ENSP00000351234, ENSP00000280559, ENSP00000313128, ENSP00000344339, ENSP00000311135, ENSP00000283195, ENSP00000334538, ENSP00000344032, ENSP00000284041, ENSP00000285415, ENSP00000328992, ENSP00000290178, ENSP00000340575, ENSP00000294520, ENSP00000316490, ENSP00000297487, ENSP00000298600, ENSP00000298875, ENSP00000299518, ENSP00000300291, ENSP00000304370, ENSP00000307525, ENSP00000353622, ENSP00000338617, ENSP00000310042, ENSP00000318297, ENSP00000345919, ENSP00000221413, ENSP00000334373, ENSP00000261182, ENSP00000313778, ENSP00000341800, ENSP00000347969, ENSP00000233156, ENSP00000342306, ENSP00000251739, ENSP00000307666, ENSP00000325582, ENSP00000350341, ENSP00000249297, ENSP00000257745, ENSP00000312379, ENSP00000327505, ENSP00000333986, ENSP00000335398, ENSP00000335599, ENSP00000337136, ENSP00000340699, ENSP00000297044, ENSP00000234697, ENSP00000230671, ENSP00000279247, ENSP00000323036, ENSP00000337471, ENSP00000263464, ENSP00000336833, ENSP00000034275, ENSP00000315894, ENSP00000341483, ENSP00000042931, ENSP00000168666, ENSP00000355031, ENSP00000266058, ENSP00000315005, ENSP00000263636, ENSP00000229452, ENSP00000265872, ENSP00000354989, ENSP00000263551, ENSP00000343108, ENSP00000262914, ENSP00000336725, ENSP00000353284, ENSP00000184772, ENSP00000352062, ENSP00000263519, ENSP00000343886, ENSP00000328157, ENSP00000015926, ENSP00000349958, ENSP00000261396, ENSP00000176763, ENSP00000261875, ENSP00000188312, ENSP00000264296, ENSP00000316347, ENSP00000301624, ENSP00000336783, ENSP00000316066, ENSP00000263646, ENSP00000314733, ENSP00000004980, ENSP00000344652, ENSP00000265713, ENSP00000348904, ENSP00000312769, ENSP00000287735, ENSP00000318259, ENSP00000334016, ENSP00000312809, ENSP00000264750, ENSP00000302830, ENSP00000264206, ENSP00000309262, ENSP00000304118, ENSP00000216044, ENSP00000334787, ENSP00000216181, ENSP00000338576, ENSP00000351200, ENSP00000216237, ENSP00000352891, ENSP00000216479, ENSP00000343745, ENSP00000217166, ENSP00000338974, ENSP00000262173, ENSP00000218364, ENSP00000299167, ENSP00000313504, ENSP00000283027, ENSP00000219789, ENSP00000220509, ENSP00000325074, ENSP00000221455, ENSP00000221482, ENSP00000346170, ENSP00000263257, ENSP00000262807, ENSP00000270066, ENSP00000222379, ENSP00000222539, ENSP00000249270, ENSP00000262177, ENSP00000223084, ENSP00000304593, ENSP00000350985, ENSP00000224050, ENSP00000225504, ENSP00000225729, ENSP00000240304, ENSP00000311535, ENSP00000263083, ENSP00000263084, ENSP00000263776, ENSP00000324948, ENSP00000336946, ENSP00000339876, ENSP00000344078, ENSP00000350470, ENSP00000228495, ENSP00000229204, ENSP00000266679, ENSP00000229239, ENSP00000229330, ENSP00000310928, ENSP00000337063, ENSP00000353916, ENSP00000230083, ENSP00000319152, ENSP00000311603, ENSP00000261812, ENSP00000265138, ENSP00000231368, ENSP00000308738, ENSP00000338141, ENSP00000264678, ENSP00000232603, ENSP00000232607, ENSP00000341587, ENSP00000234160, ENSP00000234420, ENSP00000251293, ENSP00000251544, ENSP00000353564, ENSP00000264515, ENSP00000251160, ENSP00000251159, ENSP00000339630, ENSP00000326111, ENSP00000237853, ENSP00000342723, ENSP00000346335, ENSP00000350579, ENSP00000311144, ENSP00000351100, ENSP00000337736, ENSP00000314075, ENSP00000240327, ENSP00000265346, ENSP00000311778, ENSP00000336687, ENSP00000351265, ENSP00000263222, ENSP00000263771, ENSP00000350422, ENSP00000352971, ENSP00000220592, ENSP00000348229, ENSP00000338281, ENSP00000352634, ENSP00000354445, ENSP00000350723, ENSP00000344234, ENSP00000355408, ENSP00000312086, ENSP00000244227, ENSP00000246071, ENSP00000246489, ENSP00000333912, ENSP00000334523, ENSP00000334618, ENSP00000341154, ENSP00000263697, ENSP00000261249, ENSP00000247843, ENSP00000336747, ENSP00000265302, ENSP00000263791, ENSP00000250454, ENSP00000323968, ENSP00000250635, ENSP00000340896, ENSP00000313818, ENSP00000351644, ENSP00000253363, ENSP00000344581, ENSP00000344700, ENSP00000351749, ENSP00000354437, ENSP00000253686, ENSP00000254090, ENSP00000254976, ENSP00000307341, ENSP00000255202, ENSP00000307496, ENSP00000326199, ENSP00000355371, ENSP00000255608, ENSP00000256339, ENSP00000339276, ENSP00000256897, ENSP000000257131, ENSP00000257191, ENSP00000300265, ENSP00000316051, ENSP00000350967, ENSP00000257261, ENSP00000278840, ENSP00000258418, ENSP00000310623, ENSP00000344584, ENSP00000258780, ENSP00000258975, ENSP00000260008, ENSP00000260746, ENSP00000264584, ENSP00000321997, ENSP00000338366, ENSP00000346444, ENSP00000352190, ENSP00000265148, ENSP00000266939, ENSP000000317987, ENSP00000280557, ENSP00000267229, ENSP00000327080, ENSP00000268043, ENSP00000268482, ENSP00000327179, ENSP00000339164, ENSP00000346989, ENSP00000269188, ENSP00000314602, ENSP00000337476, ENSP00000349955, ENSP00000341101, ENSP00000271588, ENSP00000271590, ENSP00000272402, ENSP00000273037, ENSP00000273668, ENSP00000274118, ENSP00000274712, ENSP00000259750, ENSP00000307357, ENSP00000275057, ENSP00000276395, ENSP00000348933, ENSP00000276461, ENSP00000335220, ENSP00000276546, ENSP00000313410, ENSP00000313983, ENSP00000346111, ENSP00000276651, ENSP00000278062, ENSP00000278198, ENSP00000278200, ENSP00000320187, ENSP00000336927, ENSP00000280665, ENSP00000280699, ENSP00000280700, ENSP00000307980, ENSP00000281092, ENSP00000281182, ENSP00000282018, ENSP00000334167, ENSP00000347087, ENSP00000350168, ENSP00000283882, ENSP00000284670, ENSP00000285106, ENSP00000287394, ENSP00000205214, ENSP00000289382, ENSP00000320768, ENSP00000290663, ENSP00000354512, ENSP00000354630, ENSP00000316054, ENSP00000334648, ENSP00000294189, ENSP00000294352, ENSP00000295066, ENSP00000345837, ENSP00000352368, ENSP00000335541, ENSP00000295315, ENSP00000346464, ENSP00000296389, ENSP00000296417, ENSP00000296456, ENSP00000296468, ENSP00000296581, ENSP00000296642, ENSP00000297330, ENSP00000346339, ENSP00000297540, ENSP00000298451, ENSP00000298452, ENSP00000304994, ENSP00000318506, ENSP00000346604, ENSP00000352712, ENSP00000342214, ENSP00000316023, ENSP00000298556, ENSP00000355367, ENSP00000298851, ENSP00000346956, ENSP00000354689, ENSP00000309577, ENSP00000312169, ENSP00000351514, ENSP00000353871, ENSP00000300291, ENSP00000300901, ENSP00000341880, ENSP00000352421, ENSP00000300917, ENSP00000307940, ENSP00000339435, ENSP00000301920, ENSP00000307545, ENSP00000320234, ENSP00000304903, ENSP00000338617, ENSP00000353622, ENSP00000304283, ENSP00000303117, ENSP00000302728, ENSP00000340734, ENSP00000305060, ENSP00000302886, ENSP00000302160, ENSP00000313350, ENSP00000306807, ENSP00000318085, ENSP00000308534, ENSP00000343005, ENSP000000000442, ENSP00000311648, ENSP00000342673, ENSP00000310596, ENSP00000320447, ENSP00000325616, ENSP00000332444, ENSP00000342323, ENSP00000348689, ENSP00000321029, ENSP00000314214, ENSP00000354018, ENSP00000313890, ENSP00000327376, ENSP00000327957, ENSP00000327957, ENSP00000333666, ENSP00000328998, ENSP00000340702, ENSP00000330442, ENSP00000333256, ENSP00000332995, ENSP00000328139, ENSP00000331310, ENSP00000340350, ENSP00000340823, ENSP00000351886, ENSP00000343344, ENSP00000344504, ENSP00000353350, ENSP00000354337, ENSP00000350911, ENSP00000351446, ENSP00000349156, ENSP00000353090, ENSP00000289032, ENSP00000347909, ENSP00000350071, ENSP00000351729, ENSP00000352331, ENSP00000296412, ENSP00000351492, ENSP00000348283, ENSP00000352069, ENSP00000347244, ENSP00000354561, ENSP00000353586, ENSP00000353578, and ENSP00000290009, or an ortholog thereof.

10. The method of claim 7, wherein said cell is a nematode cell.

11. The method of claim 7, wherein said cell is in a nematode.

12. A method for inhibiting RNAi in an organism, said method comprising contacting said organism with a nucleobase oligomer comprising a duplex of at least eight but no more than thirty consecutive nucleobases of an REG nucleic acid of claim 8 in an amount sufficient to inhibit RNAi.

13. The method of claim 12, wherein said organism is a pathogen selected from the group consisting of a bacteria, a virus, a fungus, an insect, and a nematode.

14. The method of claim 12, wherein said nucleobase oligomer is an siRNA or an shRNA.

15. A method for enhancing RNAi in an organism, said method comprising contacting said organism with a polypeptide of claim 9 in an amount sufficient to enable or enhance RNAi.

16. A method of enhancing RNAi in a subject, said method comprising co-administering to said subject an RNAi therapeutic and an REG cocktail comprising at least one REG polypeptide or at least one REG inhibitory nucleic acid, wherein said REG inhibitory nucleic acid down regulates an RNAi therapeutic.
